(19) United States
(12) Patent Application Publication
(10) Pub. No.: US 2014/0235533 A1
(43) Pub. Date: Aug. 21, 2014

(54) DIHYDROFURAN DERIVATIVES AS INSECTICIDAL COMPOUNDS

(71) Applicants: Tomas Smejkai, Stein (CH); Sebastian Volker Wendeborn, Stein (CH); Jerome Yves Cassayre, Stein (CH); Myriem El Qacemi, Stein (CH); Bernhard Breit, Freiburg (DE); Lisa Diab, Akkar el-atiqa (LB); Regis Jean Georges Mondiere, Stein (CH)

(72) Inventors: Tomas Smejkai, Stein (CH); Sebastian Volker Wendeborn, Stein (CH); Jerome Yves Cassayre, Stein (CH); Myriem El Qacemi, Stein (CH); Bernhard Breit, Freiburg (DE); Lisa Diab, Akkar el-atiqa (LB); Regis Jean Georges Mondiere, Stein (CH)

(73) Assignee: SYNGENTA PARTICIPATIONS AG, Basel (CH)

(21) Appl. No.: 14/239,271

(22) PCT Filed: Aug. 10, 2012
(86) PCT No.: PCT/EP2012/065765

§ 371 Date: Feb. 18, 2014

(30) Foreign Application Priority Data
Aug. 22, 2011 (EP) 11178221.5
Aug. 22, 2011 (EP) 11178224.9
Oct. 3, 2011 (EP) 11183687.0
Nov. 8, 2011 (EP) 11188276.7
Jul. 31, 2012 (EP) 12178614.9
Jul. 31, 2012 (EP) 12178615.6

(51) Int. Cl. A01N 43/08; A61K 31/341; A61K 45/06; A01N 43/38; A61K 31/4035; C07D 209/48; C07D 307/28; C07D 405/10

(52) U.S. Cl. CPC A01N 43/08; C07D 307/28; A61K 31/341; C07D 405/10; A01N 43/38; A61K 31/4035; C07D 209/48; A61K 45/06
USPC 514/4.5; 549/491; 514/471; 549/496; 548/465; 514/414; 548/513; 514/341; 514/229.2; 514/212.03; 514/30; 514/250; 514/202

(57) ABSTRACT

The present invention provides compounds of formula I wherein Q is Q1 or Q2; A¹, A², A³ and A⁴ are independently of each other C—H, C—R⁷, or nitrogen; R¹ is C₁-C₈haloalkyl; R² is aryl or aryl substituted by one to five R¹¹, or heteroaryl or heteroaryl substituted by one to five R¹¹; and R³, R⁴, R⁵, R⁶ and R⁷ are as defined in the claims. The invention also provides methods of controlling insects, acarines, nematodes or molluscs which methods comprise applying to a pest, to a locus of a pest, or to a plant susceptible to attack by a pest an insecticidally, acaricidally, nematicidally or molluscicidally effective amount of a compound of formula (I).

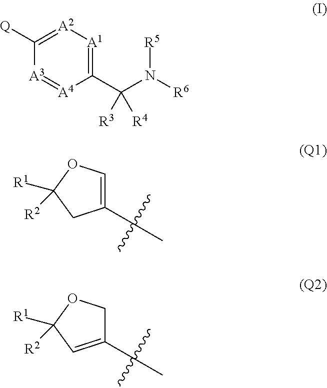

The present invention relates to certain dihydrofuran derivatives, to processes and intermediates for preparing these derivatives, to insecticidal, acaricidal, nematicidal and molluscicidal compositions comprising these derivatives and to methods of using these derivatives to control insect, acarine, nematode and mollusc pests.

Certain isoxazoline derivatives with insecticidal properties are disclosed, for example, in EP 1,731,512. However there is a continuing need to find new biologically active compounds as well as new biologically active compounds displaying superior properties for use as agrochemical active ingredients, for example greater biological activity, different spectrum of activity, increased safety profile, or increased biodegradability.

It has now surprisingly been found that certain dihydrofuran derivatives have insecticidal properties.

The present invention therefore provides compounds of formula (I)

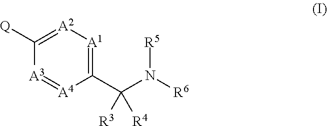

wherein

### Q is Q1 or Q2

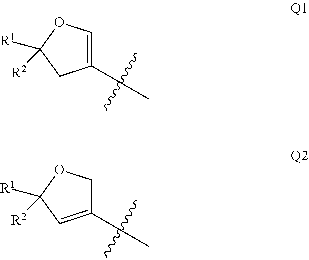

A¹, A², A³ and A⁴ are independently of each other C—H, C—R⁷, or nitrogen;
R¹ is C₁-C₈haloalkyl;
R² is aryl or aryl substituted by one to five R¹¹, or heteroaryl or heteroaryl substituted by one to five R¹¹;
R³ and R⁴ are each independently hydrogen, C₁-C₁₂alkyl or C₁-C₁₂alkyl substituted by one to five R⁸, C₃-C₈cycloalkyl or C₃-C₈cycloalkyl substituted by one to five R⁹, C₂-C₁₂alkenyl or C₂-C₁₂alkenyl substituted by one to five R⁸, C₂-C₁₂alkynyl or C₂-C₁₂alkynyl substituted by one to five R⁸, cyano, C₁-C₁₂alkoxycarbonyl or C₁-C₁₂alkoxycarbonyl substituted by one to five R⁸, C₁-C₁₂alkoxythiocarbonyl or C₁-C₁₂alkoxythiocarbonyl substituted by one to five R⁸, or R³ and R⁴ together with the carbon atom to which they are attached may form a 3 to 6-membered carbocyclic ring;
or when A¹ is C—R⁷, the R⁷ attached to A¹, R³ and fragment to which they are attached may together form a 5- to 7-membered carbocyclic ring, optionally substituted by one to five R¹⁶;
R⁵ is hydrogen, NH₂, hydroxyl, C₁-C₁₂ alkoxy or C₁-C₁₂alkoxy substituted by one to five R⁸, C₁-C₁₂alkylcarbonylamino or C₁-C₁₂alkylcarbonylamino wherein the alkyl is substituted by one to five R⁸, C₁-C₁₂alkylamino or C₁-C₁₂alkylamino wherein the alkyl is substituted by one to five R⁸, C₁-C₁₂alkyl or C₁-C₁₂alkyl substituted by one to five R⁸, C₃-C₈cycloalkyl or C₃-C₈cycloalkyl substituted by one to five R⁹, cyano, C₂-C₁₂alkenyl or C₂-C₁₂alkenyl substituted by one to five R⁸, C₂-C₁₂alkynyl or C₂-C₁₂alkynyl substituted by one to five R⁸, C₁-C₁₂alkylcarbonyl or C₁-C₁₂alkylcarbonyl substituted by one to five R⁸, C₁-C₁₂alkoxycarbonyl or C₁-C₁₂alkoxycarbonyl substituted by one to five R⁸ or is selected from CH₂—R¹³, C(═O)R¹³ and C(═S)R¹³;
R⁶ is hydrogen, cyano, carbonyl, thiocarbonyl, C₁-C₁₂alkylcarbonyl or C₁-C₁₂ alkylcarbonyl substituted by one to five R⁸, C₁-C₁₂alkylthiocarbonyl or C₁-C₁₂alkylthiocarbonyl substituted by one to five R⁸, C₁-C₁₂alkylaminocarbonyl or C₁-C₁₂alkylaminocarbonyl wherein the alkyl is substituted by one to five R⁸, C₁-C₁₂alkylaminothiocarbonyl or C₁-C₁₂alkylaminothiocarbonyl wherein the alkyl is substituted by one to five R⁸, C₂-C₂₄ (total carbon number) dialkylaminocarbonyl or C₂-C₂₄ (total carbon number) dialkylaminocarbonyl wherein one or both alkyl is substituted by one to five R⁸, C₂-C₂₄ (total carbon number) dialkylaminothiocarbonyl or C₂-C₂₄ (total carbon number) dialkylaminothiocarbonyl wherein one or both alkyl is substituted by one to five R⁸, C₁-C₁₂alkoxyaminocarbonyl or C₁-C₁₂alkoxyaminocarbonyl wherein the alkoxy is substituted by one to five R⁸, C₁-C₁₂alkoxyaminothiocarbonyl or C₁-C₁₂alkoxyaminothiocarbonyl wherein the alkoxy is substituted by one to five R⁸, C₁-C₁₂alkoxycarbonyl or C₁-C₁₂alkoxycarbonyl substituted by one to five R⁸, C₁-C₁₂alkoxythiocarbonyl or C₁-C₁₂alkoxythiocarbonyl substituted by one to five R⁸, C₁-C₁₂thioalkoxycarbonyl or C₁-C₁₂thioalkoxycarbonyl substituted by one to five R⁸, C₁-C₁₂thioalkoxythiocarbonyl or C₁-C₁₂thioalkoxythiocarbonyl substituted by one to five R⁸, C₁-C₁₂alkylsulfonyl or C₁-C₁₂alkylsulfonyl substituted by one to five R⁸, C₃-C₁₂cycloalkylcarbonyl or C₃-C₁₂cycloalkylcarbonyl substituted by one to five R⁹, C₂-C₁₂alkenylcarbonyl or C₂-C₁₂alkenylcarbonyl substituted by one to five R⁸, C₂-C₁₂alkynylcarbonyl or C₂-C₁₂alkynylcarbonyl substituted by one to five R⁸, C₃-C₁₂cycloalkyl-C₁-C₁₂alkylcarbonyl or C₃-C₁₂cycloalkyl-C₁-C₁₂alkylcarbonyl substituted by one to five R⁹, C₁-C₁₂alkylsulfenyl-C₁-C₁₂ alkylcarbonyl or C₁-C₁₂alkylsulfenyl-C₁-C₁₂alkylcarbonyl substituted by one to five R⁸, C₁-C₁₂alkylsulfinyl-C₁-C₁₂ alkylcarbonyl or C₁-C₁₂alkylsulfinyl-C₁-C₁₂ alkylcarbonyl substituted by one to five R⁸, C₁-C₁₂ alkylsulfonyl-C₁-C₁₂alkylcarbonyl or C₁-C₁₂alkylsulfonyl-C₁-C₁₂alkylcarbonyl substituted by one to five R⁸, C₁-C₁₂alkylcarbonyl-C₁-C₁₂alkylcarbonyl or C₁-C₁₂alkylcarbonyl-C₁-C₁₂alkylcarbonyl substituted by one to five R⁸, C₃-C₁₂cycloalkylaminocarbonyl or C₃-C₁₂cycloalkylaminocarbonyl wherein the cycloalkyl is substituted by one to five R⁹, C₂-C₁₂alkenylaminocarbonyl or C₂-C₁₂alkenylaminocarbonyl wherein the alkenyl is substituted by one to five R⁸, C₂-C₁₂alkynylaminocarbonyl or C₂-C₁₂alkynylaminocarbonyl wherein the alkynyl is substituted by one to five R⁸, or is selected from C(═O)R¹³ and C(═S)R¹³; or R⁵ and R⁶ together with the nitrogen atom to which they are bound, form a 3- to 6-membered heterocyclic ring which may be substituted by one to five R¹⁴, or may be substituted with a keto, thioketo or nitroimino group;
each R⁷ is independently halogen, cyano, nitro, C₁-C₈alkyl, C₃-C₈cycloalkyl, C₁-C₈haloalkyl, C₂-C₈alkenyl, C₂-C₈haloalkenyl, C₂-C₈alkynyl, C₂-C₈haloalkynyl, C₁-C₈alkoxy, C₁-C₈haloalkoxy, C₁-C₈alkoxycarbonyl-, or two R⁷ on adjacent carbon atoms together form a —CH═CH—CH═CH— bridge or a —N═CH—CH═CH— bridge; each R⁸ is independently halogen, cyano, nitro, hydroxy, NH₂, mercapto, C₁-C₈alkyl, C₁-C₈haloalkyl, C₁-C₈alkoxy, C₁-C₈haloalkoxy, C₁-C₈alkylthio, C₁-C₈haloalkylthio, C₁-C₈alkylsulfinyl, C₁-C₈haloalkylsulfinyl, C₁-C₈alkylsulfonyl, C₁-C₈haloalkylsulfonyl, C₁-C₈alkylamino, C₂-C₈dialkylamino, C₃-C₈cycloalkylamino, C₁-C₈alkylcarbonyl, C₁-C₈alkoxycarbonyl, C₁-C₈alkylaminocarbonyl, C₁-C₈dialkylaminocarbonyl, C₁-C₈haloalkylcarbonyl, C₁-C₈haloalkoxycarbonyl, C₁-C₈haloalkylaminocarbonyl, C₁-C₈halodialkylaminocarbonyl;
each R⁹ is independently halogen, cyano or C₁-C₈alkyl;
each R¹⁰ is independently halogen, cyano, nitro, C₁-C₈alkyl, C₁-C₈haloalkyl, C₂-C₈alkenyl, C₂-C₈haloalkenyl, C₂-C₈alkynyl, C₂-C₈haloalkynyl, hydroxy, C₁-C₈alkoxy, C₁-C₈haloalkoxy, mercapto, C₁-C₈alkylthio, C₁-C₈haloalkylthio, C₁-C₈alkylsulfinyl, C₁-C₈haloalkylsulfinyl, C₁-C₈alkylsulfonyl, C₁-C₈haloalkylsulfonyl, C₁-C₈alkylcarbonyl, C₁-C₈alkoxycarbonyl, aryl or aryl substituted by one to five R¹², or heterocyclyl or heterocyclyl substituted by one to five R¹²;
each R¹¹ is independently halogen, cyano, nitro, C₁-C₈alkyl, C₁-C₈haloalkyl, C₂-C₈alkenyl, C₂-C₈haloalkenyl, C₂-C₈alkynyl, C₂-C₈haloalkynyl, hydroxy, C₁-C₈alkoxy, C₁-C₈haloalkoxy, mercapto, C₁-C₈alkylthio, C₁-C₈haloalkylthio, C₁-C₈alkylsulfinyl, C₁-C₈haloalkylsulfinyl, C₁-C₈alkylsulfonyl, C₁-C₈haloalkylsulfonyl, C₁-C₈alkylcarbonyl, C₁-C₈alkoxycarbonyl, aryl or aryl substituted by one to five R¹², or heterocyclyl or heterocyclyl substituted by one to five R¹²;
each R¹² is independently halogen, cyano, nitro, C₁-C₄alkyl, C₁-C₄haloalkyl, C₁-C₄alkoxy-, or C₁-C₄haloalkoxy-;
R¹³ is aryl or aryl substituted by one to five R¹⁰, heterocyclyl or heterocyclyl substituted by one to five R¹⁰;
each R¹⁴ is independently halogen, cyano, nitro, C₁-C₈alkyl, C₁-C₈haloalkyl, C₁-C₈alkoxy, C₁-C₈haloalkoxy or C₁-C₈alkoxycarbonyl;
each R¹⁶ is independently hydrogen, halogen, cyano, nitro, C₁-C₈alkyl, C₁-C₈haloalkyl, C₂-C₈alkenyl, C₂-C₈haloalkenyl, C₂-C₈alkynyl, C₂-C₈haloalkynyl, hydroxy, C₁-C₈alkoxy, C₁-C₈haloalkoxy, mercapto, C₁-C₈alkylthio, C₁-C₈haloalkylthio, C₁-C₈alkylsulfinyl, C₁-C₈haloalkylsulfinyl, C₁-C₈alkylsulfonyl, C₁-C₈haloalkylsulfonyl, C₁-C₈alkylcarbonyl, C₁-C₈alkoxycarbonyl, aryl or aryl substituted by one to five R¹², or heterocyclyl or heterocyclyl substituted by one to five R¹²;
or a salt or N-oxide thereof

The compounds of formula (I) may exist in different geometric or optical isomers or tautomeric forms. This invention covers all such isomers and tautomers and mixtures thereof in all proportions as well as isotopic forms such as deuterated compounds. The invention also covers salts and N-oxides.

The compounds of the invention may contain one or more asymmetric carbon atoms, for example, at the —CR¹R²— group, and may exist as enantiomers (or as pairs of diastereoisomers) or as mixtures of such.

Alkyl groups (either alone or as part of a larger group, such as alkoxy-, alkylthio-, alkylsulfinyl-, alkylsulfonyl-, alkylcarbonyl- or alkoxycarbonyl-) can be in the form of a straight or branched chain and are, for example, methyl, ethyl, propyl, prop-2-yl, butyl, but-2-yl, 2-methyl-prop-1-yl or 2-methyl-prop-2-yl. The alkyl groups are preferably C₁-C₆, more preferably C₁-C₄, most preferably C₁-C₃ alkyl groups. Where an alkyl moiety is said to be substituted, the alkyl moiety is preferably substituted by one to four substituents, most preferably by one to three substituents.

Alkylene groups can be in the form of a straight or branched chain and are, for example, —CH₂—, —CH₂—CH₂—, —CH(CH₃)—, —CH₂—CH₂—CH₂—, —CH(CH₃)—CH₂—, or —CH(CH₂CH₃)—. The alkylene groups are preferably C₁-C₃, more preferably C₁-C₂, most preferably C₁ alkylene groups.

Alkenyl groups can be in the form of straight or branched chains, and can be, where appropriate, of either the (E)- or (Z)-configuration. Examples are vinyl and allyl. The alkenyl groups are preferably C₂-C₆, more preferably C₂-C₄, most preferably C₂-C₃ alkenyl groups.

Alkynyl groups can be in the form of straight or branched chains. Examples are ethynyl and propargyl. The alkynyl groups are preferably C₂-C₆, more preferably C₂-C₄, most preferably C₂-C₃ alkynyl groups.

Halogen is fluorine, chlorine, bromine or iodine.

Haloalkyl groups (either alone or as part of a larger group, such as haloalkoxy-, haloalkylthio-, haloalkylsulfinyl- or haloalkylsulfonyl-) are alkyl groups which are substituted by one or more of the same or different halogen atoms and are, for example, difluoromethyl, trifluoromethyl, chlorodifluoromethyl or 2,2,2-trifluoro-ethyl.

Haloalkenyl groups are alkenyl groups which are substituted by one or more of the same or different halogen atoms and are, for example, 2,2-difluoro-vinyl or 1,2-dichloro-2-fluoro-vinyl.

Haloalkynyl groups are alkynyl groups which are substituted by one or more of the same or different halogen atoms and are, for example, 1-chloro-prop-2-ynyl.

Cycloalkyl groups or carbocyclic rings can be in mono- or bi-cyclic form and are, for example, cyclopropyl, cyclobutyl, cyclohexyl and bicyclo[2.2.1]heptan-2-yl. The cycloalkyl groups are preferably C₃-C₈, more preferably C₃-C₆ cycloalkyl groups. Where a cycloalkyl moiety is said to be substituted, the cycloalkyl moiety is preferably substituted by one to four substituents, most preferably by one to three substituents.

Aryl groups (either alone or as part of a larger group, such as aryl-alkylene-) are aromatic ring systems which can be in mono-, bi- or tricyclic form. Examples of such rings include phenyl, naphthyl, anthracenyl, indenyl or phenanthrenyl. Preferred aryl groups are phenyl and naphthyl, phenyl being most preferred. Where an aryl moiety is said to be substituted, the aryl moiety is preferably substituted by one to four substituents, most preferably by one to three substituents.

Heteroaryl groups (either alone or as part of a larger group, such as heteroaryl-alkylene-) are aromatic ring systems containing at least one heteroatom and consisting either of a single ring or of two or more fused rings. Preferably, single rings will contain up to three heteroatoms and bicyclic systems up to four heteroatoms which will preferably be chosen from nitrogen, oxygen and sulfur. Examples of monocyclic groups include pyridyl, pyridazinyl, pyrimidinyl, pyrazinyl, pyrrolyl, pyrazolyl, imidazolyl, triazolyl (e.g. 1.2.4 triazoyl), furanyl, thiophenyl, oxazolyl, isoxazolyl, oxadiazolyl, thiazolyl, isothiazolyl and thiadiazolyl. Examples of bicyclic groups include purinyl, quinolinyl, cinnolinyl, quinoxalinyl, indolyl, indazolyl, benzimidazolyl, benzothiophenyl and benzothiazolyl. Monocyclic heteroaryl groups are preferred, pyridyl being most preferred. Where a heteroaryl moiety is said to be substituted, the heteroaryl moiety is preferably substituted by one to four substituents, most preferably by one to three substituents.

Heterocyclyl groups or heterocyclic rings (either alone or as part of a larger group, such as heterocyclyl-alkylene-) are defined to include heteroaryl groups and in addition their unsaturated or partially unsaturated analogues. Examples of monocyclic groups include isoxazolyl, thietanyl, pyrrolidinyl, tetrahydrofuranyl, [1,3]dioxolanyl, piperidinyl, piperazinyl, [1,4]dioxanyl, and morpholinyl or their oxidised versions such as 1-oxo-thietanyl and 1,1-dioxo-thietanyl. Examples of bicyclic groups include 2,3-dihydro-benzofuranyl, benzo[1,4]dioxolanyl, benzo[1,3]dioxolanyl, chromenyl, and 2,3-dihydro-benzo[1,4]dioxinyl. Where a heterocyclyl moiety is said to be substituted, the heterocyclyl moiety is preferably substituted by one to four substituents, most preferably by one to three substituents.

Preferred values of A¹, A², A³, A⁴, R¹, R², R³, R⁴, R⁵, R⁶, R⁷, R⁸, R⁹, R¹⁰, R¹¹, R¹², R¹³, R¹⁴ and R¹⁶ are, in any combination, as set out below.

Preferably A¹ is C—H or C—R⁷ and no more than two of A², A³ and A⁴ are nitrogen, more preferably no more than two of A², A³ and A⁴ are nitrogen and A³ and A⁴ are not both nitrogen. Even more preferably A¹ is C—H or C—R⁷, A² is C—H, C—R⁷ or nitrogen, A³ and A⁴ are independently C—H or nitrogen, wherein no more than two of A², A³ and A⁴ are nitrogen, and A³ and A⁴ are not both nitrogen, and wherein when A² is C—R⁷ then the R⁷ of A¹ and the R⁷ of A² together form a —CH═CH—CH═CH— bridge. Yet even more preferably A¹ is C—R⁷, A² is C—H, C—R⁷ or nitrogen, A³ and A⁴ are independently C—H or nitrogen, wherein no more than two of A², A³ and A⁴ are nitrogen, and A³ and A⁴ are not both nitrogen, and wherein when A² is C—R⁷ then the R⁷ of A¹ and the R⁷ of A² together form a —CH═CH—CH═CH— bridge. Yet even more preferably A¹ is C—R⁷, A² is C—H, and one of A³ and A⁴ is C—H and the other is nitrogen.

In one group of compounds A¹ is C—H or C—R⁷, most preferably A¹ is C—R⁷.

In one group of compounds A² is C—H or C—R⁷, most preferably A² is C—H.

In one group of compounds A³ is C—H or C—R⁷, most preferably A³ is C—H.

In one group of compounds A⁴ is C—H or C—R⁷, most preferably A⁴ is C—H.

Preferably R¹ is chlorodifluoromethyl, difluoromethyl or trifluoromethyl, more preferably chlorodifluoromethyl or trifluoromethyl, most preferably trifluoromethyl.

Preferably R² is aryl or aryl substituted by one to three R¹¹, more preferably R² is phenyl or phenyl substituted by one to three R¹¹, pyridyl or pyridyl substituted by one to three R¹¹, more preferably R² is phenyl substituted by one to three R¹¹ or pyridyl substituted by one to three R¹¹, more preferably R² is group P

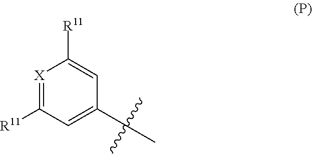

wherein X is N or C—R¹¹, preferably X is C—R¹¹.

More preferably R² is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 3-bromo-5-chlorophenyl, 4-fluoro-3,5-dichlorophenyl or 3,4,5-trichloro-phenyl, 3-chloro-4-fluorophenyl, 3-fluoro-4-chlorophenyl, 4-bromo-3,5-dichlorophenyl, 4-iodo-3,5-dichlorophenyl, 3,4,5-trifluorophenyl, 3-chloro-5-fluorophenyl, 3,4-dichloro-5-trifluoromethylphenyl or 4-chloro-3,5-bis-(trifluoromethyl)-phenyl, more preferably 3,5-bis-(trifluoromethyl)-phenyl, 3-chloro-5-trifluoromethyl-phenyl, 3,5-dichloro-phenyl, 3-trifluoromethyl-phenyl, 4-bromo-3,5-dichlorophenyl, 3-bromo-5-chlorophenyl, 4-fluoro-3,5-dichlorophenyl, 3,4,5-trichloro-phenyl, 4-iodo-3,5-dichlorophenyl, 3,4-dichloro-5-trifluoromethylphenyl, 4-chloro-3,5-bis-(trifluoromethyl)-phenyl, most preferably R² is 3,5-dichloro-phenyl.

Preferably, R³ and R⁴ are each independently hydrogen, C₁-C₁₂alkyl, C₁-C₁₂haloalkyl, C₃-C₈cycloalkyl, C₃-C₈halocycloalkyl, C₂-C₁₂alkenyl or C₂-C₁₂haloalkenyl, C₂-C₁₂alkynyl, C₂-C₁₂haloalkynyl cyano, C₁-C₁₂alkoxycarbonyl, C₁-C₁₂haloalkoxycarbonyl, C₁-C₁₂alkoxythiocarbonyl, C₁-C₁₂haloalkoxythiocarbonyl, or R³ and R⁴ together with the carbon atom to which they are attached may form a 3 to 6-membered carbocyclic ring. Preferably, R³ and R⁴ are each independently hydrogen, halogen, cyano, C₁-C₄alkyl, C₁-C₄haloalkyl, or C₃-C₆ cycloalkyl, or R³ and R⁴ together form a 3-6 membered carbocyclic ring, more preferably R³ and R⁴ are each independently hydrogen, halogen, cyano, C₁-C₄alkyl, C₁-C₄haloalkyl or C₃-C₆ cycloalkyl. More preferably at least one of R³ and R⁴ is hydrogen and the other is hydrogen, halogen, cyano, C₁-C₄alkyl, C₁-C₄haloalkyl or C₃-C₆ cycloalkyl, more preferably at least one of R³ and R⁴ is hydrogen and the other is hydrogen, methyl, ethyl or cyclopropyl.

When A¹ is C—R⁷, the R⁷ attached to A¹, R³ and fragment to which they are attached may together for a 5- to 7-membered carbocyclic ring optionally substituted by one to five R¹⁶. For example, the R⁷ attached to A¹ and R³ may together represent the fragment —C(R¹⁶)(R¹⁶)—C(R¹⁶)(R¹⁶)—, —C(R¹⁶)═C(R¹⁶)—, —C(R¹⁶)(R¹⁶)—C(R¹⁶)(R¹⁶)—C(R¹⁶)(R¹⁶)—, —C(R¹⁶)═C(R¹⁶)—C(R¹⁶)(R¹⁶)— or —C(R¹⁶)(R¹⁶)—C(R¹⁶)═C(R¹⁶)—; more preferably —C(R¹⁶)(R¹⁶)—C(R¹⁶)(R¹⁶)— or —C(R¹⁶)(R¹⁶)—C(R¹⁶)(R¹⁶)—C(R¹⁶)(R¹⁶)—. When R¹⁶ is hydrogen these ring fragments are —CH₂—CH₂—, —CH═CH—, —CH₂—CH₂—CH₂—, —CH═CH—CH₂— or —CH₂—CH═CH—, preferably —CH₂—CH₂— or —CH₂—CH₂—CH₂—.

Preferably, R⁵ is hydrogen, NH₂, hydroxyl, C₁-C₁₂alkoxy, C₁-C₁₂haloalkoxy, C₁-C₁₂alkylcarbonylamino, C₁-C₁₂haloalkylcarbonylamino, C₁-C₁₂alkylamino, C₁-C₁₂haloalkylamino, C₁-C₁₂alkyl, C₁-C₁₂haloalkyl, C₃-C₈cycloalkyl, C₃-C₈halocycloalkyl, cyano, C₁-C₁₂alkenyl, C₁-C₁₂haloalkenyl, C₂-C₁₂alkynyl, C₂-C₁₂haloalkynyl, C₁-C₁₂alkylcarbonyl, C₁-C₁₂haloalkylcarbonyl, C₁-C₈alkoxycarbonyl, or C₁-C₈haloalkoxycarbonyl. More preferably, R⁵ is hydrogen, C₁-C₈alkyl, C₁-C₈haloalkyl, C₁-C₈alkoxy, C₁-C₈haloalkoxy, C₁-C₈alkylcarbonyl, C₁-C₈haloalkylcarbonyl, C₁-C₈alkoxycarbonyl, or C₁-C₈haloalkoxycarbonyl. Even more preferably R⁵ is hydrogen, C₁-C₄alkyl or C₁-C₄haloalkyl, most preferably hydrogen.

Preferably R⁶ is hydrogen, cyano, carbonyl, thiocarbonyl, C₁-C₁₂alkylcarbonyl, C₁-C₁₂haloalkylcarbonyl, C₁-C₁₂alkylthiocarbonyl, C₁-C₁₂haloalkylthiocarbonyl, C₁-C₁₂alkylaminocarbonyl, C₁-C₁₂alkylaminothiocarbonyl, C₂-C₂₄ (total carbon number) dialkylaminocarbonyl, C₂-C₂₄ (total carbon number) dialkylaminothiocarbonyl, C₁-C₁₂alkoxyaminocarbonyl, C₁-C₁₂alkoxyaminothiocarbonyl, C₁-C₁₂alkoxycarbonyl, C₁-C₁₂haloalkoxycarbonyl, C₁-C₁₂alkoxythiocarbonyl, C₁-C₁₂haloalkoxythiocarbonyl, C₁-C₁₂thioalkoxycarbonyl, C₁-C₁₂thioalkoxythiocarbonyl, C₁-C₁₂alkoxy-C₁-C₄alkylcarbonyl, C₁-C₁₂haloalkoxy-C₁-C₄alkylcarbonyl, C₁-C₁₂alkylsulfonyl, C₁-C₁₂haloalkylsulfonyl, C₃-C₁₂cycloalkylcarbonyl, C₃-C₁₂halocycloalkylcarbonyl, C₂-C₁₂alkenylcarbonyl, C₂-C₁₂haloalkenylcarbonyl, C₂-C₁₂ alkynylcarbonyl, C₂-C₁₂haloalkynylcarbonyl, C₃-C₁₂cycloalkyl-C₁-C₁₂alkylcarbonyl, C₃-C₁₂halocycloalkyl-C₁-C₁₂alkylcarbonyl, C₂-C₁₂alkylsulfenyl-C₁-C₁₂alkylcarbonyl, C₁-C₁₂haloalkylsulfenyl-C₁-C₁₂alkylcarbonyl, C₁-C₁₂alkylsulfinyl-C₁-C₁₂alkylcarbonyl, C₁-C₁₂haloalkylsulfinyl-C₁-C₁₂alkylcarbonyl, C₁-C₁₂alkylsulfonyl-C₁-C₁₂alkylcarbonyl, C₁-C₁₂haloalkylsulfonyl-C₁-C₁₂alkylcarbonyl, C₁-C₁₂alkylcarbonyl-C₁-C₁₂alkylcarbonyl, C₁-C₁₂haloalkylcarbonyl-C₁-C₁₂alkylcarbonyl, C₃-C₁₂cycloalkylaminocarbonyl, C₂-C₁₂alkenylaminocarbonyl, C₂-C₁₂alkynylaminocarbonyl or C(═O)R¹³.

More preferably R⁶ is C₁-C₁₂alkylcarbonyl, C₁-C₁₂haloalkylcarbonyl, C₁-C₁₂alkylthiocarbonyl, C₁-C₁₂haloalkylthiocarbonyl, C₁-C₁₂alkylaminocarbonyl, C₁-C₁₂alkylaminothiocarbonyl, C₂-C₂₄ (total carbon number) dialkylaminocarbonyl, C₂-C₂₄ (total carbon number) dialkylaminothiocarbonyl, C₁-C₁₂alkoxyaminocarbonyl, C₁-C₁₂alkoxyaminothiocarbonyl, C₁-C₁₂alkoxycarbonyl, C₁-C₁₂haloalkoxycarbonyl, C₁-C₁₂alkoxythiocarbonyl, C₁-C₁₂haloalkoxythiocarbonyl, C₁-C₁₂thioalkoxycarbonyl, C₁-C₁₂thioalkoxythiocarbonyl, C₁-C₁₂alkoxy-C₁-C₄alkylcarbonyl, C₁-C₁₂haloalkoxy-C₁-C₄alkylcarbonyl, C₁-C₁₂alkylsulfonyl, C₁-C₁₂haloalkylsulfonyl, C₃-C₁₂cycloalkylcarbonyl, C₃-C₁₂halocycloalkylcarbonyl, C₂-C₁₂alkenylcarbonyl, C₂-C₁₂haloalkenylcarbonyl, C₂-C₁₂ alkynylcarbonyl, C₂-C₁₂haloalkynylcarbonyl, C₃-C₁₂cycloalkyl-C₁-C₁₂alkylcarbonyl, C₃-C₁₂halocycloalkyl-C₁-C₁₂alkylcarbonyl, C₂-C₁₂alkylsulfenyl-C₁-C₁₂alkylcarbonyl, C₁-C₁₂haloalkylsulfenyl-C₁-C₁₂alkylcarbonyl, C₁-C₁₂alkylsulfinyl-C₁-C₁₂alkylcarbonyl, C₁-C₁₂haloalkylsulfinyl-C₁-C₁₂alkylcarbonyl, C₁-C₁₂alkylsulfonyl-C₁-C₁₂alkylcarbonyl, C₁-C₁₂haloalkylsulfonyl-C₁-C₁₂alkylcarbonyl, C₁-C₁₂alkylcarbonyl-C₁-C₁₂alkylcarbonyl, C₁-C₁₂haloalkylcarbonyl-C₁-C₁₂alkylcarbonyl, C₃-C₁₂cycloalkylaminocarbonyl, C₂-C₁₂alkenylaminocarbonyl, C₂-C₁₂alkynylaminocarbonyl or C(═O)R¹³ wherein R¹³ is phenyl or phenyl substituted by one to five R¹⁴, or pyridyl or pyridyl substituted by one to four R¹⁴.

More preferably R⁶ is C₁-C₁₂alkylcarbonyl, C₁-C₁₂haloalkylcarbonyl, C₃-C₁₂cycloalkylcarbonyl, C₃-C₁₂halocycloalkylcarbonyl, C₃-C₁₂cycloalkyl-C₁-C₁₂alkylcarbonyl, C₃-C₁₂halocycloalkyl-C₁-C₁₂alkylcarbonyl, C₁-C₁₂alkoxy-C₁-C₄alkylcarbonyl, C₁-C₁₂haloalkoxy-C₁-C₄alkylcarbonyl, C₁-C₁₂alkylsulfenyl-C₁-C₁₂alkylcarbonyl, C₁-C₁₂haloalkylsulfenyl-C₁-C₁₂alkylcarbonyl, C₁-C₁₂alkylsulfinyl-C₁-C₁₂alkylcarbonyl, C₁-C₁₂haloalkylsulfinyl-C₁-C₁₂alkylcarbonyl, C₁-C₁₂alkylsulfonyl-C₁-C₁₂alkylcarbonyl, C₁-C₁₂haloalkylsulfonyl-C₁-C₁₂alkylcarbonyl, C₁-C₁₂alkylaminocarbonyl, C₃-C₁₂cycloalkylaminocarbonyl, or C(═O)R¹³ wherein R¹³ is phenyl or phenyl substituted by one to five R¹⁴, or pyridyl or pyridyl substituted by one to four R¹⁴.

More preferably R⁶ is C₁-C₈alkylcarbonyl, C₁-C₈haloalkylcarbonyl, C₃-C₈cycloalkylcarbonyl, C₃-C₈halocycloalkylcarbonyl, C₃-C₈cycloalkyl-CH₂-carbonyl, C₃-C₈halocycloalkyl-CH₂-carbonyl, C₁-C₁₂alkoxy-CH₂-carbonyl, C₁-C₁₂haloalkoxy-CH₂-carbonyl, C₁-C₈alkylsulfenyl-CH₂-carbonyl, C₁-C₈haloalkylsulfenyl-CH₂-carbonyl, C₁-C₈alkylsulfinyl-CH₂-alkylcarbonyl, C₁-C₈haloalkylsulfinyl-CH₂-carbonyl, C₁-C₈alkylsulfonyl-CH₂-alkylcarbonyl, or C₁-C₈haloalkylsulfonyl-CH₂-carbonyl, C₁-C₈alkylaminocarbonyl, C₃-C₈cycloalkylaminocarbonyl, or C(═O)R¹³, wherein R¹³ is phenyl or phenyl substituted by one to five R¹⁴, or pyridyl or pyridyl substituted by one to four R¹⁴, or tetrahydrofuranyl or tetrahydrofuranyl substituted by one to five R¹⁴.

In one group of compounds R⁶ is hydrogen, cyano, carbonyl, thiocarbonyl, C₁-C₁₂alkylcarbonyl, C₁-C₁₂haloalkylcarbonyl, C₁-C₁₂alkylthiocarbonyl, C₁-C₁₂haloalkylthiocarbonyl, C₁-C₁₂alkylaminocarbonyl, C₁-C₁₂alkylaminothiocarbonyl, C₂-C₂₄ (total carbon number) dialkylaminocarbonyl, C₂-C₂₄ (total carbon number) dialkylaminothiocarbonyl, C₁-C₁₂alkoxyaminocarbonyl, C₁-C₁₂alkoxyaminothiocarbonyl, C₁-C₁₂alkoxycarbonyl, C₁-C₁₂haloalkoxycarbonyl, C₁-C₁₂alkoxythiocarbonyl, C₁-C₁₂haloalkoxythiocarbonyl, C₁-C₁₂thioalkoxycarbonyl, C₁-C₁₂thioalkoxythiocarbonyl, C₁-C₁₂alkylsulfonyl, C₁-C₁₂haloalkylsulfonyl, C₃-C₁₂cycloalkylcarbonyl, C₃-C₁₂halocycloalkylcarbonyl, C₂-C₁₂alkenylcarbonyl, C₂-C₁₂haloalkenylcarbonyl, C₂-C₁₂ alkynylcarbonyl, C₂-C₁₂haloalkynylcarbonyl, C₃-C₁₂cycloalkyl-CH₂-carbonyl, C₃-C₁₂halocycloalkyl-CH₂-carbonyl, C₁-C₁₂alkoxy-CH₂-carbonyl, C₂-C₁₂alkylsulfenyl-CH₂-carbonyl, C₁-C₁₂haloalkylsulfenyl-CH₂-carbonyl, C₁-C₁₂alkylsulfinyl-CH₂-carbonyl, C₁-C₁₂haloalkylsulfinyl-CH₂-carbonyl, C₁-C₁₂alkylsulfonyl-CH₂-carbonyl, C₁-C₁₂haloalkylsulfonyl-CH₂-carbonyl, C₁-C₁₂alkylcarbonyl-CH₂-carbonyl, C₁-C₁₂haloalkylcarbonyl-CH₂-carbonyl, C₃-C₁₂cycloalkylaminocarbonyl, C₂-C₁₂alkenylaminocarbonyl, C₂-C₁₂alkynylaminocarbonyl.

In one group of compounds R⁵ and R⁶ together with the nitrogen atom to which they are bound form a ring, preferably it is a 3- to 6-membered heterocyclic ring which may be substituted by one to five R¹⁴, or may be substituted with a keto, thioketo or nitroimino group.

In one group of compounds R⁶ is C(═O)—R¹⁵, wherein R¹⁵ is C₁-C₄alkyl, C₁-C₄haloalkyl, C₃.C₆ cycloalkyl, C₃.C₆cycloalkyl-C₁-C₄alkyl, C₃.C₆halocycloalkyl, C₁-C₄alkoxy, C₁-C₄alkoxy-C₁-C₄alkyl, C₁-C₄haloalkoxy-C₁-C₄alkyl, C₁-C₄ alkylthio-C₁-C₄alkyl, C₁-C₄alkylsulfinyl-C₁-C₄alkyl, C₁-C₄alkylsulfonyl-C₁-C₄alkyl, C₁-C₄haloalkylthio-C₁-C₄alkyl, C₁-C₄haloalkylsulfinyl-C₁-C₄alkyl, C₁-C₄haloalkylsulfonyl-C₁-C₄alkyl, C₁-C₄alkylamino, C₃-C₈cycloalkylamino, halogen substituted phenyl or pyridylmethyl; preferably R¹⁵ is methyl, ethyl, cyclopropyl, cyclopropylmethyl, 2,2,2-trifluoroethyl, 2-methoxyethyl, methylthiomethyl, methylsulfinylmethyl, methylsulfonylmethyl, methylamino, ethylamino, 2,2,2-trifluoroethylamino, cyclopropylamino, cyclopropylmethylamino, 2,4,6-trifluorophenyl or pyridylmethyl; more preferably R¹⁵ is methyl, ethyl, 2,2,2-trifluoroethyl or cyclopropyl.

Preferably each R⁷ is independently halogen, cyano, nitro, C₁-C₈alkyl, C₃-C₈cycloalkyl, C₁-C₈haloalkyl, C₂-C₈alkenyl, C₁-C₈alkoxy or C₁-C₈haloalkoxy, or two R⁷ on adjacent carbon atoms together form a —CH═CH—CH═CH— bridge, more preferably halogen, cyano, nitro, C₁-C₈alkyl, C₂-C₈ alkenyl, C₃-C₈cycloalkyl, C₁-C₈haloalkyl, C₁-C₈alkoxy or C₁-C₈haloalkoxy, even more preferably bromo, chloro, fluoro, cyano, nitro, methyl, ethyl, trifluoromethyl, cyclopropyl, vinyl, methoxy, trifluoromethoxy, yet even more preferably bromo, chloro, fluoro, cyclopropyl, trifluoromethyl, vinyl, or methyl, ethyl, nitro, cyano, most preferably bromo, chloro, fluoro, or methyl.

Preferably, each R⁸ is independently halogen, cyano, nitro, hydroxy, C₁-C₈alkoxy, C₁-C₈haloalkoxy, C₁-C₈alkylcarbonyl, C₁-C₈alkoxycarbonyl, mercapto, C₁-C₈alkylthio, C₁-C₈haloalkylthio, C₁-C₈alkylsulfinyl, C₁-C₈haloalkylsulfinyl, C₁-C₈alkylsulfonyl. More preferably, each R⁸ is independently halogen, cyano, nitro, hydroxy, C₁-C₈alkoxy, C₁-C₈haloalkoxy, mercapto, C₁-C₈alkylthio, C₁-C₈haloalkylthio, more preferably bromo, chloro, fluoro, methoxy, or methylthio, most preferably chloro, fluoro, or methoxy.

Preferably, each R⁹ is independently cyano, chloro, fluoro or methyl, most preferably each R⁹ is methyl.

Preferably each R¹⁰ is independently halogen, cyano, nitro, C₁-C₈alkyl, C₁-C₈haloalkyl, C₁-C₈alkoxy, C₁-C₈haloalkoxy, more preferably bromo, chloro, fluoro, cyano, nitro, methyl, ethyl, trifluoromethyl, methoxy, difluoromethoxy, or trifluoromethoxy, most preferably bromo, chloro, fluoro, cyano or methyl.

Preferably each R¹¹ is independently halogen, cyano, nitro, C₁-C₈alkyl, C₁-C₈haloalkyl, C₁-C₈alkoxy, C₁-C₈haloalkoxy, more preferably iodo, bromo, chloro, fluoro, cyano, nitro, methyl, ethyl, trifluoromethyl, methoxy, difluoromethoxy, or trifluoromethoxy, most preferably bromo, chloro, fluoro, iodo or trifluoromethyl.

Preferably each R¹² is independently bromo, chloro, fluoro, cyano, nitro, methyl, ethyl, trifluoromethyl, methoxy, difluoromethoxy or trifluoromethoxy, more preferably bromo, chloro, fluoro, nitro or methyl, most preferably each R¹¹ is independently chloro, fluoro or methyl.

Preferably R¹³ is phenyl or phenyl substituted by one to five R¹⁴, or pyridyl or pyridyl substituted by one to five R¹⁴.

Preferably each R¹⁴ is independently bromo, chloro, fluoro, cyano, nitro, methyl, ethyl, trifluoromethyl, methoxy, difluoromethoxy or trifluoromethoxy, more preferably bromo, chloro, fluoro, nitro or methyl, more preferably each R¹⁴ is independently chloro, fluoro or methyl.

Preferably each R¹⁶ is independently hydrogen, halogen, cyano, nitro, C₁-C₈alkyl, C₁-C₈haloalkyl, C₁-C₈alkoxy, C₁-C₈haloalkoxy, more preferably hydrogen, bromo, chloro, fluoro, cyano, nitro, methyl, ethyl, trifluoromethyl, methoxy, difluoromethoxy, or trifluoromethoxy, most preferably hydrogen, bromo, chloro, fluoro, cyano or methyl. Most preferably R¹⁶ is hydrogen (such that the compounds are the same as those in which the carbocyclic ring formed by R⁷ and R³ and the fragment to which they are attached is not substituted by R¹⁶).

Optionally any embodiment of the invention may not include compounds in which, when A¹ is C—R⁷, R⁷ and R³ and the fragment to which they are attached form a 5- to 7-membered heterocyclic ring.

In one embodiment the present invention provides compounds of formula I in which Q is Q1. In one embodiment the present invention provides compounds of formula I in which Q is Q2.

In one embodiment the present invention provides compounds of formula (Ia)

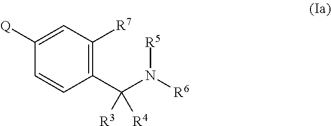

wherein Q, R³, R⁴, R⁵, R⁶ and R⁷ are as defined for compounds of formula (I); or a salt or N-oxide thereof. The preferences for Q, R³, R⁴, R⁵, R⁶ and R⁷ are the same as the preferences set out for the corresponding substituents of compounds of the formula (I).

In one embodiment the present invention provides compounds of formula (Ib)

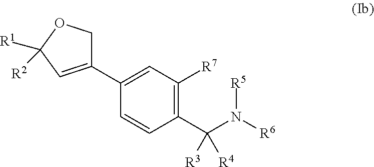

wherein R¹, R², R³, R⁴, R⁵, R⁶ and R⁷ are as defined for compounds of formula (I); or a salt or N-oxide thereof. The preferences for R¹, R², R³, R⁴, R⁵, R⁶ and R⁷ are the same as the preferences set out for the corresponding substituents of compounds of the formula (I).

In a further embodiment the present invention provides compounds of formula (Ic)

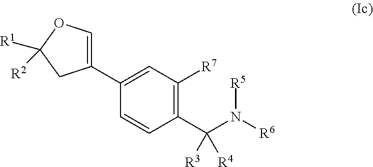

wherein R¹, R², R³, R⁴, R⁵, R⁶ and R⁷ are as defined for compounds of formula (I); or a salt or N-oxide thereof. The preferences for R¹, R², R³, R⁴, R⁵, R⁶ and R⁷ are the same as the preferences set out for the corresponding substituents of compounds of the formula (I).

In a further embodiment the present invention provides compounds of formula (Id)

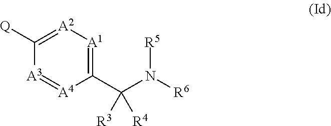

wherein

### Q is Q1 or Q2

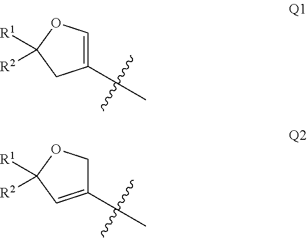

R¹ is chlorodifluoromethyl, difluoromethyl or trifluoromethyl;
R² is group P

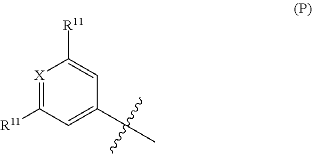

A¹ is C—R⁷, A² is C—H, C—R⁷ or nitrogen, A³ and A⁴ are independently C—H or nitrogen, wherein no more than two of A², A³ and A⁴ are nitrogen, and A³ and A⁴ are not both nitrogen, and wherein when A² is C—R⁷ then the R⁷ of A¹ and the R⁷ of A² together form a —CH═CH—CH═CH— bridge, X is C or N, and R³, R⁴, R⁵, R⁶, R⁷ and R¹¹ are as defined for compounds of formula I; or a salt or N-oxide thereof. The preferences for A¹, A², A³, A⁴, R¹, R², R³, R⁴, R⁵, R⁶, R⁷ and R¹¹ are the same as the preferences set out for the corresponding substituents of compounds of the formula (I). When the R⁷ attached to A¹, R³ and fragment to which they are attached together form a carbocyclic ring, preferably R⁷ and R³ together represent the fragment —C(R¹⁶)(R¹⁶)—C(R¹⁶)(R¹⁶)— or —C(R¹⁶)(R¹⁶)—C(R¹⁶)(R¹⁶)—C(R¹⁶)(R¹⁶)—, more preferably —CH₂—CH₂— or —CH₂—CH₂—CH₂—.

In a further embodiment the present invention provides compounds of formula (Ie)

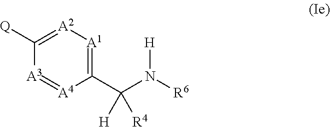

### Q is Q1 or Q2

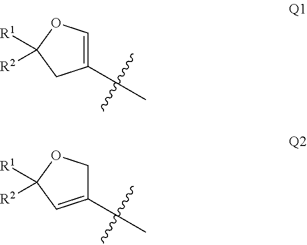

R¹ is chlorodifluoromethyl, difluoromethyl or trifluoromethyl;
R² is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 3-bromo-5-chlorophenyl, 4-fluoro-3,5-dichlorophenyl or 3,4,5-trichloro-phenyl, 3-chloro-4-fluorophenyl, 3-fluoro-4-chlorophenyl, 4-bromo-3,5-dichlorophenyl, 4-iodo-3,5-dichlorophenyl, 3,4,5-trifluorophenyl, 3-chloro-5-fluorophenyl, 3,4-dichloro-5-trifluoromethylphenyl or 4-chloro-3,5-bis-(trifluoromethyl)-phenyl, more preferably 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 3-bromo-5-chlorophenyl, 4-fluoro-3,5-dichlorophenyl or 3,4,5-trichloro-phenyl;
A¹ is C—R⁷, A² is C—H, C—R⁷ or nitrogen, A³ and A⁴ are independently C—H or nitrogen, wherein no more than two of A², A³ and A⁴ are nitrogen, and A³ and A⁴ are not both nitrogen, and wherein when A² is C—R⁷ then the R⁷ of A¹ and the R⁷ of A² together form a —CH═CH—CH═CH— bridge;
R⁴ and R⁶ are as defined for the compound of formula I;
R⁷ is halogen, cyano, nitro, C₁-C₈alkyl, C₂-C₈ alkenyl, C₃-C₈cycloalkyl, C₁-C₈haloalkyl, C₁-C₈alkoxy or C₁-C₈haloalkoxy; or a salt or N-oxide thereof. The preferences for A¹, A², A³, A⁴, R¹, R², R⁴, R⁶ and R⁷ are the same as the preferences set out for the corresponding substituents of compounds of the formula (I).

In a further embodiment the present invention provides compounds of formula (If)

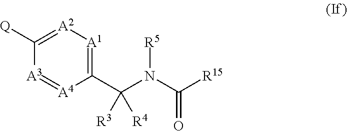

wherein Q, A¹, A², A³, A⁴, R³, R⁴, R⁵ and preferences thereof are as defined for the compound of formula I and R¹⁵ is as defined for compounds of formula I. When the R⁷ attached to A¹, R³ and fragment to which they are attached together form a carbocyclic ring, preferably R⁷ and R³ together represent the fragment —C(R¹⁶)(R¹⁶)—C(R¹⁶)(R¹⁶)— or —C(R¹⁶)(R¹⁶)—C(R¹⁶)(R¹⁶)—C(R¹⁶)(R¹⁶)—, more preferably —CH₂—CH₂— or —CH₂—CH₂—CH₂—.

In a further embodiment the present invention provides compounds of formula (Ig)

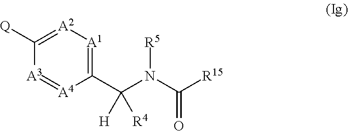

wherein Q, A¹, A², A³, A⁴, R³, R⁴, R⁵, R¹⁵ and preferences thereof are as defined for the compound of formula I. When the R⁷ attached to A¹, R³ and fragment to which they are attached together form a carbocyclic ring, preferably R⁷ and R³ together represent the fragment —C(R¹⁶)(R¹⁶)—C(R¹⁶)(R¹⁶)— or —C(R¹⁶)(R¹⁶)—C(R¹⁶)(R¹⁶)—C(R¹⁶)(R¹⁶)—, more preferably —CH₂—CH₂— or —CH₂—CH₂—CH₂—.

In a further embodiment the present invention provides compounds of formula (Ih)

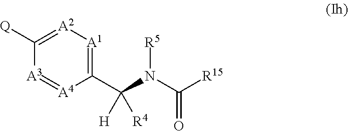

wherein Q, A¹, A², A³, A⁴, R³, R⁴, R⁵, R¹⁵ and preferences thereof are as defined for the compound of formula I, with the proviso that R⁴ is not hydrogen. Preferably R⁴ is methyl, ethyl or cyclopropyl, R⁵ is hydrogen, R¹⁵ is methyl, ethyl, cyclopropyl, cyclopropylmethyl, 2,2,2-trifluoroethyl, 2-methoxyethyl, methylthiomethyl, methylsulfinylmethyl, methylsulfonylmethyl, methylamino, ethylamino, 2,2,2-trifluoroethylamino, cyclopropylamino, cyclopropylmethylamino, 2,4,6-trifluorophenyl or pyridylmethyl. When the R⁷ attached to A¹, R³ and fragment to which they are attached together form a carbocyclic ring, preferably R⁷ and R³ together represent the fragment —C(R¹⁶)(R¹⁶)—C(R¹⁶)(R¹⁶)— or —C(R¹⁶)(R¹⁶)—C(R¹⁶)(R¹⁶)—C(R¹⁶)(R¹⁶)—, more preferably —CH₂—CH₂— or —CH₂—CH₂—CH₂—.

In a further embodiment the invention provides compounds of formula (Ij)

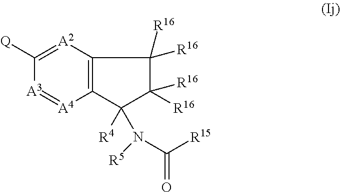

wherein Q, A², A³, A⁴, R⁴, R⁵, R¹⁵, R¹⁶ and preferences thereof are as defined for compounds of formula I.

In a further embodiment the invention provides compounds of formula (Ik)

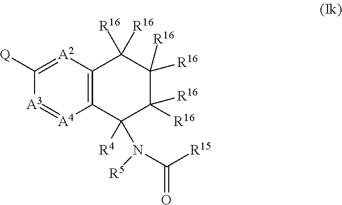

wherein Q, A², A³, A⁴, R⁴, R⁵, R¹⁵, R¹⁶ and preferences thereof are as defined for compounds of formula I.

Certain intermediates useful in the preparation of compounds of formula I are novel and form further aspects of the invention.

The invention provides compounds of formula Int-1

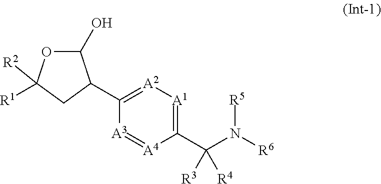

wherein A¹, A², A³, A⁴, R¹, R², R³, R⁴, R⁵ and R⁶ are as defined for compounds of formula I, or a salt of N-oxide thereof. The preferences for A¹, A², A³, A⁴, R¹, R², R³, R⁴, R⁵ and R⁶ are as defined for compounds of formula I.

The compound of formula Int-1 includes compounds of formula Int-1A within its scope

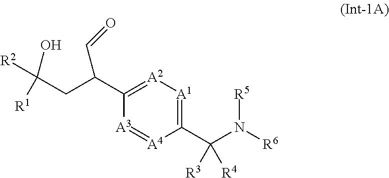

wherein A¹, A², A³, A⁴, R¹, R², R³, R⁴, R⁵ and R⁶ are as defined for compounds of formula I, or a salt of N-oxide thereof. Compounds of formula Int-1 and Int-1A usually exist in equilibrium in solution.

The invention also provides compounds of formula Int-2

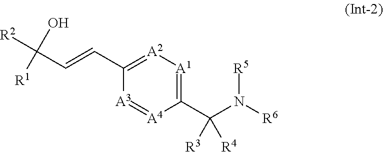

wherein A¹, A², A³, A⁴, R¹, R², R³, R⁴, R⁵ and R⁶ are as defined for compounds of formula I, or a salt of N-oxide thereof. The preferences for A¹, A², A³, A⁴, R¹, R², R³, R⁴, R⁵ and R⁶ are as defined for compounds of formula I.

The invention also provides compounds of formula Int-3

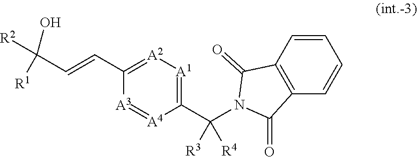

wherein A¹, A², A³, A⁴, R¹, R², R³ and R⁴ are as defined for compounds of formula I, or a salt of N-oxide thereof. The preferences for A¹, A², A³, A⁴, R¹, R², R³ and R⁴ are as defined for compounds of formula I.

The invention also provides compounds of formula Int-4

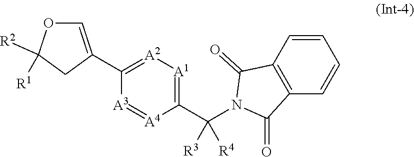

wherein A¹, A², A³, A⁴, R¹, R², R³ and R⁴ are as defined for compounds of formula I, or a salt of N-oxide thereof. The preferences for A¹, A², A³, A⁴, R¹, R², R³ and R⁴ are as defined for compounds of formula I.

The invention also provides compounds of formula Int-5

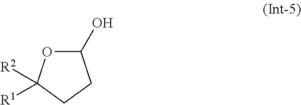

wherein R¹ and R² are as defined for compounds of formula I. The preferences for, R¹ and R² are as defined for compounds of formula I.

The compounds of formula Int-5 includes compounds of formula Int-5a which can exist in equilibrium with compounds of formula Int-5

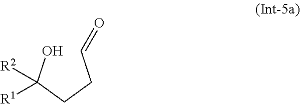

wherein R¹ and R² are as defined for compounds of formula I.

The invention also provides compounds of formula Int-6

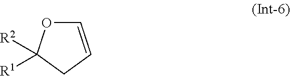

wherein R¹ and R² are as defined for compounds of formula I. The preferences for, R¹ and R² are as defined for compounds of formula I. The invention also provides compounds of formula Int-7

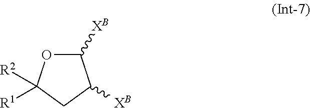

wherein R¹ and R² are as defined for compounds of formula I and each X^{B} independently represents Cl, Br, or I. The preferences for, R¹ and R² are as defined for compounds of formula I.

The invention also provides compounds of formula Int-8

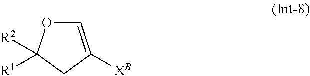

wherein R¹ and R² are as defined for compounds of formula I and X^{B} represents Cl, Br or I. The preferences for R¹ and R² are as defined for compounds of formula I.

The invention also provides compounds of formula Int-9

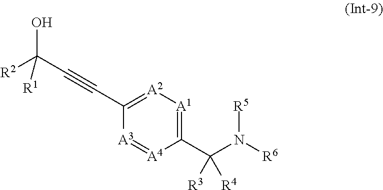

wherein A¹, A², A³, A⁴, R¹, R², R³, R⁴, R⁵ and R⁶ are as defined for compounds of formula I, or a salt of N-oxide thereof. The preferences for A¹, A², A³, A⁴, R¹, R², R³, R⁴, R⁵ and R⁶ are as defined for compounds of formula I.

The invention also provides compounds of formula Int-10

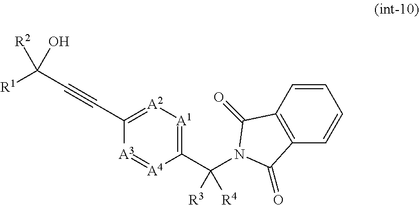

wherein A¹, A², A³, A⁴, R¹, R², R³ and R⁴ are as defined for compounds of formula I, or a salt of N-oxide thereof. The preferences for A¹, A², A³, A⁴, R¹, R², R³ and R⁴ are as defined for compounds of formula I.

The invention also provides compounds of formula Int-11

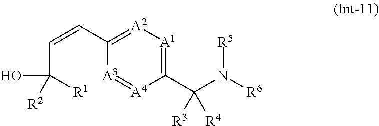

wherein A¹, A², A³, A⁴, R¹, R², R³, R⁴, R⁵ and R⁶ are as defined for compounds of formula I, or a salt of N-oxide thereof. The preferences for A¹, A², A³, A⁴, R¹, R², R³, R⁴, R⁵ and R⁶ are as defined for compounds of formula I.

The invention also provides compounds of formula Int-12

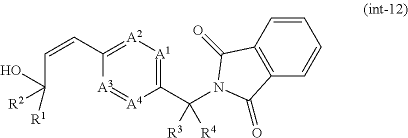

wherein A¹, A², A³, A⁴, R¹, R², R³ and R⁴ are as defined for compounds of formula I, or a salt of N-oxide thereof. The preferences for A¹, A², A³, A⁴, R¹, R², R³ and R⁴ are as defined for compounds of formula I.

The invention also provides compounds of formula Int-13

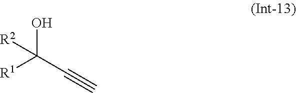

wherein R¹ and R² are as defined for compounds of formula I. The preferences for, R¹ and R² are as defined for compounds of formula I.

The invention also provides compounds of formula Int-14

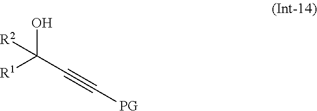

wherein R¹ and R² are as defined for compounds of formula I and PG is an organosilicon group, such as tri-C₁-C₄alkyl-silyl, e.g. trimethylsilyl. The preferences for, R¹ and R² are as defined for compounds of formula I.

The invention also provides compounds of formula Int-15

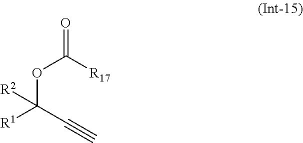

wherein R¹ and R² are as defined for compounds of formula I and R¹⁷ is C₁-C₁₂alkyl. The preferences for, R¹ and R² are as defined for compounds of formula I.

The invention also provides compounds of formula Int-2**

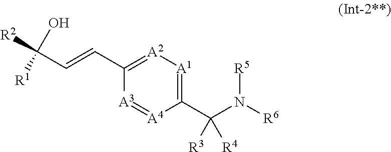

wherein A¹, A², A³, A⁴, R¹, R², R³, R⁴, R⁵ and R⁶ are as defined for compounds of formula I as defined in any one of claims 1 to 10, or a salt of N-oxide thereof. The preferences for A¹, A², A³, A⁴, R¹, R², R³, R⁴, R⁵ and R⁶ are as defined for compounds of formula I.

The invention also provides compounds of formula Int-3**

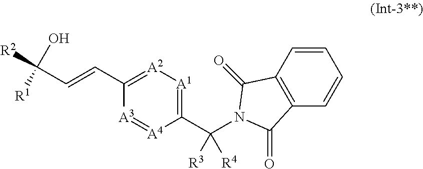

wherein A¹, A², A³, A⁴, R¹, R², R³ and R⁴ are as defined for compounds of formula I as defined in any one of claims 1 to 10, or a salt of N-oxide thereof. The preferences for A¹, A², A³, A⁴, R¹, R², R³ and R⁴ are as defined for compounds of formula I.

The invention also provides compounds of formula Int-9**

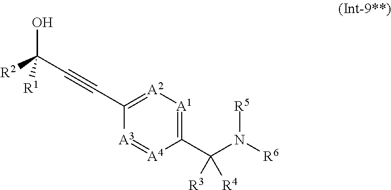

wherein A¹, A², A³, A⁴, R¹, R², R³, R⁴, R⁵ and R⁶ are as defined for compounds of formula I, or a salt of N-oxide thereof. The preferences for A¹, A², A³, A⁴, R¹, R², R³, R⁴, R⁵ and R⁶ are as defined for compounds of formula I.

The invention also provides compounds of formula Int-10**

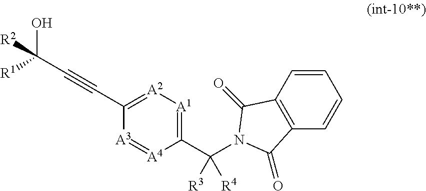

wherein A¹, A², A³, A⁴, R¹, R², R³ and R⁴ are as defined for compounds of formula I, or a salt of N-oxide thereof. The preferences for A¹, A², A³, A⁴, R¹, R², R³ and R⁴ are as defined for compounds of formula I.

The invention also provides compounds of formula Int-11**

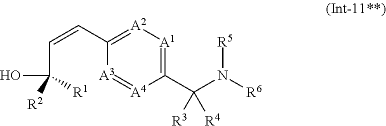

wherein A¹, A², A³, A⁴, R¹, R², R³, R⁴, R⁵ and R⁶ are as defined for compounds of formula I, or a salt of N-oxide thereof. The preferences for A¹, A², A³, A⁴, R¹, R², R³, R⁴, R⁵ and R⁶ are as defined for compounds of formula I.

The invention also provides compounds of formula Int-12**

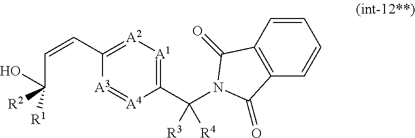

wherein A¹, A², A³, A⁴, R¹, R², R³ and R⁴ are as defined for compounds of formula I, or a salt of N-oxide thereof. The preferences for A¹, A², A³, A⁴, R¹, R², R³ and R⁴ are as defined for compounds of formula I.

The invention also provides compounds of formula Int-13**

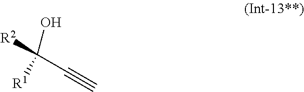

wherein R¹ and R² are as defined for compounds of formula I. The preferences for, R¹ and R² are as defined for compounds of formula I.

The invention also provides compounds of formula Int-14**

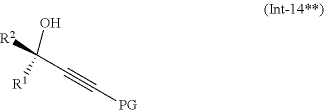

wherein R¹ and R² are as defined for compounds of formula I and PG is an organosilicon group, such as tri-C₁-C₄alkyl-silyl, e.g. trimethylsilyl. The preferences for, R¹ and R² are as defined for compounds of formula I.

The invention also provides compounds of formula Int-15**

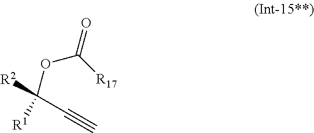

wherein R¹ and R² are as defined for compounds of formula I and R¹⁷ is C₁-C₁₂alkyl. The preferences for, Wand R² are as defined for compounds of formula I.

The invention also provides mixtures of compounds of formula Int-2* and Int-2**, wherein the molar amount of Int-2** in the mixture is more than 50% compared to the combined molar amount of Int-2* and Int-2**

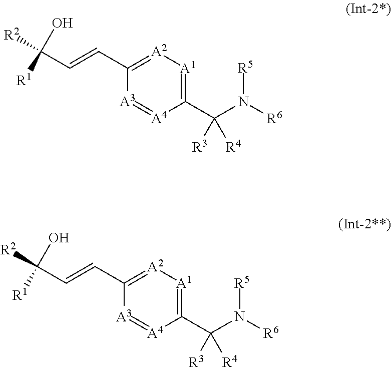

wherein A¹, A², A³, A⁴, R¹, R², R³, R⁴, R⁵ and R⁶ are as defined for compounds of formula I or a salt of N-oxide thereof. The preferences for A¹, A², A³, A⁴, R¹, R², R³, R⁴, R⁵ and R⁶ are as defined for compounds of formula I.

The invention also provides a mixture of compounds of formula Int-3* and Int-3**, wherein the molar amount of Int-3** in the mixture is more than 50% compared to the combined molar amount of Int-3* and Int-3**

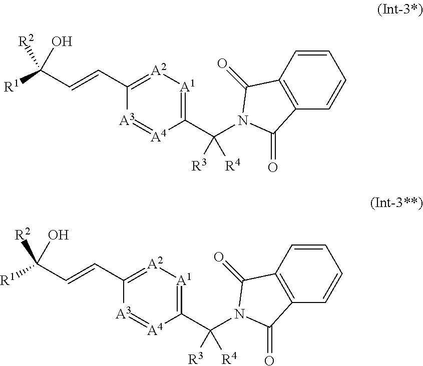

wherein A¹, A², A³, A⁴, R¹, R², R³ and R⁴ are as defined for compounds of formula I or a salt of N-oxide thereof. The preferences for A¹, A², A³, A⁴, R¹, R², R³ and R⁴ are as defined for compounds of formula I.

The invention also provides mixtures of compounds of formula Int-9* and Int-9**, wherein the molar amount of Int-9** in the mixture is more than 50% compared to the combined molar amount of Int-9* and Int-9**

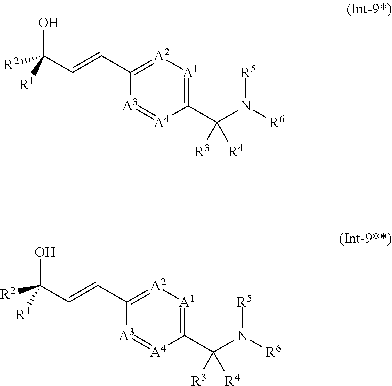

wherein A¹, A², A³, A⁴, R¹, R², R³, R⁴, R⁵ and R⁶ are as defined for compounds of formula I, or a salt of N-oxide thereof. The preferences for A¹, A², A³, A⁴, R¹, R², R³, R⁴, R⁵ and R⁶ are as defined for compounds of formula I.

The invention also provides mixtures of compounds of formula Int-10* and Int-10**, wherein the molar amount of Int-10** in the mixture is more than 50% compared to the combined molar amount of Int-10* and Int-10**

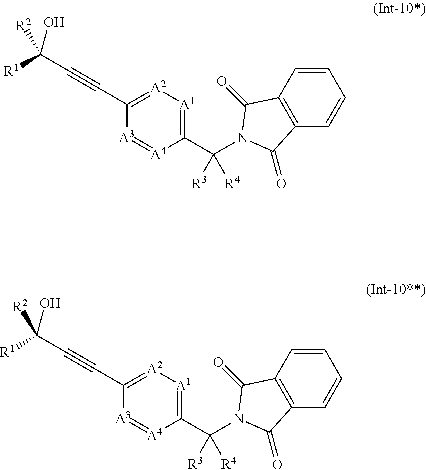

wherein A¹, A², A³, A⁴, R¹, R², R³ and R⁴ are as defined for compounds of formula I, or a salt of N-oxide thereof. The preferences for A¹, A², A³, A⁴, R¹, R², R³ and R⁴ are as defined for compounds of formula I.

The invention also provides mixtures of compounds of formula Int-11* and Int-11**, wherein the molar amount of Int-11** in the mixture is more than 50% compared to the combined molar amount of Int-11* and Int-11**

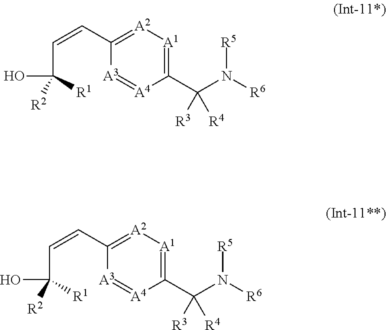

wherein A¹, A², A³, A⁴, R¹, R², R³, R⁴, R⁵ and R⁶ are as defined for compounds of formula I, or a salt of N-oxide thereof. The preferences for A¹, A², A³, A⁴, R¹, R², R³, R⁴, R⁵ and R⁶ are as defined for compounds of formula I.

The invention also provides mixtures of compounds of formula Int-12* and Int-12**, wherein the molar amount of Int-12** in the mixture is more than 50% compared to the combined molar amount of Int-12* and Int-12**

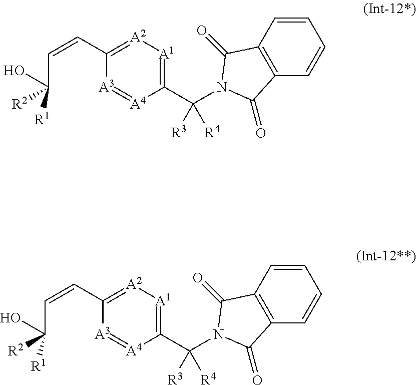

wherein A¹, A², A³, A⁴, R¹, R², R³ and R⁴ are as defined for compounds of formula I, or a salt of N-oxide thereof. The preferences for A¹, A², A³, A⁴, R¹, R², R³ and R⁴ are as defined for compounds of formula I.

The invention also provides mixtures of compounds of formula Int-13* and Int-13**, wherein the molar amount of Int-13** in the mixture is more than 50% compared to the combined molar amount of Int-13* and Int-13**

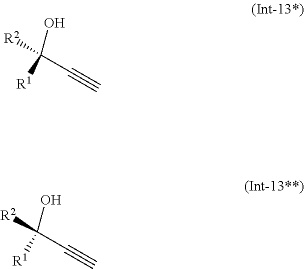

wherein R¹ and R² are as defined for compounds of formula I. The preferences for, R¹ and R² are as defined for compounds of formula I.

The invention also provides mixtures of compounds of formula Int-14* and Int-14**, wherein the molar amount of Int-14** in the mixture is more than 50% compared to the combined molar amount of Int-14* and Int-14**

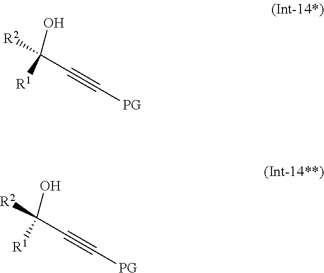

wherein R¹ and R² are as defined for compounds of formula I and PG is an organosilicon group such as tri-C₁-C₄alkyl-silyl, e.g. trimethylsilyl. The preferences for, R¹ and R² are as defined for compounds of formula I.

The invention also provides mixtures of compounds of formula Int-15* and Int-15**, wherein the molar amount of Int-15** in the mixture is more than 50% compared to the combined molar amount of Int-15* and Int-15**

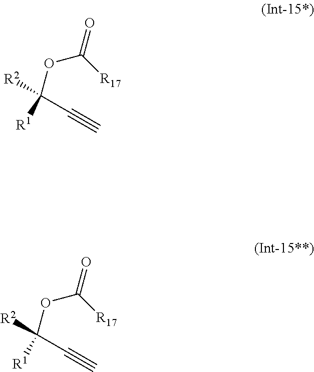

wherein R¹ and R² are as defined for compounds of formula I and R¹⁷ is C₁-C₁₂alkyl. The preferences for, Wand R² are as defined for compounds of formula I.

The tables below illustrate specific compounds of the invention. (Although the substituent identifiers are different to those of formula I above, the identity of the compounds is clear.)

TABLE G

| G | Chemical structure |
|---|---|
| G1 | 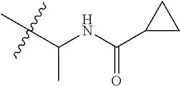 [SMILES: *C(C)NC(=O)C1CC1] |
| G2 | 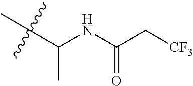 [SMILES: *C(C)NC(=O)CC(F)(F)F] |
| G3 | 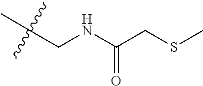 [SMILES: *CNC(=O)CSC] |
| G4 | 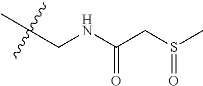 [SMILES: *CNC(=O)CS(C)=O] |
| G5 | 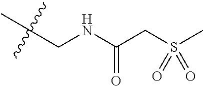 [SMILES: *CNC(=O)CS(C)(=O)=O] |
| G6 | 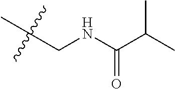 [SMILES: *CNC(=O)C(C)C] |
| G7 | 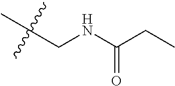 [SMILES: *CNC(=O)CC] |
| G8 | 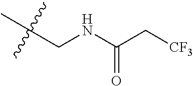 [SMILES: *CNC(=O)CC(F)(F)F] |
| G9 | 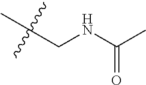 [SMILES: *CNC(C)=O] |
| G10 | 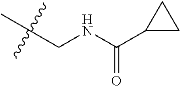 [SMILES: *CNC(=O)C1CC1] |
| G11 | 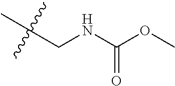 [SMILES: *CNC(=O)OC] |
| G12 | 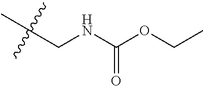 [SMILES: *CNC(=O)OCC] |
| G13 | 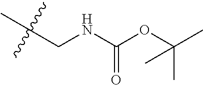 [SMILES: *CNC(=O)OC(C)(C)C] |
| G14 | 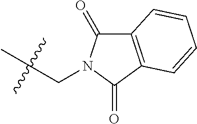 [SMILES: *CN1C(=O)c2ccccc2C1=O] |
| G53 | 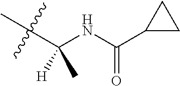 |
| G54 | 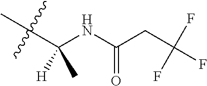 |
| G15 | 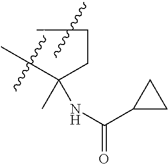 [SMILES: *CCC(*)(C)NC(=O)C1CC1] |
| G16 | 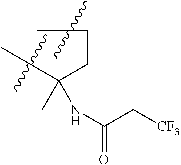 [SMILES: *CCC(*)(C)NC(=O)CC(F)(F)F] |
| G17 | 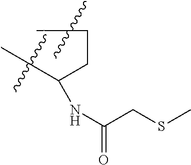 [SMILES: *CCC(*)NC(=O)CSC] |
| G18 | 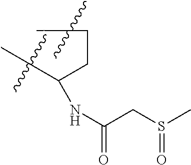 [SMILES: *CCC(*)NC(=O)CS(C)=O] |
| G19 | 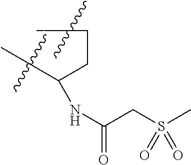 [SMILES: *CCC(*)NC(=O)CS(C)(=O)=O] |
| G20 | 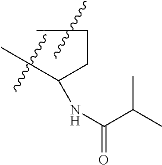 [SMILES: *CCC(*)NC(=O)C(C)C] |
| G21 | 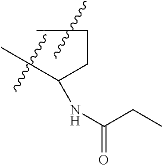 [SMILES: *CCC(*)NC(=O)CC] |
| G22 | 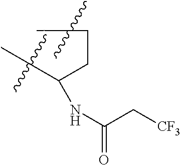 [SMILES: *CCC(*)NC(=O)CC(F)(F)F] |
| G23 | 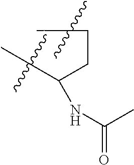 [SMILES: *CCC(*)NC(C)=O] |
| G24 | 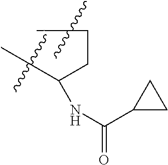 [SMILES: *CCC(*)NC(=O)C1CC1] |
| G25 | 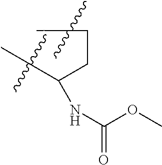 [SMILES: *CCC(*)NC(=O)OC] |
| G26 | 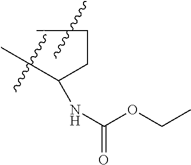 [SMILES: *CCC(*)NC(=O)OCC] |
| G27 | 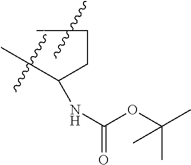 [SMILES: *CCC(*)NC(=O)OC(C)(C)C] |
| G28 | 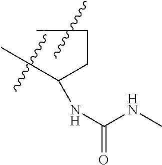 [SMILES: *CCC(*)NC(=O)NC] |
| G29 | 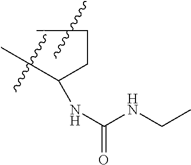 [SMILES: *CCC(*)NC(=O)NCC] |
| G30 | 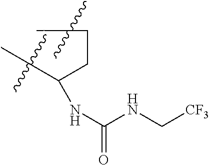 [SMILES: *CCC(*)NC(=O)NCC(F)(F)F] |
| G31 | 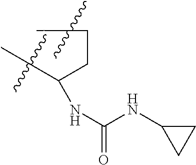 [SMILES: *CCC(*)NC(=O)NC1CC1] |
| G32 | 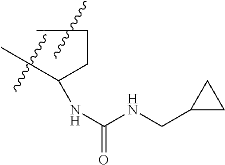 [SMILES: *CCC(*)NC(=O)NCC1CC1] |
| G33 | 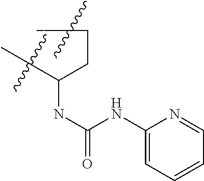 [SMILES: *CCC(*)NC(=O)Nc1ccccn1] |
| G34 | 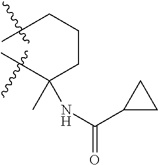 [SMILES: *CCCC(*)(C)NC(=O)C1CC1] |
| G35 | 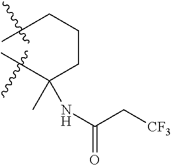 [SMILES: *CCCC(*)(C)NC(=O)CC(F)(F)F] |
| G36 | 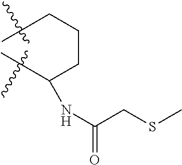 [SMILES: *CCCC(*)NC(=O)CSC] |
| G37 | 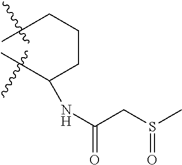 [SMILES: *CCCC(*)NC(=O)CS(C)=O] |
| G38 | 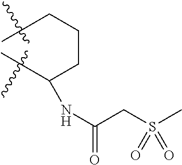 [SMILES: *CCCC(*)NC(=O)CS(C)(=O)=O] |
| G39 | 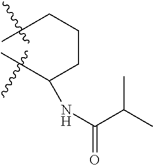 [SMILES: *CCCC(*)NC(=O)C(C)C] |
| G40 | 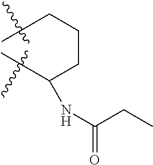 [SMILES: *CCCC(*)NC(=O)CC] |
| G41 | 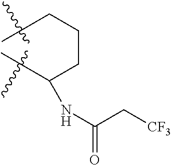 [SMILES: *CCCC(*)NC(=O)CC(F)(F)F] |
| G42 | 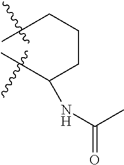 [SMILES: *CCCC(*)NC(C)=O] |
| G43 | 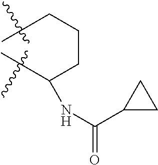 [SMILES: *CCCC(*)NC(=O)C1CC1] |
| G44 | 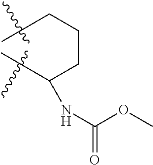 [SMILES: *CCCC(*)NC(=O)OC] |
| G45 | 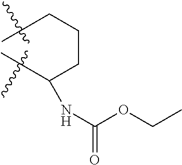 [SMILES: *CCCC(*)NC(=O)OCC] |
| G46 | 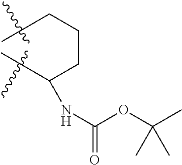 [SMILES: *CCCC(*)NC(=O)OC(C)(C)C] |
| G47 | 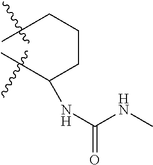 [SMILES: *CCCC(*)NC(=O)NC] |
| G48 | 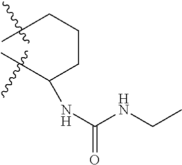 [SMILES: *CCCC(*)NC(=O)NCC] |
| G49 | 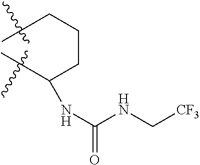 [SMILES: *CCCC(*)NC(=O)NCC(F)(F)F] |
| G50 | 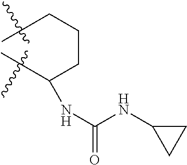 [SMILES: *CCCC(*)NC(=O)NC1CC1] |
| G51 | 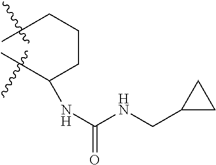 [SMILES: *CCCC(*)NC(=O)NCC1CC1] |
| G52 | 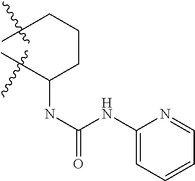 [SMILES: *CCCC(*)NC(=O)Nc1ccccn1] |

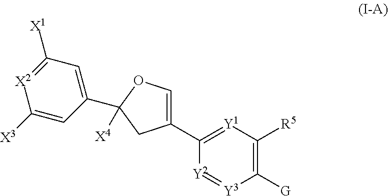

### Table 1P:

Table 1 P provides 690 compounds of formula (I-A) wherein X¹ is chloro, X² is CH, X³ is chloro, Y¹ is CH, Y² is CH, Y³ is CH and X⁴, R⁵ and G have the values listed in the table P.

### Table 2P:

Table 2 P provides 690 compounds of formula (I-A) wherein X¹ is chloro, X² is CH, X³ is chloro, Y¹ is N, Y² is CH, Y³ is CH and X⁴, R⁵ and G have the values listed in the table P.

### Table 3P:

Table 3 P provides 690 compounds of formula (I-A) wherein X¹ is chloro, X² is CH, X³ is chloro, Y¹ is N, Y² is N, Y³ is CH and X⁴, R⁵ and G have the values listed in the table P.

### Table 4P:

Table 4 P provides 690 compounds of formula (I-A) wherein X¹ is chloro, X² is CH, X³ is chloro, Y¹ is CH, Y² is N, Y³ is CH and X⁴, R⁵ and G have the values listed in the table P.

### Table 5P:

Table 5 P provides 690 compounds of formula (I-A) wherein X¹ is chloro, X² is CH, X³ is chloro, Y¹ is CH, Y² is CH, Y³ is N and X⁴, R⁵ and G have the values listed in the table P.

### Table 6P:

Table 6 P provides 690 compounds of formula (I-A) wherein X¹ is chloro, X² is C—F, X³ is hydrogen, Y¹ is CH, Y² is CH, Y³ is CH and X⁴, R⁵ and G have the values listed in the table P.

### Table 7P:

Table 7 P provides 690 compounds of formula (I-A) wherein X¹ is chloro, X² is C—F, X³ is hydrogen, Y¹ is N, Y² is CH, Y³ is CH and X⁴, R⁵ and G have the values listed in the table P.

### Table 8P:

Table 8 P provides 690 compounds of formula (I-A) wherein X¹ is chloro, X² is C—F, X³ is hydrogen, Y¹ is N, Y² is N, Y³ is CH and X⁴, R⁵ and G have the values listed in the table P.

### Table 9P:

Table 9 P provides 690 compounds of formula (I-A) wherein X¹ is chloro, X² is C—F, X³ is hydrogen, Y¹ is CH, Y² is N, Y³ is CH and X⁴, R⁵ and G have the values listed in the table P.

### Table 10P:

Table 10 P provides 690 compounds of formula (I-A) wherein X¹ is chloro, X² is C—F, X³ is hydrogen, Y¹ is CH, Y² is CH, Y³ is N and X⁴, R⁵ and G have the values listed in the table P.

### Table 11P:

Table 11 P provides 690 compounds of formula (I-A) wherein X¹ is fluoro, X² is C—Cl, X³ is hydrogen, Y¹ is CH, Y² is CH, Y³ is CH and X⁴, R⁵ and G have the values listed in the table P.

### Table 12P:

Table 12 P provides 690 compounds of formula (I-A) wherein X¹ is fluoro, X² is C—Cl, X³ is hydrogen, Y¹ is N, Y² is CH, Y³ is CH and X⁴, R⁵ and G have the values listed in the table P.

### Table 13P:

Table 13 P provides 690 compounds of formula (I-A) wherein X¹ is fluoro, X² is C—Cl, X³ is hydrogen, Y¹ is N, Y² is N, Y³ is CH and X⁴, R⁵ and G have the values listed in the table P.

### Table 14P:

Table 14 P provides 690 compounds of formula (I-A) wherein X¹ is fluoro, X² is C—Cl, X³ is hydrogen, Y¹ is CH, Y² is N, Y³ is CH and X⁴, R⁵ and G have the values listed in the table P.

### Table 15P:

Table 15 P provides 690 compounds of formula (I-A) wherein X¹ is fluoro, X² is C—Cl, X³ is hydrogen, Y¹ is CH, Y² is CH, Y³ is N and X⁴, R⁵ and G have the values listed in the table P.

### Table 16P:

Table 16 P provides 690 compounds of formula (I-A) wherein X¹ is chloro, X² is C—Cl, X³ is hydrogen, Y¹ is CH, Y² is CH, Y³ is CH and X⁴, R⁵ and G have the values listed in the table P.

### Table 17P:

Table 17 P provides 690 compounds of formula (I-A) wherein X¹ is chloro, X² is C—Cl, X³ is hydrogen, Y¹ is N, Y² is CH, Y³ is CH and X⁴, R⁵ and G have the values listed in the table P.

### Table 18P:

Table 18 P provides 690 compounds of formula (I-A) wherein X¹ is chloro, X² is C—Cl, X³ is hydrogen, Y¹ is N, Y² is N, Y³ is CH and X⁴, R⁵ and G have the values listed in the table P.

### Table 19P:

Table 19 P provides 690 compounds of formula (I-A) wherein X¹ is chloro, X² is C—Cl, X³ is hydrogen,

Y¹ is CH, Y² is N, Y³ is CH and X⁴, R⁵ and G have the values listed in the table P.

### Table 20P:

Table 20 P provides 690 compounds of formula (I-A) wherein X¹ is chloro, X² is C—Cl, X³ is hydrogen, Y¹ is CH, Y² is CH, Y³ is N and X⁴, R⁵ and G have the values listed in the table P.

### Table 21P:

Table 21 P provides 690 compounds of formula (I-A) wherein X¹ is chloro, X² is C—F, X³ is chloro, Y¹ is CH, Y² is CH, Y³ is CH and X⁴, R⁵ and G have the values listed in the table P.

### Table 22P:

Table 22 P provides 690 compounds of formula (I-A) wherein X¹ is chloro, X² is C—F, X³ is chloro, Y¹ is N, Y² is CH, Y³ is CH and X⁴, R⁵ and G have the values listed in the table P.

### Table 23P:

Table 23 P provides 690 compounds of formula (I-A) wherein X¹ is chloro, X² is C—F, X³ is chloro, Y¹ is N, Y² is N, Y³ is CH and X⁴, R⁵ and G have the values listed in the table P.
Table 24P: 50 Table 24 P provides 690 compounds of formula (I-A) wherein X¹ is chloro, X² is C—F, X³ is chloro, Y¹ is CH, Y² is N, Y³ is CH and X⁴, R⁵ and G have the values listed in the table P.

### Table 25P:

Table 25 P provides 690 compounds of formula (I-A) wherein X¹ is chloro, X² is C—F, X³ is chloro, Y¹ is

### Table 26P:

Table 26 P provides 690 compounds of formula (I-A) wherein X¹ is chloro, X² is C—Cl, X³ is chloro, Y¹ is CH, Y² is CH, Y³ is CH and X⁴, R⁵ and G have the values listed in the table P.

### Table 27P:

Table 27 P provides 690 compounds of formula (I-A) wherein X¹ is chloro, X² is C—Cl, X³ is chloro, Y¹ is

N, Y² is CH, Y³ is CH and X⁴, R⁵ and G have the values listed in the table P.

### Table 28P:

Table 28 P provides 690 compounds of formula (I-A) wherein X¹ is chloro, X² is C—Cl, X³ is chloro, Y¹ is N, Y² is N, Y³ is CH and X⁴, R⁵ and G have the values listed in the table P.

### Table 29P:

Table 29 P provides 690 compounds of formula (I-A) wherein X¹ is chloro, X² is C—Cl, X³ is chloro, Y¹ is CH, Y² is N, Y³ is CH and X⁴, R⁵ and G have the values listed in the table P.

### Table 30P:

Table 30 P provides 690 compounds of formula (I-A) wherein X¹ is chloro, X² is C—Cl, X³ is chloro, Y¹ is CH, Y² is CH, Y³ is N and X⁴, R⁵ and G have the values listed in the table P.

### Table 31P:

Table 31 P provides 690 compounds of formula (I-A) wherein X¹ is chloro, X² is C—Br, X³ is chloro, Y¹ is CH, Y² is CH, Y³ is CH and X⁴, R⁵ and G have the values listed in the table P.

### Table 32P:

Table 32 P provides 690 compounds of formula (I-A) wherein X¹ is chloro, X² is C—Br, X³ is chloro, Y¹ is

N, Y² is CH, Y³ is CH and X⁴, R⁵ and G have the values listed in the table P.

### Table 33P:

Table 33 P provides 690 compounds of formula (I-A) wherein X¹ is chloro, X² is C—Br, X³ is chloro, Y¹ is N, Y² is N, Y³ is CH and X⁴, R⁵ and G have the values listed in the table P.

### Table 34P:

Table 34 P provides 690 compounds of formula (I-A) wherein X¹ is chloro, X² is C—Br, X³ is chloro, Y¹ is CH, Y² is N, Y³ is CH and X⁴, R⁵ and G have the values listed in the table P.

### Table 35P:

Table 35 P provides 690 compounds of formula (I-A) wherein X¹ is chloro, X² is C—Br, X³ is chloro, Y¹ is CH, Y² is CH, Y³ is N and X⁴, R⁵ and G have the values listed in the table P.

### Table 36P:

Table 36 P provides 690 compounds of formula (I-A) wherein X¹ is chloro, X² is C—I, X³ is chloro, Y¹ is CH, Y² is CH, Y³ is CH and X⁴, R⁵ and G have the values listed in the table P.

### Table 37P:

Table 37 P provides 690 compounds of formula (I-A) wherein X¹ is chloro, X² is C—I, X³ is chloro, Y¹ is

N, Y² is CH, Y³ is CH and X⁴, R⁵ and G have the values listed in the table P.

### Table 38P:

Table 38 P provides 690 compounds of formula (I-A) wherein X¹ is chloro, X² is C—I, X³ is chloro, Y¹ is N, Y² is N, Y³ is CH and X⁴, R⁵ and G have the values listed in the table P.

### Table 39P:

Table 39 P provides 690 compounds of formula (I-A) wherein X¹ is chloro, X² is C—I, X³ is chloro, Y¹ is CH, Y² is N, Y³ is CH and X⁴, R⁵ and G have the values listed in the table P.

### Table 40P:

Table 40 P provides 690 compounds of formula (I-A) wherein X¹ is chloro, X² is C—I, X³ is chloro, Y¹ is CH, Y² is CH, Y³ is N and X⁴, R⁵ and G have the values listed in the table P.

### Table 41P:

Table 41 P provides 690 compounds of formula (I-A) wherein X¹ is fluoro, X² is C—F, X³ is fluoro, Y¹ is CH, Y² is CH, Y³ is CH and X⁴, R⁵ and G have the values listed in the table P.

### Table 42P:

Table 42 P provides 690 compounds of formula (I-A) wherein X¹ is fluoro, X² is C—F, X³ is fluoro, Y¹ is N, Y² is CH, Y³ is CH and X⁴, R⁵ and G have the values listed in the table P.

### Table 43P:

Table 43 P provides 690 compounds of formula (I-A) wherein X¹ is fluoro, X² is C—F, X³ is fluoro, Y¹ is N, Y² is N, Y³ is CH and X⁴, R⁵ and G have the values listed in the table P.

### Table 44P:

Table 44 P provides 690 compounds of formula (I-A) wherein X¹ is fluoro, X² is C—F, X³ is fluoro, Y¹ is CH, Y² is N, Y³ is CH and X⁴, R⁵ and G have the values listed in the table P.

### Table 45P:

Table 45 P provides 690 compounds of formula (I-A) wherein X¹ is fluoro, X² is C—F, X³ is fluoro, Y¹ is CH, Y² is CH, Y³ is N and X⁴, R⁵ and G have the values listed in the table P.

### Table 46P:

Table 46 P provides 690 compounds of formula (I-A) wherein X¹ is chloro, X² is CH, X³ is bromo, Y¹ is CH, Y² is CH, Y³ is CH and X⁴, R⁵ and G have the values listed in the table P.

### Table 47P:

Table 47 P provides 690 compounds of formula (I-A) wherein X¹ is chloro, X² is CH, X³ is bromo, Y¹ is N, Y² is CH, Y³ is CH and X⁴, R⁵ and G have the values listed in the table P.

### Table 48P:

Table 48 P provides 690 compounds of formula (I-A) wherein X¹ is chloro, X² is CH, X³ is bromo, Y¹ is N, Y² is N, Y³ is CH and X⁴, R⁵ and G have the values listed in the table P.

### Table 49P:

Table 49 P provides 690 compounds of formula (I-A) wherein X¹ is chloro, X² is CH, X³ is bromo, Y¹ is CH, Y² is N, Y³ is CH and X⁴, R⁵ and G have the values listed in the table P.

### Table 50P:

Table 50 P provides 690 compounds of formula (I-A) wherein X¹ is chloro, X² is CH, X³ is bromo, Y¹ is CH, Y² is CH, Y³ is N and X⁴, R⁵ and G have the values listed in the table P.

### Table 51P:

Table 51 P provides 690 compounds of formula (I-A) wherein X¹ is chloro, X² is CH, X³ is fluoro, Y¹ is CH, Y² is CH, Y³ is CH and X⁴, R⁵ and G have the values listed in the table P.

### Table 52P:

Table 52 P provides 690 compounds of formula (I-A) wherein X¹ is chloro, X² is CH, X³ is fluoro, Y¹ is N, Y² is CH, Y³ is CH and X⁴, R⁵ and G have the values listed in the table P.

### Table 53P:

Table 53 P provides 690 compounds of formula (I-A) wherein X¹ is chloro, X² is CH, X³ is fluoro, Y¹ is N, Y² is N, Y³ is CH and X⁴, R⁵ and G have the values listed in the table P.

### Table 54P:

Table 54 P provides 690 compounds of formula (I-A) wherein X¹ is chloro, X² is CH, X³ is fluoro, Y¹ is CH, Y² is N, Y³ is CH and X⁴, R⁵ and G have the values listed in the table P.

### Table 55P:

Table 55 P provides 690 compounds of formula (I-A) wherein X¹ is chloro, X² is CH, X³ is fluoro, Y¹ is CH, Y² is CH, Y³ is N and X⁴, R⁵ and G have the values listed in the table P.

### Table 56P:

Table 56 P provides 690 compounds of formula (I-A) wherein X¹ is chloro, X² is CH, X³ is trifluoromethyl, Y¹ is CH, Y² is CH, Y³ is CH and X⁴, R⁵ and G have the values listed in the table P.

### Table 57P:

Table 57 P provides 690 compounds of formula (I-A) wherein X¹ is chloro, X² is CH, X³ is trifluoromethyl, Y¹ is N, Y² is CH, Y³ is CH and X⁴, R⁵ and G have the values listed in the table P.

### Table 58P:

Table 58 P provides 690 compounds of formula (I-A) wherein X¹ is chloro, X² is CH, X³ is trifluoromethyl, Y¹ is N, Y² is N, Y³ is CH and X⁴, R⁵ and G have the values listed in the table P.

### Table 59P:

Table 59 P provides 690 compounds of formula (I-A) wherein X¹ is chloro, X² is CH, X³ is trifluoromethyl, Y¹ is CH, Y² is N, Y³ is CH and X⁴, R⁵ and G have the values listed in the table P.
Table 60P: 50 Table 60 P provides 690 compounds of formula (I-A) wherein X¹ is chloro, X² is CH, X³ is trifluoromethyl, Y¹ is CH, Y² is CH, Y³ is N and X⁴, R⁵ and G have the values listed in the table P.

### Table 61P:

Table 61 P provides 690 compounds of formula (I-A) wherein X¹ is chloro, X² is C—Cl, X³ is trifluoromethyl, Y¹ is CH, Y² is CH, Y³ is CH and X⁴, R⁵ and G have the values listed in the table P.

### Table 62P:

Table 62 P provides 690 compounds of formula (I-A) wherein X¹ is chloro, X² is C—Cl, X³ is trifluoromethyl, Y¹ is N, Y² is CH, Y³ is CH and X⁴, R⁵ and G have the values listed in the table P.

### Table 63P:

Table 63 P provides 690 compounds of formula (I-A) wherein X¹ is chloro, X² is C—Cl, X³ is trifluoromethyl, Y¹ is N, Y² is N, Y³ is CH and X⁴, R⁵ and G have the values listed in the table P.

### Table 64P:

Table 64 P provides 690 compounds of formula (I-A) wherein X¹ is chloro, X² is C—Cl, X³ is trifluoromethyl, Y¹ is CH, Y² is N, Y³ is CH and X⁴, R⁵ and G have the values listed in the table P.

### Table 65P:

Table 65 P provides 690 compounds of formula (I-A) wherein X¹ is chloro, X² is C—Cl, X³ is trifluoromethyl, Y¹ is CH, Y² is CH, Y³ is N and X⁴, R⁵ and G have the values listed in the table P.

### Table 66P:

Table 66 P provides 690 compounds of formula (I-A) wherein X¹ is trifluoromethyl, X² is CH, X³ is trifluoromethyl, Y¹ is CH, Y² is CH, Y³ is CH and X⁴, R⁵ and G have the values listed in the table P.

### Table 67P:

Table 67 P provides 690 compounds of formula (I-A) wherein X¹ is trifluoromethyl, X² is CH, X³ is trifluoromethyl, Y¹ is N, Y² is CH, Y³ is CH and X⁴, R⁵ and G have the values listed in the table P.

### Table 68P:

Table 68 P provides 690 compounds of formula (I-A) wherein X¹ is trifluoromethyl, X² is CH, X³ is trifluoromethyl, Y¹ is N, Y² is N, Y³ is CH and X⁴, R⁵ and G have the values listed in the table P.

### Table 69P:

Table 69 P provides 690 compounds of formula (I-A) wherein X¹ is trifluoromethyl, X² is CH, X³ is trifluoromethyl, Y¹ is CH, Y² is N, Y³ is CH and X⁴, R⁵ and G have the values listed in the table P.

### Table 70P:

Table 70 P provides 690 compounds of formula (I-A) wherein X¹ is trifluoromethyl, X² is CH, X³ is trifluoromethyl, Y¹ is CH, Y² is CH, Y³ is N and X⁴, R⁵ and G have the values listed in the table P.

### Table 71P:

Table 71 P provides 690 compounds of formula (I-A) wherein X¹ is trifluoromethyl, X² is C—Cl, X³ is trifluoromethyl, Y¹ is CH, Y² is CH, Y³ is CH and X⁴, R⁵ and G have the values listed in the table P.

### Table 72P:

Table 72 P provides 690 compounds of formula (I-A) wherein X¹ is trifluoromethyl, X² is C—Cl, X³ is trifluoromethyl, Y¹ is N, Y² is CH, Y³ is CH and X⁴, R⁵ and G have the values listed in the table P.

### Table 73P:

Table 73 P provides 690 compounds of formula (I-A) wherein X¹ is trifluoromethyl, X² is C—Cl, X³ is trifluoromethyl, Y¹ is N, Y² is N, Y³ is CH and X⁴, R⁵ and G have the values listed in the table P.

### Table 74P:

Table 74 P provides 690 compounds of formula (I-A) wherein X¹ is trifluoromethyl, X² is C—Cl, X³ is trifluoromethyl, Y¹ is CH, Y² is N, Y³ is CH and X⁴, R⁵ and G have the values listed in the table P. Table 75P:
Table 75 P provides 690 compounds of formula (I-A) wherein X¹ is trifluoromethyl, X² is C—Cl, X³ is trifluoromethyl, Y¹ is CH, Y² is CH, Y³ is N and X⁴, R⁵ and G have the values listed in the table P.

### Table 76P:

Table 76 P provides 690 compounds of formula (I-A) wherein X¹ is trifluoromethyl, X² is CH, X³ is hydrogen, Y¹ is CH, Y² is CH, Y³ is CH and X⁴, R⁵ and G have the values listed in the table P.

### Table 77P:

Table 77 P provides 690 compounds of formula (I-A) wherein X¹ is trifluoromethyl, X² is CH, X³ is hydrogen, Y¹ is N, Y² is CH, Y³ is CH and X⁴, R⁵ and G have the values listed in the table P.
Table 78P: 50 Table 78 P provides 690 compounds of formula (I-A) wherein X¹ is trifluoromethyl, X² is CH, X³ is hydrogen, Y¹ is N, Y² is N, Y³ is CH and X⁴, R⁵ and G have the values listed in the table P.

### Table 79P:

Table 79 P provides 690 compounds of formula (I-A) wherein X¹ is trifluoromethyl, X² is CH, X³ is hydrogen, Y¹ is CH, Y² is N, Y³ is CH and X⁴, R⁵ and G have the values listed in the table P.

### Table 80P:

Table 80 P provides 690 compounds of formula (I-A) wherein X¹ is trifluoromethyl, X² is CH, X³ is hydrogen, Y¹ is CH, Y² is CH, Y³ is N and X⁴, R⁵ and G have the values listed in the table P.

### Table 81P:

Table 81 P provides 690 compounds of formula (I-A) wherein X¹ is chloro, X² is N, X³ is chloro, Y¹ is CH, Y² is CH, Y³ is CH and X⁴, R⁵ and G have the values listed in the table P.

### Table 82P:

Table 82 P provides 690 compounds of formula (I-A) wherein X¹ is chloro, X² is N, X³ is chloro, Y¹ is N, Y² is CH, Y³ is CH and X⁴, R⁵ and G have the values listed in the table P.

### Table 83P:

Table 83 P provides 690 compounds of formula (I-A) wherein X¹ is chloro, X² is N, X³ is chloro, Y¹ is N, Y² is N, Y³ is CH and X⁴, R⁵ and G have the values listed in the table P.

### Table 84P:

Table 84 P provides 690 compounds of formula (I-A) wherein X¹ is chloro, X² is N, X³ is chloro, Y¹ is CH, Y² is N, Y³ is CH and X⁴, R⁵ and G have the values listed in the table P.

### Table 85P:

Table 85 P provides 690 compounds of formula (I-A) wherein X¹ is chloro, X² is N, X³ is chloro, Y¹ is CH, Y² is CH, Y³ is N and X⁴, R⁵ and G have the values listed in the table P.

### Table 86P:

Table 86 P provides 690 compounds of formula (I-A) wherein X¹ is trifluoromethyl, X² is N, X³ is trifluoromethyl, Y¹ is CH, Y² is CH, Y³ is CH and X⁴, R⁵ and G have the values listed in the table P.

### Table 87P:

Table 87 P provides 690 compounds of formula (I-A) wherein X¹ is trifluoromethyl, X² is N, X³ is trifluoromethyl, Y¹ is N, Y² is CH, Y³ is CH and X⁴, R⁵ and G have the values listed in the table P.

### Table 88P:

Table 88 P provides 690 compounds of formula (I-A) wherein X¹ is trifluoromethyl, X² is N, X³ is trifluoromethyl, Y¹ is N, Y² is N, Y³ is CH and X⁴, R⁵ and G have the values listed in the table P.

### Table 89P:

Table 89 P provides 690 compounds of formula (I-A) wherein X¹ is trifluoromethyl, X² is N, X³ is trifluoromethyl, Y¹ is CH, Y² is N, Y³ is CH and X⁴, R⁵ and G have the values listed in the table P.

### Table 90P:

Table 90 P provides 690 compounds of formula (I-A) wherein X¹ is trifluoromethyl, X² is N, X³ is trifluoromethyl, Y¹ is CH, Y² is CH, Y³ is N and X⁴, R⁵ and G have the values listed in the table P.

TABLE P

| | X4 | R5 | G |
|---|---|---|---|
| P.001 | chlorodifluoromethyl | bromo | G1 |
| P.002 | chlorodifluoromethyl | chloro | G1 |
| P.003 | chlorodifluoromethyl | cyano | G1 |
| P.004 | chlorodifluoromethyl | cyclopropyl | G1 |
| P.005 | chlorodifluoromethyl | ethyl | G1 |
| P.006 | chlorodifluoromethyl | fluoro | G1 |
| P.007 | chlorodifluoromethyl | hydrogen | G1 |
| P.008 | chlorodifluoromethyl | methoxy | G1 |
| P.009 | chlorodifluoromethyl | methyl | G1 |
| P.010 | chlorodifluoromethyl | nitro | G1 |
| P.011 | chlorodifluoromethyl | trifluoromethoxy | G1 |
| P.012 | chlorodifluoromethyl | trifluoromethyl | G1 |
| P.013 | difluoromethyl | bromo | G1 |
| P.014 | difluoromethyl | chloro | G1 |
| P.015 | difluoromethyl | cyano | G1 |
| P.016 | difluoromethyl | cyclopropyl | G1 |
| P.017 | difluoromethyl | ethyl | G1 |
| P.018 | difluoromethyl | fluoro | G1 |
| P.019 | difluoromethyl | hydrogen | G1 |
| P.020 | difluoromethyl | methoxy | G1 |
| P.021 | difluoromethyl | methyl | G1 |
| P.022 | difluoromethyl | nitro | G1 |
| P.023 | difluoromethyl | trifluoromethoxy | G1 |
| P.024 | difluoromethyl | trifluoromethyl | G1 |
| P.025 | trifluoromethyl | bromo | G1 |
| P.026 | trifluoromethyl | chloro | G1 |
| P.027 | trifluoromethyl | cyano | G1 |
| P.028 | trifluoromethyl | cyclopropyl | G1 |
| P.029 | trifluoromethyl | ethyl | G1 |
| P.030 | trifluoromethyl | fluoro | G1 |
| P.031 | trifluoromethyl | hydrogen | G1 |
| P.032 | trifluoromethyl | methoxy | G1 |
| P.033 | trifluoromethyl | methyl | G1 |
| P.034 | trifluoromethyl | nitro | G1 |
| P.035 | trifluoromethyl | trifluoromethoxy | G1 |
| P.036 | trifluoromethyl | trifluoromethyl | G1 |
| P.037 | chlorodifluoromethyl | bromo | G2 |
| P.038 | chlorodifluoromethyl | chloro | G2 |
| P.039 | chlorodifluoromethyl | cyano | G2 |
| P.040 | chlorodifluoromethyl | cyclopropyl | G2 |
| P.041 | chlorodifluoromethyl | ethyl | G2 |
| P.042 | chlorodifluoromethyl | fluoro | G2 |
| P.043 | chlorodifluoromethyl | hydrogen | G2 |
| P.044 | chlorodifluoromethyl | methoxy | G2 |
| P.045 | chlorodifluoromethyl | methyl | G2 |
| P.046 | chlorodifluoromethyl | nitro | G2 |
| P.047 | chlorodifluoromethyl | trifluoromethoxy | G2 |
| P.048 | chlorodifluoromethyl | trifluoromethyl | G2 |
| P.049 | difluoromethyl | bromo | G2 |
| P.050 | difluoromethyl | chloro | G2 |
| P.051 | difluoromethyl | cyano | G2 |
| P.052 | difluoromethyl | cyclopropyl | G2 |
| P.053 | difluoromethyl | ethyl | G2 |
| P.054 | difluoromethyl | fluoro | G2 |
| P.055 | difluoromethyl | hydrogen | G2 |
| P.056 | difluoromethyl | methoxy | G2 |
| P.057 | difluoromethyl | methyl | G2 |
| P.058 | difluoromethyl | nitro | G2 |
| P.059 | difluoromethyl | trifluoromethoxy | G2 |
| P.060 | difluoromethyl | trifluoromethyl | G2 |
| P.061 | trifluoromethyl | bromo | G2 |
| P.062 | trifluoromethyl | chloro | G2 |
| P.063 | trifluoromethyl | cyano | G2 |
| P.064 | trifluoromethyl | cyclopropyl | G2 |
| P.065 | trifluoromethyl | ethyl | G2 |
| P.066 | trifluoromethyl | fluoro | G2 |
| P.067 | trifluoromethyl | hydrogen | G2 |
| P.068 | trifluoromethyl | methoxy | G2 |
| P.069 | trifluoromethyl | methyl | G2 |
| P.070 | trifluoromethyl | nitro | G2 |
| P.071 | trifluoromethyl | trifluoromethoxy | G2 |
| P.072 | trifluoromethyl | trifluoromethyl | G2 |
| P.073 | chlorodifluoromethyl | bromo | G3 |
| P.074 | chlorodifluoromethyl | chloro | G3 |
| P.075 | chlorodifluoromethyl | cyano | G3 |
| P.076 | chlorodifluoromethyl | cyclopropyl | G3 |
| P.077 | chlorodifluoromethyl | ethyl | G3 |
| P.078 | chlorodifluoromethyl | fluoro | G3 |
| P.079 | chlorodifluoromethyl | hydrogen | G3 |
| P.080 | chlorodifluoromethyl | methoxy | G3 |
| P.081 | chlorodifluoromethyl | methyl | G3 |
| P.082 | chlorodifluoromethyl | nitro | G3 |
| P.083 | chlorodifluoromethyl | trifluoromethoxy | G3 |
| P.084 | chlorodifluoromethyl | trifluoromethyl | G3 |
| P.085 | difluoromethyl | bromo | G3 |
| P.086 | difluoromethyl | chloro | G3 |
| P.087 | difluoromethyl | cyano | G3 |
| P.088 | difluoromethyl | cyclopropyl | G3 |
| P.089 | difluoromethyl | ethyl | G3 |
| P.090 | difluoromethyl | fluoro | G3 |
| P.091 | difluoromethyl | hydrogen | G3 |
| P.092 | difluoromethyl | methoxy | G3 |
| P.093 | difluoromethyl | methyl | G3 |
| P.094 | difluoromethyl | nitro | G3 |
| P.095 | difluoromethyl | trifluoromethoxy | G3 |
| P.096 | difluoromethyl | trifluoromethyl | G3 |
| P.097 | trifluoromethyl | bromo | G3 |
| P.098 | trifluoromethyl | chloro | G3 |
| P.099 | trifluoromethyl | cyano | G3 |
| P.100 | trifluoromethyl | cyclopropyl | G3 |
| P.101 | trifluoromethyl | ethyl | G3 |
| P.102 | trifluoromethyl | fluoro | G3 |
| P.103 | trifluoromethyl | hydrogen | G3 |
| P.104 | trifluoromethyl | methoxy | G3 |
| P.105 | trifluoromethyl | methyl | G3 |
| P.106 | trifluoromethyl | nitro | G3 |
| P.107 | trifluoromethyl | trifluoromethoxy | G3 |
| P.108 | trifluoromethyl | trifluoromethyl | G3 |
| P.109 | chlorodifluoromethyl | bromo | G4 |
| P.110 | chlorodifluoromethyl | chloro | G4 |
| P.111 | chlorodifluoromethyl | cyano | G4 |
| P.112 | chlorodifluoromethyl | cyclopropyl | G4 |
| P.113 | chlorodifluoromethyl | ethyl | G4 |
| P.114 | chlorodifluoromethyl | fluoro | G4 |
| P.115 | chlorodifluoromethyl | hydrogen | G4 |
| P.116 | chlorodifluoromethyl | methoxy | G4 |
| P.117 | chlorodifluoromethyl | methyl | G4 |
| P.118 | chlorodifluoromethyl | nitro | G4 |
| P.119 | chlorodifluoromethyl | trifluoromethoxy | G4 |
| P.120 | chlorodifluoromethyl | trifluoromethyl | G4 |
| P.121 | difluoromethyl | bromo | G4 |
| P.122 | difluoromethyl | chloro | G4 |
| P.123 | difluoromethyl | cyano | G4 |
| P.124 | difluoromethyl | cyclopropyl | G4 |
| P.125 | difluoromethyl | ethyl | G4 |
| P.126 | difluoromethyl | fluoro | G4 |
| P.127 | difluoromethyl | hydrogen | G4 |
| P.128 | difluoromethyl | methoxy | G4 |
| P.129 | difluoromethyl | methyl | G4 |
| P.130 | difluoromethyl | nitro | G4 |
| P.131 | difluoromethyl | trifluoromethoxy | G4 |
| P.132 | difluoromethyl | trifluoromethyl | G4 |
| P.133 | trifluoromethyl | bromo | G4 |
| P.134 | trifluoromethyl | chloro | G4 |
| P.135 | trifluoromethyl | cyano | G4 |
| P.136 | trifluoromethyl | cyclopropyl | G4 |
| P.137 | trifluoromethyl | ethyl | G4 |
| P.138 | trifluoromethyl | fluoro | G4 |
| P.139 | trifluoromethyl | hydrogen | G4 |
| P.140 | trifluoromethyl | methoxy | G4 |
| P.141 | trifluoromethyl | methyl | G4 |
| P.142 | trifluoromethyl | nitro | G4 |
| P.143 | trifluoromethyl | trifluoromethoxy | G4 |
| P.144 | trifluoromethyl | trifluoromethyl | G4 |
| P.145 | chlorodifluoromethyl | bromo | G5 |
| P.146 | chlorodifluoromethyl | chloro | G5 |
| P.147 | chlorodifluoromethyl | cyano | G5 |
| P.148 | chlorodifluoromethyl | cyclopropyl | G5 |
| P.149 | chlorodifluoromethyl | ethyl | G5 |
| P.150 | chlorodifluoromethyl | fluoro | G5 |
| P.151 | chlorodifluoromethyl | hydrogen | G5 |
| P.152 | chlorodifluoromethyl | methoxy | G5 |
| P.153 | chlorodifluoromethyl | methyl | G5 |
| P.154 | chlorodifluoromethyl | nitro | G5 |
| P.155 | chlorodifluoromethyl | trifluoromethoxy | G5 |
| P.156 | chlorodifluoromethyl | trifluoromethyl | G5 |
| P.157 | difluoromethyl | bromo | G5 |
| P.158 | difluoromethyl | chloro | G5 |
| P.159 | difluoromethyl | cyano | G5 |
| P.160 | difluoromethyl | cyclopropyl | G5 |
| P.161 | difluoromethyl | ethyl | G5 |
| P.162 | difluoromethyl | fluoro | G5 |
| P.163 | difluoromethyl | hydrogen | G5 |
| P.164 | difluoromethyl | methoxy | G5 |
| P.165 | difluoromethyl | methyl | G5 |
| P.166 | difluoromethyl | nitro | G5 |
| P.167 | difluoromethyl | trifluoromethoxy | G5 |
| P.168 | difluoromethyl | trifluoromethyl | G5 |
| P.169 | trifluoromethyl | bromo | G5 |
| P.170 | trifluoromethyl | chloro | G5 |
| P.171 | trifluoromethyl | cyano | G5 |
| P.172 | trifluoromethyl | cyclopropyl | G5 |
| P.173 | trifluoromethyl | ethyl | G5 |
| P.174 | trifluoromethyl | fluoro | G5 |
| P.175 | trifluoromethyl | hydrogen | G5 |
| P.176 | trifluoromethyl | methoxy | G5 |
| P.177 | trifluoromethyl | methyl | G5 |
| P.178 | trifluoromethyl | nitro | G5 |
| P.179 | trifluoromethyl | trifluoromethoxy | G5 |
| P.180 | trifluoromethyl | trifluoromethyl | G5 |
| P.181 | chlorodifluoromethyl | bromo | G6 |
| P.182 | chlorodifluoromethyl | chloro | G6 |
| P.183 | chlorodifluoromethyl | cyano | G6 |
| P.184 | chlorodifluoromethyl | cyclopropyl | G6 |
| P.185 | chlorodifluoromethyl | ethyl | G6 |
| P.186 | chlorodifluoromethyl | fluoro | G6 |
| P.187 | chlorodifluoromethyl | hydrogen | G6 |
| P.188 | chlorodifluoromethyl | methoxy | G6 |
| P.189 | chlorodifluoromethyl | methyl | G6 |
| P.190 | chlorodifluoromethyl | nitro | G6 |
| P.191 | chlorodifluoromethyl | trifluoromethoxy | G6 |
| P.192 | chlorodifluoromethyl | trifluoromethyl | G6 |
| P.193 | difluoromethyl | bromo | G6 |
| P.194 | difluoromethyl | chloro | G6 |
| P.195 | difluoromethyl | cyano | G6 |
| P.196 | difluoromethyl | cyclopropyl | G6 |
| P.197 | difluoromethyl | ethyl | G6 |
| P.198 | difluoromethyl | fluoro | G6 |
| P.199 | difluoromethyl | hydrogen | G6 |
| P.200 | difluoromethyl | methoxy | G6 |
| P.201 | difluoromethyl | methyl | G6 |
| P.202 | difluoromethyl | nitro | G6 |
| P.203 | difluoromethyl | trifluoromethoxy | G6 |
| P.204 | difluoromethyl | trifluoromethyl | G6 |
| P.205 | trifluoromethyl | bromo | G6 |
| P.206 | trifluoromethyl | chloro | G6 |
| P.207 | trifluoromethyl | cyano | G6 |
| P.208 | trifluoromethyl | cyclopropyl | G6 |
| P.209 | trifluoromethyl | ethyl | G6 |
| P.210 | trifluoromethyl | fluoro | G6 |
| P.211 | trifluoromethyl | hydrogen | G6 |
| P.212 | trifluoromethyl | methoxy | G6 |
| P.213 | trifluoromethyl | methyl | G6 |
| P.214 | trifluoromethyl | nitro | G6 |
| P.215 | trifluoromethyl | trifluoromethoxy | G6 |
| P.216 | trifluoromethyl | trifluoromethyl | G6 |
| P.217 | chlorodifluoromethyl | bromo | G7 |
| P.218 | chlorodifluoromethyl | chloro | G7 |
| P.219 | chlorodifluoromethyl | cyano | G7 |
| P.220 | chlorodifluoromethyl | cyclopropyl | G7 |
| P.221 | chlorodifluoromethyl | ethyl | G7 |
| P.222 | chlorodifluoromethyl | fluoro | G7 |
| P.223 | chlorodifluoromethyl | hydrogen | G7 |
| P.224 | chlorodifluoromethyl | methoxy | G7 |
| P.225 | chlorodifluoromethyl | methyl | G7 |
| P.226 | chlorodifluoromethyl | nitro | G7 |
| P.227 | chlorodifluoromethyl | trifluoromethoxy | G7 |
| P.228 | chlorodifluoromethyl | trifluoromethyl | G7 |
| P.229 | difluoromethyl | bromo | G7 |
| P.230 | difluoromethyl | chloro | G7 |
| P.231 | difluoromethyl | cyano | G7 |
| P.232 | difluoromethyl | cyclopropyl | G7 |
| P.233 | difluoromethyl | ethyl | G7 |
| P.234 | difluoromethyl | fluoro | G7 |
| P.235 | difluoromethyl | hydrogen | G7 |
| P.236 | difluoromethyl | methoxy | G7 |
| P.237 | difluoromethyl | methyl | G7 |
| P.238 | difluoromethyl | nitro | G7 |
| P.239 | difluoromethyl | trifluoromethoxy | G7 |
| P.240 | difluoromethyl | trifluoromethyl | G7 |
| P.241 | trifluoromethyl | bromo | G7 |
| P.242 | trifluoromethyl | chloro | G7 |
| P.243 | trifluoromethyl | cyano | G7 |
| P.244 | trifluoromethyl | cyclopropyl | G7 |
| P.245 | trifluoromethyl | ethyl | G7 |
| P.246 | trifluoromethyl | fluoro | G7 |
| P.247 | trifluoromethyl | hydrogen | G7 |
| P.248 | trifluoromethyl | methoxy | G7 |
| P.249 | trifluoromethyl | methyl | G7 |
| P.250 | trifluoromethyl | nitro | G7 |
| P.251 | trifluoromethyl | trifluoromethoxy | G7 |
| P.252 | trifluoromethyl | trifluoromethyl | G7 |
| P.253 | chlorodifluoromethyl | bromo | G8 |
| P.254 | chlorodifluoromethyl | chloro | G8 |
| P.255 | chlorodifluoromethyl | cyano | G8 |
| P.256 | chlorodifluoromethyl | cyclopropyl | G8 |
| P.257 | chlorodifluoromethyl | ethyl | G8 |
| P.258 | chlorodifluoromethyl | fluoro | G8 |
| P.259 | chlorodifluoromethyl | hydrogen | G8 |
| P.260 | chlorodifluoromethyl | methoxy | G8 |
| P.261 | chlorodifluoromethyl | methyl | G8 |
| P.262 | chlorodifluoromethyl | nitro | G8 |
| P.263 | chlorodifluoromethyl | trifluoromethoxy | G8 |
| P.264 | chlorodifluoromethyl | trifluoromethyl | G8 |
| P.265 | difluoromethyl | bromo | G8 |
| P.266 | difluoromethyl | chloro | G8 |
| P.267 | difluoromethyl | cyano | G8 |
| P.268 | difluoromethyl | cyclopropyl | G8 |
| P.269 | difluoromethyl | ethyl | G8 |
| P.270 | difluoromethyl | fluoro | G8 |
| P.271 | difluoromethyl | hydrogen | G8 |
| P.272 | difluoromethyl | methoxy | G8 |
| P.273 | difluoromethyl | methyl | G8 |
| P.274 | difluoromethyl | nitro | G8 |
| P.275 | difluoromethyl | trifluoromethoxy | G8 |
| P.276 | difluoromethyl | trifluoromethyl | G8 |
| P.277 | trifluoromethyl | bromo | G8 |
| P.278 | trifluoromethyl | chloro | G8 |
| P.279 | trifluoromethyl | cyano | G8 |
| P.280 | trifluoromethyl | cyclopropyl | G8 |
| P.281 | trifluoromethyl | ethyl | G8 |
| P.282 | trifluoromethyl | fluoro | G8 |
| P.283 | trifluoromethyl | hydrogen | G8 |
| P.284 | trifluoromethyl | methoxy | G8 |
| P.285 | trifluoromethyl | methyl | G8 |
| P.286 | trifluoromethyl | nitro | G8 |
| P.287 | trifluoromethyl | trifluoromethoxy | G8 |
| P.288 | trifluoromethyl | trifluoromethyl | G8 |
| P.289 | chlorodifluoromethyl | bromo | G9 |
| P.290 | chlorodifluoromethyl | chloro | G9 |
| P.291 | chlorodifluoromethyl | cyano | G9 |
| P.292 | chlorodifluoromethyl | cyclopropyl | G9 |
| P.293 | chlorodifluoromethyl | ethyl | G9 |
| P.294 | chlorodifluoromethyl | fluoro | G9 |
| P.295 | chlorodifluoromethyl | hydrogen | G9 |
| P.296 | chlorodifluoromethyl | methoxy | G9 |
| P.297 | chlorodifluoromethyl | methyl | G9 |
| P.298 | chlorodifluoromethyl | nitro | G9 |
| P.299 | chlorodifluoromethyl | trifluoromethoxy | G9 |
| P.300 | chlorodifluoromethyl | trifluoromethyl | G9 |
| P.301 | difluoromethyl | bromo | G9 |
| P.302 | difluoromethyl | chloro | G9 |
| P.303 | difluoromethyl | cyano | G9 |
| P.304 | difluoromethyl | cyclopropyl | G9 |
| P.305 | difluoromethyl | ethyl | G9 |
| P.306 | difluoromethyl | fluoro | G9 |
| P.307 | difluoromethyl | hydrogen | G9 |
| P.308 | difluoromethyl | methoxy | G9 |
| P.309 | difluoromethyl | methyl | G9 |
| P.310 | difluoromethyl | nitro | G9 |
| P.311 | difluoromethyl | trifluoromethoxy | G9 |
| P.312 | difluoromethyl | trifluoromethyl | G9 |
| P.313 | trifluoromethyl | bromo | G9 |
| P.314 | trifluoromethyl | chloro | G9 |
| P.315 | trifluoromethyl | cyano | G9 |
| P.316 | trifluoromethyl | cyclopropyl | G9 |
| P.317 | trifluoromethyl | ethyl | G9 |
| P.318 | trifluoromethyl | fluoro | G9 |
| P.319 | trifluoromethyl | hydrogen | G9 |
| P.320 | trifluoromethyl | methoxy | G9 |
| P.321 | trifluoromethyl | methyl | G9 |
| P.322 | trifluoromethyl | nitro | G9 |
| P.323 | trifluoromethyl | trifluoromethoxy | G9 |
| P.324 | trifluoromethyl | trifluoromethyl | G9 |
| P.325 | chlorodifluoromethyl | bromo | G10 |
| P.326 | chlorodifluoromethyl | chloro | G10 |
| P.327 | chlorodifluoromethyl | cyano | G10 |
| P.328 | chlorodifluoromethyl | cyclopropyl | G10 |
| P.329 | chlorodifluoromethyl | ethyl | G10 |
| P.330 | chlorodifluoromethyl | fluoro | G10 |
| P.331 | chlorodifluoromethyl | hydrogen | G10 |
| P.332 | chlorodifluoromethyl | methoxy | G10 |
| P.333 | chlorodifluoromethyl | methyl | G10 |
| P.334 | chlorodifluoromethyl | nitro | G10 |
| P.335 | chlorodifluoromethyl | trifluoromethoxy | G10 |
| P.336 | chlorodifluoromethyl | trifluoromethyl | G10 |
| P.337 | difluoromethyl | bromo | G10 |
| P.338 | difluoromethyl | chloro | G10 |
| P.339 | difluoromethyl | cyano | G10 |
| P.340 | difluoromethyl | cyclopropyl | G10 |
| P.341 | difluoromethyl | ethyl | G10 |
| P.342 | difluoromethyl | fluoro | G10 |
| P.343 | difluoromethyl | hydrogen | G10 |
| P.344 | difluoromethyl | methoxy | G10 |
| P.345 | difluoromethyl | methyl | G10 |
| P.346 | difluoromethyl | nitro | G10 |
| P.347 | difluoromethyl | trifluoromethoxy | G10 |
| P.348 | difluoromethyl | trifluoromethyl | G10 |
| P.349 | trifluoromethyl | bromo | G10 |
| P.350 | trifluoromethyl | chloro | G10 |
| P.351 | trifluoromethyl | cyano | G10 |
| P.352 | trifluoromethyl | cyclopropyl | G10 |
| P.353 | trifluoromethyl | ethyl | G10 |
| P.354 | trifluoromethyl | fluoro | G10 |
| P.355 | trifluoromethyl | hydrogen | G10 |
| P.356 | trifluoromethyl | methoxy | G10 |
| P.357 | trifluoromethyl | methyl | G10 |
| P.358 | trifluoromethyl | nitro | G10 |
| P.359 | trifluoromethyl | trifluoromethoxy | G10 |
| P.360 | trifluoromethyl | trifluoromethyl | G10 |
| P.361 | chlorodifluoromethyl | bromo | G11 |
| P.362 | chlorodifluoromethyl | chloro | G11 |
| P.363 | chlorodifluoromethyl | cyano | G11 |
| P.364 | chlorodifluoromethyl | cyclopropyl | G11 |
| P.365 | chlorodifluoromethyl | ethyl | G11 |
| P.366 | chlorodifluoromethyl | fluoro | G11 |
| P.367 | chlorodifluoromethyl | hydrogen | G11 |
| P.368 | chlorodifluoromethyl | methoxy | G11 |
| P.369 | chlorodifluoromethyl | methyl | G11 |
| P.370 | chlorodifluoromethyl | nitro | G11 |
| P.371 | chlorodifluoromethyl | trifluoromethoxy | G11 |
| P.372 | chlorodifluoromethyl | trifluoromethyl | G11 |
| P.373 | difluoromethyl | bromo | G11 |
| P.374 | difluoromethyl | chloro | G11 |
| P.375 | difluoromethyl | cyano | G11 |
| P.376 | difluoromethyl | cyclopropyl | G11 |
| P.377 | difluoromethyl | ethyl | G11 |
| P.378 | difluoromethyl | fluoro | G11 |
| P.379 | difluoromethyl | hydrogen | G11 |
| P.380 | difluoromethyl | methoxy | G11 |
| P.381 | difluoromethyl | methyl | G11 |
| P.382 | difluoromethyl | nitro | G11 |
| P.383 | difluoromethyl | trifluoromethoxy | G11 |
| P.384 | difluoromethyl | trifluoromethyl | G11 |
| P.385 | trifluoromethyl | bromo | G11 |
| P.386 | trifluoromethyl | chloro | G11 |
| P.387 | trifluoromethyl | cyano | G11 |
| P.388 | trifluoromethyl | cyclopropyl | G11 |
| P.389 | trifluoromethyl | ethyl | G11 |
| P.390 | trifluoromethyl | fluoro | G11 |
| P.391 | trifluoromethyl | hydrogen | G11 |
| P.392 | trifluoromethyl | methoxy | G11 |
| P.393 | trifluoromethyl | methyl | G11 |
| P.394 | trifluoromethyl | nitro | G11 |
| P.395 | trifluoromethyl | trifluoromethoxy | G11 |
| P.396 | trifluoromethyl | trifluoromethyl | G11 |
| P.397 | chlorodifluoromethyl | bromo | G12 |
| P.398 | chlorodifluoromethyl | chloro | G12 |
| P.399 | chlorodifluoromethyl | cyano | G12 |
| P.400 | chlorodifluoromethyl | cyclopropyl | G12 |
| P.401 | chlorodifluoromethyl | ethyl | G12 |
| P.402 | chlorodifluoromethyl | fluoro | G12 |
| P.403 | chlorodifluoromethyl | hydrogen | G12 |
| P.404 | chlorodifluoromethyl | methoxy | G12 |
| P.405 | chlorodifluoromethyl | methyl | G12 |
| P.406 | chlorodifluoromethyl | nitro | G12 |
| P.407 | chlorodifluoromethyl | trifluoromethoxy | G12 |
| P.408 | chlorodifluoromethyl | trifluoromethyl | G12 |
| P.409 | difluoromethyl | bromo | G12 |
| P.410 | difluoromethyl | chloro | G12 |
| P.411 | difluoromethyl | cyano | G12 |
| P.412 | difluoromethyl | cyclopropyl | G12 |
| P.413 | difluoromethyl | ethyl | G12 |
| P.414 | difluoromethyl | fluoro | G12 |
| P.415 | difluoromethyl | hydrogen | G12 |
| P.416 | difluoromethyl | methoxy | G12 |
| P.417 | difluoromethyl | methyl | G12 |
| P.418 | difluoromethyl | nitro | G12 |
| P.419 | difluoromethyl | trifluoromethoxy | G12 |
| P.420 | difluoromethyl | trifluoromethyl | G12 |
| P.421 | trifluoromethyl | bromo | G12 |
| P.422 | trifluoromethyl | chloro | G12 |
| P.423 | trifluoromethyl | cyano | G12 |
| P.424 | trifluoromethyl | cyclopropyl | G12 |
| P.425 | trifluoromethyl | ethyl | G12 |
| P.426 | trifluoromethyl | fluoro | G12 |
| P.427 | trifluoromethyl | hydrogen | G12 |
| P.428 | trifluoromethyl | methoxy | G12 |
| P.429 | trifluoromethyl | methyl | G12 |
| P.430 | trifluoromethyl | nitro | G12 |
| P.431 | trifluoromethyl | trifluoromethoxy | G12 |
| P.432 | trifluoromethyl | trifluoromethyl | G12 |
| P.433 | chlorodifluoromethyl | bromo | G13 |
| P.434 | chlorodifluoromethyl | chloro | G13 |
| P.435 | chlorodifluoromethyl | cyano | G13 |
| P.436 | chlorodifluoromethyl | cyclopropyl | G13 |
| P.437 | chlorodifluoromethyl | ethyl | G13 |
| P.438 | chlorodifluoromethyl | fluoro | G13 |
| P.439 | chlorodifluoromethyl | hydrogen | G13 |
| P.440 | chlorodifluoromethyl | methoxy | G13 |
| P.441 | chlorodifluoromethyl | methyl | G13 |
| P.442 | chlorodifluoromethyl | nitro | G13 |
| P.443 | chlorodifluoromethyl | trifluoromethoxy | G13 |
| P.444 | chlorodifluoromethyl | trifluoromethyl | G13 |
| P.445 | difluoromethyl | bromo | G13 |
| P.446 | difluoromethyl | chloro | G13 |
| P.447 | difluoromethyl | cyano | G13 |
| P.448 | difluoromethyl | cyclopropyl | G13 |
| P.449 | difluoromethyl | ethyl | G13 |
| P.450 | difluoromethyl | fluoro | G13 |
| P.451 | difluoromethyl | hydrogen | G13 |
| P.452 | difluoromethyl | methoxy | G13 |
| P.453 | difluoromethyl | methyl | G13 |
| P.454 | difluoromethyl | nitro | G13 |
| P.455 | difluoromethyl | trifluoromethoxy | G13 |
| P.456 | difluoromethyl | trifluoromethyl | G13 |
| P.457 | trifluoromethyl | bromo | G13 |
| P.458 | trifluoromethyl | chloro | G13 |
| P.459 | trifluoromethyl | cyano | G13 |
| P.460 | trifluoromethyl | cyclopropyl | G13 |
| P.461 | trifluoromethyl | ethyl | G13 |
| P.462 | trifluoromethyl | fluoro | G13 |
| P.463 | trifluoromethyl | hydrogen | G13 |
| P.464 | trifluoromethyl | methoxy | G13 |
| P.465 | trifluoromethyl | methyl | G13 |
| P.466 | trifluoromethyl | nitro | G13 |
| P.467 | trifluoromethyl | trifluoromethoxy | G13 |
| P.468 | trifluoromethyl | trifluoromethyl | G13 |
| P.469 | chlorodifluoromethyl | bromo | G14 |
| P.470 | chlorodifluoromethyl | chloro | G14 |
| P.471 | chlorodifluoromethyl | cyano | G14 |
| P.472 | chlorodifluoromethyl | cyclopropyl | G14 |
| P.473 | chlorodifluoromethyl | ethyl | G14 |
| P.474 | chlorodifluoromethyl | fluoro | G14 |
| P.475 | chlorodifluoromethyl | hydrogen | G14 |
| P.476 | chlorodifluoromethyl | methoxy | G14 |
| P.477 | chlorodifluoromethyl | methyl | G14 |
| P.478 | chlorodifluoromethyl | nitro | G14 |
| P.479 | chlorodifluoromethyl | trifluoromethoxy | G14 |
| P.480 | chlorodifluoromethyl | trifluoromethyl | G14 |
| P.481 | difluoromethyl | bromo | G14 |
| P.482 | difluoromethyl | chloro | G14 |
| P.483 | difluoromethyl | cyano | G14 |
| P.484 | difluoromethyl | cyclopropyl | G14 |
| P.485 | difluoromethyl | ethyl | G14 |
| P.486 | difluoromethyl | fluoro | G14 |
| P.487 | difluoromethyl | hydrogen | G14 |
| P.488 | difluoromethyl | methoxy | G14 |
| P.489 | difluoromethyl | methyl | G14 |
| P.490 | difluoromethyl | nitro | G14 |
| P.491 | difluoromethyl | trifluoromethoxy | G14 |
| P.492 | difluoromethyl | trifluoromethyl | G14 |
| P.493 | trifluoromethyl | bromo | G14 |
| P.494 | trifluoromethyl | chloro | G14 |
| P.495 | trifluoromethyl | cyano | G14 |
| P.496 | trifluoromethyl | cyclopropyl | G14 |
| P.497 | trifluoromethyl | ethyl | G14 |
| P.498 | trifluoromethyl | fluoro | G14 |
| P.499 | trifluoromethyl | hydrogen | G14 |
| P.500 | trifluoromethyl | methoxy | G14 |
| P.501 | trifluoromethyl | methyl | G14 |
| P.502 | trifluoromethyl | nitro | G14 |
| P.503 | trifluoromethyl | trifluoromethoxy | G14 |
| P.504 | trifluoromethyl | trifluoromethyl | G14 |
| P.505 | chlorodifluoromethyl | G15 | |
| P.506 | difluoromethyl | G15 | |
| P.507 | trifluoromethyl | G15 | |
| P.508 | chlorodifluoromethyl | G16 | |
| P.509 | difluoromethyl | G16 | |
| P.510 | trifluoromethyl | G16 | |
| P.511 | chlorodifluoromethyl | G17 | |
| P.512 | difluoromethyl | G17 | |
| P.513 | trifluoromethyl | G17 | |
| P.514 | chlorodifluoromethyl | G18 | |
| P.515 | difluoromethyl | G18 | |
| P.516 | trifluoromethyl | G18 | |
| P.517 | chlorodifluoromethyl | G19 | |
| P.518 | difluoromethyl | G19 | |
| P.519 | trifluoromethyl | G19 | |
| P.520 | chlorodifluoromethyl | G20 | |
| P.521 | difluoromethyl | G20 | |
| P.522 | trifluoromethyl | G20 | |
| P.523 | chlorodifluoromethyl | G21 | |
| P.524 | difluoromethyl | G21 | |
| P.525 | trifluoromethyl | G21 | |
| P.526 | chlorodifluoromethyl | G22 | |
| P.527 | difluoromethyl | G22 | |
| P.528 | trifluoromethyl | G22 | |
| P.529 | chlorodifluoromethyl | G23 | |
| P.530 | difluoromethyl | G23 | |
| P.531 | trifluoromethyl | G23 | |
| P.532 | chlorodifluoromethyl | G24 | |
| P.533 | difluoromethyl | G24 | |
| P.534 | trifluoromethyl | G24 | |
| P.535 | chlorodifluoromethyl | G25 | |
| P.536 | difluoromethyl | G25 | |
| P.537 | trifluoromethyl | G25 | |
| P.538 | chlorodifluoromethyl | G26 | |
| P.539 | difluoromethyl | G26 | |
| P.540 | trifluoromethyl | G26 | |
| P.541 | chlorodifluoromethyl | G27 | |
| P.542 | difluoromethyl | G27 | |
| P.543 | trifluoromethyl | G27 | |
| P.544 | chlorodifluoromethyl | G28 | |
| P.545 | difluoromethyl | G28 | |
| P.546 | trifluoromethyl | G28 | |
| P.547 | chlorodifluoromethyl | G29 | |
| P.548 | difluoromethyl | G29 | |
| P.549 | trifluoromethyl | G29 | |
| P.550 | chlorodifluoromethyl | G30 | |
| P.551 | difluoromethyl | G30 | |
| P.552 | trifluoromethyl | G30 | |
| P.553 | chlorodifluoromethyl | G31 | |
| P.554 | difluoromethyl | G31 | |
| P.555 | trifluoromethyl | G31 | |
| P.556 | chlorodifluoromethyl | G32 | |
| P.557 | difluoromethyl | G32 | |
| P.558 | trifluoromethyl | G32 | |
| P.559 | chlorodifluoromethyl | G33 | |
| P.560 | difluoromethyl | G33 | |
| P.561 | trifluoromethyl | G33 | |
| P.562 | chlorodifluoromethyl | G34 | |
| P.563 | difluoromethyl | G34 | |
| P.564 | trifluoromethyl | G34 | |
| P.565 | chlorodifluoromethyl | G35 | |
| P.566 | difluoromethyl | G35 | |
| P.567 | trifluoromethyl | G35 | |
| P.568 | chlorodifluoromethyl | G36 | |
| P.569 | difluoromethyl | G36 | |
| P.570 | trifluoromethyl | G36 | |
| P.571 | chlorodifluoromethyl | G37 | |
| P.572 | difluoromethyl | G37 | |
| P.573 | trifluoromethyl | G37 | |
| P.574 | chlorodifluoromethyl | G38 | |
| P.575 | difluoromethyl | G38 | |
| P.576 | trifluoromethyl | G38 | |
| P.577 | chlorodifluoromethyl | G39 | |
| P.578 | difluoromethyl | G39 | |
| P.579 | trifluoromethyl | G39 | |
| P.580 | chlorodifluoromethyl | G40 | |
| P.581 | difluoromethyl | G40 | |
| P.582 | trifluoromethyl | G40 | |
| P.583 | chlorodifluoromethyl | G41 | |
| P.584 | difluoromethyl | G41 | |
| P.585 | trifluoromethyl | G41 | |
| P.586 | chlorodifluoromethyl | G42 | |
| P.587 | difluoromethyl | G42 | |
| P.588 | trifluoromethyl | G42 | |
| P.589 | chlorodifluoromethyl | G43 | |
| P.590 | difluoromethyl | G43 | |
| P.591 | trifluoromethyl | G43 | |
| P.592 | chlorodifluoromethyl | G44 | |
| P.593 | difluoromethyl | G44 | |
| P.594 | trifluoromethyl | G44 | |
| P.595 | chlorodifluoromethyl | G45 | |
| P.596 | difluoromethyl | G45 | |
| P.597 | trifluoromethyl | G45 | |
| P.598 | chlorodifluoromethyl | G46 | |
| P.599 | difluoromethyl | G46 | |
| P.600 | trifluoromethyl | G46 | |
| P.601 | chlorodifluoromethyl | G47 | |
| P.602 | difluoromethyl | G47 | |
| P.603 | trifluoromethyl | G47 | |
| P.604 | chlorodifluoromethyl | G48 | |
| P.605 | difluoromethyl | G48 | |
| P.606 | trifluoromethyl | G48 | |
| P.607 | chlorodifluoromethyl | G49 | |
| P.608 | difluoromethyl | G49 | |
| P.609 | trifluoromethyl | G49 | |
| P.610 | chlorodifluoromethyl | G50 | |
| P.611 | difluoromethyl | G50 | |
| P.612 | trifluoromethyl | G50 | |
| P.613 | chlorodifluoromethyl | G51 | |
| P.614 | difluoromethyl | G51 | |
| P.615 | trifluoromethyl | G51 | |
| P.616 | chlorodifluoromethyl | G52 | |
| P.617 | difluoromethyl | G52 | |
| P.618 | trifluoromethyl | G52 | |
| P.619 | chlorodifluoromethyl | bromo | G53 |
| P.620 | chlorodifluoromethyl | chloro | G53 |
| P.621 | chlorodifluoromethyl | cyano | G53 |
| P.622 | chlorodifluoromethyl | cyclopropyl | G53 |
| P.623 | chlorodifluoromethyl | ethyl | G53 |
| P.624 | chlorodifluoromethyl | fluoro | G53 |
| P.625 | chlorodifluoromethyl | hydrogen | G53 |
| P.626 | chlorodifluoromethyl | methoxy | G53 |
| P.627 | chlorodifluoromethyl | methyl | G53 |
| P.628 | chlorodifluoromethyl | nitro | G53 |
| P.629 | chlorodifluoromethyl | trifluoromethoxy | G53 |
| P.630 | chlorodifluoromethyl | trifluoromethyl | G53 |
| P.631 | difluoromethyl | bromo | G53 |
| P.632 | difluoromethyl | chloro | G53 |
| P.633 | difluoromethyl | cyano | G53 |
| P.634 | difluoromethyl | cyclopropyl | G53 |
| P.635 | difluoromethyl | ethyl | G53 |
| P.636 | difluoromethyl | fluoro | G53 |
| P.637 | difluoromethyl | hydrogen | G53 |
| P.638 | difluoromethyl | methoxy | G53 |
| P.639 | difluoromethyl | methyl | G53 |
| P.640 | difluoromethyl | nitro | G53 |
| P.641 | difluoromethyl | trifluoromethoxy | G53 |
| P.642 | difluoromethyl | trifluoromethyl | G53 |
| P.643 | trifluoromethyl | bromo | G53 |
| P.644 | trifluoromethyl | chloro | G53 |
| P.645 | trifluoromethyl | cyano | G53 |
| P.646 | trifluoromethyl | cyclopropyl | G53 |
| P.647 | trifluoromethyl | ethyl | G53 |
| P.648 | trifluoromethyl | fluoro | G53 |
| P.649 | trifluoromethyl | hydrogen | G53 |
| P.650 | trifluoromethyl | methoxy | G53 |
| P.651 | trifluoromethyl | methyl | G53 |
| P.652 | trifluoromethyl | nitro | G53 |
| P.653 | trifluoromethyl | trifluoromethoxy | G53 |
| P.654 | trifluoromethyl | trifluoromethyl | G53 |
| P.655 | chlorodifluoromethyl | bromo | G54 |
| P.656 | chlorodifluoromethyl | chloro | G54 |
| P.657 | chlorodifluoromethyl | cyano | G54 |
| P.658 | chlorodifluoromethyl | cyclopropyl | G54 |
| P.659 | chlorodifluoromethyl | ethyl | G54 |
| P.660 | chlorodifluoromethyl | fluoro | G54 |
| P.661 | chlorodifluoromethyl | hydrogen | G54 |
| P.662 | chlorodifluoromethyl | methoxy | G54 |
| P.663 | chlorodifluoromethyl | methyl | G54 |
| P.664 | chlorodifluoromethyl | nitro | G54 |
| P.665 | chlorodifluoromethyl | trifluoromethoxy | G54 |
| P.666 | chlorodifluoromethyl | trifluoromethyl | G54 |
| P.667 | difluoromethyl | bromo | G54 |
| P.668 | difluoromethyl | chloro | G54 |
| P.669 | difluoromethyl | cyano | G54 |
| P.670 | difluoromethyl | cyclopropyl | G54 |
| P.671 | difluoromethyl | ethyl | G54 |
| P.672 | difluoromethyl | fluoro | G54 |
| P.673 | difluoromethyl | hydrogen | G54 |
| P.674 | difluoromethyl | methoxy | G54 |
| P.675 | difluoromethyl | methyl | G54 |
| P.676 | difluoromethyl | nitro | G54 |
| P.677 | difluoromethyl | trifluoromethoxy | G54 |
| P.678 | difluoromethyl | trifluoromethyl | G54 |
| P.679 | trifluoromethyl | bromo | G54 |
| P.680 | trifluoromethyl | chloro | G54 |
| P.681 | trifluoromethyl | cyano | G54 |
| P.682 | trifluoromethyl | cyclopropyl | G54 |
| P.683 | trifluoromethyl | ethyl | G54 |
| P.684 | trifluoromethyl | fluoro | G54 |
| P.685 | trifluoromethyl | hydrogen | G54 |
| P.686 | trifluoromethyl | methoxy | G54 |
| P.687 | trifluoromethyl | methyl | G54 |
| P.688 | trifluoromethyl | nitro | G54 |
| P.689 | trifluoromethyl | trifluoromethoxy | G54 |
| P.690 | trifluoromethyl | trifluoromethyl | G54 |

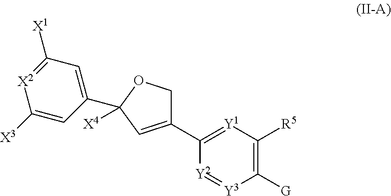

### Table 91P:

Table 91 P provides 690 compounds of formula (II-A) wherein X¹ is chloro, X² is CH, X³ is chloro, Y¹ is CH, Y² is CH, Y³ is CH and X⁴, R⁵ and G have the values listed in the table P.

### Table 92 P:

Table 92 P provides 690 compounds of formula (II-A) wherein X¹ is chloro, X² is CH, X³ is chloro, Y¹ is N, Y² is CH, Y³ is CH and X⁴, R⁵ and G have the values listed in the table P.

### Table 93 P:

Table 93 P provides 690 compounds of formula (II-A) wherein X¹ is chloro, X² is CH, X³ is chloro, Y¹ is N, Y² is N, Y³ is CH and X⁴, R⁵ and G have the values listed in the table P.

### Table 94 P:

Table 94 P provides 690 compounds of formula (II-A) wherein X¹ is chloro, X² is CH, X³ is chloro, Y¹ is CH, Y² is N, Y³ is CH and X⁴, R⁵ and G have the values listed in the table P.

### Table 95 P:

Table 95 P provides 690 compounds of formula (II-A) wherein X¹ is chloro, X² is CH, X³ is chloro, Y¹ is

CH, Y² is CH, Y³ is N and X⁴, R⁵ and G have the values listed in the table P.

### Table 96 P:

Table 96 P provides 690 compounds of formula (II-A) wherein X¹ is chloro, X² is C—F, X³ is hydrogen, Y¹ is CH, Y² is CH, Y³ is CH and X⁴, R⁵ and G have the values listed in the table P.

### Table 97 P:

Table 97 P provides 690 compounds of formula (II-A) wherein X¹ is chloro, X² is C—F, X³ is hydrogen, Y¹ is N, Y² is CH, Y³ is CH and X⁴, R⁵ and G have the values listed in the table P.

### Table 98 P:

Table 98 P provides 690 compounds of formula (II-A) wherein X¹ is chloro, X² is C—F, X³ is hydrogen, Y¹ is N, Y² is N, Y³ is CH and X⁴, R⁵ and G have the values listed in the table P.

### Table 99 P:

Table 99 P provides 690 compounds of formula (II-A) wherein X¹ is chloro, X² is C—F, X³ is hydrogen, Y¹ is CH, Y² is N, Y³ is CH and X⁴, R⁵ and G have the values listed in the table P.

### Table 100 P:

Table 100 P provides 690 compounds of formula (II-A) wherein X¹ is chloro, X² is C—F, X³ is hydrogen, Y¹ is CH, Y² is CH, Y³ is N and X⁴, R⁵ and G have the values listed in the table P.

### Table 101 P:

Table 101 P provides 690 compounds of formula (II-A) wherein X¹ is fluoro, X² is C—Cl, X³ is hydrogen, Y¹ is CH, Y² is CH, Y³ is CH and X⁴, R⁵ and G have the values listed in the table P.

### Table 102 P:

Table 102 P provides 690 compounds of formula (II-A) wherein X¹ is fluoro, X² is C—Cl, X³ is hydrogen, Y¹ is N, Y² is CH, Y³ is CH and X⁴, R⁵ and G have the values listed in the table P.

### Table 103 P:

Table 103 P provides 690 compounds of formula (II-A) wherein X¹ is fluoro, X² is C—Cl, X³ is hydrogen, Y¹ is N, Y² is N, Y³ is CH and X⁴, R⁵ and G have the values listed in the table P.

### Table 104 P:

Table 104 P provides 690 compounds of formula (II-A) wherein X¹ is fluoro, X² is C—Cl, X³ is hydrogen, Y¹ is CH, Y² is N, Y³ is CH and X⁴, R⁵ and G have the values listed in the table P.

### Table 105 P:

Table 105 P provides 690 compounds of formula (II-A) wherein X¹ is fluoro, X² is C—Cl, X³ is hydrogen,

Y¹ is CH, Y² is CH, Y³ is N and X⁴, R⁵ and G have the values listed in the table P.

### Table 106 P:

Table 106 P provides 690 compounds of formula (II-A) wherein X¹ is chloro, X² is C—Cl, X³ is hydrogen, Y¹ is CH, Y² is CH, Y³ is CH and X⁴, R⁵ and G have the values listed in the table P.

### Table 107 P:

Table 107 P provides 690 compounds of formula (II-A) wherein X¹ is chloro, X² is C—Cl, X³ is hydrogen, Y¹ is N, Y² is CH, Y³ is CH and X⁴, R⁵ and G have the values listed in the table P.

### Table 108 P:

Table 108 P provides 690 compounds of formula (II-A) wherein X¹ is chloro, X² is C—Cl, X³ is hydrogen, Y¹ is N, Y² is N, Y³ is CH and X⁴, R⁵ and G have the values listed in the table P.

### Table 109 P:

Table 109 P provides 690 compounds of formula (II-A) wherein X¹ is chloro, X² is C—Cl, X³ is hydrogen, Y¹ is CH, Y² is N, Y³ is CH and X⁴, R⁵ and G have the values listed in the table P.

### Table 110 P:

Table 110 P provides 690 compounds of formula (II-A) wherein X¹ is chloro, X² is C—Cl, X³ is hydrogen, Y¹ is CH, Y² is CH, Y³ is N and X⁴, R⁵ and G have the values listed in the table P.

### Table 111P:

Table 111 P provides 690 compounds of formula (II-A) wherein X¹ is chloro, X² is C—F, X³ is chloro, Y¹ is CH, Y² is CH, Y³ is CH and X⁴, R⁵ and G have the values listed in the table P. Table 112 P:
Table 112 P provides 690 compounds of formula (II-A) wherein X¹ is chloro, X² is C—F, X³ is chloro, Y¹ is N, Y² is CH, Y³ is CH and X⁴, R⁵ and G have the values listed in the table P.

### Table 113 P:

Table 113 P provides 690 compounds of formula (II-A) wherein X¹ is chloro, X² is C—F, X³ is chloro, Y¹ is N, Y² is N, Y³ is CH and X⁴, R⁵ and G have the values listed in the table P.

### Table 114 P:

Table 114 P provides 690 compounds of formula (II-A) wherein X¹ is chloro, X² is C—F, X³ is chloro, Y¹ is CH, Y² is N, Y³ is CH and X⁴, R⁵ and G have the values listed in the table P.

### Table 115 P:

Table 115 P provides 690 compounds of formula (II-A) wherein X¹ is chloro, X² is C—F, X³ is chloro, Y¹ is CH, Y² is CH, Y³ is N and X⁴, R⁵ and G have the values listed in the table P.

### Table 116 P:

Table 116 P provides 690 compounds of formula (II-A) wherein X¹ is chloro, X² is C—Cl, X³ is chloro, Y¹ is CH, Y² is CH, Y³ is CH and X⁴, R⁵ and G have the values listed in the table P.

### Table 117 P:

Table 27 P provides 690 compounds of formula (II-A) wherein X¹ is chloro, X² is C—Cl, X³ is chloro, Y¹ is N, Y² is CH, Y³ is CH and X⁴, R⁵ and G have the values listed in the table P.

### Table 118 P:

Table 118 P provides 690 compounds of formula (II-A) wherein X¹ is chloro, X² is C—Cl, X³ is chloro, Y¹ is N, Y² is N, Y³ is CH and X⁴, R⁵ and G have the values listed in the table P.

### Table 119 P:

Table 119 P provides 690 compounds of formula (II-A) wherein X¹ is chloro, X² is C—Cl, X³ is chloro, Y¹ is CH, Y² is N, Y³ is CH and X⁴, R⁵ and G have the values listed in the table P.

### Table 120 P:

Table 120 P provides 690 compounds of formula (II-A) wherein X¹ is chloro, X² is C—Cl, X³ is chloro, Y¹ is CH, Y² is CH, Y³ is N and X⁴, R⁵ and G have the values listed in the table P.

### Table 121 P:

Table 121 P provides 690 compounds of formula (II-A) wherein X¹ is chloro, X² is C—Br, X³ is chloro, Y¹ is CH, Y² is CH, Y³ is CH and X⁴, R⁵ and G have the values listed in the table P.

### Table 122 P:

Table 122 P provides 690 compounds of formula (II-A) wherein X¹ is chloro, X² is C—Br, X³ is chloro, Y¹ is N, Y² is CH, Y³ is CH and X⁴, R⁵ and G have the values listed in the table P.

### Table 123 P:

Table 123 P provides 690 compounds of formula (II-A) wherein X¹ is chloro, X² is C—Br, X³ is chloro, Y¹ is N, Y² is N, Y³ is CH and X⁴, R⁵ and G have the values listed in the table P.

### Table 124 P:

Table 124 P provides 690 compounds of formula (II-A) wherein X¹ is chloro, X² is C—Br, X³ is chloro, Y¹ is CH, Y² is N, Y³ is CH and X⁴, R⁵ and G have the values listed in the table P.

### Table 125 P:

Table 125 P provides 690 compounds of formula (II-A) wherein X¹ is chloro, X² is C—Br, X³ is chloro, Y¹ is CH, Y² is CH, Y³ is N and X⁴, R⁵ and G have the values listed in the table P.

### Table 126 P:

Table 126 P provides 690 compounds of formula (II-A) wherein X¹ is chloro, X² is C—I, X³ is chloro, Y¹ is CH, Y² is CH, Y³ is CH and X⁴, R⁵ and G have the values listed in the table P.

### Table 127 P:

Table 127 P provides 690 compounds of formula (II-A) wherein X¹ is chloro, X² is C—I, X³ is chloro, Y¹ is N, Y² is CH, Y³ is CH and X⁴, R⁵ and G have the values listed in the table P.

### Table 128 P:

Table 128 P provides 690 compounds of formula (II-A) wherein X¹ is chloro, X² is C—I, X³ is chloro, Y¹ is N, Y² is N, Y³ is CH and X⁴, R⁵ and G have the values listed in the table P.

### Table 129 P:

Table 129 P provides 690 compounds of formula (II-A) wherein X¹ is chloro, X² is C—I, X³ is chloro, Y¹ is CH, Y² is N, Y³ is CH and X⁴, R⁵ and G have the values listed in the table P.

### Table 130 P:

Table 130 P provides 690 compounds of formula (II-A) wherein X¹ is chloro, X² is C—I, X³ is chloro, Y¹ is CH, Y² is CH, Y³ is N and X⁴, R⁵ and G have the values listed in the table P.

### Table 131 P:

Table 131 P provides 690 compounds of formula (II-A) wherein X¹ is fluoro, X² is C—F, X³ is fluoro, Y¹ is CH, Y² is CH, Y³ is CH and X⁴, R⁵ and G have the values listed in the table P.

### Table 132 P:

Table 132 P provides 690 compounds of formula (II-A) wherein X¹ is fluoro, X² is C—F, X³ is fluoro, Y¹ is

N, Y² is CH, Y³ is CH and X⁴, R⁵ and G have the values listed in the table P.

### Table 133 P:

Table 133 P provides 690 compounds of formula (II-A) wherein X¹ is fluoro, X² is C—F, X³ is fluoro, Y¹ is N, Y² is N, Y³ is CH and X⁴, R⁵ and G have the values listed in the table P.

### Table 134 P:

Table 134 P provides 690 compounds of formula (II-A) wherein X¹ is fluoro, X² is C—F, X³ is fluoro, Y¹ is CH, Y² is N, Y³ is CH and X⁴, R⁵ and G have the values listed in the table P.

### Table 135 P:

Table 135 P provides 690 compounds of formula (II-A) wherein X¹ is fluoro, X² is C—F, X³ is fluoro, Y¹ is CH, Y² is CH, Y³ is N and X⁴, R⁵ and G have the values listed in the table P.

### Table 136 P:

Table 136 P provides 690 compounds of formula (II-A) wherein X¹ is chloro, X² is CH, X³ is bromo, Y¹ is CH, Y² is CH, Y³ is CH and X⁴, R⁵ and G have the values listed in the table P.

### Table 137 P:

Table 137 P provides 690 compounds of formula (II-A) wherein X¹ is chloro, X² is CH, X³ is bromo, Y¹ is N, Y² is CH, Y³ is CH and X⁴, R⁵ and G have the values listed in the table P.

### Table 138 P:

Table 138 P provides 690 compounds of formula (II-A) wherein X¹ is chloro, X² is CH, X³ is bromo, Y¹ is N, Y² is N, Y³ is CH and X⁴, R⁵ and G have the values listed in the table P.

### Table 139 P:

Table 139 P provides 690 compounds of formula (II-A) wherein X¹ is chloro, X² is CH, X³ is bromo, Y¹ is CH, Y² is N, Y³ is CH and X⁴, R⁵ and G have the values listed in the table P.

### Table 140 P:

Table 140 P provides 690 compounds of formula (II-A) wherein X¹ is chloro, X² is CH, X³ is bromo, Y¹ is CH, Y² is CH, Y³ is N and X⁴, R⁵ and G have the values listed in the table P.

### Table 141 P:

Table 141 P provides 690 compounds of formula (II-A) wherein X¹ is chloro, X² is CH, X³ is fluoro, Y¹ is CH, Y² is CH, Y³ is CH and X⁴, R⁵ and G have the values listed in the table P.

### Table 142 P:

Table 142 P provides 690 compounds of formula (II-A) wherein X¹ is chloro, X² is CH, X³ is fluoro, Y¹ is N, Y² is CH, Y³ is CH and X⁴, R⁵ and G have the values listed in the table P.

### Table 143 P:

Table 143 P provides 690 compounds of formula (II-A) wherein X¹ is chloro, X² is CH, X³ is fluoro, Y¹ is N, Y² is N, Y³ is CH and X⁴, R⁵ and G have the values listed in the table P.

### Table 144 P:

Table 144 P provides 690 compounds of formula (II-A) wherein X¹ is chloro, X² is CH, X³ is fluoro, Y¹ is CH, Y² is N, Y³ is CH and X⁴, R⁵ and G have the values listed in the table P.

### Table 145 P:

Table 145 P provides 690 compounds of formula (II-A) wherein X¹ is chloro, X² is CH, X³ is fluoro, Y¹ is CH, Y² is CH, Y³ is N and X⁴, R⁵ and G have the values listed in the table P.

### Table 146 P:

Table 146 P provides 690 compounds of formula (II-A) wherein X¹ is chloro, X² is CH, X³ is trifluoromethyl, Y¹ is CH, Y² is CH, Y³ is CH and X⁴, R⁵ and G have the values listed in the table P.
Table 147 P: 50 Table 147 P provides 690 compounds of formula (II-A) wherein X¹ is chloro, X² is CH, X³ is trifluoromethyl, Y¹ is N, Y² is CH, Y³ is CH and X⁴, R⁵ and G have the values listed in the table P.

### Table 148 P:

Table 148 P provides 690 compounds of formula (II-A) wherein X¹ is chloro, X² is CH, X³ is trifluoromethyl, Y¹ is N, Y² is N, Y³ is CH and X⁴, R⁵ and G have the values listed in the table P.

### Table 149 P:

Table 149 P provides 690 compounds of formula (II-A) wherein X¹ is chloro, X² is CH, X³ is trifluoromethyl, Y¹ is CH, Y² is N, Y³ is CH and X⁴, R⁵ and G have the values listed in the table P.

### Table 150 P:

Table 150 P provides 690 compounds of formula (II-A) wherein X¹ is chloro, X² is CH, X³ is trifluoromethyl, Y¹ is CH, Y² is CH, Y³ is N and X⁴, R⁵ and G have the values listed in the table P.

### Table 151 P:

Table 151 P provides 690 compounds of formula (II-A) wherein X¹ is chloro, X² is C—Cl, X³ is trifluoromethyl, Y¹ is CH, Y² is CH, Y³ is CH and X⁴, R⁵ and G have the values listed in the table P.

### Table 152 P:

Table 152 P provides 690 compounds of formula (II-A) wherein X¹ is chloro, X² is C—Cl, X³ is trifluoromethyl, Y¹ is N, Y² is CH, Y³ is CH and X⁴, R⁵ and G have the values listed in the table P.

### Table 153 P:

Table 153 P provides 690 compounds of formula (II-A) wherein X¹ is chloro, X² is C—Cl, X³ is trifluoromethyl, Y¹ is N, Y² is N, Y³ is CH and X⁴, R⁵ and G have the values listed in the table P.

### Table 154 P:

Table 154 P provides 690 compounds of formula (II-A) wherein X¹ is chloro, X² is C—Cl, X³ is trifluoromethyl, Y¹ is CH, Y² is N, Y³ is CH and X⁴, R⁵ and G have the values listed in the table P.

### Table 155 P:

Table 155 P provides 690 compounds of formula (II-A) wherein X¹ is chloro, X² is C—Cl, X³ is trifluoromethyl, Y¹ is CH, Y² is CH, Y³ is N and X⁴, R⁵ and G have the values listed in the table P.

### Table 156 P:

Table 156 P provides 690 compounds of formula (II-A) wherein X¹ is trifluoromethyl, X² is CH, X³ is trifluoromethyl, Y¹ is CH, Y² is CH, Y³ is CH and X⁴, R⁵ and G have the values listed in the table P.

### Table 157 P:

Table 157 P provides 690 compounds of formula (II-A) wherein X¹ is trifluoromethyl, X² is CH, X³ is trifluoromethyl, Y¹ is N, Y² is CH, Y³ is CH and X⁴, R⁵ and G have the values listed in the table P.

### Table 158 P:

Table 158 P provides 690 compounds of formula (II-A) wherein X¹ is trifluoromethyl, X² is CH, X³ is trifluoromethyl, Y¹ is N, Y² is N, Y³ is CH and X⁴, R⁵ and G have the values listed in the table P.

### Table 159 P:

Table 159 P provides 690 compounds of formula (II-A) wherein X¹ is trifluoromethyl, X² is CH, X³ is trifluoromethyl, Y¹ is CH, Y² is N, Y³ is CH and X⁴, R⁵ and G have the values listed in the table P.

### Table 160 P:

Table 160 P provides 690 compounds of formula (II-A) wherein X¹ is trifluoromethyl, X² is CH, X³ is trifluoromethyl, Y¹ is CH, Y² is CH, Y³ is N and X⁴, R⁵ and G have the values listed in the table P.

### Table 161 P:

Table 161 P provides 690 compounds of formula (II-A) wherein X¹ is trifluoromethyl, X² is C—Cl, X³ is trifluoromethyl, Y¹ is CH, Y² is CH, Y³ is CH and X⁴, R⁵ and G have the values listed in the table P.

### Table 162 P:

Table 162 P provides 690 compounds of formula (II-A) wherein X¹ is trifluoromethyl, X² is C—Cl, X³ is trifluoromethyl, Y¹ is N, Y² is CH, Y³ is CH and X⁴, R⁵ and G have the values listed in the table P.

### Table 163 P:

Table 163 P provides 690 compounds of formula (II-A) wherein X¹ is trifluoromethyl, X² is C—Cl, X³ is trifluoromethyl, Y¹ is N, Y² is N, Y³ is CH and X⁴, R⁵ and G have the values listed in the table P.

### Table 164 P:

Table 164 P provides 690 compounds of formula (II-A) wherein X¹ is trifluoromethyl, X² is C—Cl, X³ is trifluoromethyl, Y¹ is CH, Y² is N, Y³ is CH and X⁴, R⁵ and G have the values listed in the table P.

### Table 165 P:

Table 165 P provides 690 compounds of formula (II-A) wherein X¹ is trifluoromethyl, X² is C—Cl, X³ is trifluoromethyl, Y¹ is CH, Y² is CH, Y³ is N and X⁴, R⁵ and G have the values listed in the table P.

### Table 166 P:

Table 166 P provides 690 compounds of formula (II-A) wherein X¹ is trifluoromethyl, X² is CH, X³ is hydrogen, Y¹ is CH, Y² is CH, Y³ is CH and X⁴, R⁵ and G have the values listed in the table P.

### Table 167 P:

Table 167 P provides 690 compounds of formula (II-A) wherein X¹ is trifluoromethyl, X² is CH, X³ is hydrogen, Y¹ is N, Y² is CH, Y³ is CH and X⁴, R⁵ and G have the values listed in the table P.

### Table 168 P:

Table 168 P provides 690 compounds of formula (II-A) wherein X¹ is trifluoromethyl, X² is CH, X³ is hydrogen, Y¹ is N, Y² is N, Y³ is CH and X⁴, R⁵ and G have the values listed in the table P.

### Table 169 P:

Table 169 P provides 690 compounds of formula (II-A) wherein X¹ is trifluoromethyl, X² is CH, X³ is hydrogen, Y¹ is CH, Y² is N, Y³ is CH and X⁴, R⁵ and G have the values listed in the table P.

### Table 170 P:

Table 170 P provides 690 compounds of formula (II-A) wherein X¹ is trifluoromethyl, X² is CH, X³ is hydrogen, Y¹ is CH, Y² is CH, Y³ is N and X⁴, R⁵ and G have the values listed in the table P.

### Table 171 P:

Table 171 P provides 690 compounds of formula (II-A) wherein X¹ is chloro, X² is N, X³ is chloro, Y¹ is CH, Y² is CH, Y³ is CH and X⁴, R⁵ and G have the values listed in the table P.

### Table 172 P:

Table 172 P provides 690 compounds of formula (II-A) wherein X¹ is chloro, X² is N, X³ is chloro, Y¹ is N, Y² is CH, Y³ is CH and X⁴, R⁵ and G have the values listed in the table P.

### Table 173 P:

Table 173 P provides 690 compounds of formula (II-A) wherein X¹ is chloro, X² is N, X³ is chloro, Y¹ is N, Y² is N, Y³ is CH and X⁴, R⁵ and G have the values listed in the table P.

### Table 174 P:

Table 174 P provides 690 compounds of formula (II-A) wherein X¹ is chloro, X² is N, X³ is chloro, Y¹ is

CH, Y² is N, Y³ is CH and X⁴, R⁵ and G have the values listed in the table P.

### Table 175 P:

Table 175 P provides 690 compounds of formula (II-A) wherein X¹ is chloro, X² is N, X³ is chloro, Y¹ is CH, Y² is CH, Y³ is N and X⁴, R⁵ and G have the values listed in the table P.

### Table 176 P:

Table 176 P provides 690 compounds of formula (II-A) wherein X¹ is trifluoromethyl, X² is N, X³ is trifluoromethyl, Y¹ is CH, Y² is CH, Y³ is CH and X⁴, R⁵ and G have the values listed in the table P.

### Table 177 P:

Table 177 P provides 690 compounds of formula (II-A) wherein X¹ is trifluoromethyl, X² is N, X³ is trifluoromethyl, Y¹ is N, Y² is CH, Y³ is CH and X⁴, R⁵ and G have the values listed in the table P.

### Table 178 P:

Table 178 P provides 690 compounds of formula (II-A) wherein X¹ is trifluoromethyl, X² is N, X³ is trifluoromethyl, Y¹ is N, Y² is N, Y³ is CH and X⁴, R⁵ and G have the values listed in the table P.

### Table 179 P:

Table 179 P provides 690 compounds of formula (II-A) wherein X¹ is trifluoromethyl, X² is N, X³ is trifluoromethyl, Y¹ is CH, Y² is N, Y³ is CH and X⁴, R⁵ and G have the values listed in the table P.

### Table 180 P:

Table 180 P provides 690 compounds of formula (II-A) wherein X¹ is trifluoromethyl, X² is N, X³ is trifluoromethyl, Y¹ is CH, Y² is CH, Y³ is N and X⁴, R⁵ and G have the values listed in the table P.

TABLE Q

| | X4 | G |
|---|---|---|
| Q.001 | Chlorodifluoromethyl | G1 |
| Q.002 | difluoromethyl | G1 |
| Q.003 | trifluoromethyl | G1 |
| Q.004 | Chlorodifluoromethyl | G2 |
| Q.005 | difluoromethyl | G2 |
| Q.006 | trifluoromethyl | G2 |
| Q.007 | Chlorodifluoromethyl | G3 |
| Q.008 | difluoromethyl | G3 |
| Q.009 | trifluoromethyl | G3 |
| Q.010 | Chlorodifluoromethyl | G4 |
| Q.011 | difluoromethyl | G4 |
| Q.012 | trifluoromethyl | G4 |
| Q.013 | Chlorodifluoromethyl | G5 |
| Q.014 | difluoromethyl | G5 |
| Q.015 | trifluoromethyl | G5 |
| Q.016 | Chlorodifluoromethyl | G6 |
| Q.017 | difluoromethyl | G6 |
| Q.018 | trifluoromethyl | G6 |
| Q.019 | Chlorodifluoromethyl | G7 |
| Q.020 | difluoromethyl | G7 |
| Q.021 | trifluoromethyl | G7 |
| Q.022 | Chlorodifluoromethyl | G8 |
| Q.023 | difluoromethyl | G8 |
| Q.024 | trifluoromethyl | G8 |
| Q.025 | Chlorodifluoromethyl | G9 |
| Q.026 | difluoromethyl | G9 |
| Q.027 | trifluoromethyl | G9 |
| Q.028 | Chlorodifluoromethyl | G10 |
| Q.029 | difluoromethyl | G10 |
| Q.030 | trifluoromethyl | G10 |
| Q.031 | Chlorodifluoromethyl | G11 |
| Q.032 | difluoromethyl | G11 |
| Q.033 | trifluoromethyl | G11 |
| Q.034 | Chlorodifluoromethyl | G12 |
| Q.035 | difluoromethyl | G12 |
| Q.036 | trifluoromethyl | G12 |
| Q.037 | Chlorodifluoromethyl | G13 |
| Q.038 | difluoromethyl | G13 |
| Q.039 | trifluoromethyl | G13 |
| Q.040 | Chlorodifluoromethyl | G14 |
| Q.041 | difluoromethyl | G14 |
| Q.042 | trifluoromethyl | G14 |
| Q.043 | Chlorodifluoromethyl | G53 |
| Q.044 | difluoromethyl | G53 |
| Q.045 | trifluoromethyl | G53 |
| Q.046 | Chlorodifluoromethyl | G54 |
| Q.047 | difluoromethyl | G54 |
| Q.048 | trifluoromethyl | G54 |

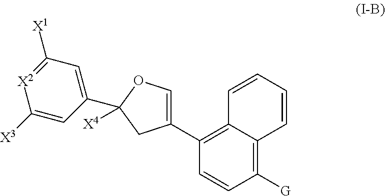

### Table 1Q

Table 1Q provides 48 compounds of formula (I-B) wherein X¹ is chloro, X² is CH, X³ is chloro, X⁴ and G have the values listed in the table Q.

### Table 2 Q

Table 2Q provides 48 compounds of formula (I-B) wherein X¹ is chloro, X² is C—F, X³ is hydrogen, X⁴ and G have the values listed in the table Q.

### Table 3 Q

Table 3Q provides 48 compounds of formula (I-B) wherein X¹ is fluoro, X² is C—Cl, X³ is hydrogen, X⁴ and G have the values listed in the table Q.

### Table 4 Q

Table 4Q provides 48 compounds of formula (I-B) wherein X¹ is chloro, X² is C—Cl, X³ is hydrogen, X⁴ and G have the values listed in the table Q.

### Table 5 Q

Table 5Q provides 48 compounds of formula (I-B) wherein X¹ is chloro, X² is C—Br, X³ is chloro, X⁴ and G have the values listed in the table Q.

### Table 6 Q

Table 6Q provides 48 compounds of formula (I-B) wherein X¹ is chloro, X² is C—F, X³ is chloro, X⁴ and G have the values listed in the table Q.

### Table 7 Q

Table 7Q provides 48 compounds of formula (I-B) wherein X¹ is chloro, X² is C—Cl, X³ is chloro, X⁴ and G have the values listed in the table Q.

### Table 8 Q

Table 8Q provides 48 compounds of formula (I-B) wherein X¹ is chloro, X² is C—I, X³ is chloro, X⁴ and G have the values listed in the table Q.

### Table 9 Q

Table 9Q provides 48 compounds of formula (I-B) wherein X¹ is fluoro, X² is C—F, X³ is fluoro, X⁴ and G have the values listed in the table Q.

### Table 10 Q

Table 10Q provides 48 compounds of formula (I-B) wherein X¹ is chloro, X² is CH, X³ is bromo, X⁴ and G have the values listed in the table Q.

### Table 11 Q

Table 11Q provides 48 compounds of formula (I-B) wherein X¹ is chloro, X² is CH, X³ is fluoro, X⁴ and G have the values listed in the table Q.

### Table 12 Q

Table 2Q provides 48 compounds of formula (I-B) wherein X¹ is chloro, X² is CH, X³ is trifluoromethyl, X⁴ and G have the values listed in the table Q.

### Table 13 Q

Table 13Q provides 48 compounds of formula (I-B) wherein X¹ is chloro, X² is C—Cl, X³ is trifluoromethyl, X⁴ and G have the values listed in the table Q.

### Table 14 Q

Table 14Q provides 48 compounds of formula (I-B) wherein X¹ is trifluoromethyl, X² is CH, X³ is trifluoromethyl, X⁴ and G have the values listed in the table Q.

### Table 15 Q

Table 15Q provides 48 compounds of formula (I-B) wherein X¹ is trifluoromethyl, X² is C—Cl, X³ is trifluoromethyl, X⁴ and G have the values listed in the table Q.

### Table 16 Q

Table 16Q provides 48 compounds of formula (I-B) wherein X¹ is trifluoromethyl, X² is CH, X³ is hydrogen, X⁴ and G have the values listed in the table Q.

### Table 17 Q

Table 17Q provides 48 compounds of formula (I-B) wherein X¹ is chloro, X² is N, X³ is chloro, X⁴ and G have the values listed in the table Q.

### Table 18 Q

Table 18Q provides 48 compounds of formula (I-B) wherein X¹ is trifluoromethyl, X² is N, X³ is trifluoromethyl, X⁴ and G have the values listed in the table Q.

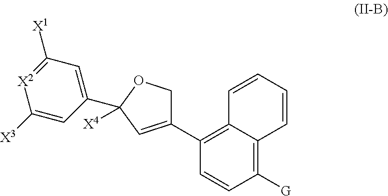

### Table 19 Q

Table 19Q provides 48 compounds of formula (II-B) wherein X¹ is chloro, X² is CH, X³ is chloro, X⁴ and G have the values listed in the table Q.

### Table 20 Q

Table 20Q provides 48 compounds of formula (II-B) wherein X¹ is chloro, X² is C—F, X³ is hydrogen, X⁴ and G have the values listed in the table Q.

### Table 21 Q

Table 21Q provides 48 compounds of formula (II-B) wherein X¹ is fluoro, X² is C—Cl, X³ is hydrogen, X⁴ and G have the values listed in the table Q.

### Table 22 Q

Table 22Q provides 48 compounds of formula (II-B) wherein X¹ is chloro, X² is C—Cl, X³ is hydrogen, X⁴ and G have the values listed in the table Q.

### Table 23 Q

Table 23Q provides 48 compounds of formula (II-B) wherein X¹ is chloro, X² is C—Br, X³ is chloro, X⁴ and G have the values listed in the table Q.

### Table 24 Q

Table 24Q provides 48 compounds of formula (II-B) wherein X¹ is chloro, X² is C—F, X³ is chloro, X⁴ and G have the values listed in the table Q.

### Table 25 Q

Table 25Q provides 48 compounds of formula (II-B) wherein X¹ is chloro, X² is C—Cl, X³ is chloro, X⁴ and G have the values listed in the table Q.

### Table 26 Q

Table 26Q provides 48 compounds of formula (II-B) wherein X¹ is chloro, X² is C—I, X³ is chloro, X⁴ and G have the values listed in the table Q.

### Table 27 Q

Table 27Q provides 48 compounds of formula (II-B) wherein X¹ is fluoro, X² is C—F, X³ is fluoro, X⁴ and G have the values listed in the table Q.

### Table 28 Q

Table 28Q provides 48 compounds of formula (II-B) wherein X¹ is chloro, X² is CH, X³ is bromo, X⁴ and

G have the values listed in the table Q.

### Table 29 Q

Table 29Q provides 48 compounds of formula (II-B) wherein X¹ is chloro, X² is CH, X³ is fluoro, X⁴ and G have the values listed in the table Q.

### Table 30 Q

Table 30Q provides 48 compounds of formula (II-B) wherein X¹ is chloro, X² is CH, X³ is trifluoromethyl, X⁴ and G have the values listed in the table Q.

### Table 31 Q

Table 31Q provides 48 compounds of formula (II-B) wherein X¹ is chloro, X² is C—Cl, X³ is trifluoromethyl, X⁴ and G have the values listed in the table Q.

### Table 32 Q

Table 32Q provides 48 compounds of formula (II-B) wherein X¹ is trifluoromethyl, X² is CH, X³ is trifluoromethyl, X⁴ and G have the values listed in the table Q.

### Table 33 Q

Table 33Q provides 48 compounds of formula (II-B) wherein X¹ is trifluoromethyl, X² is C—Cl, X³ is trifluoromethyl, X⁴ and G have the values listed in the table Q.

### Table 34 Q

Table 34Q provides 48 compounds of formula (II-B) wherein X¹ is trifluoromethyl, X² is CH, X³ is hydrogen, X⁴ and G have the values listed in the table Q.

### Table 35 Q

Table 35Q provides 48 compounds of formula (II-B) wherein X¹ is chloro, X² is N, X³ is chloro, X⁴ and G have the values listed in the table Q.

### Table 36 Q

Table 36Q provides 48 compounds of formula (II-B) wherein X¹ is trifluoromethyl, X² is N, X³ is trifluoromethyl, X⁴ and G have the values listed in the table Q.

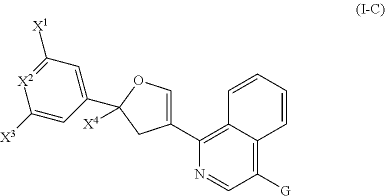

### Table 37 Q

Table 37Q provides 48 compounds of formula (I-C) wherein X¹ is chloro, X² is CH, X³ is chloro, X⁴ and G have the values listed in the table Q.

### Table 38 Q

Table 38Q provides 48 compounds of formula (I-C) wherein X¹ is chloro, X² is C—F, X³ is hydrogen, X⁴ and G have the values listed in the table Q.

### Table 39 Q

Table 39Q provides 48 compounds of formula (I-C) wherein X¹ is fluoro, X² is C—Cl, X³ is hydrogen, X⁴ and G have the values listed in the table Q.

### Table 40 Q

Table 40Q provides 48 compounds of formula (I-C) wherein X¹ is chloro, X² is C—Cl, X³ is hydrogen, X⁴ and G have the values listed in the table Q.

### Table 41 Q

Table 41Q provides 48 compounds of formula (I-C) wherein X¹ is chloro, X² is C—Br, X³ is chloro, X⁴ and G have the values listed in the table Q.

### Table 42 Q

Table 42Q provides 48 compounds of formula (I-C) wherein X¹ is chloro, X² is C—F, X³ is chloro, X⁴ and G have the values listed in the table Q.

### Table 43Q

Table 43Q provides 48 compounds of formula (I-C) wherein X¹ is chloro, X² is C—Cl, X³ is chloro, X⁴ and G have the values listed in the table Q.

### Table 44 Q

Table 44Q provides 48 compounds of formula (I-C) wherein X¹ is chloro, X² is C—I, X³ is chloro, X⁴ and G have the values listed in the table Q.

### Table 45 Q

Table 45Q provides 48 compounds of formula (I-C) wherein X¹ is fluoro, X² is C—F, X³ is fluoro, X⁴ and G have the values listed in the table Q.

### Table 46 Q

Table 46Q provides 48 compounds of formula (I-C) wherein X¹ is chloro, X² is CH, X³ is bromo, X⁴ and G have the values listed in the table Q.

### Table 47 Q

Table 47Q provides 48 compounds of formula (I-C) wherein X¹ is chloro, X² is CH, X³ is fluoro, X⁴ and G have the values listed in the table Q.

### Table 48 Q

Table 48Q provides 48 compounds of formula (I-C) wherein X¹ is chloro, X² is CH, X³ is trifluoromethyl, X⁴ and G have the values listed in the table Q.

### Table 49 Q

Table 49Q provides 48 compounds of formula (I-C) wherein X¹ is chloro, X² is C—Cl, X³ is trifluoromethyl, X⁴ and G have the values listed in the table Q.

### Table 50 Q

Table 50Q provides 48 compounds of formula (I-C) wherein X¹ is trifluoromethyl, X² is CH, X³ is trifluoromethyl, X⁴ and G have the values listed in the table Q.

### Table 51 Q

Table 51Q provides 48 compounds of formula (I-C) wherein X¹ is trifluoromethyl, X² is C—Cl, X³ is trifluoromethyl, X⁴ and G have the values listed in the table Q.

### Table 52 Q

Table 52Q provides 48 compounds of formula (I-C) wherein X¹ is trifluoromethyl, X² is CH, X³ is hydrogen, X⁴ and G have the values listed in the table Q.

### Table 53 Q

Table 53Q provides 48 compounds of formula (I-C) wherein X¹ is chloro, X² is N, X³ is chloro, X⁴ and G have the values listed in the table Q.

### Table 54 Q

Table 54Q provides 48 compounds of formula (I-C) wherein X¹ is trifluoromethyl, X² is N, X³ is trifluoromethyl, X⁴ and G have the values listed in the table Q.

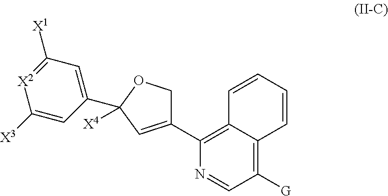

### Table 55 Q

Table 55Q provides 48 compounds of formula (II-C) wherein X¹ is chloro, X² is CH, X³ is chloro, X⁴ and G have the values listed in the table Q.

### Table 56 Q

Table 56Q provides 48 compounds of formula (II-C) wherein X¹ is chloro, X² is C—F, X³ is hydrogen, X⁴ and G have the values listed in the table Q.

### Table 57 Q

Table 57Q provides 48 compounds of formula (II-C) wherein X¹ is fluoro, X² is C—Cl, X³ is hydrogen, X⁴ and G have the values listed in the table Q.

### Table 58 Q

Table 58Q provides 48 compounds of formula (II-C) wherein X¹ is chloro, X² is C—Cl, X³ is hydrogen, X⁴ and G have the values listed in the table Q.

### Table 59 Q

Table 59Q provides 48 compounds of formula (II-C) wherein X¹ is chloro, X² is C—Br, X³ is chloro, X⁴ and G have the values listed in the table Q.

### Table 60 Q

Table 60Q provides 48 compounds of formula (II-C) wherein X¹ is chloro, X² is C—F, X³ is chloro, X⁴ and G have the values listed in the table Q.

### Table 61 Q

Table 61Q provides 48 compounds of formula (II-C) wherein X¹ is chloro, X² is C—Cl, X³ is chloro, X⁴ and G have the values listed in the table Q.

### Table 62 Q

Table 62Q provides 48 compounds of formula (II-C) wherein X¹ is chloro, X² is C—I, X³ is chloro, X⁴ and G have the values listed in the table Q.

### Table 63 Q

Table 63Q provides 48 compounds of formula (II-C) wherein X¹ is fluoro, X² is C—F, X³ is fluoro, X⁴ and G have the values listed in the table Q.

### Table 64 Q

Table 64Q provides 48 compounds of formula (II-C) wherein X¹ is chloro, X² is CH, X³ is bromo, X⁴ and G have the values listed in the table Q.

### Table 65 Q

Table 65Q provides 48 compounds of formula (II-C) wherein X¹ is chloro, X² is CH, X³ is fluoro, X⁴ and G have the values listed in the table Q.

### Table 66 Q

Table 66Q provides 48 compounds of formula (II-C) wherein X¹ is chloro, X² is CH, X³ is trifluoromethyl, X⁴ and G have the values listed in the table Q.

### Table 67 Q

Table 67Q provides 48 compounds of formula (II-C) wherein X¹ is chloro, X² is C—Cl, X³ is trifluoromethyl, X⁴ and G have the values listed in the table Q.

### Table 68 Q

Table 68Q provides 48 compounds of formula (II-C) wherein X¹ is trifluoromethyl, X² is CH, X³ is trifluoromethyl, X⁴ and G have the values listed in the table Q.

### Table 69 Q

Table 69Q provides 48 compounds of formula (II-C) wherein X¹ is trifluoromethyl, X² is C—Cl, X³ is trifluoromethyl, X⁴ and G have the values listed in the table Q.

### Table 70 Q

Table 70Q provides 48 compounds of formula (II-C) wherein X¹ is trifluoromethyl, X² is CH, X³ is hydrogen, X⁴ and G have the values listed in the table Q.

### Table 71 Q

Table 71Q provides 48 compounds of formula (II-C) wherein X¹ is chloro, X² is N, X³ is chloro, X⁴ and G have the values listed in the table Q.

### Table 72 Q

Table 72Q provides 48 compounds of formula (II-C) wherein X¹ is trifluoromethyl, X² is N, X³ is trifluoromethyl, X⁴ and G have the values listed in the table Q.

Compounds of formula I include at least one chiral centre and may exist as compounds of formula I* or compounds of formula I**. Compounds I* and I** are enantiomers if there is no other chiral center or epimers otherwise.

### Compound of Formula I*

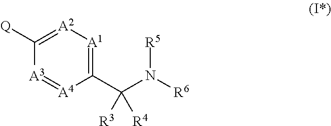

wherein

### Q is Q1* or Q2*

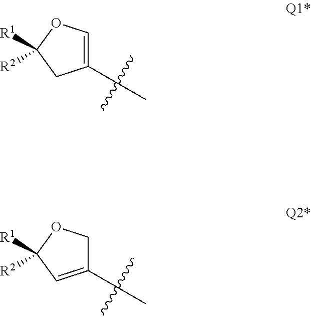

### Compound of formula I**

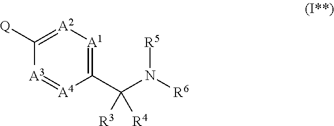

wherein

### Q is Q1** or Q2**

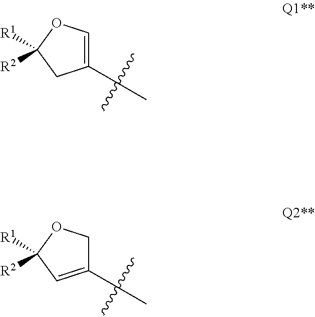

Generally compounds of formula I** are more biologically active than compounds of formula I*. The invention includes mixtures of compounds I* and I** in any ratio e.g. in a molar ratio of 1:99 to 99:1, e.g. 10:1 to 1:10, e.g. a substantially 50:50 molar ratio. In an enantiomerically (or epimerically) enriched mixture of formula I**, the molar proportion of compound I** compared to the total amount of both enantiomers is for example greater than 50%, e.g. at least 55, 60, 65, 70, 75, 80, 85, 90, 95, 96, 97, 98, or at least 99%. Likewise, in enantiomerically (or epimerically) enriched mixture of formula I*, the molar proportion of the compound of formula I* compared to the total amount of both enantiomers (or epimerically) is for example greater than 50%, e.g. at least 55, 60, 65, 70, 75, 80, 85, 90, 95, 96, 97, 98, or at least 99%. Enantiomerically (or epimerically) enriched mixtures of formula I** are preferred.

Each of the compounds disclosed in Tables 1P to 90P, 1Q to 18Q and 37Q to 54Q represents a compound of formula I* in which Q is Q1*, and a compound of formula I** in which Q is Q1**. Likewise, each of the compounds disclosed in Tables 91P to 180P, 19Q to 36Q and 55Q to 72Q represents a compound of formula I* in which Q is Q2*, and a compound of formula I** in which Q is Q2**. In one embodiment the invention provides a compound selected from Tables 1P to 90P, 1Q to 18Q and 37Q to 54Q for use in controlling and/or preventing insects of the family Curculionidae, preferably in for use in controlling and/or preventing Anthonomus grandis.

Additional examples of insects from the family of Curculionidae are Anthonomus corvulus, Anthonomus elutus, Anthonomus elongatus, Anthonomus eugenii, Anthonomus consors, Anthonomus haematopus, Anthonomus lecontei, Anthonomus molochinus, Anthonomus morticinus, Anthonomus musculus, Anthonomus nigrinus, Anthonomus phyllocola, Anthonomus pictus, Anthonomus pomorum, Anthonomus quadrigibbus, Anthonomus rectirostris, Anthonomus rubi, Anthonomus santacruzi, Anthonomus signatus, Anthonomus subfasciatus, and Anthonomus tenebrosus.

In one embodiment the invention provides a compound selected from Tables 1P to 90P, 1Q to 18Q and 37Q to 54Q for use against Anthonomus grandis in cotton.

In one embodiment the invention provides a compound selected from Tables 1P to 90P, 1Q to 18Q and 37Q to 54Q for use in controlling and/or preventing soil pests.

In one embodiment the invention provides a compound selected from Tables 1P to 90P, 1Q to 18Q and 37Q to 54Q for use in controlling and/or preventing corn rootworm, in particular for use against corn root worm from the genus Diabrotica.

In one embodiment the invention provides a compound selected from Tables 1P to 90P, 1Q to 18Q and 37Q to 54Q for use in controlling and/or preventing Diabrotica virgifera.

In one embodiment the invention provides a compound selected from Tables 1P to 90P, 1Q to 18Q and 37Q to 54Q for use in controlling and/or preventing Diabrotica barberi.

In one embodiment the invention provides a compound selected from Tables 1P to 90P, 1Q to 18Q and 37Q to 54Q for use in controlling and/or preventing Diabrotica undecimpunctata howardi.

In one embodiment the invention provides a compound selected from Tables 1P to 90P, 1Q to 18Q and 37Q to 54Q for use in controlling and/or preventing wireworms, in particular Agriotes spp.

In one embodiment the invention provides a compound selected from Tables 1P to 90P, 1Q to 18Q and 37Q to 54Q for use in controlling and/or preventing Agriotes spp. in cereals, potato or corn.

Additional examples of Agriotes spp. include Agriotes lineatus, Agriotes obscurus, Agriotes brevis, Agriotes gurgistanus, Agriotes sputator, Agriotes ustulatus, Ctenicera destructor, and Limonius californicus.

In one embodiment the invention provides a compound selected from Tables 1P to 90P, 1Q to 18Q and 37Q to 54Q for use in controlling and/or preventing grubs, in particular white grubs.

In one embodiment the invention provides a compound selected from Tables 1P to 90P, 1Q to 18Q and 37Q to 54Q for use in controlling and/or preventing Phyllophaga spp., particularly on corn, soybean or cotton.

In one embodiment the invention provides a compound selected from Tables 1P to 90P, 1Q to 18Q and 37Q to 54Q for use in controlling and/or preventing Diloboderus spp. particularly on corn, soybean or cotton.

In one embodiment the invention provides a compound selected from Tables 1P to 90P, 1Q to 18Q and 37Q to 54Q for use in controlling and/or preventing Popillia japonica, particularly on corn, soybean or cotton.

Additional examples of white grubs include Phyllophaga anxia, Phyllophaga crinite, Phyllophaga subnitida, Diloboderus abderus.

In one embodiment the invention provides a compound selected from Tables 1P to 90P, 1Q to 18Q and 37Q to 54Q for use in controlling and/or preventing termites, e.g. on sugarcane.

Examples of termites include Reticulitermes, Coptotermes, Macrotermes, Microtermes, Globitermes. Specific of subterranean termites include Reticulitermes flavipes, Reticulitermes hesperus, Reticulitermes verginicus, Reticulitermes hageni, Reticulitermes speratus, Reticulitermes lucifugus, Heterotermes aureus, Coptotermes formosanus, Coptotermes acinaciformis, Coptotermes curvignathus, Nasutitermes exitiosus, Nasutitermes walkeri, Mastotermes darwiniensis, Schedorhinotermes spp, Macrotermes bellicosus, Macrotermes spp., Globitermes sulphureus, Odontotermes spp. Specific examples of dry wood termites include Incisitermes minor, Marginitermes hubbardi, Cryptotermes brevis, Kalotermes flavicollis. Additional examples of termites include procornitermes spp. and procornitermes araujoi

In one embodiment the invention provides a compound selected from Tables 1P to 90P, 1Q to 18Q and 37Q to 54Q for use in controlling and/or preventing subterraneous stinkbugs, e.g. Scaptocoris spp.

In one embodiment the invention provides a compound selected from Tables 1P to 90P, 1Q to 18Q and 37Q to 54Q for use in controlling and/or preventing Scaptocoris castaneus, in particular on cereals, soybean or corn.

In one embodiment the invention provides a compound selected from Tables 1P to 90P, 1Q to 18Q and 37Q to 54Q for use in controlling and/or preventing cutworms, e.g. agrotis spp.

In one embodiment the invention provides a compound selected from Tables 1P to 90P, 1Q to 18Q and 37Q to 54Q for use in controlling and/or preventing Agrotis ipsilon, particularly on cereals, canola, soybean or corn.

In one embodiment the invention provides a compound selected from Tables 1P to 90P, 1Q to 18Q and 37Q to 54Q for use in controlling and/or preventing millipedes, e.g. Julus spp.

In one embodiment the invention provides a compound selected from Tables 1P to 90P, 1Q to 18Q and 37Q to 54Q for use in controlling and/or preventing Julus spp., particularly on cereals, canola, soybean & corn.

In one embodiment the invention provides a compound selected from Tables 1P to 90P, 1Q to 18Q and 37Q to 54Q for use in controlling and/or preventing broca gigante, e.g. Telchin licus, particularly on sugarcane.

In one embodiment the invention provides a compound selected from Tables 1P to 90P, 1Q to 18Q and 37Q to 54Q for use in controlling and/or preventing whitefly.

In one embodiment the invention provides a compound selected from Tables 1P to 90P, 1Q to 18Q and 37Q to 54Q for use in controlling and/or preventing Bemisia tabaci, particularly on vegetables, cotton, soybean, or potatoes.

In one embodiment the invention provides a compound selected from Tables 1P to 90P, 1Q to 18Q and 37Q to 54Q for use in controlling and/or preventing Trialeurodes vaporariorum, particularly on vegetables, cotton, soybean, or potatoes.

In one embodiment the invention provides a compound selected from Tables 1P to 90P, 1Q to 18Q and 37Q to 54Q for use in controlling and/or preventing stinkbugs, in particular Euschistus spp.

In one embodiment the invention provides a compound selected from Tables 1P to 90P, 1Q to 18Q and 37Q to 54Q for use in controlling and/or preventing Euschistus spp., particularly in soybean.

Examples of stinkbugs include Nezara spp. (e.g. Nezara viridula, Nezara antennata, Nezara hilare), Piezodorus spp. (e.g. Piezodorus guildinii), Acrosternum spp. Euchistus spp. (e.g. Euchistus heros, Euschistus serous), Halyomorpha halys, Plautia crossota, Riptortus clavatus, Rhopalus msculatus, Antestiopsis orbitalus, Dichelops spp. (e.g. Dichelops furcatus, Dichelops melacanthus), Eurygaster spp. (e.g. Eurygaster intergriceps, Eurygaster maura), Oebalus spp. (e.g. Oebalus mexicana, Oebalus poecilus, Oebalus pugnase, Scotinophara spp. (e.g. Scotinophara lurida, Scotinophara coarctata). Preferred targets include Antestiopsis orbitalus, Dichelops furcatus, Dichelops melacanthus, Euchistus heros, Euschistus serous, Nezara viridula, Nezara hilare, Piezodorus guildinii, Halyomorpha halys. In one embodiment the stinkbug target is Nezara viridula, Piezodorus spp., Acrosternum spp, Euchistus heros.

In one embodiment the invention provides a compound selected from Tables 1P to 90P, 1Q to 18Q and 37Q to 54Q for use against rice pests.

In one embodiment the invention provides a compound selected from Tables 1P to 90P, 1Q to 18Q and 37Q to 54Q for use against stemborer, particularly in rice.

Examples of stemborers include Chilo sp, Chilo suppressalis, Chilo polychrysus, Chilo auricilius, Scirpophaga spp., Scirpophaga incertulas, Scirpophaga innotata, Scirpophaga nivella Sesamia sp, Sesamia inferens.

In one embodiment the invention provides a compound selected from Tables 1P to 90P, 1Q to 18Q and 37Q to 54Q for use against leaffolder, particularly in rice.

Examples of leaffolders include Cnaphalocrocis spp., Cnaphalocrocis medinalis, Marasmia spp., Marasmia patnalis, Marasmia exigua.

In one embodiment the invention provides a compound selected from Tables 1P to 90P, 1Q to 18Q and 37Q to 54Q for use against hoppers, particularly in rice.

Examples of Hoppers include Nephotettix spp., Nephotettix virescens, Nephotettix nigropictus, Nephotettix malayanus, Nephotettix cincticeps, Nilaparvata lugens, Sogatella furcifera.

In one embodiment the invention provides a compound selected from Tables 1P to 90P, 1Q to 18Q and 37Q to 54Q for use against gallmidge, particularly in rice.

Examples of Gall midge include Orseolia sp, Orseolia oryzae.

In one embodiment the invention provides a compound selected from Tables 1P to 90P, 1Q to 18Q and 37Q to 54Q for use against whorl maggot, particularly in rice.

Examples of whorl maggots include Hydrellia sp, Hydrellia philippina.

In one embodiment the invention provides a compound selected from Tables 1P to 90P, 1Q to 18Q and 37Q to 54Q for use against Rice bugs, particularly in rice.

Examples of rice bugs include Leptocorisa sp, Leptocorisa oratorius, Leptocorisa chinensis, Leptocorisa acuta.

In one embodiment the invention provides a compound selected from Tables 1P to 90P, 1Q to 18Q and 37Q to 54Q for use against Black bugs, particularly in rice.

Examples of Black bugs include Scotinophara sp, Scotinophara coarctata, Scotinophara lurida, Scotinophara latiuscula.

In one embodiment the invention provides a compound selected from Tables 1P to 90P, 1Q to 18Q and 37Q to 54Q for use against plutella spp.

In one embodiment the invention provides a compound selected from Tables 1P to 90P, 1Q to 18Q and 37Q to 54Q for use against Plutella xylostella, particularly in brassica crops.

In one embodiment the invention provides a compound selected from Tables 91P to 180P, 19Q to 36Q and 55Q to 72Q for use in controlling and/or preventing insects of the family Curculionidae, preferably in for use in controlling and/or preventing Anthonomus grandis.

Additional examples of insects from the family of Curculionidae are Anthonomus corvulus, Anthonomus elutus, Anthonomus elongatus, Anthonomus eugenii, Anthonomus consors, Anthonomus haematopus, Anthonomus lecontei, Anthonomus molochinus, Anthonomus morticinus, Anthonomus musculus, Anthonomus nigrinus, Anthonomus phyllocola, Anthonomus pictus, Anthonomus pomorum, Anthonomus quadrigibbus, Anthonomus rectirostris, Anthonomus rubi, Anthonomus santacruzi, Anthonomus signatus, Anthonomus subfasciatus, and Anthonomus tenebrosus.

In one embodiment the invention provides a compound selected from Tables 91P to 180P, 19Q to 36Q and 55Q to 72Q for use against Anthonomus grandis in cotton.

In one embodiment the invention provides a compound selected from Tables 91P to 180P, 19Q to 36Q and 55Q to 72Q for use in controlling and/or preventing soil pests.

In one embodiment the invention provides a compound selected from Tables 91P to 180P, 19Q to 36Q and 55Q to 72Q for use in controlling and/or preventing corn rootworm, in particular for use against corn root worm from the genus Diabrotica.

In one embodiment the invention provides a compound selected from Tables 91P to 180P, 19Q to 36Q and 55Q to 72Q for use in controlling and/or preventing corn Diabrotica virgifera.

In one embodiment the invention provides a compound selected from Tables 91P to 180P, 19Q to 36Q and 55Q to 72Q for use in controlling and/or preventing corn Diabrotica barberi.

In one embodiment the invention provides a compound selected from Tables 91P to 180P, 19Q to 36Q and 55Q to 72Q for use in controlling and/or preventing corn Diabrotica undecimpunctata howardi.

In one embodiment the invention provides a compound selected from Tables 91P to 180P, 19Q to 36Q and 55Q to 72Q for use in controlling and/or preventing wireworms, in particular Agriotes spp.

In one embodiment the invention provides a compound selected from Tables 91P to 180P, 19Q to 36Q and 55Q to 72Q for use in controlling and/or preventing Agriotes spp. in cereals, potato or corn.

Additional examples of Agriotes spp. include Agriotes lineatus, Agriotes obscurus, Agriotes brevis, Agriotes gurgistanus, Agriotes sputator, Agriotes ustulatus, Ctenicera destructor, and Limonius californicus.

In one embodiment the invention provides a compound selected from Tables 91P to 180P, 19Q to 36Q and 55Q to 72Q for use in controlling and/or preventing grubs, in particular white grubs.

In one embodiment the invention provides a compound selected from Tables 91P to 180P, 19Q to 36Q and 55Q to 72Q for use in controlling and/or preventing Phyllophaga spp., particularly on corn, soybean or cotton.

In one embodiment the invention provides a compound selected from Tables 91P to 180P, 19Q to 36Q and 55Q to 72Q for use in controlling and/or preventing Diloboderus spp. particularly on corn, soybean or cotton.

In one embodiment the invention provides a compound selected from Tables 91P to 180P, 19Q to 36Q and 55Q to 72Q for use in controlling and/or preventing Popillia japonica, particularly on corn, soybean or cotton.

Additional examples of white grubs include Phyllophaga anxia, Phyllophaga crinite, Phyllophaga subnitida, Diloboderus abderus.

In one embodiment the invention provides a compound selected from Tables 91P to 180P, 19Q to 36Q and 55Q to 72Q for use in controlling and/or preventing termites, e.g. on sugarcane.

Examples of termites include Reticulitermes, Coptotermes, Macrotermes, Microtermes, Globitermes. Specific of subterranean termites include Reticulitermes flavipes, Reticulitermes hesperus, Reticulitermes verginicus, Reticulitermes hageni, Reticulitermes speratus, Reticulitermes lucifugus, Heterotermes aureus, Coptotermes formosanus, Coptotermes acinaciformis, Coptotermes curvignathus, Nasutitermes exitiosus, Nasutitermes walkeri, Mastotermes darwiniensis, Schedorhinotermes spp, Macrotermes bellicosus, Macrotermes spp., Globitermes sulphureus, Odontotermes spp. Specific examples of dry wood termites include Incisitermes minor, Marginitermes hubbardi, Cryptotermes brevis, Kalotermes flavicollis. Additional examples of termites include procornitermes spp. and procornitermes araujoi

In one embodiment the invention provides a compound selected from Tables 91P to 180P, 19Q to 36Q and 55Q to 72Q for use in controlling and/or preventing subterraneous stinkbugs, e.g. Scaptocoris spp.

In one embodiment the invention provides a compound selected from Tables 91P to 180P, 19Q to 36Q and 55Q to 72Q for use in controlling and/or preventing Scaptocoris castaneus, in particular on cereals, soybean or corn.

In one embodiment the invention provides a compound selected from Tables 91P to 180P, 19Q to 36Q and 55Q to 72Q for use in controlling and/or preventing cutworms, e.g. agrotis spp.

In one embodiment the invention provides a compound selected from Tables 91P to 180P, 19Q to 36Q and 55Q to 72Q for use in controlling and/or preventing Agrotis ipsilon, particularly on cereals, canola, soybean or corn.

In one embodiment the invention provides a compound selected from Tables 91P to 180P, 19Q to 36Q and 55Q to 72Q for use in controlling and/or preventing millipedes, e.g. Julus spp.

In one embodiment the invention provides a compound selected from Tables 91P to 180P, 19Q to 36Q and 55Q to 72Q for use in controlling and/or preventing Julus spp., particularly on cereals, canola, soybean & corn.

In one embodiment the invention provides a compound selected from Tables 91P to 180P, 19Q to 36Q and 55Q to 72Q for use in controlling and/or preventing broca gigante, e.g. Telchin licus, particularly on sugarcane.

In one embodiment the invention provides a compound selected from Tables 91P to 180P, 19Q to 36Q and 55Q to 72Q for use in controlling and/or preventing whitefly.

In one embodiment the invention provides a compound selected from Tables 91P to 180P, 19Q to 36Q and 55Q to 72Q for use in controlling and/or preventing Bemisia tabaci, particularly on vegetables, cotton, soybean, or potatoes.

In one embodiment the invention provides a compound selected from Tables 91P to 180P, 19Q to 36Q and 55Q to 72Q for use in controlling and/or preventing Trialeurodes vaporariorum, particularly on vegetables, cotton, soybean, or potatoes.

In one embodiment the invention provides a compound selected from Tables 91P to 180P, 19Q to 36Q and 55Q to 72Q for use in controlling and/or preventing stinkbugs, in particular Euschistus spp.

In one embodiment the invention provides a compound selected from Tables 91P to 180P, 19Q to 36Q and 55Q to 72Q for use in controlling and/or preventing Euschistus spp., particularly in soybean.

Examples of stinkbugs include Nezara spp. (e.g. Nezara viridula, Nezara antennata, Nezara hilare), Piezodorus spp. (e.g. Piezodorus guildinii), Acrosternum spp. Euchistus spp. (e.g. Euchistus heros, Euschistus servus), Halyomorpha halys, Plautia crossota, Riptortus clavatus, Rhopalus msculatus, Antestiopsis orbitalus, Dichelops spp. (e.g. Dichelops furcatus, Dichelops melacanthus), Eurygaster spp. (e.g. Eurygaster intergriceps, Eurygaster maura), Oebalus spp. (e.g. Oebalus mexicana, Oebalus poecilus, Oebalus pugnase, Scotinophara spp. (e.g. Scotinophara lurida, Scotinophara coarctata). Preferred targets include Antestiopsis orbitalus, Dichelops furcatus, Dichelops melacanthus, Euchistus heros, Euschistus servus, Nezara viridula, Nezara hilare, Piezodorus guildinii, Halyomorpha halys. In one embodiment the stinkbug target is Nezara viridula, Piezodorus spp., Acrosternum spp, Euchistus heros.

In one embodiment the invention provides a compound selected from Tables 91P to 180P, 19Q to 36Q and 55Q to 72Q for use against rice pests.

In one embodiment the invention provides a compound selected from Tables 91P to 180P, 19Q to 36Q and 55Q to 72Q for use against stemborer, particularly in rice.

Examples of stemborers include Chilo sp, Chilo suppressalis, Chilo polychrysus, Chilo auricilius, Scirpophaga spp., Scirpophaga incertulas, Scirpophaga innotata, Scirpophaga nivella Sesamia sp, Sesamia inferens.

In one embodiment the invention provides a compound selected from Tables 91P to 180P, 19Q to 36Q and 55Q to 72Q for use against leaffolder, particularly in rice.

Examples of leaffolders include Cnaphalocrocis spp., Cnaphalocrocis medinalis, Marasmia spp., Marasmia patnalis, Marasmia exigua.

In one embodiment the invention provides a compound selected from Tables 91P to 180P, 19Q to 36Q and 55Q to 72Q for use against hoppers, particularly in rice.

Examples of Hoppers include Nephotettix spp., Nephotettix virescens, Nephotettix nigropictus, Nephotettix malayanus, Nephotettix cincticeps, Nilaparvata lugens, Sogatella furcifera.

In one embodiment the invention provides a compound selected from Tables 91P to 180P, 19Q to 36Q and 55Q to 72Q for use against gallmidge, particularly in rice.

Examples of Gall midge include Orseolia sp, Orseolia oryzae.

In one embodiment the invention provides a compound selected from Tables 91P to 180P, 19Q to 36Q and 55Q to 72Q for use against whorl maggot, particularly in rice.

Examples of whorl maggots include Hydrellia sp, Hydrellia philippina.

In one embodiment the invention provides a compound selected from Tables 91P to 180P, 19Q to 36Q and 55Q to 72Q for use against Rice bugs, particularly in rice.

Examples of rice bugs include Leptocorisa sp, Leptocorisa oratorius, Leptocorisa chinensis, Leptocorisa acuta.

In one embodiment the invention provides a compound selected from Tables 91P to 180P, 19Q to 36Q and 55Q to 72Q for use against Black bugs, particularly in rice.

Examples of Black bugs include Scotinophara sp, Scotinophara coarctata, Scotinophara lurida, Scotinophara latiuscula.

In one embodiment the invention provides a compound selected from Tables 91P to 180P, 19Q to 36Q and 55Q to 72Q for use against plutella spp.

In one embodiment the invention provides a compound selected from Tables 91P to 180P, 19Q to 36Q and 55Q to 72Q for use against Plutella xylostella, particularly in brassica crops.

The compounds of the invention may be made by a variety of methods as shown in the following Schemes.

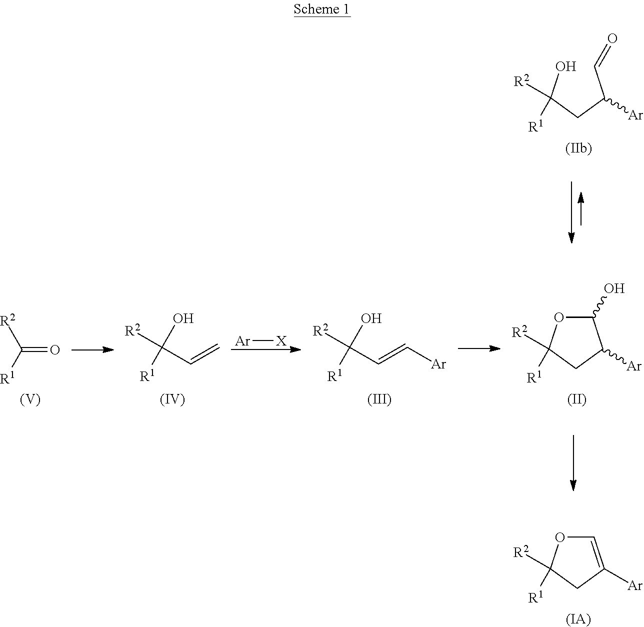

In scheme 1 Ar stands for group A or group A1

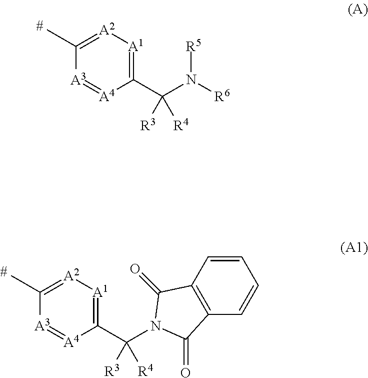

wherein A¹, A², A³, A⁴, R³, R⁴, R⁵ and R⁶ are as defined for compounds of formula I.

1) Compounds of formula IV wherein R¹ and R² are as defined for compounds of formula I can be prepared by addition of vinylnucleophiles (e.g. vinylmagnesium bromide, vinylmagnesium chloride, vinyl zinc or vinylsilanes) to the ketone of formula V, e.g. using similar conditions as described in Journal of Organic Chemistry, 56(17), 5143-6; 1991.

2) Compounds of formula III can be obtained from compounds of formula IV via the Heck reaction, e.g. by treating compounds of formula IV, with a reactant Ar—X, wherein Ar are as defined above and X represents a halogen (Cl, Br, I) or a pseudohalogen (OTf, OTs, diazonium) in the presence of a base, a catalyst and optionally in the presence of a suitable ligand and solvent. Suitable catalysts are e.g. palladium catalysts such as Pd(OAc)₂, PdCl₂, Pd₂(dba)₃, Pd₂(dba)₃.CHCl₃, [Pd(PPh₃)₄], [Pd(Cl)₂(H₃CCN)₂)], [(allyl)Pd(Cl)]₂, [Pd(PPh₃)₂(Cl)₂], [Pd(DPPF)(Cl)₂], Trans-di-μ-acetatobis[2-(di-o-tolylphosphino)benzyl]dipalladium(II) (Herrmanns catalyst), Pd/C. Suitable ligands are e.g. phosphine ligands such as P(tBu)₃, tris(ortho-tolyl)phosphine, BINAP, PPh₃. Suitable bases are e.g. trialkyl amine, metal carbonate or acetate, including tetralkylamonium acetate. Examples of additives are e.g. R₄N⁺X⁻ (R is e.g. alkyl) Ag₂CO₃. Suitable solvents include polar and non-polar organic solvents e.g. water, DMF, DMA, dioxane, NMP, toluene, xylene, AcCN, THF, ionic liquids. The reaction temperature is usually in the range 0° C. to 200° C., more preferably 50° C. to 150° C. The reaction time is usually in the range 1 h to 100 h.

3) Compounds of formula II, wherein Ar is as defined above, may be prepared via hydroformylation of compounds of formula III, e.g. by reacting compounds of formula III with CO and H₂ in the presence of a suitable catalyst. Structure II comprises any composition of cyclic stereo-isomers and or of open chain structure IIb isomers.

Suitable catalysts for the hydroformylation reaction are complexes of transition metals (rhodium, cobalt, platinum, palladium, iridium) preferably rhodium, preferably with a suitable ligand. Particularly preferred ligands include hydride, carbonyl, halogen, substituted and unsubstituted cyclopentadienyls, 2,4-alkanedionates (e.g. acetylacetonate), phosphorus derivatives and mixtures thereof. Phosphorus derivatives are preferred and are typically represented by the formula P(R)₃ wherein R is an aryl, alkyl, alkoxy, aryloxy, alkylamino, arylamino or a bidentate ligand of the formula (R)₂P—Y—P(R)₂, Y represents a 1-20 atom linker. Each R groups may be the same or different.

Preferred ligands are bulky, π-acceptor phosphines, phosphites, phosphinite, phosphabenzenes, phosphabarrelenes, PArₓR₃, (x=0-2; R=pyrrolyl, indolyl, carbazolyl; Ar=aryl, e.g. phenyl), preferably phosphites, phosphabenzenes, phosphinolines and phosphaadamantanes. Preferred specific ligands are e.g. Triphenyl phosphite, BIPHEPHOS, tris(hexafluoroisopropyl) phosphite, Tris(2,4-bis(1,1-dimethylethyl)phenyl)-phosphite, Tris(2-(1,1-dimethylethyl)phenyl)-phosphite, Tris(2-(1,1-dimethylethyl)-4-methyl-phenyl)-phosphite, 2,4,6-Triphenylphosphabenzene, 2,3,4,5,6-pentaphenylphosphabenzene, 2,3,5,6-tetraphenylphosphabenzene, 2,6-bis(2,4-dimethylphenyl)-4-phenylphosphabenzene, 2,6-bis(2-methylphenyl)-4-phenylphosphabenzene, 4-phenyl-2,6-bis(2,4,5trimethylphenyl)phosphabenzene, 2,6-di-2-naphthalenyl-4-phenylphosphabenzene, 2-(2-naphthalenyl)-4,6-diphenylphosphabenzene, 2,6-bis(1-methylethyl)-4-phenylphosphabenzene, 2,4,6-tris(1,1-dimethylethyl)phosphabenzene, 2,6-dimethyl-4-phenylphosphabenzene, 2,4,10-triphenyl-4H-1,4-ethenophosphinoline, 2,10-bis(1-methylethyl)-4-phenyl-4H-1,4-ethenophosphinoline, 2,10-bis(2,4-dimethylphenyl)-4-phenyl-4H-1,4-ethenophosphinoline, 2,10-bis(2,4-dimethylphenyl)-6-methyl-4-phenyl-4H-1,4-ethenophosphinoline, 2,10-bis(2,4-dimethylphenyl)-7-methyl-4-phenyl-4H-1,4-ethenophosphinoline, 1,3,5,7-tetramethyl-6-phenyl-2,4,8-trioxa-6-phosphaadamantane, 1,3,5,7-tetraethyl-6-phenyl-2,4,8-trioxa-6-phosphaadamantane.

The catalyst may be formed in situ from a catalyst precursor (such as (acetylacetonato)dicarbonyl rhodium, tris(triphenylphosphine)rhodium carbonyl hydride, Rh₆(CO)₁₆, Rh₂O₃, RhCl₃, [Rh(OMe)COD]₂, [Rh₂(OAc)₄], [RhCl(COD)]_{z}) and a suitable ligands or preformed in a separate step. A preferred catalyst precursor to ligand ratio is between 1:1 to 1:100 more preferably between 1:5 to 1:50.

The reaction temperature is preferably in the range of 0-250° C. more preferably at 50-150° C. The reaction pressure is preferably in the range of 1-200 bar more preferably 10-100 bar (an atmosphere of carbon monoxide and hydrogen). The reaction time is usually in the range 1 h to 100 h.

The molar ration of CO:H₂ is preferably 1:100 to 100:1 more preferably 1:5 to 5:1. Optionally, CO and/or H₂ reactants may be generated in situ from formaldehyde, formic acid derivatives, metal carbonyls or other suitable precursors.

Preferred solvents include C₅-C₂₀ aliphatic hydrocarbons, C₆-C₂₀ aromatic hydrocarbons, halogenated hydrocarbons, alcohols, ethers, esters, amides, and mixtures thereof. For liquid substrates the reaction may be performed neat.

4) Compounds of formula I, wherein Ar is as defined above, may be prepared from compounds of formula II by dehydration (elimination of water) in the presence of a suitable acidic catalyst or a suitable activation agent (carboxylic or sulfonic acid chloride or anhydride) and a suitable base (Et₃N, pyridine, DBU). The acid catalyst is preferably p-toluenesulfonic acid or pyridinium p-toluenesulfonate. Relative amount of the catalyst to substrate is preferably 1-100 mol % more preferably 1:10-30 mol %. The reaction may be further facilitated by the presence of a drying agent (Na₂SO₄, molecular sieves), azeotropic distillation, gas flow through the reaction mixture, application of vacuum or other means of removing the water formed. Reaction temperature is in the range 0° C. to 200° C., more preferably 50° C. to 150° C. Reaction pressure is preferably between 0.1 mbar and atmospheric, most preferably atmospheric. The reaction time is usually in the range 1 h to 100 h. The product of the hydroformylation reaction (II) may be isolated and or purified before the dehydratation or alternatively the conversion to (I) may be carried in the same pot as the hydroformylation reaction (one pot reaction).

Hydroformation reactions, including reaction conditions and suitable catalylst, are described in Breit et al., Chem. Comm, 2004, 694-695, Fuchs et al., Chem. Eur. J., 2006, 12, 6930-6939, and Breit et al., Chem. Eur. J., 2001, 7, No. 14, each of which is incorporated by reference.

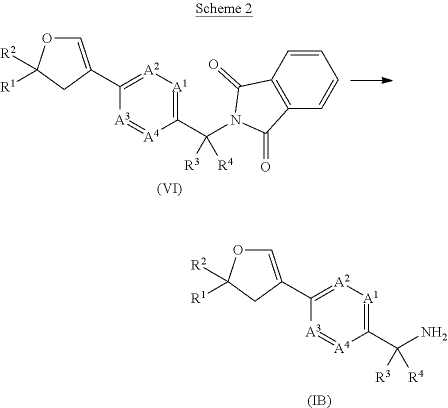

5) Compounds of formula IB may be prepared from compounds of formula VI by cleavage of the phtalimide protecting group (T. W. Green, P. G. M. Wuts, Protective Groups in Organic Synthesis, Wiley-Interscience, New York, 1999, 564-566, 740-743.). Preferred reagents for this transformation are hydrazine or hydrazine hydrate in a suitable solvent (methanol, ethanol, tetrahydrofurane, toluene and others). Reaction temperature is in the range 0° C. to 200° C., more preferably 25° C. to 150° C. The reaction time is usually in the range 0.1 h to 100 h. Other methods employing for example methylamine, sodium hydroxide, lithium hydroxide, potassium hydroxide, ethylene diamine, methylhydrazine, ethanolamine and others or a two-step procedures may be used as well (S. E. Sen, S. L. Roach, Synthesis, 1995, 756-758; J. O. Osby, M. G. Martin, B. Ganem, Tetrahedron Lett., 1984, 25, 2093-2096.)

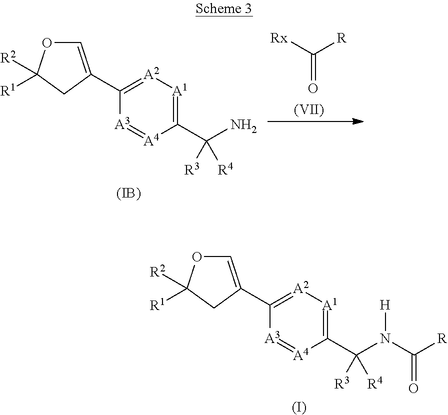

6) Compounds of formula (I) can be prepared by reacting a compound of formula (VII) wherein Rx is OH, C₁-C₆alkoxy or Cl, F or Br, with an amine of formula (IB) as shown in Scheme 3. When Rx is OH such reactions are usually carried out in the presence of a coupling reagent, such as N,N′-dicyclohexylcarbodiimide (“DCC”), 1-ethyl-3-(3-dimethylamino-propyl)carbodiimide hydrochloride (“EDC”) or bis(2-oxo-3-oxazolidinyl)phosphonic chloride (“BOP-Cl”), in the presence of a base, and optionally in the presence of a nucleophilic catalyst, such as hydroxybenzotriazole (“HOBT”). When Rx is Cl, such reactions are usually carried out in the presence of a base, and optionally in the presence of a nucleophilic catalyst. It is possible to conduct the reaction in a biphasic system comprising an organic solvent, preferably ethyl acetate, and an aqueous solvent, preferably a solution of sodium hydrogen carbonate. When Rx is C₁-C₆alkoxy it is sometimes possible to convert the ester directly to the amide by heating the ester and amine together in a thermal process. Suitable bases include pyridine, triethylamine, 4-(dimethylamino)-pyridine (“DMAP”) or diisopropylethylamine (Hunig's base). Preferred solvents are N,N-dimethylacetamide, tetrahydrofuran, dioxane, 1,2-dimethoxyethane, ethyl acetate and toluene. The reaction is carried out at a temperature of from 0° C. to 100° C., preferably from 15° C. to 30° C., in particular at ambient temperature.

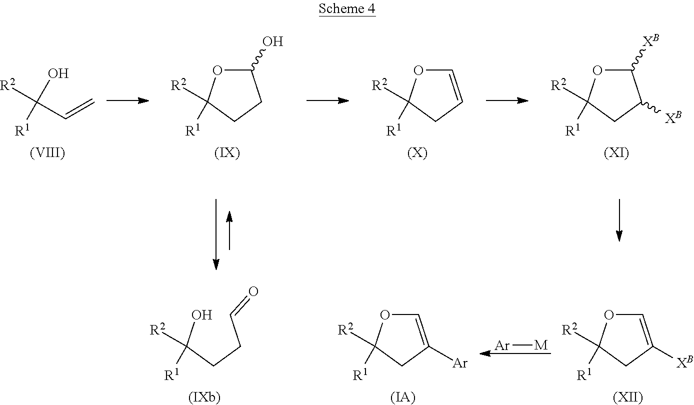

In scheme 4 Ar stands for group A or group A1

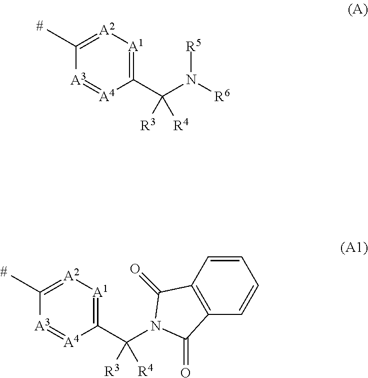

wherein A¹, A², A³, A⁴, R³, R⁴, R⁵ and R⁶ are as defined for compounds of formula I. In scheme 4 X^{B} stands for a halogen (X^{B}=Cl, Br, I); M stands for a derivative of B, Si, Sn, Mg, Zn, Mn.

7) Compounds of formula IX can be obtained from compounds of formula VIII via hydroformylation, e.g. by reacting compounds of formula VIII with CO and H₂ in the presence of a suitable catalyst. Structure IX comprises any composition of cyclic stereo-isomers and or of open chain structure IXb isomers.

Suitable catalysts for the hydroformylation reaction are complexes of transition metals (rhodium, cobalt, platinum, palladium, iridium) preferably rhodium, preferably with a suitable ligand. Particularly preferred ligands include hydride, carbonyl, halogen, substituted and unsubstituted cyclopentadienyls, 2,4-alkanedionates (e.g. acetyacetonate), phosphorus derivatives and mixtures thereof. Phosphorus derivatives are preferred and are typically represented by the formula P(R)₃ wherein R is an aryl, alkyl, alkoxy, aryloxy, alkylamino, arylamino or a bidentate ligand of the formula (R)₂P—Y—P(R)₂, Y represents a 1-20 atom linker. Each R groups may be the same or different.

Preferred ligands are monodentate and bidentate phospines, phosphites, phosphinites. Preferred specific ligands are e.g. triphenyl phosphine, triphenyl phosphite, BIPHEPHOS (2,2′-Bis[(1,1′-biphenyl-2,2′-diyl)phosphite]-3,3′-di-tert-butyl-5,5′-dimethoxy-1,1′-biphenyl), 6-DPPon (6-(diphenylphosphino)-2(1H)-pyridinone), BISBI (2,2′-Bis[(diphenylphosphino)methyl]-1,1′-biphenyl), NAPHOS (2,2′-Bis(diphenylphosphinomethyl)-1,1′-binaphthalene), XANTPHOS (9,9-Dimethyl-4,5-bis(diphenylphosphino)xanthene), tBu-XANTPHOS (1,1′-[2,7-bis(1,1-dimethylethyl)-9,9-dimethyl-9H-xanthene-4,5-diyl]bis[1,1-diphenylphosphine]), TPPTS (3,3′,3″-phosphinidynetris[benzenesulfonic acid] trisodium salt), Tris(2,4-bis(1,1-dimethylethyl)phenyl)-phosphite.

The catalyst may be formed in situ from a catalyst precursor (such as acetylacetonato)dicarbonyl rhodium, tris(triphenylphosphine)rhodium carbonyl hydride, Rh₆(CO)₁₆, Rh₂O₃, RhCl₃, [Rh(OMe)COD]₂, [Rh₂(OAc)₄], [RhCl(COD)]₂) and a suitable ligands or preformed in a separate step. A preferred catalyst precursor to ligand ratio is between 1:1 to 1:100 more preferably between 1:5 to 1:50.

The reaction temperature is preferably in the range of 0-250° C. more preferably at 50-150° C. The reaction pressure is preferably in the range of 1-200 bar more preferably 10-100 bar (an atmosphere of carbon monoxide and hydrogen). The reaction time is usually in the range 1 h to 100 h.

The molar ration of CO:H₂ is preferably 1:100 to 100:1 more preferably 1:5 to 5:1. Optionally, CO and/or H₂ reactants may be generated in situ from formaldehyde, formic acid derivates, metal carbonyls or other suitable precursors.

Preferred solvents include C₅-C₂₀ aliphatic hydrocarbons, C₆-C₂₀ aromatic hydrocarbons, halogenated hydrocarbons, alcohols, ethers, esters, amides, and mixtures thereof

8) Compounds of formula X, may be prepared from compounds of formula IX by dehydration (elimination of water) in the presence of a suitable acidic catalyst or a suitable activation agent (carboxylic or sulfonic acid chloride or anhydride) and a suitable base (Et₃N, pyridine, DBU). The acid catalyst is preferably p-toluenesulfonic acid, methane sulfonic acid or pyridinium p-toluenesulfonate. Relative amount of the catalyst to substrate is preferably 1-100 mol %. The reaction may be further facilitated by the presence of a drying agent (Na₂SO₄, molecular sieves), azeotropic distillation, gas flow through the reaction mixture, application of vacuum (vacuum distillation, flash vacuum pyrolysis) or other means of removing the water formed. Reaction temperature is in the range 0° C. to 1000° C., more preferably 50° C. to 200° C. Reaction pressure is preferably between 0.1 mbar and atmospheric, most between 0.1 to 200 mbar. The reaction time is usually in the range 0.1 h to 100 h. The product of the hydroformylation reaction IX may be isolated and or purified before the dehydratation or alternatively the conversion to X may be carried in the same pot as the hydroformylation reaction (one pot reaction).

9) Compounds of formula XI, wherein X^{B} represents Cl or Br or I, may be prepared from compounds of formula X using an eletrophilic halogen source, such as N-bromosuccinimide, bromine, iodine, chlorine, N-bromosuccinimide, N-chloroosuccinimide, N-iodosuccinimide Structure XI comprises any composition of cyclic stereo-isomers. Suitable solvents include polar and non-polar organic solvents e.g. dichloromethane, chloroform, dichloroethane, dioxane, ethyl acetate, acetonitrile, THF. The reaction temperature is usually in the range −78° C. to 100° C., more preferably −78° C. to 0° C. The reaction time is usually in the range 0.1 h to 100 h.

10) Compounds of formula XII, may be prepared from compounds of formula XI by elimination of HX^{B}, preferably in the presence of a suitable base and solvent. Suitable bases include Et₃N, diisopropyl ethyl amine, pyridine, DBU, DBM, iPrMgCl, iPrMgBr, LDA. Suitable solvents include polar and non-polar organic solvents e.g. dichloroethane, dioxane, THF, toluene, DMF, NMP, acetonitrile. The reaction temperature is usually in the range −30° C. to 200° C., more preferably 0° C. to 150° C. The reaction time is usually in the range 0.1 h to 100 h.

11) Compounds of formula IA can be obtained from compounds of formula XI via a coupling reaction (e.g. Suzuki, Stille, Hiyama, Kumada, Negishi) e.g. by treating compounds of formula XI, with a reactant Ar-M, wherein Ar are as defined above and M represents a suitable derivative of B, Si, Sn, Mg, Zn, Mn (e.g. boronic acid, boronic ester, trifluoroborate, dialkyl-hydroxysilane, trialkyltin, MgCl, MgBr, ZnCl, ZnBr, MnCl) in presence of a catalyst and optionally in the presence of a suitable ligand, solvent and additive. Suitable catalysts are e.g. palladium catalysts such as Pd(OAc)₂, PdCl₂, Pd₂(dba)₃, Pd₂(dba)₃.CHCl₃, [Pd(PPh₃)₄], [Pd(Cl)₂(H₃CCN)₂)], [(allyl)Pd(Cl)]₂, [Pd(PPh₃)₂(Cl)₂], [Pd(DPPF)(Cl)₂], PEPPSI, nickel catalysts such as NiCl₂, Ni(OAc)₂, Ni(acac)₂, [Ni(PPh₃)₂Cl₂], [Ni(DPPP)Cl₂]. Suitable ligands are e.g. phosphine ligands such as P(tBu)₃, tris(ortho-tolyl)phosphine, BINAP, PPh₃, PCy₃, S-Phos, X-Phos, Ru-Phos, trifuryl phosphine, Tris(2,4-bis(1,1-dimethylethyl)phenyl)-phosphite, DPEphos, Josiphos and carbine ligands such as IMes, SIMes, IPr, SIPr. Suitable solvents include polar and non-polar organic solvents e.g. DMF, DMA, DME, dioxane, NMP, toluene, xylene, water, AcCN, THF, ionic liquids. Suitable additives are e.g. trialkyl amine, metal carbonate or acetate or phosphate or fluoride. Examples of additives are e.g. Et₃N, Na₂CO₃, K₂CO₃, Cs₂CO₃, K₃PO₄, KF, CsF. The reaction temperature is usually in the range 0° C. to 200° C., more preferably 50° C. to 150° C. The reaction time is usually in the range 1 h to 100 h.

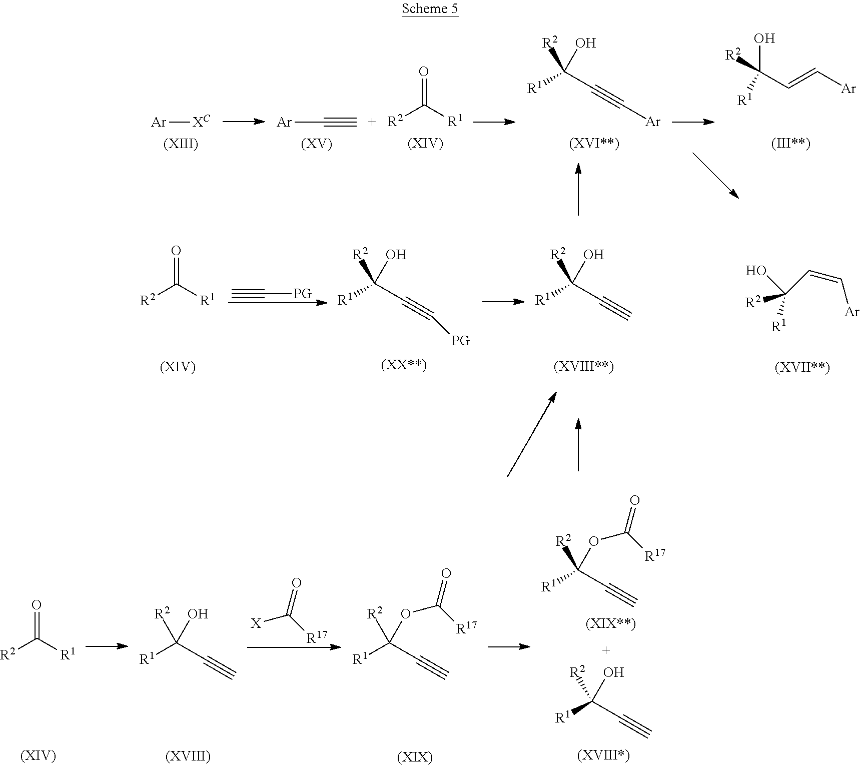

In scheme 5 Ar stands for group A or group A1

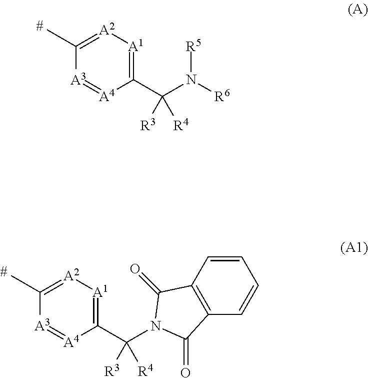

wherein A¹, A², A³, A⁴, R³, R⁴, R⁵ and R⁶ are as defined for compounds of formula I.

12) Compounds of formula XV can be obtained from the corresponding aryl halide XIII via a coupling reaction (e.g. Suzuki, Stille, Hiyama, Kumada, Negishi, Sonigashira) e.g. by treating the Ar—X^{C}, wherein Ar are as defined above and X^{C} represents a halogen (Cl, Br, I) or a pseudohalogen (OTf, OTs, diazonium) with an ethynyl-M, wherein Ar are as defined above and M represents a suitable derivative of B, Si, Sn, Mg, Zn, Cu (formed in situ from corresponding terminal alkyne) in presence of a catalyst and optionally in the presence of a suitable ligand, solvent and additive. Suitable catalysts are e.g. palladium catalysts such as Pd(OAc)₂, PdCl₂, Pd₂(dba)₃, Pd₂(dba)₃.CHCl₃, [Pd(PPh₃)₄], [Pd(Cl)₂(H₃CCN)₂)], [(allyl)Pd(Cl)]₂, [Pd(PPh₃)₂(Cl)₂], [Pd(DPPF)(Cl)₂], PEPPSI, Suitable solvents include polar and non-polar organic solvents e.g. DMF, DMA, DME, dioxane, NMP, toluene, xylene, water, AcCN, THF, ionic liquids. Suitable additives are e.g. trialkyl amine, metal carbonate or acetate or phosphate or fluoride. Examples of additives are e.g. Et₃N, Na₂CO₃, K₂CO₃, Cs₂CO₃, K₃PO₄, KF, CsF. The reaction temperature is usually in the range 0° C. to 200° C., more preferably 50° C. to 150° C. The reaction time is usually in the range 1 h to 100 h.

13) Enantiomerically enriched compounds of formula XVI** wherein R¹ and R² are as defined for compounds of formula I and wherein Ar are as defined above can be prepared by deprotonating compounds of formula XV using a suitable base in a suitable aprotic organic solvent between −90° C. and 80° C., followed by reaction with a titanium alkoxide or chloroalkoxide e.g. Ti(OiPr)₄, Ti(OEt)₄, ClTi(OiPr)₃ between −40° C. and 60° C. in the presence of chiral amino alcohols ligands or chiral diol ligands, a suitable additive and compounds of formula XIV, as described in Angewandte Chemie, International Edition (2011), 50(15), 3538-3542. Suitable base are BuLi, sec-BuLi, tert-BuLi, Me₂Zn, Et₂Zn, Me₃Al, Et₃Al. Preferred base are Me₂Zn and Et₂Zn. Suitable solvents are xylenes, toluene, THF, DME, CH₂Cl₂, C₂H₄Cl₂. The preferred solvent is toluene. Suitable chiral ligands are Cinchona alkaloids (e g quinine, quinidine, cinchonidine, cinchonine), N,N-dialkylephedrine, N,N-dialkylpseudoephedrine, (R)-binol, (R)—H₈-binol. Preferred ligands are Cinchona alkaloids. Suitable additives are CaH₂ and BaF₂. The preferred reaction temperature is between −30° C. and 50° C.

14) Enantiomerically enriched compounds of formula XVI** wherein R¹ and R² are as defined for compounds of formula I and wherein Ar are as defined above can be prepared by deprotonating compounds of formula XV using a suitable organolithium base e.g. BuLi between −100 and −40° C. in an aprotic organic solvent (e.g. toluene, tetrahydrofuran, 1,2-dimethoxyethane, 1,4-dioxane, diethylether, CH₂Cl₂, C₂H₄Cl₂) in presence of a chiral diol ligand (e.g. (S)-1-(2-hydroxy-3-phenyl-1-naphthyl)-3-phenyl-naphthalen-2-ol) and compounds of formula XIV as described in Chem. Commun. 2011, 47, 5614.

15) Enantiomerically enriched compounds of formula III** wherein R¹ and R² are as defined for compounds of formula I and wherein Ar are as defined above can be obtained by reduction of compounds of XVI** by sodium bis(2-methoxyethoxy)aluminumhydride as described in Tetrahedron, 66(39), 7726-7731; 2010. Suitable solvents for this reaction are toluene, tetrahydrofuran, 1,2-dimethoxyethane, 1,4-dioxane and diethylether. The reaction is run between −78° C. and 25° C. and preferably between −50° C. and −10° C.

16) Enantiomerically enriched compounds of formula XVII** wherein R¹ and R² are as defined for compounds of formula I and wherein Ar are as defined above can be obtained by reduction of compounds of XVI** by deactivated palladium catalysts (e.g. Lindlar's catalyst) as described in Tetrahedron, 53(11), 3879-3916; 1997. The suitable solvents for this reaction are ethyl acetate, tetrahydrofuran, 1,2-dimethoxyethane, 1,4-dioxane, diethylether, methanol and ethanol. The reaction is run between −0° C. and 100° C. and preferably between 10 and 60° C.

17) Compounds of formula XVIII wherein R¹ and R² are as defined for compounds of formula (I) can be prepared by addition of ethynylnucleophiles (e.g. ethynylmagnesium bromide, ethynylmagnesium chloride) to the ketone of formula XIV, e.g. using similar conditions as described in Advanced Synthesis & Catalysis, 349(8+9), 1393-1398; 2007.

18) Compounds of formula XIX wherein R¹ and R² are as defined for compounds of formula I and R¹⁷ is C₁-C₁₂alkyl, preferably C₁-C₈ alkyl, are prepared by reacting compounds of formula XVIII using similar conditions as described in Advanced Synthesis & Catalysis, 349(8+9), 1393-1398; 2007.

19) Enantiomerically enriched compounds of formula XVIII* and XIX** wherein R¹ and R² are as defined for compounds of formula I and R¹⁷ is C₁-C₁₂alkyl, preferably C₁-C₈ alkyl are prepared by treating compounds of formula XIX with a suitable hydrolase enzyme in a suitable aqueous system in presence of a suitable buffer, pH 5-9, between 10° C. and 80° C. Suitable enzymes are Pig liver esterase (Roche), Novozyme 398 (Novozymes), Novozymes 435 (supported lipase, Novozymes), Alcalase from Bacillus licheniformis (Merck), Alcalase (Novozymes), Protease type XIII from Aspergillus oryzae (Sigma), Lipase from Candida rugosa (Sigma), Lipase type VII from Candida rugosa (Sigma), Palatase, lipase from Rhizomucor miehei (Sigma), Wheat germ lipase (Sigma), Lipase PS from Burkholderia cepacia (Amano), Lipase AK from Pseudomonas fluorescens (Amano), Lipase from porcine pancreas (Sigma), Esterase ECS-Es 01 (Enzymicals), Esterase ECS-Es 06 (Enzymicals), Esterase ECS-Es 08 (Enzymicals), Esterase ECS-Es 09 (Enzymicals), Esterase ECS-Es 10 (Enzymicals), Lipase MY from Candida rugosa (Meito Sangyo), Lipase OF from Candida rugosa (Meito Sangyo), Lipase SL from Burkholderia cepacia (Meito Sangyo), Lipase TL from Pseudomonas stutzeri (Meito Sangyo). The preferred enzymes are Lipase from Candida rugosa (Sigma), Lipase type VII from Candida rugosa (Sigma), Lipase MY from Candida rugosa (Meito Sangyo), Lipase OF from Candida rugosa (Meito Sangyo). The suitable solvents systems are water, water/dimethylsulfoxide, water/toluene, water/acetone, water/methanol, water/acetonitrile, water/1,4-dioxane, water/n-hexane, water/cyclohexane, water/methyl-tert-butylether, water/diisopropylether. The preferred solvent systems are water/dimethylsulfoxide, water/methanol, water/acetone, water/n-hexane, water/cyclohexane. The preferred buffers are NaH₂PO₄/Na₂HPO₄ and KH₂PO₄/K₂HPO₄. The preferred pH is 7.4. The preferred temperature is between and 55° C.

20) Enantiomerically enriched compounds of formula XVIII** wherein R¹ and R² are as defined for compounds of formula I and R¹⁷ is C₁-C₁₂alkyl, preferably C₁-C₈ alkyl are prepared by treating compounds of formula XIX with a suitable hydrolase enzyme in a suitable aqueous system in presence of a suitable buffer, pH 5-9, between 10° C. and 80° C. Preferred enzymes are Lipase QLM from Alcaligenes sp. (Meito Sangyo) and Lipase PL from Alcaligenes sp. (Meito Sangyo). The preferred solvent systems are water/dimethylsulfoxide, water/methanol, water/acetone, water/n-hexane, water/cyclohexane. The preferred buffers are NaH₂PO₄/Na₂HPO₄ and KH₂PO₄/K₂HPO₄. The preferred pH is 7.4. The preferred temperature is between 35 and 55° C.

21) Enantiomerically enriched compounds of formula XX** wherein R¹ and R² are as defined for compounds of formula (I) and wherein PG is an organosilicon, preferably trialkylsilyl and most preferably trimethylsilyl, are prepared by deprotonation of ethynyl-PG with a suitable organolithium (e.g. BuLi) in presence of a suitable chiral modifier, preferably aminoalcohols ligand in a aprotic organic solvent between −80° C. and 25° C. The preferred chiral ligands are dialkylephedrine and dialkylpseudoephedrine. The most preferred chiral ligand is (1R,2S)-1-phenyl-2-pyrrolidin-1-yl-propan-1-ol. The preferred solvent is tetrahydrofuran. The preferred temperature is between −70° C. and 20° C.

22) Enantiomerically enriched compounds of formula XVIII** wherein R¹ and R² are as defined for compounds of formula I and wherein PG is an organosilicon, preferably trialkylsilyl and most preferably trimethylsilyl, are prepared by reactions of compounds of formula XX** with a suitable base in a suitable organic solvent. Preferred bases are tetrabutylammionium fluoride, potassium carbonate and sodium carbonate. Preferred solvent are tetrahydrofuran, ethanol and methanol. Suitable reaction temperatures is between −10° C. and 60° C.

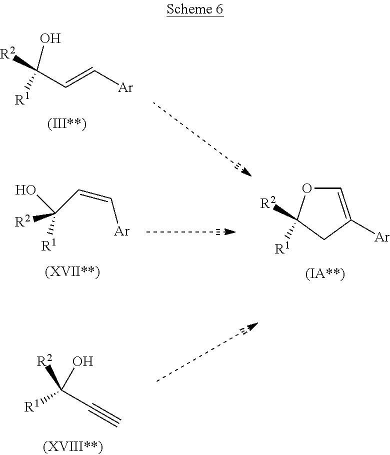

23) Compounds of formula IA** may be prepared from compounds of formula III**, XVII** and XVIII** following the procedures set out in respect of schemes 1 and 4. The synthesis route described in Scheme 1 may also be followed to produce other insecticidally active compounds containing a dihydrofuran moiety, e.g. as described in PCT/EP2011/051284 (incorporated herein by reference), as well as intermediates useful in the preparation of these compounds.

Accordingly, in a further aspect the invention provides a process for preparing a compound of formula IA′

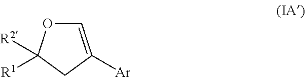

wherein
R¹ is C₁-C₈haloalkyl;
R² is optionally substituted aryl or optionally substituted heteroaryl;
Ar is optionally substituted aryl or optionally substituted heteroaryl;
comprising dehydrating a compound of formula II′

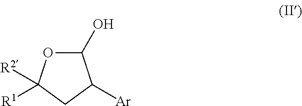

wherein R¹, R² and Ar are as defined for the compound of formula IA′;
with a suitable acidic catalyst or a suitable activation agent and a suitable base. Examples of preferred reaction conditions are described in paragraph 4 above.

The compound of formula II* may be prepared by reacting a compound of formula III′

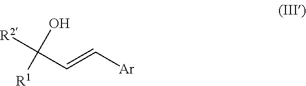

wherein R¹, R² and Ar are as defined for the compound of formula IA′;
with a source of H₂ and CO in the presence of a catalyst comprising a complex of a transition metal and a suitable ligand. Examples of preferred reaction conditions are described in paragraph 3 above.

In a further aspect the invention provides a process for the preparation of a compound of formula II′ comprising reacting a compound of formula III′ with a source of H₂ and CO in the presence of a catalyst comprising a complex of a transition metal and a suitable ligand. Examples of preferred reaction conditions are described in paragraph 3 above. Preferences for the reaction conditions are described above in respect of compounds of formula I, II and III. In particular, the complex of a transition metal is preferably a rhodium complex and the ligand is preferably a phosphite, phosphabenzene, phosphinoline or phosphaadamantane ligand. The source of hydrogen and CO reactants may be gaseous CO and/or H₂ or generated in situ e.g. from formaldehyde, formic acid derivates, metal carbonyls or other suitable precursors. R^{2′} is preferably as defined for R². Ar is preferably as defined under scheme 1, with preferred definitions of A¹, A², A³, A⁴, R⁵ and R⁶ as defined for compounds of formula I.

Preferably the process is for preparing a compound of formula I. R² is preferably as defined for R², with further preferences for R^{2′} as defined for R². Ar is preferably as defined under scheme 1, with preferred definitions of A¹, A², A³, A⁴, R⁵ and R⁶ as defined for compounds of formula I. R¹ and preferences thereof are as defined for the compound of formula I.

In a further aspect the invention provides a process for preparing a compound of formula X′

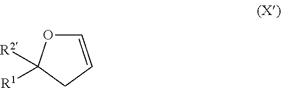

wherein
R¹ is C₁-C₈haloalkyl;
R^{2′} is optionally substituted aryl or optionally substituted heteroaryl;
Ar is optionally substituted aryl or optionally substituted heteroaryl;
comprising dehydrating a compound of formula IX′

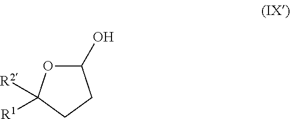

with a suitable acidic catalyst or a suitable activation agent and a suitable base. Examples of preferred reaction conditions are described in paragraph 8 above.

The compound of formula IX′ may be prepared by reacting a compound of formula VIII′

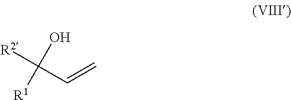

wherein R¹ and R^{2′} are as defined for the compound of formula X′;
with a source of H₂ and CO in the presence of a catalyst comprising a complex of a transition metal and a suitable ligand. Examples of preferred reaction conditions are described in paragraph 7 above.

The process may include the additional step of reacting the compound of formula X′ with chlorine, bromine or iodine to give a compound of formula XI′

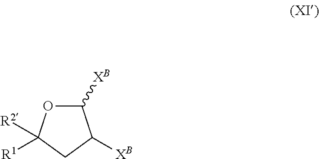

wherein R¹ and R^{2′} are as defined for the compound of formula X′ and X^{B} is Cl, Br or I. Examples of preferred reaction conditions are described in paragraph 9 above.

The process may include the additional step of eliminating HX^{B} from the compound of formula XI′, e.g. in the presence of a suitable base, to give a compound of compound of formula XII′

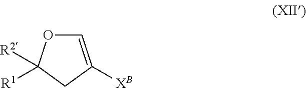

wherein R¹ and R^{2′} are as defined for the compound of formula X′ and X^{B} is Cl, Br or I. Examples of preferred reaction conditions are described in paragraph 10 above.

The process may also include the additional step of reacting a compound of formula XII′ with a compound of formula Ar-M, wherein Ar is optionally substituted aryl or optionally substituted heteroaryl and M is a derivative of B, Si, Sn, Mg, Zn, Mn, to give a compound of formula IA′

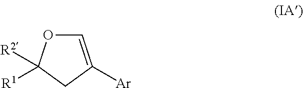

wherein
wherein R¹ and R^{2′} are as defined for the compound of formula X′ and Ar is optionally substituted aryl or optionally substituted heteroaryl. Examples of preferred reaction conditions are described in paragraph 9 above 11.

In a further aspect the invention provides a process for the preparation of a compound of formula IX′ comprising reacting a compound of formula VIII′ with a source of H₂ and CO in the presence of a catalyst comprising a complex of a transition metal and a suitable ligand. Examples of preferred reaction conditions are described in paragraph 7 above.

R^{2′} is preferably as defined for R², with further preferences for R^{2′} as defined for R². Ar is preferably as defined under scheme 1, with preferred definitions of A¹, A², A³, A⁴, R⁵ and R⁶ as defined for compounds of formula I. R¹ and preferences thereof are as defined for the compound of formula I.

Preferences for the reaction conditions are described above in respect of compounds of formula I, VIII, IX, X, XI and XII. In particular, the complex of a transition metal is preferably a rhodium complex and the ligand is preferably a phosphite, phosphabenzene, phosphinoline or phosphaadamantane ligand. The source of hydrogen and CO reactants may be gaseous CO and/or H₂ or generated in situ e.g. from formaldehyde, formic acid derivates, metal carbonyls or other suitable precursors. The compounds of formula (I) can be used to combat and control infestations of insect pests such as Lepidoptera, Diptera, Hemiptera, Thysanoptera, Orthoptera, Dictyoptera, Coleoptera, Siphonaptera, Hymenoptera and Isoptera and also other invertebrate pests, for example, acarine, nematode and mollusc pests. Insects, acarines, nematodes and molluscs are hereinafter collectively referred to as pests. The pests which may be combated and controlled by the use of the compounsd of the invention include those pests associated with agriculture (which term includes the growing of crops for food and fiber products), horticulture and animal husbandry, companion animals, forestry and the storage of products of vegetable origin (such as fruit, grain and timber); those pests associated with the damage of man-made structures and the transmission of diseases of man and animals; and also nuisance pests (such as flies). The compounds of the invention may be used for example on turf, ornamentals, such as flowers, shrubs, broad-leaved trees or evergreens, for example conifers, as well as for tree injection, pest management and the like. Compositions comprising the compound of formula I may be used on ornamental garden plants (e.g. flowers, shrubs, broad-leaved trees or evergreens), e.g. to control aphids, whitefly, scales, meelybug, beetles and caterpillars. Compositions comprising the compound of formula I may be used on garden plants (e.g. flowers, shrubs, broad-leaved trees or evergreens), on indoor plants (e.g. flowers and shrubs) and on indoor pest e.g. to control aphids, whitefly, scales, meelybug, beetles and caterpillars.

Furthermore, the compounds of the invention may be effective against harmful insects, without substantially imposing any harmful side effects to cultivated plants. Application of the compounds of the invention may increase the harvest yields, and may improve the quality of the harvested material. The compounds of the invention may have favourable properties with respect to amount appled, residue formulation, selectivity, toxicity, production methodology, high activity, wide spectrum of control, safety, control of resistant organisms, e.g. pests that are resistant to organic phosphorus agents and/or carbamate agents.

Examples of pest species which may be controlled by the compounds of formula (I) include: coleopterans, for example, Callosobruchus chinensis, Sitophilus zeamais, Tribolium castaneum, Epilachna vigintioctomaculata, Agriotes fuscicollis, Anomala rufocuprea, Leptinotarsa decemlineata, Diabrotica spp., Monochamus alternatus, Lissorhoptrus oryzophilus, Lyctus bruneus, Aulacophora femoralis; lepidopterans, for example, Lymantria dispar, Malacosoma neustria), Pieris rapae, Spodoptera litura, Mamestra brassicae, Chilo suppressalis), Pyrausta nubilalis, Ephestia cautella, Adoxophyes orana, Carpocapsa pomonella, Agrotisfucosa, Galleria mellonella, Plutella maculipennis, Heliothis virescens, Phyllocnistis citrella; hemipterans, for example, Nephotettix cincticeps, Nilaparvata lugens, Pseudococcus comstocki, Unaspis yanonensis, Myzus persicas, Aphis pomi, Aphis gossypii, Rhopalosiphum pseudobrassicas, Stephanitis nashi, Nezara spp., Trialeurodes vaporariorm, Psylla spp.; thysanopterans, for example, Thrips palmi, Franklinella occidental; orthopterans, for example, Blatella germanica, Periplaneta americana, Gryllotalpa Africana, Locusta migratoria migratoriodes; isopterans, for example, Reticulitermes speratus, Coptotermes formosanus; dipterans, for example, Musca domestica, Aedes aegypti, Hylemia platura, Culex pipiens, Anopheles sinensis, Culex tritaeniorhynchus, Liriomyza trifolii; acari, for example, Tetranychus cinnabarinus, Tetranychus urticae, Panonychus citri, Aculops pelekassi, Tarsonemus spp.; nematodes, for example, Meloidogyne incognita, Bursaphelenchus lignicolus Mamiya et Kiyohara, Aphelenchoides besseyi, Heterodera glycines, Pratylenchus spp.

Examples of further pest species which may be controlled by the compounds of formula (I) include: from the order of the Anoplura (Phthiraptera), for example, Damalinia spp., Haematopinus spp., Linognathus spp., Pediculus spp., Trichodectes spp.; from the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici; from the class of the Bivalva, for example, Dreissena spp.; from the order of the Chilopoda, for example, Geophilus spp., Scutigera spp.; from the order of the Coleoptera, for example, Acanthoscehdes obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.; from the order of the Collembola, for example, Onychiurus armatus; from the order of the Dermaptera, for example, Forficula auricularia; from the order of the Diplopoda, for example, Blaniulus guttulatus; from the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.; from the class of the Gastropoda, for example, Anion spp., Biomphalaria spp., Bulinus spp., Deroceras spp., Galba spp., Lymnaea spp., Oncomelania spp., Succinea spp.; from the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti; ft may be furthermore possible to control protozoa, such as Eimeria; from the order of the Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.; from the order of the Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma pini, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlonita onukii, Chromaphis juglandicola, Chiysomphalus ficus, Cicadulina mbila, Coccomytilus halli, Coccus spp., Cryptomyzus nibis, Dalbulus spp., Dialeurodes spp., Diaphorina spp., Diaspis spp., Doralis spp., Drosicha spp., Dysaphis spp., Dysmicoccus spp., Empoasca spp., Eniosoma spp., Erythroneura spp., Euscelis bilobatus, Geococcus coffeae, Homalodisca coagulata, Hyalopterus arundinis, Icerya spp., Idiocenus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii; from the order of the Hymenoptera, for example, Diprion spp., Hoplocampa spp., Lasius spp., Monomorium pharaonic, Vespa spp.; from the order of the Isopoda, for example, Armadillidium vulgare, Oniscus asellus, Porcellio scaber; from the order of the Isoptera, for example, Reticulitermes spp., Odontotermes spp.; from the order of the Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Cheimatobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.; from the order of the Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria; from the order of the Siphonaptera, for example, Ceratophyllus spp., Xenopsylla cheopis. From the order of the Symphyla, for example, Scutigerella immaculata; from the order of the Thysanoptera, for example, Baliothrips biformis, Enneothrips flavens, Frankliniella spp., Heliothrips spp., Hercinothrips femoralis, Kakothrips spp., Rhipiphorothrips cruentatus, Scirtothrips spp., Taeniothrips cardamoni, Thrips spp.; from the order of the Thysanura, for example, Lepisma saccharina. The phytoparasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

In particular, the compounds of the invention may be used to control the following pest spcies:

Myzus persicae (aphid), Aphis gossypii (aphid), Aphis fabae (aphid), Lygus spp. (capsids), Dysdercus spp. (capsids), Nilaparvata lugens (planthopper), Nephotettixc incticeps (leafhopper), Nezara spp. (stinkbugs), Euschistus spp. (stinkbugs), Leptocorisa spp. (stinkbugs), Frankliniella occidentalis (thrip), Thrips spp. (thrips), Leptinotarsa decemlineata (Colorado potato beetle), Anthonomus grandis (boll weevil), Aonidiella spp. (scale insects), Trialeurodes spp. (white flies), Bemisia tabaci (white fly), Ostrinia nubilalis (European corn borer), Spodoptera littoralis (cotton leafworm), Heliothis virescens (tobacco budworm), Helicoverpa armigera (cotton bollworm), Helicoverpa zea (cotton bollworm), Sylepta derogata (cotton leaf roller), Pieris brassicae (white butterfly), Plutella xylostella (diamond back moth), Agrotis spp. (cutworms), Chilo suppressalis (rice stem borer), Locusta_{—}migratoria (locust), Chortiocetes terminifera (locust), Diabrotica spp. (rootworms), Panonychus ulmi (European red mite), Panonychus citri (citrus red mite), Tetranychus urticae (two-spotted spider mite), Tetranychus cinnabarinus (carmine spider mite), Phyllocoptruta oleivora (citrus rust mite), Polyphagotarsonemus latus (broad mite), Brevipalpus spp. (flat mites), Boophilus microplus (cattle tick), Dermacentor variabilis (American dog tick), Ctenocephalides felis (cat flea), Liriomyza spp. (leafminer), Musca domestica (housefly), Aedes aegypti (mosquito), Anopheles spp. (mosquitoes), Culex spp. (mosquitoes), Lucillia spp. (blowflies), Blattella germanica (cockroach), Periplaneta americana (cockroach), Blatta orientalis (cockroach), termites of the Mastotermitidae (for example Mastotermes spp.), the Kalotermitidae (for example Neotermes spp.), the Rhinotermitidae (for example Coptotermes formosanus, Reticulitermes flavipes, R. speratu, R. virginicus, R. hesperus, and R. santonensis) and the Termitidae (for example Globitermes sulfureus), Solenopsis geminata (fire ant), Monomorium pharaonis (pharaoh's ant), Damalinia spp. and Linognathus spp. (biting and sucking lice), Meloidogyne spp. (root knot nematodes), Globodera spp. and Heterodera spp. (cyst nematodes), Pratylenchus spp. (lesion nematodes), Rhodopholus spp. (banana burrowing nematodes), Tylenchulus spp. (citrus nematodes), Haemonchus contortus (barber pole worm), Caenorhabditis elegans (vinegar eelworm), Trichostrongylus spp. (gastro intestinal nematodes) and Deroceras reticulatum (slug).

The compound of formula I may be used for pest control on various plants, including soybean (e.g. in some cases 10-70 g/ha), corn (e.g. in some cases 10-70 g/ha), sugarcane (e.g. in some cases 20-200 g/ha), alfalfa (e.g. in some cases 10-70 g/ha), brassicas (e.g. in some cases 10-50 g/ha), oilseed rape (e.g. canola) (e.g. in some cases 20-70 g/ha), potatoes (including sweet potatoes) (e.g. in some cases 10-70 g/ha), cotton (e.g. in some cases 10-70 g/ha), rice (e.g. in some cases 10-70 g/ha), coffee (e.g. in some cases 30-150 g/ha), citrus (e.g. in some cases 60-200 g/ha), almonds (e.g. in some cases 40-180 g/ha), fruiting vegetables, cucurbits and pulses (e.g. tomatoes, pepper, chili, eggplant, cucumber, squash etc.) (e.g. in some cases 10-80 g/ha), tea (e.g. in some cases 20-150 g/ha), bulb vegetables (e.g. onion, leek etc.) (e.g. in some cases 30-90 g/ha), grapes (e.g. in some cases 30-180 g/ha), pome fruit (e.g. apples, pears etc.) (e.g. in some cases 30-180 g/ha), and stone fruit (e.g. pears, plums etc.) (e.g. in some cases 30-180 g/ha).

The compounds of the invention may be used for pest control on various plants, including soybean, corn, sugarcane, alfalfa, brassicas, oilseed rape (e.g. canola), potatoes (including sweet potatoes), cotton, rice, coffee, citrus, almonds, fruiting vegetables, cucurbits and pulses (e.g. tomatoes, pepper, chili, eggplant, cucumber, squash etc.), tea, bulb vegetables (e.g. onion, leek etc.), grapes, pome fruit (e.g. apples, pears etc.), stone fruit (e.g. pears, plums etc.), and cereals.

The compounds of the invention may be used on soybean to control, for example, Elasmopalpus lignosellus, Diloboderus abderus, Diabrotica speciosa, Trialeurodes spp., Bemisia spp., aphids, Sternechus subsignatus, Formicidae, Agrotis ypsilon, Julus spp., Murgantia spp., Halyomorpha spp., Thyanta spp., Megascelis ssp., Procornitermes ssp., Giyllotalpidae, Nezara viridula, Piezodorus spp., Acrosternum spp., Neomegalotomus spp., Cerotoma trifurcata, Popillia japonica, Edessa spp., Liogenys fuscus, stalk borer, Scaptocoris castanea, phyllophaga spp., Migdolus spp., Pseudoplusia includens, Anticarsia gemmatalis, Epinotia spp., Rachiplusia spp., Spodoptera spp. (e.g. Spodoptera frugiperda), Bemisia tabaci, Tetranychus spp., Agriotes spp., Euschistus spp. (e.g. Euschistus heros). The compounds of the invention are preferably used on soybean to control Diloboderus abderus, Diabrotica speciosa, Trialeurodes spp., Bemisia spp., Nezara viridula, Piezodorus spp., Acrosternum spp., Cerotoma trifurcata, Popillia japonica, Euschistus heros, Scaptocoris castanea, phyllophaga spp., Migdolus spp., Agriotes spp., Euschistus spp.

The compounds of the invention may be used on corn to control, for example, Euschistus spp. (e.g. Euschistus heros), Dichelops furcatus, Diloboderus abderus, Thyanta spp., Elasmopalpus lignosellus, Halyomorpha spp., Spodoptera frugiperda, Nezara viridula, Cerotoma trifurcata, Popillia japonica, Agrotis ypsilon, Diabrotica speciosa, aphids, Heteroptera, Procornitermes spp., Scaptocoris castanea, Formicidae, Julus ssp., Dalbulus maidis, Diabrotica spp. (e.g. Diabrotica virgifera), Mocis latipes, Bemisia tabaci, heliothis spp., Tetranychus spp., thrips spp., phyllophaga spp., Migdolus spp., scaptocoris spp., Liogenys fuscus, Spodoptera spp., Ostrinia spp., Sesamia spp., wireworms, Agriotes spp., Halotydeus destructor. The compounds of the invention are preferably used on corn to control Euschistus spp., (e.g. Euschistus heros), Dichelops furcatus, Diloboderus abderus, Nezara viridula, Cerotoma trifurcata, Popillia japonica, Diabrotica spp. (e.g. Diabrotica speciosa, Diabrotica virgifera), Tetranychus spp., Thrips spp., Phyllophaga spp., Migdolus spp., Scaptocoris spp., Agriotes spp.

The compounds of the invention may be used on sugar cane to control, for example, Sphenophorus spp., termites, Migdolus spp., Diloboderus spp., Telchin licus, Diatrea saccharalis, Mahanarva spp., Mealybugs, Chilo spp.

The compounds of the invention may be used on alfalfa to control, for example, Hypera brunneipennis, Hypera postica, Colias emytheme, Collops spp., Empoasca solana, Epitrix spp., Geocoris spp., Lygus hesperus, Lygus lineolaris, Spissistilus spp., Spodoptera spp., Aphids, Trichoplusia ni. The compounds of the invention are preferably used on alfalfa to control Hypera brunneipennis, Hypera postica, Empoasca solana, Epitrix spp., Lygus hesperus, Lygus lineolaris, Trichoplusia ni.

The compounds of the invention may be used on brassicas to control, for example, Chrysodeixis spp., Plutella xylostella, Pieris spp. (e.g. Pieris brassicae, Pieris rapae, Pieris napi), Mamestra spp. (e.g. Mamestra brassicae), Plusia spp., Trichoplusia spp. (e.g. Trichoplusia ni), Phyllotreta spp. (e.g. Phyllotreta cruciferae, Phyllotreta striolata), Spodoptera spp., Empoasca spp., thrips spp., Delia spp., Murgantia spp., Trialeurodes spp., Bemisia spp., Microtheca spp., Aphids, Chaetocnema spp., Psylliodes spp. (e.g. Psylliodes chrysocephala). The compounds of the invention are preferably used on brassicas to control Plutella xylostella, Pieris spp., Plusia spp., Trichoplusia ni, Phyllotreta spp., Thrips spp., Chaetocnema spp.

The compounds of the invention may be used on oil seed rape, e.g. canola, to control, for example, Meligethes spp. (e.g. Meligethes aeneus), Ceutorhynchus spp., (e.g. Ceutorhynchus assimilis, Ceutorhynchus napi), Halotydeus destructor, Psylloides spp. (e.g. Psylliodes chrysocephala), Phyllotreta spp. (e.g. Phyllotreta cruciferae, Phyllotreta striolata), Chaetocnema spp.

The compounds of the invention may be used on potatoes, including sweet potatoes, to control, for example, Empoasca spp., Leptinotarsa spp., Diabrotica speciosa, Phthorimaea spp., Paratrioza spp., Maladera matrida, Agriotes spp., Aphids, wireworms. The compounds of the invention are preferably used on potatoes, including sweet potatoes, to control Empoasca spp., Leptinotarsa spp., Diabrotica speciosa, Phthorimaea spp., Paratrioza spp., Agriotes spp.

The compounds of the invention may be used on cotton to control, for example, Anthonomus grandis, Pectinophora spp., heliothis spp., Spodoptera spp., Tetranychus spp. (e.g. Tetranychus urticae), Empoasca spp., Thrips spp. (e.g. Thrips tabaci, Thrips palmi), Bemisia tabaci, Trialeurodes spp., Aphids, Lygus spp. (e.g. Lygus lineolaris, Lygus Hesperus), phyllophaga spp., Scaptocoris spp., Austroasca viridigrisea, Creontiades spp., Nezara spp., Piezodorus spp., Halotydeus destructor, Oxycaraenus hyalinipennis, Dysdercus cingulatus, Amrasca spp. (e.g. Amrasca biguttula biguttula), Frankliniella spp. (e.g. Frankliniella schultzei), Scirtothrips spp. (e.g. Scirtothrips dorsali), Anaphothrips spp., Polyphagotarsonemus latus. The compounds of the invention are preferably used on cotton to control Anthonomus grandis, Tetranychus spp., Empoasca spp., thrips spp., Lygus spp., phyllophaga spp., Scaptocoris spp.

The compounds of the invention may be used on rice to control, for example, Leptocorisa spp. (e.g. Leptocorisa oratorius, Leptocorisa chinensis, Leptocorisa acuta), Cnaphalocrosis spp., Chilo spp. (e.g. Chilo suppressalis, Chilo polychrysus, Chilo auricilius), Scirpophaga spp. (e.g. Scirpophaga incertulas, Scirpophaga innotata, Scirpophaga nivella), Lissorhoptrus spp., Oebalus pugnax, Scotinophara spp. (e.g. Scotinophara coarctata, Scotinophara lurida, Scotinophara latiuscula), Nephotettix spp. (e.g. Nephotettix malayanus, Nephotettix nigropictus, Nephotettix parvus, Nephottetix virescens, Nephotettix cincticeps), Mealybugs, Sogatella furcifera, Nilaparvata lugens, Orseolia spp. (e.g. Orseolia oryzae), Cnaphalocrocis medinalis, Marasmia spp. (e.g. Marasmia patnalis, Marasmia exigua), Stenchaetothrips biformis, Thrips spp., Hydrellia spp. (e.g. Hydrellia philippina), Grasshoppers, Pomacea canaliculata, Scirpophaga innotata, Sesamia inferens, Laodelphax striatellus, Nymphula depunctalis, Oulema oryzae, Stinkbugs. The compounds of the invention are preferably used on rice to control Leptocorisa spp., Lissorhoptrus spp., Oebalus pugnax, Nephotettix spp. (e.g. Nephotettix malayanus, Nephotettix nigropictus, Nephotettix parvus, Nephottetix virescens, Nephotettix cincticeps), Sogatella furcifera, Stenchaetothrips biformis, Thrips spp., Hydrellia spp. (e.g. Hydrellia philippina), Grasshoppers, Pomacea canaliculata, Scirpophaga innotata, Chilo spp., Oulema oryzae.

The compounds of the invention may be used on coffee to control, for example, Hypothenemus spp. (e.g. Hypothenemus Hampei), Perileucoptera Coffeella, Tetranychus spp., Brevipalpus spp., Mealybugs. The compounds of the invention are preferably used on coffee to control Hypothenemus Hampei, Perileucoptera Coffeella.

The compounds of the invention may be used on citrus to control, for example, Panonychus citri, Phyllocoptruta oleivora, Brevipalpus spp. (e.g. Brevipalpus californicus, Brevipalpus phoenicis), Diaphorina citri, Scirtothrips spp. (e.g. Scirtothrips dorsalis), Thrips spp., Unaspis spp., Ceratitis capitata, Phyllocnistis spp. (e.g. Phyllocnistis citrella), Aphids, Hardscales, Softscales, Mealybugs. The compounds of the invention are preferably used on citrus to control Panonychus citri, Phyllocoptruta oleivora, Brevipalpus spp., Diaphorina citri, Scirtothrips spp., thrips spp., Phyllocnistis spp.

The compounds of the invention may be used on almonds to control, for example, Amyelois transitella, Tetranychus spp.

The compounds of the invention may be used on fruiting vegetables, cucurbits and pulses, including tomatoes, pepper, chili, eggplant, cucumber, squash etc., to control, for example, Thrips spp., Tetranychus spp. (e.g. Tetranychus urticae), Polyphagotarsonemus spp. (e.g. Polyphagotarsonemus latus), Aculops spp. (e.g. Aculops lycopersici), Empoasca spp. (e.g. Empoasca fabae), Spodoptera spp., heliothis spp., Tuta absoluta, Liriomyza spp. (e.g. Liriomyza brassicae, Liriomyza bryoniae, Liriomyza huidobrensis, Liriomyza sativae, Liriomyza trifolii), Bemisia tabaci, Trialeurodes spp., Aphids, Paratrioza spp., Frankliniella spp. (e.g. Frankliniella occidentalis, Frankliniella intonsa, Frankliniella bispinosa), Spodoptera spp. (e.g. Spodoptera exigua, Spodoptera littoralis, Spodoptera litura, Spodoptera frugiperda, Spodoptera eridania), Anthonomus spp., Phyllotreta spp., Amrasca spp. (e.g. Amrasca biguttula biguttula), Epilachna spp., Halyomorpha spp., Scirtothrips spp., Leucinodes spp. (e.g. Leucinodes orbonalis), Neoleucinodes spp. (e.g. Neoleucinodes elegantalis), Maruca spp., Fruit flies, Stinkbugs, Lepidopteras, Coleopteras, Helicoverpa spp. (e.g. Helicoverpa armigera), Heliothis spp. (e.g. Heliothis virescens), Paratrioza spp. (e.g. Paratrioza cockerelli), The compounds of the invention are preferably used on fruiting vegetables, cucurbits and pulses, including tomatoes, pepper, chili, eggplant, cucumber, squash etc., to control Thrips spp., Tetranychus spp., Polyphagotarsonemus spp., Aculops spp., Empoasca spp., Spodoptera spp., heliothis spp., Tuta absoluta, Liriomyza spp., Paratrioza spp., Frankliniella occidentalis, Frankliniella spp., Amrasca spp., Scirtothrips spp., Leucinodes spp., Neoleucinodes spp.

The compounds of the invention may be used on tea to control, for example, Pseudaulacaspis spp., Empoasca spp., Scirtothrips spp., Caloptilia theivora, Tetranychus spp. The compounds of the invention are preferably used on tea to control Empoasca spp., Scirtothrips spp.

The compounds of the invention may be used on bulb vegetables, including onion, leek etc. to control, for example, Thrips spp., Spodoptera spp., heliothis spp. The compounds of the invention are preferably used on bulb vegetables, including onion, leek etc. to control Thrips spp.

The compounds of the invention may be used on grapes to control, for example, Empoasca spp., Lobesia spp., Eupoecilia ambiguella, Frankliniella spp., Thrips spp., Tetranychus spp., Rhipiphorothrips Cruentatus, Eotetranychus Willamettei, Erythroneura Elegantula, Scaphoides spp., Scelodonta strigicollis, Mealybugs. The compounds of the invention are preferably used on grapes to control Frankliniella spp., Thrips spp., Tetranychus spp., Rhipiphorothrips Cruentatus, Scaphoides spp.

The compounds of the invention may be used on pome fruit, including apples, pears etc., to control, for example, Cacopsylla spp., Psylla spp., Panonychus ulmi, Cydia pomonella, Lepidopteras, Aphids, Hardscales, Softscales. The compounds of the invention are preferably used on pome fruit, including apples, pears etc., to control Cacopsylla spp., Psylla spp., Panonychus ulmi.

The compounds of the invention may be used on stone fruit to control, for example, Grapholita molesta, Scirtothrips spp., Thrips spp., Frankliniella spp., Tetranychus spp., Aphids, Hardscales, Softscales, Mealybugs. The compounds of the invention are preferably used on stone fruit to control Scirtothrips spp., Thrips spp., Frankliniella spp., Tetranychus spp.

The compounds of the invention may be used on cereals to control, for example, Aphids, Stinkbugs, earthmites, Eurygaster integriceps, Zabrus tenebrioides, Anisoplia austriaca, Chaetocnema aridula, Phyllotreta spp., Oulema melanopus, Oscinella spp., Delia spp., Mayetiola spp., Contarinia spp., Cephus spp., Steneotarsonemus spp., Apamea spp.

In another embodiment compounds of formula I and mixtures of the invention may be used on rice to control Baliothrips biformis (Thrips), Chilo spp. (e.g. Chilo polychrysus (Dark headed striped borer), Chilo suppressalis (Rice stemborer), Chilo indicus (Paddy stem borer), Chilo polychrysus (Dark-headed rice borer), Chilo suppressalis (Stripe stem borer)), Cnaphalocrocis medinalis (Rice leaf folder), Dicladispa armigera (Hispa), Hydrellia philipina (Rice whorl-maggot), Laodelphax spp. (Smaller brown planthopper) (e.g. Laodelphax striatellus), Lema oryzae (Rice leafbeetle), Leptocorsia acuta (Rice bug), Leptocorsia oratorius (rice bug), Lissorhoptrus oryzophilus (rice water weevil), Mythemina separata (armyworm), Nephottetix spp. (Green leafhopper) (e.g. Nephotettix cincticeps, Nephotettix malayanus, Nephotettix nigropictus, Nephotettix parvus, Nephottetix virescens), Nilaparvata lugens (Brown Planthopper), Nymphula depunctalis (Rice caseworm), Orseolia oryzae (Rice Gall midge), Oulema oryzae (Rice leafbeetle), Scirpophaga incertulas (Yellow Stemborer), Scirpophaga innotata (White Stemborer), Scotinophara coarctata (Rice black bug), Sogaella frucifera (White-backed planthopper), Steneotarsonemus spinki.

The compounds of the invention may be used to control animal housing pests including: Ants, Bedbugs (adult), Bees, Beetles, Boxelder Bugs, Carpenter Bees, Carpet Beetles, Centipedes, Cigarette, Beetles, Clover Mites, Cockroaches, Confused Flour Beetle, Crickets, Earwigs, Firebrats, Fleas, Flies, Lesser Grain Borers, Millipedes, Mosquitoes, Red Flour Beetles, Rice Weevils, Saw-toothed Grain Beetles, Silverfish, Sowbugs, Spiders, Termites, Ticks, Wasps, Cockroaches, Crickets, Flies, Litter Beetles (such as Darkling, Hide, and Carrion), Mosquitoes, Pillbugs, Scorpions, Spiders, Spider Mites (Twospotted, Spruce), Ticks.

The compounds of the invention may be used to control ornamental pests including: Ants (Including Imported fire ants), Armyworms, Azalea caterpillars, Aphids, Bagworms, Black vine weevils (adult), Boxelder bugs, Budworms, California oakworms, Cankerworms, Cockroaches, Crickets, Cutworms, Eastern tent caterpillars, Elm leaf beetles, European sawflies, Fall webworms, Flea beetles, Forest tent caterpillars, Gypsy moth larvae, Japanese beetles (adults), June beetles (adults), Lace bugs, Leaf-feeding caterpillars, Leafhoppers, Leafminers (adults), Leaf rollers, Leaf skeletonizers, Midges, Mosquitoes, Oleander moth larvae, Pillbugs, Pine sawflies, Pine shoot beetles, Pinetip moths, Plant bugs, Root weevils, Sawflies, Scale insects (crawlers), Spiders, Spittlebugs, Striped beetles, Striped oakworms, Thrips, Tip moths, Tussock moth larvae, Wasps, Broadmites, Brown softscales, California redscales (crawlers), Clover mites, Mealybugs, Pineneedlescales (crawlers), Spider mites, Whiteflies

The compounds of the invention may be used to control turf pests including: Ants (Including Imported fire ants, Armyworms, Centipedes, Crickets, Cutworms, Earwigs, Fleas (adult), Grasshoppers, Japanese beetles (adult), Millipedes, Mites, Mosquitoes (adult), Pillbugs, Sod webworms, Sow bugs, Ticks (including species which transmit Lyme disease), Bluegrass billbugs (adult), Black turfgrass ataenius (adult), Chiggers, Fleas (adult), Grubs (suppression), Hyperodes weevils (adult), Mole crickets (nymphs and young adults), Mole Crickets (mature adults), Chinch Bugs.

The compounds of formula (I) and mixture of the invention, in particular those in the tables above, may be used for soil applications, including as a seed application, to target at least the following: sucking pests such as aphids, thrips, brown plant hopper (e.g. on rice), sting bugs, white flies (e.g. on cotton and vegetables), mites; on soil pests such as corn root worm, wireworms, white grubs, zabrus, termites (e.g. on sugar cane, soy, pasture), maggots, cabbage root fly, red legged earth mite; on lepidoptera, such as spodoptera, cutworms, elasmoplpus, plutella (e.g. brassica), stem borers, leaf miners, flea beetle, Sternechus; on nematicides, such as Heterodera glycines (e.g. on soybean), Pratylenchus brachyurus (e.g. on corn), P. zeae (e.g. oncorn), P. penetrans (e.g. on corn), Meloidogyne incognita (e.g. on vegetables), Heterodera schachtii (e.g. on sugar beet), Rotylenchus reniformis (e.g. on cotton), Heterodera avenae (e.g. on cereals), Pratylenchus neglectus (e.g. on cereals), thornei (e.g. on cereals).

The compounds of formula (I) and mixture of the invention, in particular those in the tables above may be used for seed applications at least on the following: soil grubs for corn, soybeans, sugarcane: Migdolus spp; Phyllophaga spp.; Diloboderus spp; Cyclocephala spp; Lyogenys fuscus; sugarcane weevils: Sphenophorus levis & Metamasius hemtpterus; termites for soybeans, sugarcane, pasture, others: Heterotermes tenuis; Heterotermes longiceps; Cornitermes cumulans; Procornitermes triacifer; Neocapritermes opacus; Neocapritermes parvus; corn root worms for corn and potatoes: Diabrotica spp., seed Maggot: Delia platura; soil stinkbugs: Scaptocoris castanea; wireworms: Agriotes spp; Athous spp Hipnodes bicolor; Ctenicera destructor; Limonius canu; Limonius californicus; rice water weevil: Lissorhoptrus oryzophilus; Red Legged earth mites: Halotydeus destructor.

The invention therefore provides a method of combating and/or controlling an animal pest, e.g. an invertebrate animal pest, which comprises applying to the pest, to a locus of the pest, or to a plant susceptible to attack by the pest a pesticidally effective amount of a compound of formula (I). In particular, the invention provides a method of combating and/or controlling insects, acarines, nematodes or molluscs which comprises applying an insecticidally, acaricidally, nematicidally or molluscicidally effective amount of a compound of formula (I), or a composition containing a compound of formula (I), to a pest, a locus of pest, preferably a plant, or to a plant susceptible to attack by a pest, The compounds of formula (I) are preferably used against insects, acarines or nematodes.

The term “plant” as used herein includes seedlings, bushes and trees. Crops are to be understood as also including those crops which have been rendered tolerant to herbicides or classes of herbicides (e.g. ALS-, GS-, EPSPS-, PPO- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

The compounds of the invention may be applied to plant parts. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds. Treatment according to the invention of the plants and plant parts with the active compounds is carried out directly or by allowing the compounds to act on their surroundings, habitat or storage space by the customary treatment methods, for example by immersion, spraying, evaporation, fogging, scattering, painting on, injecting and, in the case of propagation material, in particular in the case of seed, also by applying one or more coats.

Compounds of formula I may be used on transgenic plants (including cultivars) obtained by genetic engineering methods and/or by conventional methods. These are understood as meaning plants having novel properties (“traits”) which have been obtained by conventional breeding, by mutagenesis or by recombinant DNA techniques. Depending on the plant species or plant cultivars, their location and growth conditions (soils, climate, vegetation period, diet), the treatment according to the invention may also result in superadditive “synergistic”) effects.

Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The preferred transgenic plants or plant cultivars which are to be treated according to the invention include all plants which, by virtue of the genetic modification, received genetic material which imparts particularly advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products.

Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds.

Examples of transgenic plants which may be mentioned are the important crop plants, such as cereals (wheat, rice), maize, soybean, potatoes, sugar beet, tomatoes, peas and other vegetable varieties, cotton, tobacco, oilseed rape and also fruit plants (with the fruits apples, pears, citrus fruits and grapes).

Compounds of formula I may be used on transgenic plants that are capable of producing one or more pesticidal proteins which confer upon the transgenic plant tolerance or resistance to harmful pests, e.g. insect pests, nematode pests and the like. Such pesticidal proteins include, without limitation, Cry proteins from Bacillus thuringiensis Cry1Ab, Cry1Ac, Cry1F, Cry2Ab, Cry2Ae, Cry3A, Cry3Bb, or Cry9C; engineered proteins such as modified Cry3A (U.S. Pat. No. 7,030,295) or Cry1A.105; or vegetative insecticidal proteins such as Vipl, Vip2 or Vip3. A full list of Bt Cry proteins and VIPs useful in the invention can be found on the worldwide web at Bacillus thuringiensis Toxin Nomenclature Database maintained by the University of Sussex (see also, Crickmore et al. (1998) Microbiol. Mol. Biol. Rev. 62:807-813). Other pesticidal proteins useful in the invention include proteins of bacteria colonizing nematodes, e.g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. Further examples of such pesticidal proteins or transgenic plants capable of synthesizing such proteins are disclosed, e.g., in EP-A 374753, WO 93/007278, WO 95/34656, EP-A 427529, EP-A 451878, WO 03/18810 and WO 03/52073. The methods for producing such transgenic plants are generally known to the person skilled in the art and some of which are commercially available such as Agrisure®CB (P1) (corn producing Cry1Ab), Agrisure®RW (P2) (corn producing mCry3A), Agrisure® Viptera (P3) (corn hybrids producing Vip3Aa); Agrisure300GT (P4) (corn hybrids producing Cry1Ab and mCry3A); YieldGard® (P5) (corn hybrids producing the Cry1Ab protein), YieldGard® Plus (P6) (corn hybrids producing Cry1Ab and Cry3Bb1), Genuity® SmartStax® (P7) (corn hybrids with Cry1A.105, Cry2Ab2, Cry1F, Cry34/35, Cry3Bb); Herculex® I (P8) (corn hybrids producing Cry1Fa) and Herculex®RW (P9) (corn hybrids producing Cry34Ab1, Cry35Ab1 and the enzyme Phosphinothricin-N-Acetyltransferase [PAT]); NuCOTN®33B (P10) (cotton cultivars producing Cry1Ac), Bollgard®I (P11) (cotton cultivars producing Cry1Ac), Bollgard®II (P12) (cotton cultivars producing Cry1Ac and Cry2Ab2) and VIPCOT® (P13) (cotton cultivars producing a Vip3Aa). Soybean Cyst Nematode resistance soybean (SCN®—Syngenta (P14)) and soybean with Aphid resistant trait (AMT® (P15)) are also of interest.

Further examples of such transgenic crops are:

1. Bt11 Maize from Syngenta Seeds SAS, Chemin de l'Hobit 27, F-31 790 St. Sauveur, France, registration number C/FR/96/05/10 (P16). Genetically modified Zea mays which has been rendered resistant to attack by the European corn borer (Ostrinia nubilalis and Sesamia nonagrioides) by transgenic expression of a truncated CryIA(b) toxin. Bt11 maize also transgenically expresses the enzyme PAT to achieve tolerance to the herbicide glufosinate ammonium.

2. Bt176 Maize from Syngenta Seeds SAS, Chemin de l'Hobit 27, F-31 790 St. Sauveur, France, registration number C/FR/96/05/10 (P17). Genetically modified Zea mays which has been rendered resistant to attack by the European corn borer (Ostrinia nubilalis and Sesamia nonagrioides) by transgenic expression of a CryIA(b) toxin. Btl 76 maize also transgenically expresses the enzyme PAT to achieve tolerance to the herbicide glufosinate ammonium.

3. MIR604 Maize from Syngenta Seeds SAS, Chemin de l'Hobit 27, F-31 790 St. Sauveur, France, registration number C/FR/96/05/10 (P18). Maize which has been rendered insect-resistant by transgenic expression of a modified CryIIIA toxin. This toxin is Cry3A055 modified by insertion of a cathepsin-D-protease recognition sequence. The preparation of such transgenic maize plants is described in WO 03/018810.

4. MON 863 Maize from Monsanto Europe S.A. 270-272 Avenue de Tervuren, B-1150 Brussels, Belgium, registration number C/DE/02/9 (P19). MON 863 expresses a CryIIIB(b1) toxin and has resistance to certain Coleoptera insects.

5. IPC 531 Cotton from Monsanto Europe S.A. 270-272 Avenue de Tervuren, B-1150 Brussels, Belgium, registration number C/ES/96/02. (P20)

6. 1507 Maize from Pioneer Overseas Corporation, Avenue Tedesco, 7 B-1160 Brussels, Belgium, registration number C/NL/00/10. (P21) Genetically modified maize for the expression of the protein Cry1F for achieving resistance to certain Lepidoptera insects and of the PAT protein for achieving tolerance to the herbicide glufosinate ammonium.

7. NK603×MON 810 Maize from Monsanto Europe S.A. 270-272 Avenue de Tervuren, B-1150 Brussels, Belgium, registration number C/GB/02/M3/03 (P22). Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a CryIA(b) toxin obtained from Bacillus thuringiensis subsp. kurstaki which brings about tolerance to certain Lepidoptera, include the European corn borer.

Further examples of transgenic plants, and of very high interest, are those carrying traits conferring resistance to 2.4D (e.g. Enlist®) (e.g. WO 2011066384) (P23), glyphosate (e.g. Roundup Ready® (P24), Roundup Ready 2 Yield® (P25)), sulfonylurea (e.g. STS®) (P26), glufosinate (e.g. Liberty Link® (P27), Ignite® (P28)), Dicamba (P29) (Monsanto), HPPD tolerance (P30) (e.g. isoxaflutole herbicide) (Bayer CropScience, Syngenta). Double or triple stacks of any of the traits described here are also of interest, including glyphosate and sulfonyl-urea tolerance ((e.g. Optimum GAT®) (P31), plants stacked with STS® and Roundup Ready® (P32) or plants stacked with STS® and Roundup Ready 2 Yield® (P33)), dicamba and glyphosate tolerance (P34) (Monsanto). Of particular interest are soybean plants carrying trains conferring resistance to 2.4D (e.g. Enlist®), glyphosate (e.g. Roundup Ready®, Roundup Ready 2 Yield®), sulfonylurea (e.g. STS®), glufosinate (e.g. Liberty Link®, Ignite®), Dicamba (Monsanto) HPPD tolerance (e.g. isoxaflutole herbicide) (Bayer CropScience, Syngenta). Double or triple stack in soybean plants of any of the traits described here are also of interest, including glyphosate and sulfonyl-urea tolerance (e.g. Optimum GAT®, plants stacked with STS® and Roundup Ready® or Roundup Ready 2 Yield®), dicamba and glyphosate tolerance (Monsanto). Transgenic crops of insect-resistant plants are also described in BATS (Zentrum für Biosicherheit and Nachhaltigkeit, Zentrum BATS, Clarastrasse 13, 4058 Basel, Switzerland) Report 2003, (http://bats.ch).

Examples of cotton transgenic events include MON 531/757/1076 (Bollgard I®—Monsanto), MON1445 (Roundup ready Cotton®—Monsanto), MON531×MON1445 (Bollgard I+RR®—Monsanto), MON15985 (Genuity Bollgard II Cotton®—Monsanto), MON88913 (Genuity RR FLEX Cotton®—Monsanto), MON15985×MON1445 (Genuity Bollgard II+RR FELX Cotton®—Monsanto), MON15983×MON88913 (Genuity Bollgard II+RR FLEX Cotton®—Monsanto), MON15985 (FibreMax Bollgard II Cotton®—Monsanto), LL25 (FibreMax LL Cotton®—BCS Stoneville), GHB614 (FibreMax GlyTol Cotton®—BCS Stoneville), LL25×MON15985 (FibreMax LL Bollgard II Cotton®—BCS Stoneville/Monsanto), GHB614×LL25 (FibreMax LL GlyTol Cotton®—BCS Stoneville), GHB614×LL25×MON15985 (FibreMax RR GlyTol Bollgard II Cotton®—BCS Stoneville), MON88913×MON15985 (FibreMax LL GlyTol Bollgard II Cotton®—Monsanto), MON88913 (FibreMax RR Flex Cotton®—Monsanto), GHB119+T304-40 (Twinlink®—BCS Stoneville), GHB119+T304-40×LL25×GHB614 (Twinlink LL GT®—BCS Stoneville), 3006-210-23×281-24-236 (PhytoGen Widestrike Insect Protection®—Dow), 3006-210-23×281-24-236×MON88913 (PhytoGen Widestrike Insect Protection+RR FLEX®—Dow/Monsanto), 3006-210-23×281-24-236×MON1445 ((PhytoGen Widestrike Insect Protection+RR®—Dow/Monsanto), MON1445 (PhytoGen Roundup Ready®—Monsanto), MON88913 (PhytoGen Roundup Ready FLEX®—Monsanto), COT102×COT67B (Vipcot®—Syngenta), COT102×COT67B x MON88913 (Vipcot RR FLEX®—Syngenta/Monsanto), 281-24-236 (Dow), 3006-210-23 (Dow), COT102 (Syngenta), COT67B (Syngenta), T304-40 (BCS Stoneville).

Examples of Soy transgenic events include MON87701×MON89788 (Genuity Roundup ready 2 Yield Soybeans®—Monsanto), MON89788 (Roundup Ready2Yield®, RR2Y®—Monsanto), MON87708 (Monsanto), 40-3-2 (Roundup Ready®, RR1®—Monsanto), MON87701 (Monsanto), DAS-68416 (Enlist Weed Control System®—Dow), DP356043 (Optimum GAT®—Pioneer), A5547-127 (LibertyLink Soybean®—Bayercropscience), A2704-12 (Bayercropscience), GU262 (Bayercropscience), W62 W98 (Bayercropscience), CRV127 (Cultivance®—BASF/EMBRAPA).

Examples of Maize transgenic events include T25 (LibertyLink®, LL®—Bayerscropscience), DHT-1 (Dow), TC1507 (Herculex I®—Dow), DAS59122-7 (Herculex RW®—Dow), TC1507+DAS59122-7—Herculex Xtra®—Dow), TC1507×DAS-59122-7×NK603 (Herculex Xtra+RR®—Dow), TC1507×DAS-59122−×MON88017×MON89034 (Genuity Smartstax Corn®, Genuity Smartstax RIB Complete®—Monsanto/Dow), MON89034×NK603 (Genuity VT double PRO®—Monsanto), MON89034+MON88017 (Genuity VT Triple PRO®—Monsanto), NK603 (Roundup Ready 2®, RR2®—Monsanto), MON810 (YieldGard BT®, Yieldgard Cornborer®—Monsanto), MON810×NK603 (YieldGard cornborer RR Corn 2®—Monasnto), MON810×MON863 (YieldGard Plus®—Monsanto), MON863×MON810×NK603 (YieldGard Plus+RR Corn2®/YieldGard RR Maize®—Monsanto), MON863×NK603 (YieldGard Rotworm+RR Corn 2®—Monsanto), MON863 (YieldBard RW®—Monsanto), MON89034 (YieldGard RW®—Monsanto), MON88017 (YieldGard VT RW®—Monsanto), MON810+MON88017 (YieldGard VT Triple®—Monsanto), MON88017+MON89034 (YieldGard VT Triple Pro®—Monsanto), Bt11+MIR604+GA21 (Agrisure 3000®—Syngenta), Bt11+TC1507+MIR604+5307+GA21 (Syngenta), Bt11+TC1507+MIR604+DAS59122+GA21 (Agrisure 3122®—Syngenta), BT11 (Agrisure CB®—Syngenta), GA21—(Agrisure GT®—Syngenta), MIR604 (Agrisure RW®—Syngenta), Bt11+MIR162 (Agrisure TL VIP®—Syngenta), BT11+MIR162+GA21 (Agrisure Viptra 3110®—Syngenta), BT11+MIR162+MIR604 (Agrisure™ 3100®—Syngenta), Event3272+BT11+MIR604+GA21 (Syngenta), BT11+MIR1692+MIR604+GA21 (Agrisure Viptera 3111®—Syngenta), BT11+MIR 162+TC1507+GA21 (Agrisure Viptera 3220®—Syngenta), BT1 1+MIR162+TC1507+MIR604+5307+GA21 (Agrisure Viptera 3222®—Syngenta), MIR162 (Syngenta), BT11+GA21+MIR162+MIR604+5307 (Syngenta), 5307 (Syngenta).

In order to apply a compound of formula (I) as an insecticide, acaricide, nematicide or molluscicide to a pest, a locus of pest, or to a plant susceptible to attack by a pest, a compound of formula (I) is usually formulated into a composition which includes, in addition to the compound of formula (I), a suitable inert diluent or carrier and, optionally, a surface active agent (SFA). SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a compound of formula (I). The composition is generally used for the control of pests such that a compound of formula (I) is applied at a rate of from 0.1 g to 10 kg per hectare, preferably from 1 g to 6 kg per hectare, more preferably from 1 g to 1 kg per hectare.

When used in a seed dressing, a compound of formula (I) is generally used at a rate of 0.0001 g to 1 Og (for example 0.001 g or 0.05 g), preferably 0.005 g to 10 g, more preferably 0.005 g to 4 g, per kilogram of seed.

In another aspect the present invention provides a composition comprising a pesticidally effective amount of a compound of formula (I), in particular an insecticidal, acaricidal, nematicidal or molluscicidal composition comprising an insecticidally, acaricidally, nematicidally or molluscicidally effective amount of a compound of formula (I) and a suitable carrier or diluent therefor. The composition is preferably an insecticidal, acaricidal, nematicidal or molluscicidal composition.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of formula (I).

Dustable powders (DP) may be prepared by mixing a compound of formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers) and mechanically grinding the mixture to a fine powder.

Soluble powders (SP) may be prepared by mixing a compound of formula (I) with one or more water-soluble inorganic salts (such as sodium bicarbonate, sodium carbonate or magnesium sulfate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula (I) and one or more powdered solid diluents or carriers, or from pre-formed blank granules by absorbing a compound of formula (I) (or a solution thereof, in a suitable agent) in a porous granular material (such as pumice, attapulgite clays, fuller's earth, kieselguhr, diatomaceous earths or ground corn cobs) or by adsorbing a compound of formula (I) (or a solution thereof, in a suitable agent) on to a hard core material (such as sands, silicates, mineral carbonates, sulfates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula (I) in water or an organic solvent, such as a ketone, alcohol or glycol ether. These solutions may contain a surface active agent (for example to improve water dilution or prevent crystallization in a spray tank).

Emulsifiable concentrates (EC) or oil-in-water emulsions (EW) may be prepared by dissolving a compound of formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula (I) either as a liquid (if it is not a liquid at room temperature, it may be melted at a reasonable temperature, typically below 70° C.) or in solution (by dissolving it in an appropriate solvent) and then emulsifiying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of formula (I) is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula (I). SCs may be prepared by ball or bead milling the solid compound of formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of formula (I) may be dry milled and added to water, containing agents hereinbefore described, to produce the desired end product.

Aerosol formulations comprise a compound of formula (I) and a suitable propellant (for example n-butane). A compound of formula (I) may also be dissolved or dispersed in a suitable medium (for example water or a water miscible liquid, such as n-propanol) to provide compositions for use in non-pressurized, hand-actuated spray pumps.

A compound of formula (I) may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerization stage such that an aqueous dispersion of oil droplets is obtained, in which each oil droplet is encapsulated by a polymeric shell and contains a compound of formula (I) and, optionally, a carrier or diluent therefor. The polymeric shell may be produced by either an interfacial polycondensation reaction or by a coacervation procedure. The compositions may provide for controlled release of the compound of formula (I) and they may be used for seed treatment. A compound of formula (I) may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of a compound of formula (I)). Such additives include surface active agents, spray additives based on oils, for example certain mineral oils or natural plant oils (such as soy bean and rape seed oil), and blends of these with other bio-enhancing adjuvants (ingredients which may aid or modify the action of a compound of formula (I)).

A compound of formula (I) may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

Wetting agents, dispersing agents and emulsifying agents may be surface SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulfuric acid (for example sodium lauryl sulfate), salts of sulfonated aromatic compounds (for example sodium dodecylbenzenesulfonate, calcium dodecylbenzenesulfonate, butylnaphthalene sulfonate and mixtures of sodium di-isopropyl- and tri-isopropyl-naphthalene sulfonates), ether sulfates, alcohol ether sulfates (for example sodium laureth-3-sulfate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulfosuccinamates, paraffin or olefine sulfonates, taurates and lignosulfonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of formula (I) may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapor or applied through distribution or incorporation of a composition (such as a granular composition or a composition packed in a water-soluble bag) in soil or an aqueous environment.

A compound of formula (I) may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs, SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula (I) (for example 0.0001 to 10%, by weight) depending upon the purpose for which they are to be used.

A compound of formula (I) may be used in mixtures with fertilizers (for example nitrogen-, potassium- or phosphorus-containing fertilizers). Suitable formulation types include granules of fertilizer. The mixtures preferably contain up to 25% by weight of the compound of formula (I).

The invention therefore also provides a fertilizer composition comprising a fertilizer and a compound of formula (I).

The compositions of this invention may contain other compounds having biological activity, for example micronutrients or compounds having fungicidal activity or which possess plant growth regulating, herbicidal, insecticidal, nematicidal or acaricidal activity.

The compound of formula (I) may be the sole active ingredient of the composition or it may be admixed with one or more additional active ingredients such as a pesticide, e.g. a insecticide, fungicide or herbicide, or a synergist or plant growth regulator where appropriate. An additional active ingredient may provide a composition having a broader spectrum of activity or increased persistence at a locus; synergize the activity or complement the activity (for example by increasing the speed of effect or overcoming repellency) of the compound of formula (I); or help to overcome or prevent the development of resistance to individual components. The particular additional active ingredient will depend upon the intended utility of the composition. Examples of suitable pesticides include the following:

a) Pyrethroids, such as permethrin, cypermethrin, fenvalerate, esfenvalerate, deltamethrin, cyhalothrin (in particular lambda-cyhalothrin and gamma cyhalothrin), bifenthrin, fenpropathrin, cyfluthrin, tefluthrin, fish safe pyrethroids (for example ethofenprox), natural pyrethrin, tetramethrin, S-bioallethrin, fenfluthrin, prallethrin, acrinathirin, etofenprox or 5-benzyl-3-furylmethyl-(E)-(1R,3S)-2,2-dimethyl-3-(2-oxothiolan-3-ylidenemethyl)cyclopropane carboxylate;
b) Organophosphates, such as profenofos, sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate or diazinon;
c) Carbamates (including aryl carbamates), such as pirimicarb, triazamate, cloethocarb, carbofuran, furathiocarb, ethiofencarb, aldicarb, thiofurox, carbosulfan, bendiocarb, fenobucarb, propoxur, methomyl or oxamyl;
d) Benzoyl ureas, such as diflubenzuron, triflumuron, hexaflumuron, flufenoxuron, diafenthiuron, lufeneron, novaluron, noviflumuron or chlorfluazuron;
e) Organic tin compounds, such as cyhexatin, fenbutatin oxide or azocyclotin;
f) Pyrazoles, such as tebufenpyrad, tolfenpyrad, ethiprole, pyriprole, fipronil, and fenpyroximate;
g) Macrolides, such as avermectins or milbemycins, for example abamectin, emamectin benzoate, ivermectin, milbemycin, spinosad, azadirachtin, milbemectin, lepimectin or spinetoram;
h) Hormones or pheromones;
i) Organochlorine compounds, such as endosulfan (in particular alpha-endosulfan), benzene hexachloride, DDT, chlordane or dieldrin;
j) Amidines, such as chlordimeform or amitraz;
k) Fumigant agents, such as chloropicrin, dichloropropane, methyl bromide or metam;
l) Neonicotinoid compounds, such as imidacloprid, thiacloprid, acetamiprid, nitenpyram, dinotefuran, thiamethoxam, clothianidin, or nithiazine;
m) Diacylhydrazines, such as tebufenozide, chromafenozide or methoxyfenozide;
n) Diphenyl ethers, such as diofenolan or pyriproxifen;
o) Pyrazolines such as Indoxacarb or metaflumizone;
p) Ketoenols, such as Spirotetramat, spirodiclofen or spiromesifen;
q) Diamides, such as flubendiamide, chlorantraniliprole (Rynaxypyr®) or cyantraniliprole;
r) Essential oils such as Bugoil®—(PlantImpact); or
s) a comopund selected from buprofezine, flonicamid, acequinocyl, bifenazate, cyenopyrafen, cyflumetofen, etoxazole, flometoquin, fluacrypyrim, fluensulfone, flufenerim, flupyradifuone, harpin, iodomethane, dodecadienol, pyridaben, pyridalyl, pyrimidifen, flupyradifurone, 4-[(6-Chloro-pyridin-3-ylmethyl)-(2,2-difluoro-ethyl)-amino]-5H-furan-2-one (DE 102006015467), CAS: 915972-17-7 (WO 2006129714; WO2011/147953; WO2011/147952), CAS: 26914-55-8 (WO 2007020986), chlorfenapyr, pymetrozine, sulfoxaflor and pyrifluqinazon.

In addition to the major chemical classes of pesticide listed above, other pesticides having particular targets may be employed in the composition, if appropriate for the intended utility of the composition. For instance, selective insecticides for particular crops, for example stemborer specific insecticides (such as cartap) or hopper specific insecticides (such as buprofezin) for use in rice may be employed. Alternatively insecticides or acaricides specific for particular insect species/stages may also be included in the compositions (for example acaricidal ovo-larvicides, such as clofentezine, flubenzimine, hexythiazox or tetradifon; acaricidal motilicides, such as dicofol or propargite; acaricides, such as bromopropylate or chlorobenzilate; or growth regulators, such as hydramethylnon, cyromazine, methoprene, chlorfluazuron or diflubenzuron).

Examples of fungicidal compounds which may be included in the composition of the invention are (E)-N-methyl-2-[2-(2,5-dimethylphenoxymethyl)phenyl]-2-methoxy-iminoacetamide (SSF-129), 4-bromo-2-cyano-N,N-dimethyl-6-trifluoromethylbenzimidazole-1-sulfonamide, α-[N-(3-chloro-2,6-xylyl)-2-methoxyacetamido]-γ-butyrolactone, 4-chloro-2-cyano-N,N-dimethyl-5-p-tolylimidazole-1-sulfonamide (IKF-916, cyamidazosulfamid), 3-5-dichloro-N-(3-chloro-1-ethyl-1-methyl-2-oxopropyl)-4-methylbenzamide (RH-7281, zoxamide), N-allyl-4,5,-dimethyl-2-trimethylsilylthiophene-3-carboxamide (MON65500), N-(1-cyano-1,2-dimethylpropyl)-2-(2,4-dichlorophenoxy)propionamide (AC382042), N-(2-methoxy-5-pyridyl)-cyclopropane carboxamide, acibenzolar (CGA245704) (e.g. acibenzolar-S-methyl), alanycarb, aldimorph, anilazine, azaconazole, azoxystrobin, benalaxyl, benomyl, benthiavalicarb, biloxazol, bitertanol, bixafen, blasticidin S, boscalid, bromuconazole, bupirimate, captafol, captan, carbendazim, carbendazim chlorhydrate, carboxin, carpropamid, carvone, CGA41396, CGA41397, chinomethionate, chlorothalonil, chlorozolinate, clozylacon, copper containing compounds such as copper oxychloride, copper oxyquinolate, copper sulfate, copper tallate and Bordeaux mixture, cyclufenamid, cymoxanil, cyproconazole, cyprodinil, debacarb, di-2-pyridyl disulfide 1,1′-dioxide, dichlofluanid, diclomezine, dicloran, diethofencarb, difenoconazole, difenzoquat, diflumetorim, O,O-di-iso-propyl-S-benzyl thiophosphate, dimefluazole, dimetconazole, dimethomorph, dimethirimol, diniconazole, dinocap, dithianon, dodecyl dimethyl ammonium chloride, dodemorph, dodine, doguadine, edifenphos, epoxiconazole, ethirimol, ethyl-(Z)-N-benzyl-N-([methyl(methyl-thioethylideneamino-oxycarbonyl)amino]thio)-β-alaninate, etridiazole, famoxadone, fenamidone (RPA407213), fenarimol, fenbuconazole, fenfuram, fenhexamid (KBR2738), fenpiclonil, fenpropidin, fenpropimorph, fentin acetate, fentin hydroxide, ferbam, ferimzone, fluazinam, fludioxonil, flumetover, fluopyram, fluoxastrobin, fluoroimide, fluquinconazole, flusilazole, flutolanil, flutriafol, fluxapyroxad, folpet, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb (SZX0722), isopropanyl butyl carbamate, isoprothiolane, isopyrazam, kasugamycin, kresoxim-methyl, LY186054, LY211795, LY248908, mancozeb, mandipropamid, maneb, mefenoxam, metalaxyl, mepanipyrim, mepronil, metalaxyl, metconazole, metiram, metiram-zinc, metominostrobin, myclobutanil, neoasozin, nickel dimethyldithiocarbamate, nitrothal-isopropyl, nuarimol, ofurace, organomercury compounds, oxadixyl, oxasulfuron, oxolinic acid, oxpoconazole, oxycarboxin, pefurazoate, penconazole, pencycuron, penflufen, penthiopyrad, phenazin oxide, phosetyl-Al, phosphorus acids, phthalide, picoxystrobin (ZA1963), polyoxinD, polyram, probenazole, prochloraz, procymidone, propamocarb, propiconazole, propineb, propionic acid, prothioconazole, pyrazophos, pyrifenox, pyrimethanil, pyraclostrobin, pyroquilon, pyroxyfur, pyrrolnitrin, quaternary ammonium compounds, quinomethionate, quinoxyfen, quintozene, sedaxane, sipconazole (F-155), sodium pentachlorophenate, spiroxamine, streptomycin, sulfur, tebuconazole, tecloftalam, tecnazene, tetraconazole, thiabendazole, thifluzamid, 2-(thiocyanomethylthio)benzothiazole, thiophanate-methyl, thiram, timibenconazole, tolclofos-methyl, tolylfluanid, triadimefon, triadimenol, triazbutil, triazoxide, tricyclazole, tridemorph, trifloxystrobin (CGA279202), triforine, triflumizole, triticonazole, validamycin A, vapam, vinclozolin, zineb and ziram, N-[9-(dichloromethylene)-1,2,3,4-tetrahydro-1,4-methanonaphthalen-5-yl]-3-(difluoromethyl)-1-methyl-1H-pyrazole-4-carboxamide[1072957-71-1], 1-methyl-3-difluoromethyl-1H-pyrazole-4-carboxylic acid (2-dichloromethylene-3-ethyl-1-methyl-indan-4-yl)-amide, and 1-methyl-3-difluoromethyl-4H-pyrazole-4-carboxylic acid [2-(2,4-dichloro-phenyl)-2-methoxy-1-methyl-ethyl]-amide.

In addition, biological agents may be included in the composition of the invention e.g. Bacillus species such as Bacillus firmus, Bacillus cereus, Bacillus subtilis, and Pasteuria species such as Pasteuria penetrans and Pasteuria nishizawae. A suitable Bacillus firmus strain is strain CNCM 1-1582 which is commercially available as BioNem™. A suitable Bacillus cereus strain is strain CNCM 1-1562. Of both Bacillus strains more details can be found in U.S. Pat. No. 6,406,690. Other biological organisms that may be included in the compositions of the invention are bacteria such as Streptomyces spp. such as S. avermitilis, and fungi such as Pochonia spp. such as P. chlamydosporia. Also of interest are Metarhizium spp. such as M. anisopliae; Pochonia spp. such as P. chlamydosporia.

The compounds of formula (I) may be mixed with soil, peat or other rooting media for the protection of plants against seed-borne, soil-borne or foliar fungal diseases.

Examples of suitable synergists for use in the compositions include piperonyl butoxide, sesamex, safroxan and dodecyl imidazole.

Suitable herbicides and plant-growth regulators for inclusion in the compositions will depend upon the intended target and the effect required.

An example of a rice selective herbicide which may be included is propanil. An example of a plant growth regulator for use in cotton is PIX™.

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

The compounds of the invention are also useful in the field of animal health, e.g. they may be used against parasitic invertebrate pests, more preferably against parasitic invertebrate pests in or on an animal. Examples of pests include nematodes, trematodes, cestodes, flies, mites, tricks, lice, fleas, true bugs and maggots. The animal may be a non-human animal, e.g. an animal associated with agriculture, e.g. a cow, a pig, a sheep, a goat, a horse, or a donkey, or a companion animal, e.g. a dog or a cat.

In a further aspect the invention provides a compound of the invention for use in a method of therapeutic treatment.

In a further aspect the invention relates to a method of controlling parasitic invertebrate pests in or on an animal comprising administering a pesticidally effective amount of a compound of the invention. The administration may be for example oral administration, parenteral administration or external administration, e.g. to the surface of the animal body. In a further aspect the invention relates to a compound of the invention for controlling parasitic invertebrate pests in or on an animal. In a further aspect the invention relates to use of a compound of the invention in the manufacture of a medicament for controlling parasitic invertebrate pests in or on an animal

In a further aspect, the invention relates to a method of controlling parasitic invertebrate pests comprising administering a pesticidally effective amount of a compound of the invention to the environment in which an animal resides.

In a further aspect the invention relates to a method of protecting an animal from a parasitic invertebrate pest comprising administering to the animal a pesticidally effective amount of a compound of the invention. In a further aspect the invention relates to a compound of the invention for use in protecting an animal from a parasitic invertebrate pest. In a further aspect the invention relates to use of a compound of the invention in the manufacture of a medicament for protecting an animal from a parasitic invertebrate pest.

In a further aspect the invention provides a method of treating an animal suffering from a parasitic invertebrate pest comprising administering to the animal a pesticidally effective amount of a compound of the invention. In a further aspect the invention relates to a compound of the invention for use in treating an animal suffering from a parasitic invertebrate pest. In a further aspect the invention relates to use of a compound of the invention in the manufacture of a medicament for treating an animal suffering from a parasitic invertebrate pest.

In a further aspect, the invention provides a pharmaceutical composition comprising a compound of the invention and a pharmaceutically suitable excipient.

The compounds of the invention may be used alone or in combination with one or more other biologically active ingredients.

In one aspect the invention provides a combination product comprising a pesticidally effective amount of a component A and a pesticidally effective amount of component B wherein component A is a compound of the invention and component B is a compound as described below.

The compounds of the invention may be used in combination with anthelmintic agents. Such anthelmintic agents include, compounds selected from the macrocyclic lactone class of compounds such as ivermectin, avermectin, abamectin, emamectin, eprinomectin, doramectin, selamectin, moxidectin, nemadectin and milbemycin derivatives as described in EP-357460, EP-444964 and EP-594291. Additional anthelmintic agents include semisynthetic and biosynthetic avermectin/milbemycin derivatives such as those described in U.S. Pat. No. 5,015,630, WO-9415944 and WO-9522552. Additional anthelmintic agents include the benzimidazoles such as albendazole, cambendazole, fenbendazole, flubendazole, mebendazole, oxfendazole, oxibendazole, parbendazole, and other members of the class. Additional anthelmintic agents include imidazothiazoles and tetrahydropyrimidines such as tetramisole, levamisole, pyrantel pamoate, oxantel or morantel. Additional anthelmintic agents include flukicides, such as triclabendazole and clorsulon and the cestocides, such as praziquantel and epsiprantel.

The compounds of the invention may be used in combination with derivatives and analogues of the paraherquamide/marcfortine class of anthelmintic agents, as well as the antiparasitic oxazolines such as those disclosed in U.S. Pat. No. 5,478,855, U.S. Pat. No. 4,639,771 and DE-19520936.

The compounds of the invention may be used in combination with derivatives and analogues of the general class of dioxomorpholine antiparasitic agents as described in WO-9615121 and also with anthelmintic active cyclic depsipeptides such as those described in WO-9611945, WO-9319053, WO-9325543, EP-626375, EP-382173, WO-9419334, EP-382173, and EP-503538.

The compounds of the invention may be used in combination with other ectoparasiticides; for example, fipronil; pyrethroids; organophosphates; insect growth regulators such as lufenuron; ecdysone agonists such as tebufenozide and the like; neonicotinoids such as imidacloprid and the like.

The compounds of the invention may be used in combination with terpene alkaloids, for example those described in International Patent Application Publication Numbers WO95/19363 or WO04/72086, particularly the compounds disclosed therein.

Other examples of such biologically active compounds that the compounds of the invention may be used in combination with include but are not restricted to the following:

Organophosphates: acephate, azamethiphos, azinphos-ethyl, azinphos-methyl, bromophos, bromophos-ethyl, cadusafos, chlorethoxyphos, chlorpyrifos, chlorfenvinphos, chlormephos, demeton, demeton-S-methyl, demeton-S-methyl sulphone, dialifos, diazinon, dichlorvos, dicrotophos, dimethoate, disulfoton, ethion, ethoprophos, etrimfos, famphur, fenamiphos, fenitrothion, fensulfothion, fenthion, flupyrazofos, fonofos, formothion, fosthiazate, heptenophos, isazophos, isothioate, isoxathion, malathion, methacriphos, methamidophos, methidathion, methyl-parathion, mevinphos, monocrotophos, naled, omethoate, oxydemeton-methyl, paraoxon, parathion, parathion-methyl, phenthoate, phosalone, phosfolan, phosphocarb, phosmet, phosphamidon, phorate, phoxim, pirimiphos, pirimiphos-methyl, profenofos, propaphos, proetamphos, prothiofos, pyraclofos, pyridapenthion, quinalphos, sulprophos, temephos, terbufos, tebupirimfos, tetrachlorvinphos, thimeton, triazophos, trichlorfon, vamidothion.

Carbamates: alanycarb, aldicarb, 2-sec-butylphenyl methylcarbamate, benfuracarb, carbaryl, carbofuran, carbosulfan, cloethocarb, ethiofencarb, fenoxycarb, fenthiocarb, furathiocarb, HCN-801, isoprocarb, indoxacarb, methiocarb, methomyl, 5-methyl-m-cumenylbutyryl(methyl)carbamate, oxamyl, pirimicarb, propoxur, thiodicarb, thiofanox, triazamate, UC-51717.

Pyrethroids: acrinathin, allethrin, alphametrin, 5-benzyl-3-furylmethyl(E)-(1R)-cis-2,2-dimethyl-3-(2-oxothiolan-3-ylidenemethyl)cyclopropanecarboxylate, bifenthrin, beta-cyfluthrin, cyfluthrin, a-cypermethrin, beta-cypermethrin, bioallethrin, bioallethrin((S)-cyclopentylisomer), bioresmethrin, bifenthrin, NCI-85193, cycloprothrin, cyhalothrin, cythithrin, cyphenothrin, deltamethrin, empenthrin, esfenvalerate, ethofenprox, fenfluthrin, fenpropathrin, fenvalerate, flucythrinate, flumethrin, fluvalinate (D isomer), imiprothrin, cyhalothrin, lambda-cyhalothrin, permethrin, phenothrin, prallethrin, pyrethrins (natural products), resmethrin, tetramethrin, transfluthrin, theta-cypermethrin, silafluofen, t-fluvalinate, tefluthrin, tralomethrin, Zeta-cypermethrin.

Arthropod growth regulators: a) chitin synthesis inhibitors: benzoylureas: chlorfluazuron, diflubenzuron, fluazuron, flucycloxuron, flufenoxuron, hexaflumuron, lufenuron, novaluron, teflubenzuron, triflumuron, buprofezin, diofenolan, hexythiazox, etoxazole, chlorfentazine; b) ecdysone antagonists: halofenozide, methoxyfenozide, tebufenozide; c) juvenoids: pyriproxyfen, methoprene (including S-methoprene), fenoxycarb; d) lipid biosynthesis inhibitors: spirodiclofen.

Other antiparasitics: acequinocyl, amitraz, AKD-1022, ANS-118, azadirachtin, Bacillus thuringiensis, bensultap, bifenazate, binapacryl, bromopropylate, BTG-504, BTG-505, camphechlor, cartap, chlorobenzilate, chlordimeform, chlorfenapyr, chromafenozide, clothianidine, cyromazine, diacloden, diafenthiuron, DBI-3204, dinactin, dihydroxymethyldihydroxypyrrolidine, dinobuton, dinocap, endosulfan, ethiprole, ethofenprox, fenazaquin, flumite, MTI-800, fenpyroximate, fluacrypyrim, flubenzimine, flubrocythrinate, flufenzine, flufenprox, fluproxyfen, halofenprox, hydramethylnon, IKI-220, kanemite, NC-196, neem guard, nidinorterfuran, nitenpyram, SD-35651, WL-108477, pirydaryl, propargite, protrifenbute, pymethrozine, pyridaben, Buprofezine pyrimidifen, NC-1111, R-195, RH-0345, RH-2485, RYI-210, S-1283, S-1833, SI-8601, silafluofen, silomadine, spinosad, tebufenpyrad, tetradifon, tetranactin, thiacloprid, thiocyclam, thiamethoxam, tolfenpyrad, triazamate, triethoxyspinosyn, trinactin, verbutin, vertalec, YI-5301.

Fungicides: acibenzolar, aldimorph, ampropylfos, andoprim, azaconazole, azoxystrobin, benalaxyl, benomyl, bialaphos, blasticidin-S, Bordeaux mixture, bromuconazole, bupirimate, carpropamid, captafol, captan, carbendazim, chlorfenazole, chloroneb, chloropicrin, chlorothalonil, chlozolinate, copper oxychloride, copper salts, cyflufenamid, cymoxanil, cyproconazole, cyprodinil, cyprofuram, RH-7281, diclocymet, diclobutrazole, diclomezine, dicloran, difenoconazole, RP-407213, dimethomorph, domoxystrobin, diniconazole, diniconazole-M, dodine, edifenphos, epoxiconazole, famoxadone, fenamidone, fenarimol, fenbuconazole, fencaramid, fenpiclonil, fenpropidin, fenpropimorph, fentin acetate, fluazinam, fludioxonil, flumetover, flumorf/flumorlin, fentin hydroxide, fluoxastrobin, fluquinconazole, flusilazole, flutolanil, flutriafol, folpet, fosetyl-aluminium, furalaxyl, furametapyr, hexaconazole, ipconazole, iprobenfos, iprodione, isoprothiolane, kasugamycin, krsoxim-methyl, mancozeb, maneb, mefenoxam, mepronil, metalaxyl, metconazole, metominostrobin/fenominostrobin, metrafenone, myclobutanil, neo-asozin, nicobifen, orysastrobin, oxadixyl, penconazole, pencycuron, probenazole, prochloraz, propamocarb, propioconazole, proquinazid, prothioconazole, pyrifenox, pyraclostrobin, pyrimethanil, pyroquilon, quinoxyfen, spiroxamine, sulfur, tebuconazole, tetrconazole, thiabendazole, thifluzamide, thiophanate-methyl, thiram, tiadinil, triadimefon, triadimenol, tricyclazole, trifloxystrobin, triticonazole, validamycin, vinclozin.

Biological agents: Bacillus thuringiensis ssp aizawai, kurstaki, Bacillus thuringiensis delta endotoxin, baculovirus, entomopathogenic bacteria, virus and fungi.

Bactericides: chlortetracycline, oxytetracycline, streptomycin.

Other biological agents: enrofloxacin, febantel, penethamate, moloxicam, cefalexin, kanamycin, pimobendan, clenbuterol, omeprazole, tiamulin, benazepril, pyriprole, cefquinome, florfenicol, buserelin, cefovecin, tulathromycin, ceftiour, carprofen, metaflumizone, praziquarantel, triclabendazole.

When used in combination with other active ingredients, the compounds of the invention are preferably used in combination with the following: imidacloprid, enrofloxacin, praziquantel, pyrantel embonate, febantel, penethamate, moloxicam, cefalexin, kanamycin, pimobendan, clenbuterol, fipronil, ivermectin, omeprazole, tiamulin, benazepril, milbemycin, cyromazine, thiamethoxam, pyriprole, deltamethrin, cefquinome, florfenicol, buserelin, cefovecin, tulathromycin, ceftiour, selamectin, carprofen, metaflumizone, moxidectin, methoprene (including S-methoprene), clorsulon, pyrantel, amitraz, triclabendazole, avermectin, abamectin, emamectin, eprinomectin, doramectin selamectin, nemadectin, albendazole, cambendazole, fenbendazole, flubendazole, mebendazole, oxfendazole, oxibendazole, parbendazole, tetramisole, levamisole, pyrantel pamoate, oxantel, morantel, triclabendazole, epsiprantel, fipronil, lufenuron, ecdysone or tebufenozide; more preferably, enrofloxacin, praziquantel, pyrantel embonate, febantel, penethamate, moloxicam, cefalexin, kanamycin, pimobendan, clenbuterol, omeprazole, tiamulin, benazepril, pyriprole, cefquinome, florfenicol, buserelin, cefovecin, tulathromycin, ceftiour, selamectin, carprofen, moxidectin, clorsulon, pyrantel, eprinomectin, doramectin, selamectin, nemadectin, albendazole, cambendazole, fenbendazole, flubendazole, mebendazole, oxfendazole, oxibendazole, parbendazole, tetramisole, levamisole, pyrantel pamoate, oxantel, morantel, triclabendazole, epsiprantel, lufenuron or ecdysone; even more preferably enrofloxacin, praziquantel, pyrantel embonate, febantel, penethamate, moloxicam, cefalexin, kanamycin, pimobendan, clenbuterol, omeprazole, tiamulin, benazepril, pyriprole, cefquinome, florfenicol, buserelin, cefovecin, tulathromycin, ceftiour, selamectin, carprofen, moxidectin, clorsulon or pyrantel.

Examples of ratios include 100:1 to 1:6000, 50:1 to 1:50, 20:1 to 1:20, even more especially from 10:1 to 1:10, 5:1 to 1:5, 2:1 to 1:2, 4:1 to 2:1, 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are understood to include, on the one hand, ratios by weight and also, on other hand, molar ratios.

Of particular note is a combination where the additional active ingredient has a different site of action from the compound of formula I. In certain instances, a combination with at least one other parasitic invertebrate pest control active ingredient having a similar spectrum of control but a different site of action will be particularly advantageous for resistance management. Thus, a combination product of the invention may comprise a pesticidally effective amount of a compound of formula I and pesticidally effective amount of at least one additional parasitic invertebrate pest control active ingredient having a similar spectrum of control but a different site of action.

One skilled in the art recognizes that because in the environment and under physiological conditions salts of chemical compounds are in equilibrium with their corresponding non salt forms, salts share the biological utility of the non salt forms.

Thus a wide variety of salts of compounds of the invention (and active ingredients used in combination with the active ingredients of the invention) may be useful for control of invertebrate pests and animal parasites. Salts include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids.

The compounds of the invention also include N-oxides. Accordingly, the invention comprises combinations of compounds of the invention including N-oxides and salts thereof and an additional active ingredient including N-oxides and salts thereof.

The compositions for use in animal health may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compounds of the invention can be applied without other adjuvants, but most often application will be of a formulation comprising one or more active ingredients with suitable carriers, diluents, and surfactants and possibly in combination with a food depending on the contemplated end use. One method of application involves spraying a water dispersion or refined oil solution of the combination products. Compositions with spray oils, spray oil concentrations, spreader stickers, adjuvants, other solvents, and synergists such as piperonyl butoxide often enhance compound efficacy. Such sprays can be applied from spray containers such as a can, a bottle or other container, either by means of a pump or by releasing it from a pressurized container, e.g., a pressurized aerosol spray can. Such spray compositions can take various forms, for example, sprays, mists, foams, fumes or fog. Such spray compositions thus can further comprise propellants, foaming agents, etc. as the case may be. Of note is a spray composition comprising a pesticidally effective amount of a compound of the invention and a carrier. One embodiment of such a spray composition comprises a pesticidally effective amount of a compound of the invention and a propellant. Representative propellants include, but are not limited to, methane, ethane, propane, butane, isobutane, butene, pentane, isopentane, neopentane, pentene, hydrofluorocarbons, chlorofluorocarbons, dimethyl ether, and mixtures of the foregoing. Of note is a spray composition (and a method utilizing such a spray composition dispensed from a spray container) used to control at least one parasitic invertebrate pest selected from the group consisting of mosquitoes, black flies, stable flies, deer flies, horse flies, wasps, yellow jackets, hornets, ticks, spiders, ants, gnats, and the like, including individually or in combinations.

The controlling of animal parasites includes controlling external parasites that are parasitic to the surface of the body of the host animal (e.g., shoulders, armpits, abdomen, inner part of the thighs) and internal parasites that are parasitic to the inside of the body of the host animal (e.g., stomach, intestine, lung, veins, under the skin, lymphatic tissue). External parasitic or disease transmitting pests include, for example, chiggers, ticks, lice, mosquitoes, flies, mites and fleas. Internal parasites include heartworms, hookworms and helminths. The compounds of the invention may be particularly suitable for combating external parasitic pests. The compounds of the invention may be suitable for systemic and/or non-systemic control of infestation or infection by parasites on animals.

The compounds of the invention may be suitable for combating parasitic invertebrate pests that infest animal subjects including those in the wild, livestock and agricultural working animals. Livestock is the term used to refer (singularly or plurally) to a domesticated animal intentionally reared in an agricultural setting to make produce such as food or fiber, or for its labor; examples of livestock include cattle, sheep, goats, horses, pigs, donkeys, camels, buffalo, rabbits, hens, turkeys, ducks and geese (e.g., raised for meat, milk, butter, eggs, fur, leather, feathers and/or wool), cultured fish, honeybees. By combating parasites, fatalities and performance reduction (in terms of meat, milk, wool, skins, eggs, etc.) are reduced, so that applying the compounds of the invention allows more economic and simple husbandry of animals.

By controlling these pests it is intended to reduce deaths and improve performance (in the case of meat, milk, wool, hides, eggs, honey and the like) and health of the host animal. Also, controlling parasites may help to prevent the transmittance of infectious agents, the term “controlling” referring to the veterinary field, meaning that the active compounds are effective in reducing the incidence of the respective parasite in an animal infected with such parasites to innocuous levels, e.g. the active compound is effective in killing the respective parasite, inhibiting its growth, or inhibiting its proliferation.

The compounds of the invention may be suitable for combating parasitic invertebrate pests that infest companion animals and pets (e.g., dogs, cats, pet birds and aquarium fish), research and experimental animals (e.g., hamsters, guinea pigs, rats and mice), as well as animals raised for/in zoos, wild habitats and/or circuses.

In an embodiment of this invention, the animal is preferably a vertebrate, and more preferably a mammal, avian or fish. In a particular embodiment, the animal subject is a mammal (including great apes, such as humans). Other mammalian subjects include primates (e.g., monkeys), bovine (e.g., cattle or dairy cows), porcine (e.g., hogs or pigs), ovine (e.g., goats or sheep), equine (e.g., horses), canine (e.g., dogs), feline (e.g., house cats), camels, deer, donkeys, buffalos, antelopes, rabbits, and rodents (e.g., guinea pigs, squirrels, rats, mice, gerbils, and hamsters). Avians include Anatidae (swans, ducks and geese), Columbidae (e.g., doves and pigeons), Phasianidae (e.g., partridges, grouse and turkeys), Thesienidae (e.g., domestic chickens), Psittacines (e.g., parakeets, macaws, and parrots), game birds, and ratites (e.g., ostriches).

Birds treated or protected by the compounds of the invention can be associated with either commercial or noncommercial aviculture. These include Anatidae, such as swans, geese, and ducks, Columbidae, such as doves and domestic pigeons, Phasianidae, such as partridge, grouse and turkeys, Thesienidae, such as domestic chickens, and Psittacines, such as parakeets, macaws and parrots raised for the pet or collector market, among others.

For purposes of the present invention, the term “fish” is understood to include without limitation, the Teleosti grouping of fish, i.e., teleosts. Both the Salmoniformes order (which includes the Salmonidae family) and the Perciformes order (which includes the Centrarchidae family) are contained within the Teleosti grouping. Examples of potential fish recipients include the Salmonidae, Serranidae, Sparidae, Cichlidae, and Centrarchidae, among others.

Other animals are also contemplated to benefit from the inventive methods, including marsupials (such as kangaroos), reptiles (such as farmed turtles), and other economically important domestic animals for which the inventive methods are safe and effective in treating or preventing parasite infection or infestation.

Examples of parasitic invertebrate pests controlled by administering a pesticidally effective amount of the compounds of the invention to an animal to be protected include ectoparasites (arthropods, acarines, etc.) and endoparasites (helminths, e.g., nematodes, trematodes, cestodes, acanthocephalans, etc. and protozoae, such as coccidia).

The disease or group of diseases described generally as helminthiasis is due to infection of an animal host with parasitic worms known as helminths. The term ‘helminths’ is meant to include nematodes, trematodes, cestodes and acanthocephalans. Helminthiasis is a prevalent and serious economic problem with domesticated animals such as swine, sheep, horses, cattle, goats, dogs, cats and poultry.

Among the helminths, the group of worms described as nematodes causes widespread and at times serious infection in various species of animals.

Nematodes that are contemplated to be treated by the compounds of the invention include, without limitation, the following genera: Acanthocheilonema, Aelurostrongylus, Ancylostoma, Angiostrongylus, Ascaridia, Ascaris, Brugia, Bunostomum, Capillaria, Chabertia, Cooperia, Crenosoma, Dictyocaulus, Dioctophyme, Dipetalonema, Diphyllobothrium, Dirofilaria, Dracunculus, Enterobius, Filaroides, Haemonchus, Heterakis, Lagochilascaris, Loa, Mansonella, Muellerius, Necator, Nematodirus, Oesophagostomum, Ostertagia, Oxyuris, Parafilaria, Parascaris, Physaloptera, Protostrongylus, Setaria, Spirocerca, Stephanofilaria, Strongyloides, Strongylus, Thelazia, Toxascaris, Toxocara, Trichinella, Trichonema, Trichostrongylus, Trichuris, Uncinaria and Wuchereria.

Of the above, the most common genera of nematodes infecting the animals referred to above are Haemonchus, Trichostrongylus, Ostertagia, Nematodirus, Cooperia, Ascaris, Bunostomum, Oesophagostomum, Chabertia, Trichuris, Strongylus, Trichonema, Dictyocaulus, Capillaria, Heterakis, Toxocara, Ascaridia, Oxyuris, Ancylostoma, Uncinaria, Toxascaris and Parascaris. Certain of these, such as Nematodirus, Cooperia and Oesophagostomum attack primarily the intestinal tract while others, such as Haemonchus and Ostertagia, are more prevalent in the stomach while others such as Dictyocaulus are found in the lungs. Still other parasites may be located in other tissues such as the heart and blood vessels, subcutaneous and lymphatic tissue and the like.

Trematodes that are contemplated to be treated by the invention and by the inventive methods include, without limitation, the following genera: Alaria, Fasciola, Nanophyetus, Opisthorchis, Paragonimus and Schistosoma.

Cestodes that are contemplated to be treated by the invention and by the inventive methods include, without limitation, the following genera: Diphyllobothrium, Diplydium, Spirometra and Taenia.

The most common genera of parasites of the gastrointestinal tract of humans are Ancylostoma, Necator, Ascaris, Strongy hides, Trichinella, Capillaria, Trichuris and Enterobius. Other medically important genera of parasites which are found in the blood or other tissues and organs outside the gastrointestinal tract are the filarial worms such as Wuchereria, Brugia, Onchocerca and Loa, as well as Dracunculus and extra intestinal stages of the intestinal worms Strongyloides and Trichinella.

Numerous other helminth genera and species are known to the art, and are also contemplated to be treated by the compounds of the invention. These are enumerated in great detail in Textbook of Veterinary Clinical Parasitology, Volume 1, Helminths, E. J. L. Soulsby, F. A. Davis Co., Philadelphia, Pa.; Helminths, Arthropods and Protozoa, (6^{th} Edition of Monnig's Veterinary Helminthology and Entomology), E. J. L. Soulsby, Williams and Wilkins Co., Baltimore, Md.

The compounds of the invention may be effective against a number of animal ectoparasites (e.g., arthropod ectoparasites of mammals and birds in particular insects such as flies (stinging and licking), parasitic fly larvae, lice, hair lice, bird lice, fleas and the like; or acarids, such as ticks, for examples hard ticks or soft ticks, or mites, such as scab mites, harvest mites, bird mites and the like).

Insect and acarine pests include, e.g., biting insects such as flies and mosquitoes, mites, ticks, lice, fleas, true bugs, parasitic maggots, and the like.

Adult flies include, e.g., the horn fly or Haematobia irritans, the horse fly or Tabanus spp., the stable fly or Stomoxys calcitrans, the black fly or Simulium spp., the deer fly or Chrysops spp., the louse fly or Melophagus ovinus, and the tsetse fly or Glossina spp. Parasitic fly maggots include, e.g., the bot fly (Oestrus ovis and Cuterebra spp.), the blow fly or Phaenicia spp., the screwworm or Cochliomyia hominivorax, the cattle grub or Hypoderma spp., the fleeceworm and the Gastrophilus of horses. Mosquitoes include, for example, Culex spp., Anopheles spp. and Aedes spp.

Mites include Mesostigmalphatalpha spp. e.g., mesostigmatids such as the chicken mite, Dermalphanyssus galphallinalphae; itch or scab mites such as Sarcoptidae spp. for example, Salpharcoptes scalphabiei; mange mites such as Psoroptidae spp. including Chorioptes bovis and Psoroptes ovis; chiggers e.g., Trombiculidae spp. for example the North American chigger, Trombiculalpha alphalfreddugesi.

Ticks include, e.g., soft-bodied ticks including Argasidae spp. for example Argalphas spp. and Ornithodoros spp.; hard-bodied ticks including Ixodidae spp., for example Rhipicephalphalus sanguineus, Dermacentor variabilis, Dermacentor andersoni, Amblyomma americanum, Ixodes scapularis and other Rhipicephalus spp. (including the former Boophilus genera).

Lice include, e.g., sucking lice, e.g., Menopon spp. and Bovicola spp.; biting lice, e.g., Haematopinus spp., Linognathus spp. and Solenopotes spp.

Fleas include, e.g., Ctenocephalides spp., such as dog flea (Ctenocephalides canis) and cat flea (Ctenocephalides felis); Xenopsylla spp. such as oriental rat flea (Xenopsylla cheopis); and Pulex spp. such as human flea (Pulex irritans).

True bugs include, e.g., Cimicidae or e.g., the common bed bug (Cimex lectularius); Triatominae spp. including triatomid bugs also known as kissing bugs; for example Rhodnius prolixus and Triatoma spp.

Generally, flies, fleas, lice, mosquitoes, gnats, mites, ticks and helminths cause tremendous losses to the livestock and companion animal sectors. Arthropod parasites also are a nuisance to humans and can vector disease-causing organisms in humans and animals.

Numerous other parasitic invertebrate pests are known to the art, and are also contemplated to be treated by the compounds of the invention. These are enumerated in great detail in Medical and Veterinary Entomology, D. S. Kettle, John Wiley AND Sons, New York and Toronto; Control of Arthropod Pests of Livestock: A Review of Technology, R. O. Drummand, J. E. George, and S. E. Kunz, CRC Press, Boca Raton, FIa.

The compounds of the invention may also be effective against ectoparasites, e.g. insects such as flies (stinging and licking), parasitic fly larvae, lice, hair lice, bird lice, fleas and the like; or acarids, such as ticks, for examples hard ticks or soft ticks, or mites, such as scab mites, harvest mites, bird mites and the like. These include e.g. flies such as Haematobia (Lyperosia) irritans (horn fly), Simulium spp. (blackfly), Glossina spp. (tsetse flies), Hydrotaea irritans (head fly), Musca autumnalis (face fly), Musca domestica (house fly), Morellia simplex (sweat fly), Tabanus spp. (horse fly), Hypoderma bovis, Hypoderma lineatum, Lucilia sericata, Lucilia cuprina (green blowfly), Calliphora spp. (blowfly), Protophormia spp., Oestrus ovis (nasal botfly), Culicoides spp. (midges), Hippobosca equine, Gastrophilus intestinalis, Gastrophilus haemorrhoidalis and Gastrophilus nasalis; lice such as Bovicola (Damalinia) bovis, Bovicola equi, Haematopinus asini, Felicola subrostratus, Heterodoxus spiniger, Lignonathus setosus and Trichodectes canis; keds such as Melophagus ovinus; and mites such as Psoroptes spp., Sarcoptes scabei, Chorioptes bovis, Demodex equi, Cheyletiella spp., Notoedres cati, Trombicula spp. and Otodectes cyanotis (ear mites).

Examples of species of animal health pesets include those from the order of the Anoplurida, for example Haematopinus spp., Linognathus spp., Pediculus spp., Phtirus spp., Solenopotes spp.; particular examples are: Linognathus setosus, Linognathus vituli, Linognathus ovillus, Linognathus oviformis, Linognathus pedalis, Linognathus stenopsis, Haematopinus asini macrocephalus, Haematopinus eurysternus, Haematopinus suis, Pediculus humanus capitis, Pediculus humanus corporis, Phylloera vastatrix, Phthirus pubis, Solenopotes capillatus; from the order of the Mallophagida and the suborders Amblycerina and Ischnocerina, for example Trimenopon spp., Menopon spp., Trinoton spp., Bovicola spp., Werneckiella spp., Lepikentron spp., Damalina spp., Trichodectes spp., Felicola spp.; particular examples are: Bovicola bovis, Bovicola ovis, Bovicola limbata, Damalina bovis, Trichodectes canis, Felicola subrostratus, Bovicola caprae, Lepikentron ovis, Werneckiella equi; from the order of the Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; particular examples are: Aedes aegypti, Aedes albopictus, Aedes taeniorhynchus, Anopheles gambiae, Anopheles maculipennis, Calliphora erythrocephala, Chrysozona pluvialis, Culex quinquefasciatus, Culex pipiens, Culex tarsalis, Fannia canicularis, Sarcophaga carnaria, Stomoxys calcitrans, Tipula paludosa, Lucilia cuprina, Lucilia sericata, Simulium reptans, Phlebotomus papatasi, Phlebotomus longipalpis, Odagmia ornata, Wilhelmia equina, Boophthora erythrocephala, Tabanus bromius, Tabanus spodopterus, Tabanus atratus, Tabanus sudeticus, Hybomitra ciurea, Chrysops caecutiens, Chrysops relictus, Haematopota pluvialis, Haematopota italica, Musca autumnalis, Musca domestica, Haematobia irritans irritans, Haematobia irritans exigua, Haematobia stimulans, Hydrotaea irritans, Hydrotaea albipuncta, Chrysomya chloropyga, Chrysomya bezziana, Oestrus ovis, Hypoderma bovis, Hypoderma lineatum, Przhevalskiana silenus, Dermatobia hominis, Melophagus ovinus, Lipoptena capreoli, Lipoptena cervi, Hippobosca variegata, Hippobosca equina, Gasterophilus intestinalis, Gasterophilus haemorroidalis, Gasterophilus inermis, Gasterophilus nasalis, Gasterophilus nigricornis, Gasterophilus pecorum, Braula coeca; from the order of the Siphonapterida, for example Pulex spp., Ctenocephalides spp., Tunga spp., Xenopsylla spp., Ceratophyllus spp.; particular examples are: Ctenocephalides canis, Ctenocephalides felis, Pulex irritans, Tunga penetrans, Xenopsylla cheopis; from the order of the Heteropterida, for example Cimex spp., Triatoma spp., Rhodnius spp., Panstrongylus spp; from the order of the Blattarida, for example Blatta orientalis, Periplaneta americana, Blattela germanica, Supella spp. (e.g. Suppella longipalpa); from the subclass of the Acari (Acarina) and the orders of the Meta- and Mesostigmata, for example Argas spp., Ornithodorus spp., Otobius spp., Ixodes spp., Amblyomma spp., Rhipicephalus (Boophilus) spp Dermacentor spp., Haemophysalis spp., Hyalomma spp., Dermanyssus spp., Rhipicephalus spp. (the original genus of multi host ticks) Ornithonyssus spp., Pneumonyssus spp., Raillietia spp., Pneumonyssus spp., Sternostoma spp., Varroa spp., Acarapis spp.; particular examples are: Argas persicus, Argas reflexus, Ornithodorus moubata, Otobius megnini, Rhipicephalus (Boophilus) microplus, Rhipicephalus (Boophilus) decoloratus, Rhipicephalus (Boophilus) annulatus, Rhipicephalus (Boophilus) calceratus, Hyalomma anatolicum, Hyalomma aegypticum, Hyalomma marginatum, Hyalomma transiens, Rhipicephalus evertsi, Ixodes ricinus, Ixodes hexagonus, Ixodes canisuga, Ixodes pilosus, Ixodes rubicundus, Ixodes scapularis, Ixodes holocyclus, Haemaphysalis concinna, Haemaphysalis punctata, Haemaphysalis cinnabarina, Haemaphysalis otophila, Haemaphysalis leachi, Haemaphysalis longicorni, Dermacentor marginatus, Dermacentor reticulatus, Dermacentor pictus, Dermacentor albipictus, Dermacentor andersoni, Dermacentor variabilis, Hyalomma mauritanicum, Rhipicephalus sanguineus, Rhipicephalus bursa, Rhipicephalus appendiculatus, Rhipicephalus capensis, Rhipicephalus turanicus, Rhipicephalus zambeziensis, Amblyomma americanum, Amblyomma variegatum, Amblyomma maculatum, Amblyomma hebraeum, Amblyomma cajennense, Dermanyssus gallinae, Ornithonyssus bursa, Ornithonyssus sylviarum, Varroa jacobsoni; from the order of the Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.; particular examples are: Cheyletiella yasguri, Cheyletiella blakei, Demodex canis, Demodex bovis, Demodex ovis, Demodex caprae, Demodex equi, Demodex caballi, Demodex suis, Neotrombicula autumnalis, Neotrombicula desaleri, Neoschongastia xerothermobia, Trombicula akamushi, Otodectes cynotis, Notoedres cati, Sarcoptis canis, Sarcoptes bovis, Sarcoptes ovis, Sarcoptes rupicaprae (S. caprae), Sarcoptes equi, Sarcoptes suis, Psoroptes ovis, Psoroptes cuniculi, Psoroptes equi, Chorioptes bovis, Psoergates ovis, Pneumonyssoidic mange, Pneumonyssoides caninum, Acarapis woodi; Gasterophilus spp., Stomoxys spp., Trichodectes spp., Rhodnius spp., Ctenocephalides canis, Cimx lecturius, Ctenocephalides felis, Lucilia cuprina; examples of acari include Ornithodoros spp., Ixodes spp., Boophilus spp.

Treatments of the invention are by conventional means such as by enteral administration in the form of, for example, tablets, capsules, drinks, drenching preparations, granulates, pastes, boli, feed-through procedures, or suppositories; or by parenteral administration, such as, for example, by injection (including intramuscular, subcutaneous, intravenous, intraperitoneal) or implants; or by nasal administration; or by dermal application in the form of, for example, bathing or dipping, spraying, pouring-on and spotting-on, washing, dusting, and with the aid of active-compound-comprising shaped articles such as collars, ear tags, tail tags, limb bands, halters, marking devices and the like.

When compounds of the invention are applied in combination with an additional biologically active ingredient, they may be administered separately e.g. as separate compositions. In this case, the biologically active ingredients may be administered simultaneously or sequentially. Alternatively, the biologically active ingredients may be components of one composition.

The compounds of the invention may be administered in a controlled release form, for example in subcutaneous or orally adminstered slow release formulations.

Typically a parasiticidal composition according to the present invention comprises a compound of the invention, optionally in combination with an additional biologically active ingredient, or N-oxides or salts thereof, with one or more pharmaceutically or veterinarily acceptable carriers comprising excipients and auxiliaries selected with regard to the intended route of administration (e.g., oral or parenteral administration such as injection) and in accordance with standard practice. In addition, a suitable carrier is selected on the basis of compatibility with the one or more active ingredients in the composition, including such considerations as stability relative to pH and moisture content. Therefore of note are compounds of the invention for protecting an animal from an invertebrate parasitic pest comprising a parasitically effective amount of a compound of the invention, optionally in combination with an additional biologically active ingredient and at least one carrier.

For parenteral administration including intravenous, intramuscular and subcutaneous injection, the compounds of the invention can be formulated in suspension, solution or emulsion in oily or aqueous vehicles, and may contain adjuncts such as suspending, stabilizing and/or dispersing agents.

The compounds of the invention may also be formulated for bolus injection or continuous infusion. Pharmaceutical compositions for injection include aqueous solutions of water-soluble forms of active ingredients (e.g., a salt of an active compound), preferably in physiologically compatible buffers containing other excipients or auxiliaries as are known in the art of pharmaceutical formulation. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes.

Aqueous injection suspensions may contain substances that increase the viscosity of the suspension, such as sodium carboxymethyl cellulose, sorbitol, or dextran. Formulations for injection may be presented in unit dosage form, e.g., in ampoules or in multi-dose containers. Alternatively, the active ingredient may be in powder form for constitution with a suitable vehicle, e.g., sterile, pyrogen-free water, before use.

In addition to the formulations described supra, the compounds of the invention may also be formulated as a depot preparation. Such long acting formulations may be administered by implantation (for example, subcutaneously or intramuscularly) or by intramuscular or subcutaneous injection.

The compounds of the invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

For administration by inhalation, the compounds of the invention can be delivered in the form of an aerosol spray using a pressurized pack or a nebulizer and a suitable propellant, e.g., without limitation, dichlorodifluoromethane, trichlorofluoromethane, dichlorotetrafluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

The compounds of the invention may have favourable pharmacokinetic and pharmacodynamic properties providing systemic availability from oral administration and ingestion. Therefore after ingestion by the animal to be protected, parasiticidally effective concentrations of a compound of the invention in the bloodstream may protect the treated animal from blood-sucking pests such as fleas, ticks and lice. Therefore of note is a composition for protecting an animal from an invertebrate parasite pest in a form for oral administration (i.e. comprising, in addition to a parasiticidally effective amount of a compound of the invention, one or more carriers selected from binders and fillers suitable for oral administration and feed concentrate carriers).

For oral administration in the form of solutions (the most readily available form for absorption), emulsions, suspensions, pastes, gels, capsules, tablets, boluses, powders, granules, rumen-retention and feed/water/lick blocks, the compounds of the invention can be formulated with binders/fillers known in the art to be suitable for oral administration compositions, such as sugars and sugar derivatives (e.g., lactose, sucrose, mannitol, sorbitol), starch (e.g., maize starch, wheat starch, rice starch, potato starch), cellulose and derivatives (e.g., methylcellulose, carboxymethylcellulose, ethylhydroxycellulose), protein derivatives (e.g., zein, gelatin), and synthetic polymers (e.g., polyvinyl alcohol, polyvinylpyrrolidone). If desired, lubricants (e.g., magnesium stearate), disintegrating agents (e.g., cross-linked polyvinylpyrrolidinone, agar, alginic acid) and dyes or pigments can be added. Pastes and gels often also contain adhesives (e.g., acacia, alginic acid, bentonite, cellulose, xanthan gum, colloidal magnesium aluminum silicate) to aid in keeping the composition in contact with the oral cavity and not being easily ejected.

In one embodiment a composition of the present invention is formulated into a chewable and/or edible product (e.g., a chewable treat or edible tablet). Such a product would ideally have a taste, texture and/or aroma favored by the animal to be protected so as to facilitate oral administration of the compounds of the invention.

If the parasiticidal compositions are in the form of feed concentrates, the carrier is typically selected from high-performance feed, feed cereals or protein concentrates.

Such feed concentrate-containing compositions can, in addition to the parasiticidal active ingredients, comprise additives promoting animal health or growth, improving quality of meat from animals for slaughter or otherwise useful to animal husbandry.

These additives can include, for example, vitamins, antibiotics, chemotherapeutics, bacteriostats, fungistats, coccidiostats and hormones.

The compound of the invention may also be formulated in rectal compositions such as suppositories or retention enemas, using, e.g., conventional suppository bases such as cocoa butter or other glycerides.

The formulations for the method of this invention may include an antioxidant, such asBHT (butylated hydroxytoluene). The antioxidant is generally present in amounts of at 0.1-5 percent (wt/vol). Some of the formulations require a solubilizer, such as oleic acid, to dissolve the active agent, particularly if spinosad is included. Common spreading agents used in these pour-on formulations include isopropyl myristate, isopropyl palmitate, caprylic/capric acid esters of saturated C₁₂-C₁₈ fatty alcohols, oleic acid, oleyl ester, ethyl oleate, triglycerides, silicone oils and dipropylene glycol methyl ether. The pour-on formulations for the method of this invention are prepared according to known techniques. Where the pour-on is a solution, the parasiticide/insecticide is mixed with the carrier or vehicle, using heat and stirring if required. Auxiliary or additional ingredients can be added to the mixture of active agent and carrier, or they can be mixed with the active agent prior to the addition of the carrier. Pour-on formulations in the form of emulsions or suspensions are similarly prepared using known techniques.

Other delivery systems for relatively hydrophobic pharmaceutical compounds may be employed. Liposomes and emulsions are well-known examples of delivery vehicles or carriers for hydrophobic drugs. In addition, organic solvents such as dimethylsulfoxide may be used, if needed.

The rate of application required for effective parasitic invertebrate pest control (e.g. “pesticidally effective amount”) will depend on such factors as the species of parasitic invertebrate pest to be controlled, the pest's life cycle, life stage, its size, location, time of year, host crop or animal, feeding behavior, mating behavior, ambient moisture, temperature, and the like. One skilled in the art can easily determine the pesticidally effective amount necessary for the desired level of parasitic invertebrate pest control.

In general for veterinary use, the compounds of the invention are administered in a pesticidally effective amount to an animal, particularly a homeothermic animal, to be protected from parasitic invertebrate pests.

A pesticidally effective amount is the amount of active ingredient needed to achieve an observable effect diminishing the occurrence or activity of the target parasitic invertebrate pest. One skilled in the art will appreciate that the pesticidally effective dose can vary for the various compounds and compositions useful for the method of the present invention, the desired pesticidal effect and duration, the target parasitic invertebrate pest species, the animal to be protected, the mode of application and the like, and the amount needed to achieve a particular result can be determined through simple experimentation.

For oral or parenteral administration to animals, a dose of the compositions of the present invention administered at suitable intervals typically ranges from about 0.01 mg/kg to about 100 mg/kg, and preferably from about 0.01 mg/kg to about 30 mg/kg of animal body weight.

Suitable intervals for the administration of the compositions of the present invention to animals range from about daily to about yearly. Of note are administration intervals ranging from about weekly to about once every 6 months. Of particular note are monthly administration intervals (i.e. administering the compounds to the animal once every month).

The following Examples illustrate, but do not limit, the invention.

The following abbreviations were used throughout this section: s=singlet; bs=broad singlet; d=doublet; dd=double doublet; dt=double triplet; t=triplet, tt=triple triplet, q=quartet, sept=septet; m =multiplet; Me=methyl; Et=ethyl; Pr=propyl; Bu=butyl; RT=retention time; MH⁺=molecular cation.

### PREPARATION EXAMPLES

The following preparation examples describe synthesis of compounds of formula I and intermediates thereof

### Example 1

### Preparation of [2-chloro-4-[2-(3,5-dichlorophenyl)-2-(trifluoromethyl)-3H-furan-4-yl]phenyl]methanamine

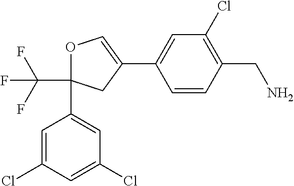 [SMILES: NCc1ccc(C2=COC(c3cc(Cl)cc(Cl)c3)(C(F)(F)F)C2)cc1Cl]

### Step 1: Preparation of 2-(3,5-dichlorophenyl)-1,1,1-trifluoro-but-3-en-2-ol

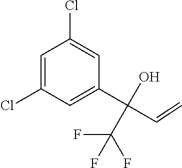 [SMILES: C=CC(O)(c1cc(Cl)cc(Cl)c1)C(F)(F)F]

Vinylmagnesium bromide 1M in THF (216.2 mL, 216.20 mmol) was added to a solution of 1-(3,5-dichlorophenyl)-2,2,2-trifluoro-ethanone (51.50 g, 211.93 mmol) in dry THF (425 mL) slowly at −75° C. to −65° C. The reaction mixture was allowed to warm to room temperature, and stirred at rt overnight. It was quenched by pouring into 2M aqueous HCl (140 mL) and extracted three times with diethyl ether. The combined organic fractions were washed successively with saturated NaHCO₃ solution, water, and brine and dried (MgSO₄). The solution was filtered and the solvent was removed under reduced pressure. Vacuum distillation (80-85° C./1 mbar) of the residue afforded 53.34 g (92.6%) of the title compound as a clear colorless liquid.

¹H-NMR (400 MHz, CDCl₃): δ 2.61 (s, 1H, OH), 5.57 (d, J=11 Hz, 1H), 5.62 (d, J=17.2 Hz, 1H), 6.36 (dd, J1=17.2 Hz, J2=11 Hz, 1H), 7.37 (t, J=1.8 Hz, 1H), 7.46-7.50 (m, 2H) ppm.

¹⁹F-NMR (377 MHz, CDCl₃): δ−78.80 ppm.

Steps 2A to 4A are reference Examples:

### Step 2A: Preparation of tert-butyl 4-[(E)-3-(3,5-dichlorophenyl)-4,4,4-trifluoro-3-hydroxy-but-1-enyl]-2-methyl-benzoate

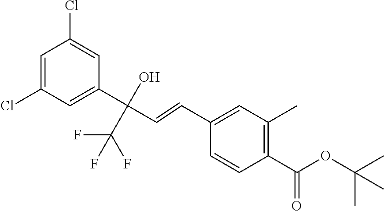 [SMILES: Cc1cc(/C=C/C(O)(c2cc(Cl)cc(Cl)c2)C(F)(F)F)ccc1C(=O)OC(C)(C)C]

A dry and with argon flushed reaction vessel was charged with tetrabutylammonium acetate (37.04 g, 122.85 mmol), palladium acetate (0.10 g, 0.443 mmol) and tert-butyl 4-bromo-2-methyl-benzoate (12.00 g, 44.27 mmol). The mixture was stirred for 15 minutes at 80° C. (black solution). It was cooled down to room temperature and 2-(3,5-dichlorophenyl)-1,1,1-trifluoro-but-3-en-2-ol (10 g, 36.89 mmol) was added. Reaction mixture was stirred for 60 h at 80° C. (became a black slurry). The reaction mixture was diluted with 200 mL of 1:2 mixture of ethyl acetate/petrol ether. The suspension formed was filtrated and the filtrate was evaporated. The crude product was purified by column chromatography (n-heptane/ethyl acetate 4:96->10:100) giving 15.24 g (89%) of the product as yellow crystals.

¹H-NMR (400 MHz, CDCl₃): δ 1.59 (s, 9H), 2.57 (s, 3H), 6.64 (d, J=16 Hz, 1H), 6.85 (d, J=16 Hz, 1H), 7.24-7.28 (m, 2H), 7.39 (t, J=1.8 Hz, 1H), 7.51-7.54 (m, 2H), 7.81 (d, J=8.1 Hz, 1H) ppm.

### Step 3A: Preparation of tert-butyl 4-[5-(3,5-dichlorophenyl)-2-hydroxy-5-(trifluoromethyl)tetrahydrofuran-3-yl]-2-methyl-benzoate

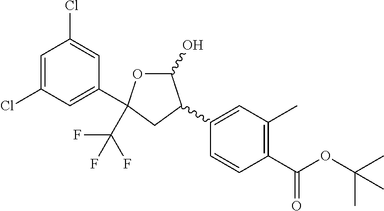 [SMILES: Cc1cc(C2CC(c3cc(Cl)cc(Cl)c3)(C(F)(F)F)OC2O)ccc1C(=O)OC(C)(C)C]

[Rh(CO)₂acac] (0.217 mmol, 0.0559 g), tris(2,4-di-tert-butylphenyl)phosphite (2.168 mmol, 1.402 g) and tert-butyl 4-[(E)-3-(3,5-dichlorophenyl)-4,4,4-trifluoro-3-hydroxy-but-1-enyl]-2-methyl-benzoate (21.680 mmol, 10.0 g) were suspended in toluene (120 mL) under argon and stirred until a homogeneous solution was obtained. The reaction mixture was then transferred into a mechanically stirred stainless steel autoclave (300 mL). The autoclave was purged three times with hydrogen (5 bar), pressurized with hydrogen and carbon monoxide to 50 bar (CO/H₂ composition=1:1). The reaction was vigorously stirred and heated (100° C.) and for 20 h. The reaction was stopped by cooling the autoclave to RT, venting and purging with argon. The reaction mixture was evaporated in vacuum and the crude product was isolated by a column chromatography (n-heptane/AcOEt gradient) as a white foam in 10.6 g (quant.) yield.

¹H-NMR (400 MHz, CDCl₃): δ 1.51-1.63 (m, 9H), 2.40-3.85 (m, 7H), 5.50-5.85 (m, 1H), 6.95-7.20 (m, 2H), 7.34-7.61 (m, 3H), 7.69-7.85 (m, 1H) ppm.

¹⁹F-NMR (377 MHz, CDCl₃): δ−79.34 (s), −78.69 (s), −78.55 (s), −77.52 (s) ppm.

### Step 4A: Preparation of 4-[2-(3,5-dichlorophenyl)-2-(trifluoromethyl)-3H-furan-4-yl]-2-methyl-benzoic acid

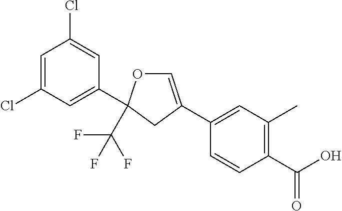 [SMILES: Cc1cc(C2=COC(c3cc(Cl)cc(Cl)c3)(C(F)(F)F)C2)ccc1C(=O)O]

tent-butyl 4-[5-(3,5-dichlorophenyl)-2-hydroxy-5-(trifluoromethyl)tetrahydrofuran-3-yl]-2-methyl benzoate (19.60 mmol, 9.65 g) and 4-methylbenzenesulfonic acid mono hydrate (3.93 mmol, 0.743 g) were heated in xylene (40 mL) under a stream of argon at 130° C. for 90 min. Reaction mixture was washed with NaHCO₃ (saturated solution) and the aqueous phase was extracted with ethyl acetate. Combined organic phases were dried (Na₂SO₄) and evaporated in vacuum. The title compound was isolated by crystallization from n-heptane in 6.70 g (82%) yield of yellowish crystals.

¹H-NMR (400 MHz, CDCl3): δ 2.64 (8, 3H), 3.32 (d, J=15 Hz, 1H), 3.75 (dd, J1=15 Hz, J2=2 Hz, 1H), 7.05-7.07 (m, 1H), 7.09-7.11 (m, 1H), 7.12-7.16 (m, 1H), 7.41 (t, J=1.8 Hz, 1H), 7.47-7.50 (m, 2H), 8.02 (d, J=8.4 Hz, 1H).

¹³C-NMR (101 MHz, CDCl₃): δ 22.30, 39.53, 87.5 (q, J=30.7 Hz), 114.66, 121.75, 125.08, 126.34, 127.7, 129.48, 132.28, 135.44, 136.52, 140.62, 142.13, 142.19, 172.64 ppm.

### Step 2B: Preparation of 2-[[2-chloro-4-[(E)-3-(3,5-dichlorophenyl)-4,4,4-trifluoro-3-hydroxy-but-1-enyl]phenyl]methyl]isoindoline-1,3-dione

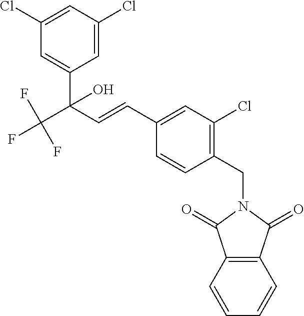 [SMILES: O=C1c2ccccc2C(=O)N1Cc1ccc(/C=C/C(O)(c2cc(Cl)cc(Cl)c2)C(F)(F)F)cc1Cl]

Trans-di-μ-acetatobis[2-(di-o-tolylphosphino)benzyl]dipalladium(II) (Herrmanns catalyst) (0.276 g, 0.285 mmol) was added to a degassed solution of 2-[(4-bromo-2-chloro-phenyl)methyl]isoindoline-1,3-dione (10.00 g, 28.52 mmol), 2-(3,5-dichlorophenyl)-1,1,1-trifluoro-but-3-en-2-ol (11.60 g, 42.78 mmol) and sodium acetate (3.51 g, 42.78 mmol) in N,N-dimethylacetamide (50 mL). Reaction mixture was stirred for 72 h at 130° C. under argon. Reaction mixture was cooled down to room temperature and poured on 1M HCl(aq) (pH=1). Aqueous layer was 3 times extracted with ethyl acetate. Combined organic layers were once washed with brine, dried (Na₂SO₄) and evaporated in vacuo. The crude product was purified by column chromatography (silica, n-heptane->n-heptanes/ethyl acetate=4:1) giving the title compounds 14.97 g (97%) as yellowish crystals.

¹H-NMR (400 MHz, CDCl₃): δ 2.90 (s, 1H, OH), 4.98 (s, 2H), 6.59 (d, J=16.1 Hz, 1H), 6.79 (d, J=16.1 Hz, 1H), 7.20-7.23 (m, 2H), 7.38 (t, J=1.8 Hz, 1H), 7.45 (s, 1H), 7.49-7.51 (m, 2H), 7.73-7.78 (m, 2H), 7.86-7.91 (m, 2H) ppm.

### Step 3B: Preparation of 2-[[2-chloro-4-[5-(3,5-dichlorophenyl)-2-hydroxy-5-(trifluoromethyl)tetrahydrofuran-3-yl]phenyl]methyl]isoindoline-1,3-dione

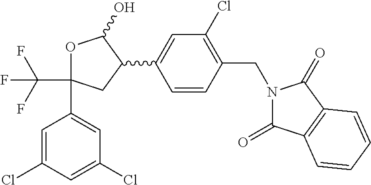 [SMILES: O=C1c2ccccc2C(=O)N1Cc1ccc(C2CC(c3cc(Cl)cc(Cl)c3)(C(F)(F)F)OC2O)cc1Cl]

[Rh(CO)2acac] (0.234 mmol, 0.060 g), tris(2,4-ditert-butylphenyl)phosphite (2.34 mmol, 1.51 g) and 2-[[2-chloro-4-[(E)-3-(3,5-dichlorophenyl)-4,4,4-trifluoro-3-hydroxy-but-1-enyl]phenyl]methyl]isoindoline-1,3-dione (23.40 mmol, 12.65 g) were dissolved in dry THF (100 mL) under argon. The reaction mixture was then transferred into a mechanically stirred stainless steel autoclave (300 mL). The autoclave was purged three times with hydrogen (5 bar), pressurized with hydrogen and carbon monoxide to 50 bar (CO/H₂ composition=1:1). The reaction was vigorously stirred and heated (100° C.) and for 36 h. The reaction mixture was evaporated in vacuum and the crude product was isolated by a column chromatography (n-heptane/AcOEt) as a white solid in 10.76 g (81%) yield.

¹H NMR (CDCl₃, 400 MHz): δ 7.93-7.88 (m, 2H), 7.79-7.76 (m, 2H), 7.59-7.34 (m, 4H), 7.25-7.12 (m, 2H), 5.80-5.54 (m, 1H), 5.03-4.95 (m, 2H), 3.75-2.42 (m, 4H) ppm

¹⁹F NMR (CDCl₃, 377 MHz): δ−77.56, −78.62, −78.74, −79.42 ppm m.p.=82-128° C.

### Step 4B: Preparation of 2-[[2-chloro-4-[2-(3,5-dichlorophenyl)-2-(trifluoromethyl)-3H-furan-4-yl]phenyl]methyl]isoindoline-1,3-dione

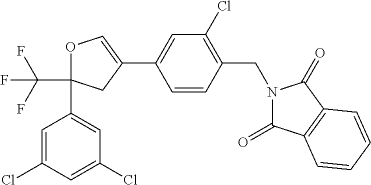 [SMILES: O=C1c2ccccc2C(=O)N1Cc1ccc(C2=COC(c3cc(Cl)cc(Cl)c3)(C(F)(F)F)C2)cc1Cl]

2-[[2-chloro-4-[5-(3,5-dichlorophenyl)-2-hydroxy-5-(trifluoromethyl)tetrahydrofuran-3-yl]phenyl]methyl]isoindoline-1,3-dione (15.77 mmol, 9.00 g) and 4-methylbenzenesulfonic acid mono hydrate (3.153 mmol, 0.597 g) were heated in xylene (33 mL) at 130° C. for 2 h under a stream of argon. Reaction mixture was extracted with NaHCO₃ (saturated solution) and the aqueous phase was extracted with ethyl acetate. Combined organic phases were dried (Na₂SO₄) and evaporated in vacuum to give 8.0 g (91%) of white solid.

¹H NMR (CDCl₃): δ 7.92-7.88 (m, 2H), 7.80-7.75 (m, 2H), 7.49 (s, 2H), 7.43 (m, 1H), 7.26-7.21 (m, 2H), 7.11-7.07 (m, 1H), 6.95 (s, 1H), 4.99 (s, 2H), 3.70 (dd, 1H, J1=15.3 Hz, J2=2.2 Hz), 3.26 (d, 1H, J=14.97 Hz) ppm.

¹³C NMR(CDCl₃, 400 MHz): δ 167.86, 141.07, 140.65, 135.40, 134.18, 133.60, 133.01, 132.00, 131.79, 129.44, 129.39, 125.49, 125.06, 123.49, 122.85, 122.84, 113.92, 87.29, 39.61, 39.21,

¹⁹F NMR (CDCl₃, 377 MHz: 6-80.85 ppm

Mp: 99.7° C.

### Step 5B: Preparation of [2-chloro-4-[2-(3,5-dichlorophenyl)-2-(trifluoromethyl)-3H-furan-4-yl]phenyl]methanamine

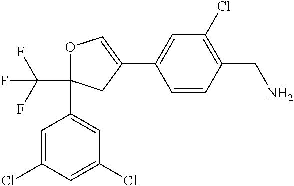 [SMILES: NCc1ccc(C2=COC(c3cc(Cl)cc(Cl)c3)(C(F)(F)F)C2)cc1Cl]

Hydrazine hydrate (72.36 mmol, 3.70 g) was added to a suspension of 2-[[2-chloro-4-[2-(3,5-dichlorophenyl)-2-(trifluoromethyl)-3H-furan-4-yl]phenyl]methyl]isoindoline-1,3-dione (14.47 mmol, 8 g) in ethanol (200 mL). The reaction mixture was stirred at 80° C. for 20 minutes (after initial dissolution some white solid precipitate formed). The solid was filtered off and washed with ethyl acetate. Combined Organic layers were washed with water (3×), dried (Na₂SO₄) and evaporated in vacuum to give 5.6 g (91%) of the title product as a white gum.

¹H NMR (CDCl₃, 400 MHz): δ=7.51 (s, 2H), 7.43 (t, 2H, J=1.9 Hz), 7.36 (d, 1H, J=7.93 Hz), 2.24 (d, 1H, J=1.83 Hz), 7.14 (dd, 1H, J1=8.02 Hz, J2=1.66 Hz), 3.94 (s, 2H), 3.73 (dd, 1H, J1=15.26 Hz, J2=2.27 Hz), 3.30 (d, 1H, J=14.60 Hz) ppm

¹³C NMR(CDCl₃): δ 140.75, 140.66, 139.01, 135.39, 133.74, 132.19, 129.41, 129.18, 125.38, 125.09, 123.04, 122.63, 114.03, 87.22, 44.16, 39.71 ppm

¹⁹F NMR(CDCl₃, 377 MHz): δ−80.83 ppm

### Example 2

### Preparation of N-[[2-chloro-4-[2-(3,5-dichlorophenyl)-2-(trifluoromethyl)-3H-furan-4-yl]phenyl]methyl]-2-methylsulfanyl-acetamide (A1)

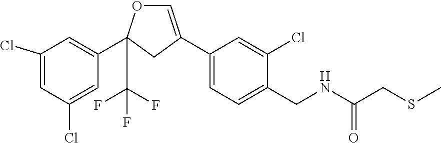 [SMILES: CSCC(=O)NCc1ccc(C2=COC(c3cc(Cl)cc(Cl)c3)(C(F)(F)F)C2)cc1Cl]

To a solution of [2-chloro-4-[2-(3,5-dichlorophenyl)-2-(trifluoromethyl)-3H-furan-4-yl]phenyl]methanamine (200 mg) in dichloromethane (5 mL) were added triethylamine (0.13 mL), 1-hydroxy-7-azabenzotriazole (71 mg), 1-(3-Dimethylaminopropyl)-3-ethylcarbodiimide hydrochloride (100 mg) and methylthioacetic acid (0.05 mL). The solution was allowed to stir at room temperature for 16 hours. After completion of the reaction, the solution was diluted with ethyl acetate and extracted with water. The combined organic layers were dried over magnesium sulfate, filtered and evaporated to give a crude residue. The residue was purified by flash column chromatography with (0-100% EtOAc/Heptane as an eluent) to give the title compound (198 mg) as a white solid.

¹H NMR (CDCl₃, 400 MHz): δ 7.48 (d, 2H), 7.40-7.43 (m, 1H), 7.35 (d, 1H), 7.24 (d, 1H), 7.12 (dd, 1H), 6.96 (s, 1H), 4.54 (d, 2H), 3.71 (dd, 1H), 3.27 (d, 1H), 3.23 (s, 2H), 2.09 (s, 3H) ppm

¹⁹F NMR(CDCl₃, 377 MHz): δ−80.87 ppm

### Example 3

### Preparation of N-[[2-chloro-4-[2-(3,5-dichlorophenyl)-2-(trifluoromethyl)-3H-furan-4-yl]phenyl]methyl]cyclopropanecarboxamide (A2)

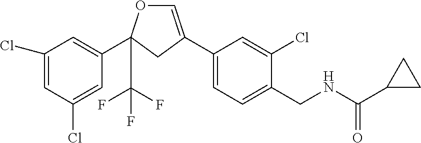 [SMILES: O=C(NCc1ccc(C2=COC(c3cc(Cl)cc(Cl)c3)(C(F)(F)F)C2)cc1Cl)C1CC1]

To a solution of [2-chloro-4-[2-(3,5-dichlorophenyl)-2-(trifluoromethyl)-3H-furan-4-yl]phenyl]methanamine (200 mg) in acetonitrile (5 mL) were added potassium carbonate (131 mg) then cyclopropanecarbonyl chloride (0.05 mL) at room temperature. The solution was allowed to stir at room temperature for 16 hours. After completion of the reaction, the solution was diluted with ethyl acetate and extracted with water. The combined organic layers were dried over magnesium sulfate, filtered and evaporated to give a crude residue. The residue was purified by flash column chromatography with (0-100% EtOAc/Heptane as an eluent) to give the title compound (155 mg) as a white solid.

¹H NMR (CDCl₃, 400 MHz): δ=7.48 (d, 2H), 7.42 (t, 1H), 7.35 (d, 1H), 7.23 (d, 1H), 7.07-7.12 (m, 1H), 6.95 (s, 1H), 6.03 (br. s, 1H), 4.51 (d, 2H), 3.70 (dd, 1H), 3.27 (d, 1H), 1.36 (ddd, 1H), 0.93-1.02 (m, 2H), 0.67-0.80 ppm (m, 2H) ppm.

¹⁹F NMR (CDCl₃, 377 MHz): δ−80.88 ppm

### General Method BOP T° C. For Preparing the Compounds of the Invention in Parallel

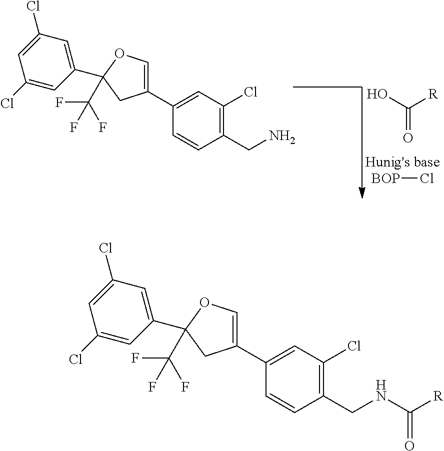

This general method was used to prepare a number of compounds in parallel.

To a solution of the appropriate carboxylic acid (1.5 eq), for example ethanoic acid, in N,N-dimethylacetamide (“DMA”) (0.37 ml) was added a solution of the appropriate amine (10 mg, 1 eq), for example [2-chloro-4-[2-(3,5-dichlorophenyl)-2-(trifluoromethyl)-3H-furan-4-yl]phenyl]methanamine, in N,N-dimethylacetamide (0.3 ml) followed by diisopropylethylamine (Hunig's base) (6 eq.) and a solution of bis(2-oxo-3-oxazolidinyl)phosphonic chloride (“BOP-Cl”) (2 eq) in N,N-dimethylacetamide (0.2 ml). The reaction mixture was stirred for 16 hours at T° C. Then the mixture was diluted with acetonitrile (0.6 ml) and a sample was used for the LC-MS analysis. The remaining mixture was further diluted with acetonitrile/N,N-dimethylformamide (4:1) (0.8 ml) and purified by HPLC to give the desired compound.

### Example 4

### Step 1: Preparation of 5-(3,5-dichlorophenyl)-5-(trifluoromethyl)tetrahydrofuran-2-ol

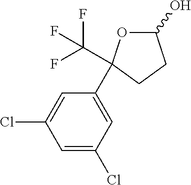 [SMILES: OC1CCC(c2cc(Cl)cc(Cl)c2)(C(F)(F)F)O1]

Rh(CO)₂acac (0.0048 g, 0.018 mmol) and 6-diphenylphosphanyl-1H-pyridin-2-one (0.026 g, 0.09 mmol) were dissolved in toluene (80 mL) under argon. 2-(3,5-dichlorophenyl)-1,1,1-trifluoro-but-3-en-2-ol (5 g, 18.45 mmol) was added and the reaction mixture was then transferred into a mechanically stirred stainless steel autoclave (300 mL). The autoclave was purged three times with hydrogen (5 bar), pressurized with hydrogen and carbon monoxide to 20 bar (CO/H₂ composition=1:1). The reaction was vigorously stirred and heated (80° C.) for 22 h.

The reaction was stopped by cooling the autoclave to RT, venting and purging with argon. The reaction mixture was evaporated in vacuum and the product was isolated by column chromatography (n-heptane/AcOEt gradient) as a brown gum in 5.0 g (11.13 mmol, 60%) yield.

¹HNMR (CDCl₃, 400 MHz): δ 2.45-2.08 (m, 4H); 2.80-2.61 (m, 3H); 5.67 (d, 1H, J=4.8 Hz); 5.75 (d, 1H, J=4.8 Hz); 7.38-7.23 (m, 6H) ppm.

### Step 2: Preparation of 2-(3,5-dichlorophenyl)-2-(trifluoromethyl)-3H-furan

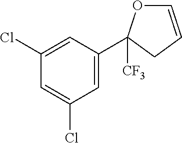 [SMILES: FC(F)(F)C1(c2cc(Cl)cc(Cl)c2)CC=CO1]

A mixture of 5-(3,5-dichlorophenyl)-5-(trifluoromethyl)tetrahydrofuran-2-ol (5 g, 11.1 mmol) and pyridinium 4-toluenesulfonate (1.68 g, 6.68 mmol) was heated to and finally distilled using a kugelrohr distillation apparatus (150° C., vacuum 100 to 4 mbar). The desired product was obtained as a white solid (2.41 g, 8.51 mmol, 76%).

¹HNMR (CDCl₃, 400 MHz): δ 2.95 (d, 1H, J=15.8 Hz); 3.40 (d, 1H, J=15.8 Hz); 5.03 (d, 1H, J=2.6 Hz); 6.43 (d, 1H, J=2.6 Hz); 7.43 (s, 2H), 7.39 (s, 1H) ppm.

### Step 3: Preparation of 4,5-dibromo-2-(3,5-dichlorophenyl)-2-(trifluoromethyl)tetrahydrofuran

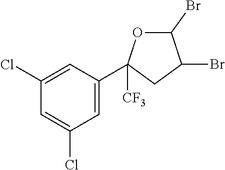 [SMILES: FC(F)(F)C1(c2cc(Cl)cc(Cl)c2)CC(Br)C(Br)O1]

A solution of bromine (1.13 g, 0.363 mL, 7.07 mmol) in dichloromethane (0.4 ml) was added to a solution of 2-(3,5-dichlorophenyl)-2-(trifluoromethyl)-3H-furan (2.00 g, 7.07 mmol) in dichloromethane (56 mL) slowly at −75° C. under argon. The reaction mixture was allowed to warm to room temperature and stirred for additional 20 minutes. Then, the reaction mixture was poured in a Na₂S₂O₃ aqueous solution and extracted twice with dichloromethane. The collected organic layers were dried (Na₂SO₄), filtered and evaporated under reduced pressure to give the title product (7.05 mmol, 3.12 g, 99%) as a white solid.

¹HNMR (CDCl₃, 400 MHz): δ 2.93 (d, 1H, J=14.7 Hz); 3.62 (dd, 1H, J=5.5 Hz, J=14.7 Hz); 4.9 (d, 1H, 5.5 Hz); 6.76 (s, 1H); 7.49 (m, 3H) ppm.

### Step 4: Preparation of 4-bromo-2-(3,5-dichlorophenyl)-2-(trifluoromethyl)-3H-furan

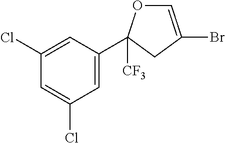 [SMILES: FC(F)(F)C1(c2cc(Cl)cc(Cl)c2)CC(Br)=CO1]

1,8-Diazabicyclo[5.4.0]undec-7-ene (0.103 g, 0.101 mL, 0.68 mmol) was dropwise added to a solution of 4,5-dibromo-2-(3,5-dichlorophenyl)-2-(trifluoromethyl)tetrahydrofuran (0.150 g, 0.34 mmol) in N,N-dimethylformamide (1 mL) at room temperature under argon. Then, the reaction mixture was warmed to 100° C. and stirred at that temperature for 20 min. The reaction mixture was quenched by pouring into a 2M HCl solution and extracted with n-hexane (3 times). The organic phase was dried (Na₂SO₄) and evaporated under reduce pressure giving the title compound (75 mg, 0.207 mmol, 61%) as a yellow oil.

¹HNMR (CDCl₃, 400 MHz): δ 3.15 (d, 1H, J=15.8 Hz); 3.56 (dd, 1H, J₁=15.8 Hz, J₂=2.6 Hz); 6.51 (t, 1H, J=2.2 Hz); 7.39 (s, 1H); 7.41 (t, 2H, J=1.5 Hz) ppm.

### Step 4b: Preparation of 2-[1-[4-(4,4,5,5-tetramethyl-1,3,2-dioxaborolan-2-yl)phenyl]ethyl]isoindoline-1,3-dione

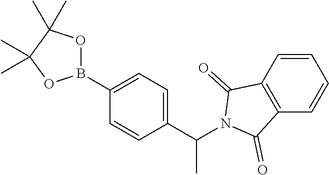 [SMILES: CC(c1ccc(B2OC(C)(C)C(C)(C)O2)cc1)N1C(=O)c2ccccc2C1=O]

Pd(DPPF)Cl₂.DCM (0.03957 g, 0.048 mmol) and potassium acetate (0.48 g, 4.85 mmol) were added to a solution of 2-[1-(4-bromophenyl)ethyl]isoindoline-1,3-dione (0.8 g, 2.423 mmol) and pinacol diborane (0.738 g, 2.91 mmol) in N,N-dimethylformamide (7 mL). The reaction mixture was stirred at 90° C. for 12 h under argon. The reaction mixture was diluted with water and ethyl acetate. Organic phase was washed 4 times with water and once with brine. It was dried and concentrated in vacuum. The crude material was purified by column chromatography (n-heptane/ethyl acetate gradient). The title product was obtained as a white solid (536 mg, 1.42 mmol, 59%).

¹HNMR (CDCl₃, 400 MHz): δ 1.32 (s, 12H); 1.93 (d, 3H, J=7.3 Hz); 5.59 (q, 1H, 7.3 Hz); 7.5 (d, 2H, J=7.7 Hz); 7.93-7.76 (m, 4H) ppm.

### Step 5: Preparation of 2-[1-[4-[2-(3,5-dichlorophenyl)-2-(trifluoromethyl)-3H-furan-4-yl]phenyl]ethyl]isoindoline-1,3-dione

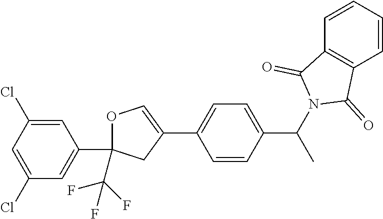 [SMILES: CC(c1ccc(C2=COC(c3cc(Cl)cc(Cl)c3)(C(F)(F)F)C2)cc1)N1C(=O)c2ccccc2C1=O]

A test tube containing a magnetic stir bar was charged with S-Phos palladacycle catalysts (CAS=1028206-58-7, STREM=46-0269) (0.018 g, 0.0026 mmol); 2-[1-[4-(4,4,5,5-tetramethyl-1,3,2-dioxaborolan-2-yl)phenyl]ethyl]isoindoline-1,3-dione (0.06232 g, 0.16 mmol) and potassium phosphate (0.055619 g, 0.26 mmol). The tube was capped with a rubber septum, evacuated and backfilled with argon (this sequence was repeated three times). Deionized water (0.02 mL) and dry toluene (0.4 mL) and were added sequentially and the resulting mixture was stirred at room temperature for ˜2 min. 4-bromo-2-(3,5-dichlorophenyl)-2-(trifluoromethyl)-3H-furan (0.046 g, 0.13 mmol) was added dropwise via syringe. The reaction mixture was stirred vigorously at 100° C. for 18 h. The reaction mixture was diluted with AcOEt, washed with water, dried (Na₂SO₄) and evaporated. The residue was purified by flash chromatography on silica gel (n-heptane/ethyl acetate gradient 9:1 to 5:5) to giving the title compound (0.034 mmol, 18 mg, 27%) as a white solid.

¹HNMR (CDCl₃, 400 MHz): δ 3.62 (dt, 1H, J₁=15.3, J₂=2.2 Hz); 5.47 (q, 1H, J=7.3 Hz); 6.83 (s, 1H); 7.11 (d, 2H, J=8.4 Hz); 7.31 (t, 1H, J=1.8 Hz); 7.37 (s, 1H); 7.39 (m, 3H); 7.61 (m, 2H); 7.72 (m, 2H) ppm.

### Example 5

### Preparation of N-[(1S)-1-[4-[2-(3,4,5-trichlorophenyl)-2-(trifluoromethyl)-3H-furan-4-yl]phenyl]ethyl]cyclopropanecarboxamide

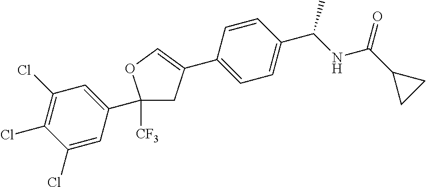 [SMILES: C[C@H](NC(=O)C1CC1)c1ccc(C2=COC(c3cc(Cl)c(Cl)c(Cl)c3)(C(F)(F)F)C2)cc1]

### Step A: 2-[(1S)-1-(4-bromophenyl)ethyl]isoindoline-1,3-dione

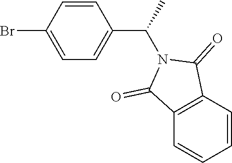 [SMILES: C[C@@H](c1ccc(Br)cc1)N1C(=O)c2ccccc2C1=O]

A mixture of (1S)-1-(4-bromophenyl)ethanamine (30.0 g) and phthalic anhydride (22.2 g) in glacial acetic acid (500 mL) was refluxed overnight. The acetic acid was removed in vacuo and the residue was dissolved in EtOAc, washed with sat. NaHCO₃ solution, and brine, dried over MgSO₄, filtered and evaporated to obtain the desired product as a white solid (37.0 g, 75% yield). LCMS (Method GR): RT 1.13 min, [M+H]⁺; 330: ¹H NMR (400 MHz, CDCl₃) 1.60 (d, 3H), 5.30 (q, 1H), 6.30 (d, 1H), 7.20-8.20 (m, 8H).

### Step B: 1,1,1-trifluoro-2-(3,4,5-trichlorophenyl)but-3-en-2-ol

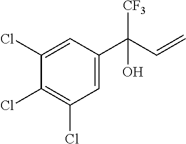 [SMILES: C=CC(O)(c1cc(Cl)c(Cl)c(Cl)c1)C(F)(F)F]

To a solution of 2,2,2-trifluoro-1-(3,4,5-trichlorophenyl)ethanone (100.92 mmol, 28 g) in THF (100 ml) was added vinylmagnesium bromide solution (1M in THF, 100.92 mmol, 85.9 g) at −78° C. dropwise under argon. After addition the mixture was warmed to room temperature and stirred overnight. The reaction mixture was concentrated, the residue was diluted with TBME, washed with HCl 0.25N, brine, dried over MgSO₄, filtered and evaporated to obtain the desired product as a pale yellow oil (30 g, 97%). LCMS (Method GR): RT 0.93 min, [M+H]⁺; 305/307: ¹H NMR (400 MHz, CDCl₃) 2.70 (s, OH), 5.60-5.70 (m, 2H), 6.30 (d, 1H), 6.30-6.40 (m, 1H), 7.65 (s, 2H)¹⁹F-NMR (CDCl₃, 376.3 MHz): −78.93.

### Step C: 2-[(1S)-1-[4-[(E)-4,4,4-trifluoro-3-hydroxy-3-(3,4,5-trichlorophenyl)but-1-enyl]phenyl]ethyl]isoindoline-1,3-dione

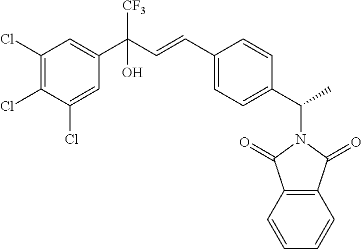 [SMILES: C[C@@H](c1ccc(/C=C/C(O)(c2cc(Cl)c(Cl)c(Cl)c2)C(F)(F)F)cc1)N1C(=O)c2ccccc2C1=O]

To a degassed solution of 2-[(1S)-1-(4-bromophenyl)ethyl]isoindoline-1,3-dione (30.28 mmol, 10 g), 1,1,1-trifluoro-2-(3,4,5-trichlorophenyl)but-3-en-2-ol (45.43 mmol, 16.33 g) and NaOAc (45.43 mmol, 3.764 g) in N,N-dimethylacetamide (0.56 mol/L) was added Herrmann's catalyst (0.2120 mmol, 0.2115 g). The reaction mixture was stirred for 15 min at 175° C. under microwave irradiation and then a further 15 min at 200° C. under microwave irradiation. The reaction mixture was cooled down to room temperature and poured into 1M HCl (aq). The aqueous layer was extracted three times with ethyl acetate and the combined organic layers were once washed with brine, dried (MgSO₄) and evaporated in vacuo. The crude product was purified over a silica gel column (eluent: cyclohexane/EtOAc) giving the titled compound 7.0 g as pale brown solid.

LCMS (Method GR): RT 1.31 min, [M+H]⁺ 552/554; ¹H-NMR (CDCl₃, 400 MHz): 1.90 (d, 3H), 5.60 (q, 1H), 6.60 (d, 1H), 6.80 (d, 1H), 7.35 (d, 2H), 7.50 (d, 2H), 7.65 (s, 2H), 7.75 (m, 2H), 7.85 (m, 2H) ¹⁹F-NMR (CDCl₃, 376.3 MHz): −79.38.

### Step D: 2-[(1S)-1-[4-[2-(3,4,5-trichlorophenyl)-2-(trifluoromethyl)-3H-furan-4-yl]phenyl]ethyl]isoindoline-1,3-dione

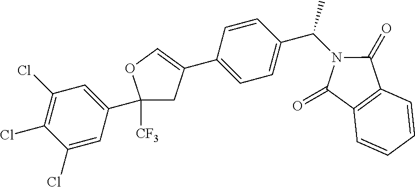 [SMILES: C[C@@H](c1ccc(C2=COC(c3cc(Cl)c(Cl)c(Cl)c3)(C(F)(F)F)C2)cc1)N1C(=O)c2ccccc2C1=O]

A homogeneous solution of [Rh(CO)₂acac] (0.01 equiv.), tris(2,4-ditert-butylphenyl) phosphite (0.1 equiv.) and 2-[(1S)-1-[4-[(E)-4,4,4-trifluoro-3-hydroxy-3-(3,4,5-trichlorophenyl)but-1-enyl]phenyl]ethyl]isoindoline-1,3-dione (13.0 mmol, 7.0 g) in toluene (63 mL) in a stainless steel autoclave was purged three times with hydrogen (5 bar), pressurized at 25 bar with H₂ followed by an additional 25 bar of CO (=50 bar CO/H₂ 1:1). The reaction was then heated at 100° C. and vigorously stirred for 16 h. The reaction was stopped by cooling the autoclave to room temperature, venting and purging with argon. The reaction was concentrated and the crude reaction mixture was dissolved in xylenes (100 mL) and 4-methylbenzenesulfonic acid (4.10 mmol, 0.714 g) was added and mixture was heated at reflux for 6 hours. The mixture was then cooled to room temperature, diluted with ethyl acetate and washed twice with NaHCO₃ sat aqueous solution, once with water and once with brine. Organic phase was then dried over magnesium sulfate, filtered and solvents were evaporated under reduced pressure. The crude product was purified over a silica gel column (eluent: cyclohexane/EtOAc) to yield 4.3 g of the titled compound. LCMS (Method GR): RT 1.41 min; ¹H-NMR (CDCl₃, 400 MHz): 1.90 (d, 3H), 3.20 (d, 2H), 3.70 (d, 2H), 5.50 (q, 1H), 7.90 (s, 1H), 7.20 (d, 2H), 7.45 (d, 2H), 7.60 (s, 2H), 7.70 (d, 2H), 7.80 (d, 2H). ¹⁹F-NMR (CDCl₃, 400 MHz): −79.52.

### Step E: (1S)-1-[4-[2-(3,4,5-trichlorophenyl)-2-(trifluoromethyl)-3H-furan-4-yl]phenyl]ethanamine

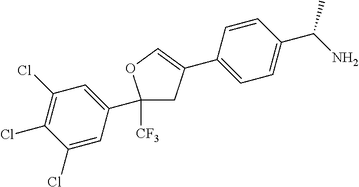 [SMILES: C[C@H](N)c1ccc(C2=COC(c3cc(Cl)c(Cl)c(Cl)c3)(C(F)(F)F)C2)cc1]

To a suspension of 2-[(1S)-1-[4-[2-(3,4,5-trichlorophenyl)-2-(trifluoromethyl)-3H-furan-4-yl]phenyl]ethyl]isoindoline-1,3-dione (4.2 g) in ethanol (90 ml) was added hydrazine hydrate (75.9 mmol, 2.43 g) and the mixture was heated at 50° C. over night. The mixture was filtered, and the cake was washed with toluene, the mother liquor was concentrated. The residue was dissolved in ethyl acetate, washed with sat. NaHCO₃ solution, water, brine, dried over MgSO₄, filtered and evaporated to obtain the desired crude product (3.0 g). LCMS (Method C): RT 1.03 min, 421/423/424; ¹H-NMR (CDCl₃, 400 MHz): 1.35-1345 (b, NH2), 3.20-3.25 (d, 1H), 3.70-375 (d, 1H), 6.90 (1, 1H), 7.90 (s, 1H), 7.10-7.30 (m, 4H), 7.45 (d, 2H), 7.60 (s, 2H), ¹⁹F-NMR (CDCl₃, 376.3 MHz): −79.84, −80.85.

### Step F: N-[(1S)-1-[4-[2-(3,4,5-trichlorophenyl)-2-(trifluoromethyl)-3H-furan-4-yl]phenyl]ethyl]cyclopropanecarboxamide: B1* and B1**

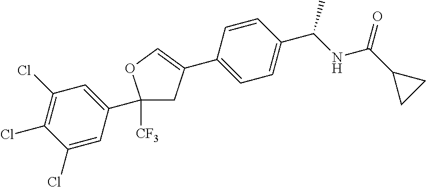 [SMILES: C[C@H](NC(=O)C1CC1)c1ccc(C2=COC(c3cc(Cl)c(Cl)c(Cl)c3)(C(F)(F)F)C2)cc1]

To a solution of (1S)-1-[4-[2-(3,4,5-trichlorophenyl)-2-(trifluoromethyl)-3H-furan-4-yl]phenyl]ethanamine (3.27 g) in dichloromethane (70 ml) was added Et₃N (21 mmol, 2.1 g) and the mixture was cooled to 0° C. Cyclopropanecarbonyl chloride (7.6 mmol, 0.79 g) was added dropwise under argon and the reaction mixture was stirred for 30 min at 0° C. The mixture was diluted with dichloromethane, washed with water, brine, dried over MgSO₄ filtered and evaporated. Purification over a silica gel column (eluent: cyclohexane/EtOAc) yielded 4.3 g of the titled compound as a light yellow solid. LCMS (Method GR): RT 1.27 min, 506/508/509;

¹H-NMR (CDCl₃, 400 MHz): 0.60 (q, 2H), 0.90 (q, 2H), 1.50 (d, 1H), 3.20 (d, 1H), 3.70 (d, 1H), 5.60 (q, 1H) (m, 4H), 5.70-5.75 (d, NH), 7.90 (s, 1H), 7.20 (d, 2H), 7.30 (d, 2H), 7.60, s, 2H).

¹⁹F-NMR (CDCl₃, 376.3 MHz): −80.79. The two diastereoisomers were separated by preparative chiral HPLC.

### Preparative HPLC Method

Autopurification System from Waters: 2767 sample Manager, 2489 UV/Visible Detector, 2545

### Quaternary Gradient Module.

Column: Daicel CHIRALPAK® IA, 1.0 cm×25 cm
Mobile phase: TBME/EtOH 95/05
Flow rate: 10 ml/min

### Detection: UV 265 nm

Sample concentration: 100 mg/mL in TBME
Injection: 300 μl-500 μl

### Analytical HPLC Method

### HPLC: Waters UPLC—Hclass, DAD Detector Waters UPLC

Column: Daicel CHIRALPAK® IA, 3 μm, 0.46 cm×10 cm
Mobile phase: TBME/EtOH 95/05
Flow rate: 1.0 ml/min

### Detection: 265 nm

Sample concentration: 1 mg/mL in DCM/iPrOH 50/50

### Injection: 2 μl

### Enzymatic Resolution

### Step A: 2-(3,5-dichlorophenyl)-1,1,1-trifluoro-but-3-yn-2-ol

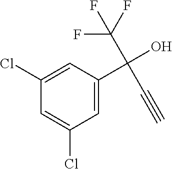 [SMILES: C#CC(O)(c1cc(Cl)cc(Cl)c1)C(F)(F)F]

In a 250 mL round bottom flask equipped with a reflux condenser, magnetic stirring and a thermometer under argon, was added bromo(ethynyl)magnesium (0.5M, 82.30 mmol). The solution was cooled to 0° C. followed by dropwise addition of 1-(3,5-dichlorophenyl)-2,2,2-trifluoro-ethanone (10 g, 41.1504 mmol) keeping the temperature under 7° C. The reaction was brought back to room temperature and was stirred overnight at room temperature. The reaction was cooled to 0° C. and quenched carefully with HCl (1M) until pH=1. The organic phase was then washed four times with water and once with brine. The combine organic phases were dried over MgSO4, filtered and solvents were evaporated under reduced pressure. The crude product was distilled under reduced pressure (1 mBar, 120° C.) to yield 9 g of a clear oil which solidified upon standing. ¹H NMR (400 MHz, CDCl₃) 2.90 (s, 1H), 3.26 (s, OH), 7.45 (d, 1H), 7.55 (d, 2H); ¹⁹F-NMR (CDCl₃, 376.3 MHz −80.40.

### Step B: [1-(3,5-dichlorophenyl)-1-(trifluoromethyl)prop-2-ynyl]butanoate

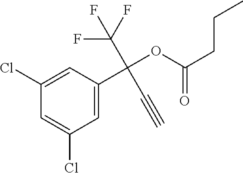 [SMILES: C#CC(OC(=O)CCC)(c1cc(Cl)cc(Cl)c1)C(F)(F)F]

In a 30 mL flask, 2-(3,5-dichlorophenyl)-1,1,1-trifluoro-but-3-yn-2-ol (2.69 g, 10 mmol), triethylamine (1.31 g, 13 mmol) and N,N-dimethylpyridin-4-amine (0.061 g, 0.5 mmol) were dissolved in dichloromethane and cooled to 0° C. Then butanoyl chloride (1.39 g, 13 mmol) was added dropwise. The mixture was stirred overnight at room temperature. The mixture was taken up in MTBE and washed with HCl (0.1 M), water, brine, dried over MgSO4, filtered on a pad of silica gel and solvents were evaporated under reduced pressure to yield 3.5 g of a clear oil. ¹H NMR (400 MHz, CDCl₃) 1.05 (m, 3H), 1.72 (m, 2H), 2.48 (m, 2H), 3.00 (s, 1H), 7.43 (d, 1H), 7.52 (d, 2H); ¹⁹F-NMR (CDCl₃, 376.3 MHz): −78.40.

### Step B: [(1S)-1-(3,5-dichlorophenyl)-1-(trifluoromethyl)prop-2-ynyl]butanoate and (2R)-2-(3,5-dichlorophenyl)-1,1,1-trifluoro-but-3-yn-2-ol

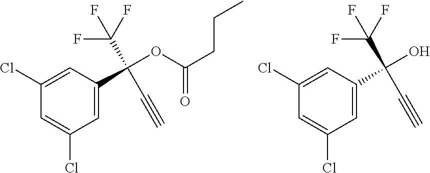 [SMILES: C#C[C@](OC(=O)CCC)(c1cc(Cl)cc(Cl)c1)C(F)(F)F ; C#C[C@@](O)(c1cc(Cl)cc(Cl)c1)C(F)(F)F]

A solution of Lipase from Candida rugosa (5 g) in phosphate buffer pH=7.4, 100 mM (100 mL) was mechanically stirred in a 250 mL glass reactor (500 rpm) at room temperature for 2 hours. Then a solution of [1-(3,5-dichlorophenyl)-1-(trifluoromethyl)prop-2-ynyl]butanoate (10 g) in DMSO (20 mL) was added to the previous solution. The reaction mixture was mechanically stirred at 55° C. (internal temperature), 500 rpm for 2 days. Aliquots were analyzed by LCMS during the course of the experiment. After 50 h, 1.742 g of K₂HPO₄ (10 mmol) was added to the mixture and stirred for a further 20 h. At this point Celite (20 g) was added and the reaction was filtered through on a Celite plug. The Celite cake was then rinsed with ethyl acetate (7×100 mL). The clear biphasic mixture was decanted and the aqueous phase was extracted with ethyl acetate (2×100 mL). The gathered organic phases were washed with brine (100 mL), dried on MgSO4, and concentrated under vacuum (40° C., 30 mbar). A viscous orange oil (m=10.80 g) was obtained. The crude product was purified by flash chromatography over a silica gel column (eluent: cyclohexane/EtOAc) to yield 4.85 g of [(1S)-1-(3,5-dichlorophenyl)-1-(trifluoromethyl)prop-2-ynyl]butanoate and 2.93 g of (2R)-2-(3,5-dichlorophenyl)-1,1,1-trifluoro-but-3-yn-2-ol.

A solution of [(1S)-1-(3,5-dichlorophenyl)-1-(trifluoromethyl)prop-2-ynyl]butanoate (108 mg in 10 mL CHCl3) in such manner was analyzed for optical rotation at 20° C. This [α]_{D} at 20° C. was −15.37°. The use of (S)-(+)-1-(9-Anthryl)-2,2,2-trifluoroethanol enabled to determine an ee=56%.

A solution of this (2R)-2-(3,5-dichlorophenyl)-1,1,1-trifluoro-but-3-yn-2-ol (57 mg in 5 mL CHCl3) in such manner was analysed for optical rotation at 20° C. This [α]_{D} at 20° C. was +7.28°.

### Chiral GC Analysis

GC was conducted on a Thermo Focus GC, with a column from Supelco Alpha DEX 120 fused silica Capillary Column: 30 m, diam: 0.25 mm, 0.25 μm, H₂ flow 1 ml/min, temp injector: 220° C., FID Detector: temp detector: 300° C., method: start at 80° C., hold 2 min, 5.5° C./min until 220° C., hold 3 min, total time 30 min

2 isomers were detected: rt=23.37 min (86.0%) 24.32 min. (14.0%).

### Example P6 (Reference Example)

### Preparation of 5-[(2S)-2-(3,5-dichlorophenyl)-2-(trifluoromethyl)-3H-furan-4-yl]-2-(1,2,4-triazol-1-yl)benzonitrile

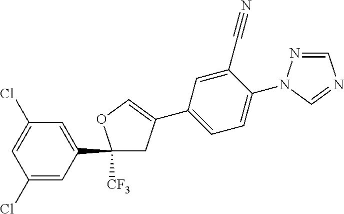 [SMILES: N#Cc1cc(C2=CO[C@@](c3cc(Cl)cc(Cl)c3)(C(F)(F)F)C2)ccc1-n1cncn1]

### Step A: Preparation of 5-iodo-2-(1,2,4-triazol-1-yl)benzonitrile

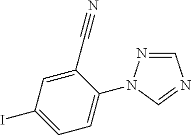 [SMILES: N#Cc1cc(I)ccc1-n1cncn1]

To a solution of 2-fluoro-5-iodo-benzonitrile (25.3 g) and 1H-1,2,4-TRIAZOLE (8.66 g) in N,N-dimethylformamide (102 mL) was added cesium carbonate (40.0 g) and the mixture was heated at 60° C. for 5 hours. The beige-brown suspension was cooled to room temperature and allowed to stand for 6 days. The mixture was dissolved in ethyl acetate, washed with a hydrochloric solution (1M). The combined organic layers were dried over magnesium sulfate, filtered and evaporated to obtain the desired product as a white solid (28 g). ¹H NMR (CDCl₃, 400 MHz): δ=8.79 (s, 1H), 8.20 (s, 1H), 8.16 (d, J=1.8 Hz, 1H), 8.04-8.12 (m, 1H), 7.55 ppm (d, J=8.4 Hz, 1H)

### Step B: Preparation of 5-[(3R)-3-(3,5-dichlorophenyl)-4,4,4-trifluoro-3-hydroxy-but-1-ynyl]-2-(1,2,4-triazol-1-yl)benzonitrile

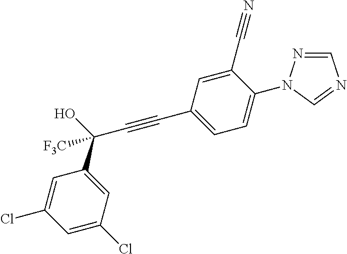 [SMILES: N#Cc1cc(C#C[C@](O)(c2cc(Cl)cc(Cl)c2)C(F)(F)F)ccc1-n1cncn1]

To a solution of [(1S)-1-(3,5-dichlorophenyl)-1-(trifluoromethyl)prop-2-ynyl]butanoate (4.0 g, mmol) and 5-iodo-2-(1,2,4-triazol-1-yl)benzonitrile (4.4 g, 15 mmol) in N,N-dimethylformamide (36 mL) is added successively at room temperature triethylamine (30 g, 41 mL, 300 mmol), cooper-(I)-iodide (1.1 g, 5.9 mmol) and dichlorobis(triphenylphosphine)palladate(II) (1.1 g, 1.5 mmol) under argon. The mixture was heated to 80° C. for 3 hours then the mixture was dissolved in ethyl acetate. The suspension was washed with a hydrochloric solution (1M) to reach ph=4. The combined organic layers were dried over magnesium sulfate, filtered and evaporated to give a residue that was suspended in dichloromethane. The suspension was filtered and the solid was washed with dichloromethane then dried under vacuo to give the titled compound as beige solid (1.25 g).

The mother liquors were further concentrated under vacuo and purified using a silica gel column (eluent: cyclohexane/EtOAc) giving the titled compound as pale brown solid (2.92 g). ¹H NMR (DMSO d₆, 400 MHz): δ=9.31 (br. s, 1H), 8.56 (s, 1H), 8.43 (d, J=1.8 Hz, 2H), 8.12 (dd, J=8.4, 1.8 Hz, 1H), 7.99 (d, J=8.8 Hz, 1H), 7.80 (t, J=1.8 Hz, 1H), 7.68-7.77 ppm (m, 2H)

### Step C: Preparation of 5-[(E,3S)-3-(3,5-dichlorophenyl)-4,4,4-trifluoro-3-hydroxy-but-1-enyl]-2-(1,2,4-triazol-1-yl)benzonitrile

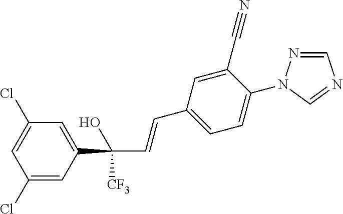 [SMILES: N#Cc1cc(/C=C/[C@](O)(c2cc(Cl)cc(Cl)c2)C(F)(F)F)ccc1-n1cncn1]

To a solution of 5-[(3R)-3-(3,5-dichlorophenyl)-4,4,4-trifluoro-3-hydroxy-but-1-ynyl]-2-(1,2,4-triazol-1-yl)benzonitrile (3.12 g) in 15.7 mL of toluene and 5.71 mL of THF cooled to −30° C., sodium bis(2-methoxyethoxy)aluminum hydride (65 mass % in Toluene) (2.56 mL) was added. The reaction was stirred at −30° C. for 3 h. The reaction mixture was carefully quenched first with acetone at −30° C. and then with NH₄Cl solution sat at −10° C. and extracted twice with ethyl acetate. The combined organic layers were dried (MgSO4), filtered and evaporated to give a yellow residue that was suspended in dichloromethane. The suspension was filtered and the solid was washed with dichloromethane then dried under vacuo to give the titled compound as yellow solid (1.49 g). The mother liquors were further concentrated under vacuo and purified using a silica gel column (eluent: cyclohexane/EtOAc) giving the titled compound (791 mg). ¹H-NMR (DMSO d₆, 400 MHz): δ=9.21 (s, 1H), 8.49 (d, J=1.8 Hz, 1H), 8.37 (s, 1H), 8.14 (dd, J=8.6, 2.0 Hz, 1H), 7.89 (d, J=8.4 Hz, 1H), 7.80 (d, J=1.8 Hz, 2H), 7.71 (t, J=1.8 Hz, 1H), 7.60 (s, 1H), 7.41 (d, J=15.8 Hz, 1H), 7.05 ppm (d, J=16.1 Hz, 1H)

### Step E: 5-[(2S)-2-(3,5-dichlorophenyl)-2-(trifluoromethyl)-3H-furan-4-yl]-2-(1,2,4-triazol-1-yl)benzonitrile

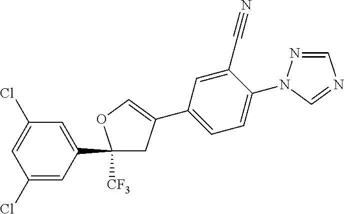 [SMILES: N#Cc1cc(C2=CO[C@@](c3cc(Cl)cc(Cl)c3)(C(F)(F)F)C2)ccc1-n1cncn1]

A homogeneous solution of [Rh(CO)₂acac] (0.0135 g), tris(2,4-ditert-butylphenyl) phosphite (0.336 g) and 5-[(E,3S)-3-(3,5-dichlorophenyl)-4,4,4-trifluoro-3-hydroxy-but-1-enyl]-2-(1,2,4-triazol-1-yl)benzonitrile (2.28 g) in tetrahydrofuran (20 mL) in a stainless steel autoclave was purged three times with hydrogen (5 bar), pressurized at 25 bar with H₂ followed by an additional 25 bar of CO (=50 bar CO/H₂ 1:1). The reaction was then heated at 100° C. and vigorously stirred for 70 h. The reaction was stopped by cooling the autoclave to room temperature, venting and purging with argon. The mixture was dissolved in ethyl acetate, washed with water. The combined organic layers were dried over magnesium sulfate, filtered and evaporated to obtain a crude residue which was purified using a silica gel column (eluent: cyclohexane/EtOAc) giving the hydrofomylated compound (2.14 g) as a beige foam that was used as such (mixture of diastereoisomers) in the next step.

The residue (2.47 g) was dissolved in xylenes (98 mL) and 4-methylbenzenesulfonic acid (1.10 g) was added. The mixture was heated to 120° C. for 15.5 hours. The mixture was then cooled to room temperature, and slowly poured on a cold saturated sodium carbonate solution. The mixture was diluted with ethyl acetate and washed twice with a saturated solution of sodium hydrogenocarbonate. The combined organic layers were dried over magnesium sulfate, filtered and evaporated to obtain a crude residue which was purified using a silica gel column (eluent: cyclohexane/EtOAc containing 1% NEt₃) to provide the titled compound (1.59 g) as a beige foam.

¹H-NMR (CDCl₃, 400 MHz): δ=8.76 (s, 1H), 8.19 (s, 1H), 7.68-7.78 (m, 1H), 7.56-7.67 (m, 2H), 7.49 (d, J=1.5 Hz, 2H), 7.40-7.47 (m, 1H), 7.13 (s, 1H), 3.78 (dd, J=15.2, 2.0 Hz, 1H), 3.36 ppm (d, J=15.4 Hz, 1H)

### Example 7 (Reference Example)

### Preparation of 4-[(2S)-2-(3,5-dichlorophenyl)-2-(trifluoromethyl)-3H-furan-4-yl]-N-(1,1-dioxothietan-3-yl)-2-methyl-benzamide

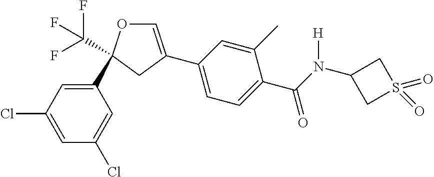 [SMILES: Cc1cc(C2=CO[C@@](c3cc(Cl)cc(Cl)c3)(C(F)(F)F)C2)ccc1C(=O)NC1CS(=O)(=O)C1]

### Step A: 2-Methyl-4-trimethylsilanylethynyl-benzoic acid tert-butyl ester

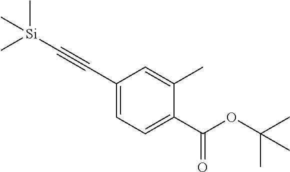 [SMILES: Cc1cc(C#C[Si](C)(C)C)ccc1C(=O)OC(C)(C)C]

To a solution of tert-butyl 4-bromo-2-methyl-benzoate (100 g, 368.8 mmol) in tetrahydrofuran (6 mL/mmol) and diisopropylamine (1.2 equiv., 442.5 mmol) at room temperature were added copper(I)-iodide (0.05 equiv., 18.44 mmol) and dichlorobis(triphenylphosphine)palladate(II) (0.05 equiv., 18.44 mmol). Argon was bubbled through the reaction for 5 minutes then ethynyl(trimethyl)silane (2.2 equiv., 811.3 mmol) was added dropwise over a 15 min period. The mixture was heated at 45° C. for 4 h. The mixture was filtered over celite and the filter cake washed with ethyl acetate. The organic phase was then washed twice with a saturated NH₄Cl solution and once with brine, dried over Na₂SO₄ and solvents were evaporated under reduced pressure. The brown oil residue was purified over a silica gel column (eluent: cyclohexane/EtOAc) to give 40 g of as a yellow oil. LCMS (Method A) 1.40 min; ¹H-NMR (CDCl₃, 400 MHz): 0.25 (s, 3H), 1.61 (s, 9H), 2.52 (s, 3H), 7.30 (s, 1H), 7.32 (d, 1H), 7.74 (d, 1H).

### Step B: 4-Ethynyl-2-methyl-benzoic acid tert-butyl ester

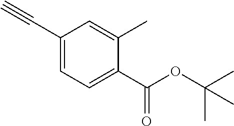 [SMILES: C#Cc1ccc(C(=O)OC(C)(C)C)c(C)c1]

To a solution of tert-butyl 2-methyl-4-(2-trimethylsilylethynyl)benzoate (103.0 g, 357.0 mmol) in methanol (500 mL) at room temperature was added potassium carbonate (75.09 g, 535.5 mmol). The resulting suspension was rapidly stirred at room temperature for 15 min and then water was added until dissolution of K₂CO₃. The mixture was extracted twice with dichloromethane. The combined organic phases were washed with brine, dried over MgSO₄ and solvent were evaporated under reduced pressure. The crude product was purified over a silica gel column (eluent: heptane/EtOAc) to give 82 g of tert-butyl 4-ethynyl-2-methyl-benzoate as a yellow oil. LCMS (Method A) RT 1.18 min; ¹H-NMR (CDCl₃, 400 MHz): 1.62 (s, 9H), 2.57 (s, 3H), 3.18 (s, 1H), 7.29 (s, 1H), 7.38 (d, 1H), 7.80 (d, 1H).

### Step C: tert-butyl 4-[(3R)-3-(3,5-dichlorophenyl)-4,4,4-trifluoro-3-hydroxy-but-1-ynyl]-2-methyl-benzoate

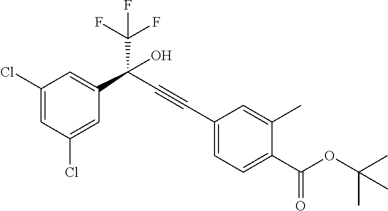 [SMILES: Cc1cc(C#C[C@](O)(c2cc(Cl)cc(Cl)c2)C(F)(F)F)ccc1C(=O)OC(C)(C)C]

To a stirred solution of quinine (0.2 equiv., 16.5 mmol), barium(2+) dihydrofluoride (0.2 equiv., 16.46 mmol) and tert-butyl 4-ethynyl-2-methyl-benzoate (2.5 equiv., 205.8 mmol) was added slowly dimethylzinc (4.0 equiv., 329.2 mmol, 2.0 mol/L) and the mixture was stirred at room temperature overnight. Tetraisopropoxytitanium (4 equiv., 329.2 mmol) was then added and stirring was continued for another 3 hours to give an orange solution. Then, the solution was treated with 1-(3,5-dichlorophenyl)-2,2,2-trifluoro-ethanone (20 g, 82.30 mmol) in one portion. The reaction mixture was stirred at room temperature for 3 days. The reaction mixture was quenched carefully with NH₄Cl sat aqueous solution at 0° C., then allowed to stir at room temperature for 20 min. The toluene phase was then filtered over celite. The aqueous phase was extracted twice with ethyl acetate and each time the organic phases were filtered over celite. Finally, the ethyl acetate phases were grouped, washed once with brine, dried over magnesium sulfate and solvents were removed under reduced pressure. The crude product was purified over a silica gel column (eluent: heptane/EtOAc) to give 33.4 g of expected tert-butyl 4-[3-(3,5-dichlorophenyl)-4,4,4-trifluoro-3-hydroxy-but-1-ynyl]-2-methyl-benzoate) as a colorless oil.

LCMS (Method A) RT 1.37 min, [M+H]⁺ 457/459/460; ¹H-NMR (CDCl₃, 400 MHz): 1.60 (s, 9H), 2.55 (s, 3H), 3.64 (s, 1H, OH), 7.27 (s, 1H), 7.33 (d, 1H), 7.41 (s, 1H), 7.68 (m, 2H), 7.79 (d, 1H). ¹¹⁹F-NMR (CDCl₃, 376.3 MHz): −80.05.

### Chiral HPLC Analysis

Column: Daicel CHIRALPAK® IB, 3 μm, 0.46 cm×10 cm
Mobile phase: Hept/DCM 50/50
Flow rate: 1.0 ml/min
2 isomers were detected: rt=1.94 min (86.2%) 2.28 min. (13.8%).

### Step D: tert-butyl 4-[(E,3S)-3-(3,5-dichlorophenyl)-4,4,4-trifluoro-3-hydroxy-but-1-enyl]-2-methyl-benzoate

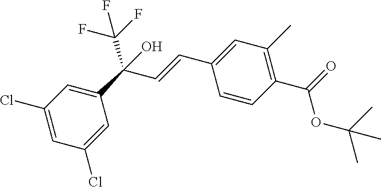 [SMILES: Cc1cc(/C=C/[C@](O)(c2cc(Cl)cc(Cl)c2)C(F)(F)F)ccc1C(=O)OC(C)(C)C]

To a solution of tert-butyl 4-[(3R)-3-(3,5-dichlorophenyl)-4,4,4-trifluoro-3-hydroxy-but-1-ynyl]-2-methyl-benzoate (43.0 g, 93.6 mmol) in 400 mL of toluene and 20 mL of THF cooled to −40° C., sodium bis(2-methoxyethoxy)aluminum hydride (70 mass % in Toluene) (Approx. 3.5 M) (2.0 equiv., 187.0 mmol) was added dropwise keeping the reaction below −30° C. (gas-evolution). The reaction was stirred at −40° C. for 1 h. The reaction mixture was carefully quenched first with acetone (10 mL) at −40° C. and then with NH₄Cl solution sat at −10° C. and extracted twice with ethyl acetate. The combined organic layers were dried (MgSO4), filtered and evaporated to give a colorless oil. The crude product was purified over a silica gel column (eluent: cyclohexane/EtOAc) to give 5.25 g of tert-butyl 4-[(E)-3-(3,5-dichlorophenyl)-4,4,4-trifluoro-3-hydroxy-but-1-enyl]-2-methyl-benzoate as a colorless oil. LCMS (Method A) RT 1.35 min, [M+H]⁺ 459/461/462; ¹H-NMR (CDCl₃, 400 MHz): 1.6 (s, 9H), 2.57 (s, 3H), 2.80 (s, 1H), 6.75 (dd, 2H), 7.25 (m, 2H), 7.49 (m, 1H), 7.53 (m, 2H), 7.8 (d, 1H). ¹⁹F-NMR (CDCl₃, 376.3 MHz): −79.4.

### Step E: 4-[(2S)-2-(3,5-dichlorophenyl)-2-(trifluoromethyl)-3H-furan-4-yl]-2-methyl-benzoic acid

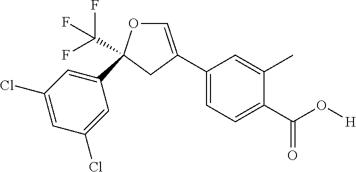 [SMILES: Cc1cc(C2=CO[C@@](c3cc(Cl)cc(Cl)c3)(C(F)(F)F)C2)ccc1C(=O)O]

A homogeneous solution of [Rh(CO)₂acac] (0.01 equiv., 0.009 mmol), tert-butyl 4-[(E)-3-(3,5-dichlorophenyl)-4,4,4-trifluoro-3-hydroxy-but-1-enyl]-2-methyl-benzoate (0.4 g, 0.88 mmol) and tris(2,4-ditert-butylphenyl) phosphite (0.1 equiv., 0.087 mmol) in toluene (8 mL) in a stainless steel autoclave was purged three times with hydrogen (5 bar), pressurized at 25 bar with H2 followed by an additional 25 bar of CO (=50 bar CO/H₂ 1:1). The reaction was then heated at 100° C. and vigorously stirred for 20 h. The reaction was stopped by cooling the autoclave to room temperature, venting and purging with argon. The crude reaction was transferred into a 30 mL vial and 4-methylbenzenesulfonic acid (0.2 equiv., 0.173 mmol) was added and mixture was heated at reflux for 5 hours. The mixture was then cooled to room temperature, diluted with ethyl acetate and washed twice with NaHCO₃ sat aqueous solution, once with water and once with brine. The organic phase was then dried over magnesium sulfate, filtered and solvents were evaporated under reduced pressure. The crude product was purified over a silica gel column (eluent: cyclohexane/EtOAc) to yield a 154 mg of 4-[2-(3,5-dichlorophenyl)-2-(trifluoromethyl)-3H-furan-4-yl]-2-methyl-benzoic acid. LCMS (Method A) RT 1.23 min; 1H-NMR (CDCl3, 400 MHz): 2.68 (s, 3H), 3.35 (d, 1H), 3.78 (d, 1H), 7.10 (m, 2H), 7.18 (m, 1H), 7.42 (m, 1H), 7.52 (m, 2H), 8.10 (d, 1H). ¹⁹F-NMR (CDCl₃, 376.3 MHz): −79.4.

### Step F: 4-[(2S)-2-(3,5-dichlorophenyl)-2-(trifluoromethyl)-3H-furan-4-yl]-N-(1,1-dioxothietan-3-yl)-2-methyl-benzamide

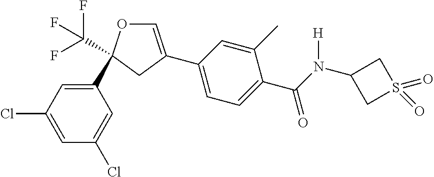 [SMILES: Cc1cc(C2=CO[C@@](c3cc(Cl)cc(Cl)c3)(C(F)(F)F)C2)ccc1C(=O)NC1CS(=O)(=O)C1]

To a stirred solution of 4-[2-(3,5-dichlorophenyl)-2-(trifluoromethyl)-3H-furan-4-yl]-2-methyl-benzoic acid (1 g, 2.40 mmol) in dry dichloromethane (50 mL) was added oxalyl chloride (1.0 equiv., 2.4 mmol) and then one drop of N,N-dimethylformamide. The reaction mixture was stirred at room temperature until no more CO formation was observed. The mixture was then evaporated to dryness and dissolved in dry dichloromethane (10 mL). This solution was then added dropwise at 0° C. to a mixture of 1,1-dioxothietan-3-amine (1.1 equiv., 2.64 mmol) and triethylamine in dry dichloromethane (20 mL). The mixture was stirred at 0° C. for 30 min and then allowed to stir at room temperature for 4 h and then quenched with water. The organic phase was washed once with brine and then solvent was evaporated under reduced pressure. The crude product was purified by flash chromatography over a silica gel column (eluent: cyclohexane/EtOAc). After removal of the solvents, a colorless oil was obtained which was dissolved in a minimum of TBME and after dilution with heptanes a white precipitate appeared which was filtered and dried under high vacuum to yield the titled compound. Mp: 90-105° C. LCMS (Method A) RT 1.16 min, [M+H]⁺ 567/569/571; ¹H-NMR (CDCl₃, 400 MHz): 1-55 (s, 2H), 2.45 (s, 3H), 3.29 (m, 1H), 3.72 (m, 1H), 4.01 (m, 2H), 4.61 (m, 2H), 4.87 (m, 1H), 6.45 (d, 1H), 7.01 (s, 1H), 7.10 (m, 2H), 7.26 (s, 1H), 7.37 (d, 1H), 7.41 (m, 1H), 7.49 (m, 2H). ¹⁹F-NMR (CDCl₃, 400 MHz): −80.87.

### Chiral HPLC Analysis

Column: Daicel CHIRALPAK® IA, 3 μm, 0.46 cm×10 cm
Mobile phase: Heptan/iPrOH/DEA 80/20/0.1%
Flow rate: 1 ml/min
2 isomers were detected: rt=8.88 min (84.4%) 10.79 min. (15.6%).

### LC/MS Method A

| MS | ACQUITY SQD Mass Spectrometer from Waters (Single quadrupole mass spectrometer) | | | | |
|---|---|---|---|---|---|
| | Ionisation method: Electrospray | | | | |
| | Polarity: positive ions | | | | |
| | Capillary (kV) 3.00, Cone (V) 20.00, Extractor (V) 3.00, Source Temperature (° C.) 150, | | | | |
| | Desolvation Temperature (° C.) 400, Cone Gas Flow (L/Hr) 60, Desolvation Gas Flow (L/Hr) | | | | |
| | 700 | | | | |
| | Mass range: 100 to 800 Da | | | | |
| | DAD Wavelength range (nm): 210 to 400 | | | | |
| LC | Method Waters ACQUITY UPLC with the following HPLC gradient conditions | | | | |
| | (Solvent A: Water/Methanol 9:1,0.1% formic acid and Solvent B: Acetonitrile,0.1% formic | | | | |
| | acid ) | | | | |
| | Time | (minutes) | A (%) | B (%) | Flow rate (ml/min) |
| | 0 | 100 | 0 | 0.75 | |
| | 2.5 | 0 | 100 | 0.75 | |
| | 2.8 | 0 | 100 | 0.75 | |
| | 3.0 | 100 | 0 | 0.75 | |

Type of column: Waters ACQUITY UPLC HSS T3; Column length: 30 mm; Internal diameter
of column: 2.1 mm; Particle Size: 1.8 micron; Temperature: 60° C.

TABLE A
experimental data obtained using LC/MS method A

| Comp | | | (M + H) + |
|---|---|---|---|
| No. | Compound name | RT | (measured) |
| A03 | N-[[2-chloro-4-[2-(3,5-dichlorophenyl)-2-(trifluoromethyl)-3H-furan-4- | 2.25 | 520.3 |
| | yl]phenyl]methyl]-2-ethyl-butanamide | | |
| A04 | N-[[2-chloro-4-[2-(3,5-dichlorophenyl)-2-(trifluoromethyl)-3H-furan-4- | 1.86 | 532.2 |
| | yl]phenyl]methyl]-2-(1H-tetrazol-5-yl)acetamide | | |
| A05 | N-[[2-chloro-4-[2-(3,5-dichlorophenyl)-2-(trifluoromethyl)-3H-furan-4- | 2.06 | 477.6 |
| | yl]phenyl]methyl]propanamide | | |
| A06 | N-[[2-chloro-4-[2-(3,5-dichlorophenyl)-2-(trifluoromethyl)-3H-furan-4- | 2.24 | 530.4 |
| | yl]phenyl]methyl]cyclohex-3-ene-l-carboxamide | | |
| A07 | N-[[2-chloro-4-[2-(3,5-dichlorophenyl)-2-(trifluoromethyl)-3H-furan-4- | 2.12 | 520.2 |
| | yl]phenyl]methyl]tetrahydrofuran-2-carboxamide | | |
| A08 | 2-benzyloxy-N-[[2-chloro-4-[2-(3,5-dichlorophenyl)-2- | 2.28 | 570.3 |
| | (trifluoromethyl)-3H-furan-4-yl]phenyl]methyl]acetamide | | |
| A09 | N-[[2-chloro-4-[2-(3,5-dichlorophenyl)-2-(trifluoromethyl)-3H-furan-4- | 2.24 | 554.2 |
| | yl]phenyl]methyl]-3-phenyl-propanamide | | |
| A10 | (E)-N-[[2-chloro-4-[2-(3,5-dichlorophenyl)-2-(trifluoromethyl)-3H- | 2.18 | 542.3 |
| | furan-4-yl]phenyl]methyl]-3-(2-furyl)prop-2-enamide | | |
| A11 | N-[[2-chloro-4-[2-(3,5-dichlorophenyl)-2-(trifluoromethyl)-3H-furan-4- | 2.38 | 546.4 |
| | yl]phenyl]methyl]-2-methyl-cyclohexanecarboxamide | | |
| A12 | 2-(1H-benzimidazol-2-ylsulfanyl)-N-[[2-chloro-4-[2-(3,5- | 2.08 | 612.2 |
| | dichlorophenyl)-2-(trifluoromethyl)-3H-furan-4-yl]phenyl]methyl]acetamide | | |
| A13 | N-[[2-chloro-4-[2-(3,5-dichlorophenyl)-2-(trifluoromethyl)-3H-furan-4- | 2.19 | 582.3 |
| | yl]phenyl]methyl]-4-oxo-4-phenyl-butanamide | | |
| A14 | N-[[2-chloro-4-[2-(3,5-dichlorophenyl)-2-(trifluoromethyl)-3H-furan-4- | 2.2 | 540.2 |
| | yl]phenyl]methyl]-2-phenyl-acetamide | | |
| A15 | N-[[2-chloro-4-[2-(3,5-dichlorophenyl)-2-(trifluoromethyl)-3H-furan-4- | 1.98 | 606.5 |
| | yl]phenyl]methyl]-2-[(2E)-3-methyl-2-methylimino-4-oxo-thiazolidin- | | |
| | 5-yl]acetamide | | |
| A16 | (E)-N-[[2-chloro-4-[2-(3,5-dichlorophenyl)-2-(trifluoromethyl)-3H- | 2.27 | 570.2 |
| | furan-4-yl]phenyl]methyl]-3-(2-fluorophenyl)prop-2-enamide | | |
| A17 | (E)-N-[[2-chloro-4-[2-(3,5-dichlorophenyl)-2-(trifluoromethyl)-3H- | 2.32 | 586.5 |
| | furan-4-yl]phenyl]methyl]-3-(2-chlorophenyl)prop-2-enamide | | |
| A18 | N-[[2-chloro-4-[2-(3,5-dichlorophenyl)-2-(trifluoromethyl)-3H-furan-4- | 2.18 | 546.2 |
| | yl]phenyl]methyl]-2-(2-thienyl)acetamide | | |
| A19 | N-[[2-chloro-4-[2-(3,5-dichlorophenyl)-2-(trifluoromethyl)-3H-furan-4- | 2.25 | 556.2 |
| | yl]phenyl]methyl]-2-phenoxy-acetamide | | |
| A20 | N-[[2-chloro-4-[2-(3,5-dichlorophenyl)-2-(trifluoromethyl)-3H-furan-4- | 2.27 | 562.2 |
| | yl]phenyl]methyl]-2,3-difluoro-benzamide | | |
| A21 | N-[[2-chloro-4-[2-(3,5-dichlorophenyl)-2-(trifluoromethyl)-3H-furan-4- | 2.25 | 583.7 |
| | yl]phenyl]methyl]-2,1,3-benzothiadiazole-5-carboxamide | | |
| A22 | N-[[2-chloro-4-[2-(3,5-dichlorophenyl)-2-(trifluoromethyl)-3H-furan-4- | 2.11 | 623.2 |
| | yl]phenyl]methyl]-3-(1,3-dioxoisoindolin-2-yl)propanamide | | |
| A23 | N-[[2-chloro-4-[2-(3,5-dichlorophenyl)-2-(trifluoromethyl)-3H-furan-4- | 2.37 | 578.4 |
| | yl]phenyl]methyl]-3-methyl-1H-indene-2-carboxamide | | |
| A24 | N-[[2-chloro-4-[2-(3,5-dichlorophenyl)-2-(trifluoromethyl)-3H-furan-4- | 2.33 | 623.2 |
| | yl]phenyl]methyl]-2-(2-phenylthiazol-4-yl)acetamide | | |
| A25 | N-[[2-chloro-4-[2-(3,5-dichlorophenyl)-2-(trifluoromethyl)-3H-furan-4- | 2.32 | 589.6 |
| | yl]phenyl]methyl]-2-(4-chlorophenoxy)acetamide | | |
| A26 | N-[[2-chloro-4-[2-(3,5-dichlorophenyl)-2-(trifluoromethyl)-3H-furan-4- | 2.19 | 556.2 |
| | yl]phenyl]methyl]-4-methoxy-benzamide | | |
| A27 | N-[[2-chloro-4-[2-(3,5-dichlorophenyl)-2-(trifluoromethyl)-3H-furan-4- | 2.27 | 572.3 |
| | yl]phenyl]methyl]-4-methylsulfanyl-benzamide | | |
| A28 | 2,5-dichloro-N-[[2-chloro-4-[2-(3,5-dichlorophenyl)-2- | 2.33 | 593.7 |
| | (trifluoromethyl)-3H-furan-4-yl]phenyl]methyl]benzamide | | |
| A29 | N-[[2-chloro-4-[2-(3,5-dichlorophenyl)-2-(trifluoromethyl)-3H-furan-4- | 2.3 | 590.9 |
| | yl]phenyl]methyl]-4-pyrrol-1-yl-benzamide | | |
| A30 | N-[[2-chloro-4-[2-(3,5-dichlorophenyl)-2-(trifluoromethyl)-3H-furan-4- | 2.38 | 602.5 |
| | yl]phenyl]methyl]-4-phenyl-benzamide | | |
| A31 | N-[[2-chloro-4-[2-(3,5-dichlorophenyl)-2-(trifluoromethyl)-3H-furan-4- | 1.83 | 516.3 |
| | yl]phenyl]methyl]-1H-imidazole-4-carboxamide | | |
| A32 | N-[[2-chloro-4-[2-(3,5-dichlorophenyl)-2-(trifluoromethyl)-3H-furan-4- | 2.22 | 598.2 |
| | yl]phenyl]methyl]-3,4-dihydro-2H-1,5-benzodioxepine-7-carboxamide | | |
| A33 | N-[[2-chloro-4-[2-(3,5-dichlorophenyl)-2-(trifluoromethyl)-3H-furan-4- | 2.35 | 612.3 |
| | yl]phenyl]methyl]-2-fluoro-3-(trifluoromethyl)benzamide | | |
| A34 | N-[[2-chloro-4-[2-(3,5-dichlorophenyl)-2-(trifluoromethyl)-3H-furan-4- | 2.1 | 578.2 |
| | yl]phenyl]methyl]cinnoline-4-carboxamide | | |
| A35 | N-[[2-chloro-4-[2-(3,5-dichlorophenyl)-2-(trifluoromethyl)-3H-furan-4- | 2.27 | 562.2 |
| | yl]phenyl]methyl]-4-methoxy-thiophene-3-carboxamide | | |
| A36 | N-[[2-chloro-4-[2-(3,5-dichlorophenyl)-2-(trifluoromethyl)-3H-furan-4- | 2.37 | 614.2 |
| | yl]phenyl]methyl]-9H-fluorene-4-carboxamide | | |
| A37 | N-[[2-chloro-4-[2-(3,5-dichlorophenyl)-2-(trifluoromethyl)-3H-furan-4- | 2.3 | 581.8 |
| | yl]phenyl]methyl]benzothiophene-5-carboxamide | | |
| A38 | N-[[2-chloro-4-[2-(3,5-dichlorophenyl)-2-(trifluoromethyl)-3H-furan-4- | 2.4 | 593.2 |
| | yl]phenyl]methyl]-5-phenyl-oxazole-4-carboxamide | | |
| A39 | N-[[2-chloro-4-[2-(3,5-dichlorophenyl)-2-(trifluoromethyl)-3H-furan-4- | 2.33 | 541.2 |
| | yl]phenyl]methyl]-3-methyl-pyridine-2-carboxamide | | |
| A40 | N-[[2-chloro-4-[2-(3,5-dichlorophenyl)-2-(trifluoromethyl)-3H-furan-4- | 2.13 | 595.2 |
| | yl]phenyl]methyl]-2,7-dimethyl-pyrazolo[1,5-a]pyrimidine-6- | | |
| | carboxamide | | |
| A41 | N-[[2-chloro-4-[2-(3,5-dichlorophenyl)-2-(trifluoromethyl)-3H-furan-4- | 2.19 | 593.7 |
| | yl]phenyl]methyl]-4-oxo-chromene-2-carboxamide | | |
| A42 | N-[[2-chloro-4-[2-(3,5-dichlorophenyl)-2-(trifluoromethyl)-3H-furan-4- | 2.23 | 609.2 |
| | yl]phenyl]methyl]-5-(2-pyridyl)thiophene-2-carboxamide | | |
| A43 | N-[[2-chloro-4-[2-(3,5-dichlorophenyl)-2-(trifluoromethyl)-3H-furan-4- | 2.24 | 606.2 |
| | yl]phenyl]methyl]-5-methyl-l-phenyl-pyrazole-4-carboxamide | | |
| A44 | N-[[2-chloro-4-[2-(3,5-dichlorophenyl)-2-(trifluoromethyl)-3H-furan-4- | 2.18 | 532.1 |
| | yl]phenyl]methyl]thiophene-2-carboxamide | | |
| A45 | N-[[2-chloro-4-[2-(3,5-dichlorophenyl)-2-(trifluoromethyl)-3H-furan-4- | 2.28 | 607.2 |
| | yl]phenyl]methyl]-5-methyl-3-phenyl-isoxazole-4-carboxamide | | |
| A46 | N-[[2-chloro-4-[2-(3,5-dichlorophenyl)-2-(trifluoromethyl)-3H-furan-4- | 2.13 | 492.2 |
| | yl]phenyl]methyl]-2-methyl-propanamide | | |
| A47 | 2-chloro-N-[[2-chloro-4-[2-(3,5-dichlorophenyl)-2-(trifluoromethyl)- | 2.24 | 526.2 |
| | 3H-furan-4-yl]phenyl]methyl]butanamide | | |
| A48 | N-[[2-chloro-4-[2-(3,5-dichlorophenyl)-2-(trifluoromethyl)-3H-furan-4- | 2.12 | 531.7 |
| | yl]phenyl]methyl]-3,3,3-trifluoro-propanamide | | |
| A49 | N-[[2-chloro-4-[2-(3,5-dichlorophenyl)-2-(trifluoromethyl)-3H-furan-4- | 2.32 | 536.2 |
| | yl]phenyl]methyl]-1-methylsulfanyl-cyclopropanecarboxamide | | |
| A50 | N-[[2-chloro-4-[2-(3,5-dichlorophenyl)-2-(trifluoromethyl)-3H-furan-4- | 2.17 | 563.6 |
| | yl]phenyl]methyl]-4-methyl-5-oxo-1,3,4-thiadiazole-2-carboxamide | | |
| A51 | N-[[2-chloro-4-[2-(3,5-dichlorophenyl)-2-(trifluoromethyl)-3H-furan-4- | 1.95 | 542.2 |
| | yl]phenyl]methyl]-2-methylsulfonyl-acetamide | | |
| A52 | N-[[2-chloro-4-[2-(3,5-dichlorophenyl)-2-(trifluoromethyl)-3H-furan-4- | 2.05 | 560.4 |
| | yl]phenyl]methyl]-3-fluoro-4-hydroxy-benzamide | | |
| A53 | N-[[2-chloro-4-[2-(3,5-dichlorophenyl)-2-(trifluoromethyl)-3H-furan-4- | 2.34 | 584.3 |
| | yl]phenyl]methyl]-2,2-bis(ethylsulfanyl)acetamide | | |

### BIOLOGICAL EXAMPLES

### Spodoptera littoralis (Egyptian Cotton Leafworm)

Cotton leaf discs were placed on agar in a 24-well microtiter plate and sprayed with test solutions at an application rate of 200 ppm. After drying, the leaf discs were infested with 5 L1 larvae. The samples were checked for mortality, feeding behavior, and growth regulation 3 days after treatment (DAT).

The following compound gave at least 80% control of Spodoptera littoralis: A1, A2, A03, A05, A06, A07, A08, A13, A14, A15, A18, A20, A22, A26, A31, A38, A39, A44, A46, A47, A48, A49, A50, A51, A53, B1**.

Heliothis virescens (Tobacco Budworm):

Eggs (0-24 h old) were placed in 24-well microtiter plate on artificial diet and treated with test solutions at an application rate of 200 ppm (concentration in well 18 ppm) by pipetting. After an incubation period of 4 days, samples were checked for egg mortality, larval mortality, and growth regulation. The following compound gave at least 80% control of Heliothis virescens: A1, A2, A03, A05, A06, A07, A08, A09, A10, A11, A12, A13, A14, A15, A16, A17, A18, A19, A20, A21, A22, A25, A26, A27, A31, A32, A34, A37, A39, A40, A42, A44, A45, A46, A47, A48, A49, A50, A51, A52, A53, B1**.

Plutella xylostella (Diamond Back Moth):

24-well microtiter plate (MTP) with artificial diet was treated with test solutions at an application rate of 200 ppm (concentration in well 18 ppm) by pipetting. After drying, the MTPs were infested with L2 larvae (7-12 per well). After an incubation period of 6 days, samples were checked for larval mortality and growth regulation.

The following compound gave at least 80% control of Plutella xylostella: A1, A2, A03, A05, A06, A07, A09, A10, A11, A12, A13, A14, A15, A18, A19, A20, A22, A26, A28, A31, A34, A39, A40, A44, A46, A47, A48, A49, A50, A51, A52, A53, B1**.

Diabrotica balteata (Corn Root Worm):

A 24-well microtiter plate (MTP) with artificial diet was treated with test solutions at an application rate of 200 ppm (concentration in well 18 ppm) by pipetting. After drying, the MTPs were infested with L2 larvae (6-10 per well). After an incubation period of 5 days, samples were checked for larval mortality and growth regulation.

The following compound gave at least 80% control of Diabrotica balteata: A1, A2, A03, A05, A06, A07, A09, A15, A19, A20, A22, A26, A27, A39, A44, A46, A47, A48, A50, A51, A53, B1**.

Thrips tabaci (Onion Thrips):

Sunflower leaf discs were placed on agar in a 24-well microtiter plate and sprayed with test solutions at an application rate of 200 ppm. After drying, the leaf discs were infested with a thrip population of mixed ages. After an incubation period of 7 days, samples were checked for mortality.

The following compounds gave at least 80% control of Thrips tabaci: A1, A2, A05, A46, A48, A51, B1**.

Tetranychus urticae (Two-Spotted Spider Mite):

Bean leaf discs on agar in 24-well microtiter plates were sprayed with test solutions at an application rate of 200 ppm. After drying, the leaf discs are infested with mite populations of mixed ages. 8 days later, discs are checked for egg mortality, larval mortality, and adult mortality.

The following compound gave at least 80% control of Tetranychus urticae: A1, A2, A03, A05, A06, A07, A08, A11, A14, A15, A18, A19, A40, A46, A48, A51, B1**.

1. A compound of formula (I) 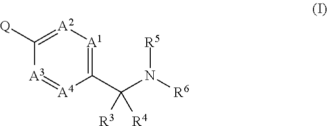
wherein
Q is Q1 or Q2 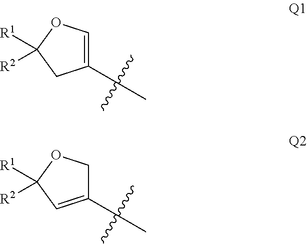
A¹, A², A³ and A⁴ are independently of each other C—H, C—R⁷, or nitrogen;
R¹ is C₁-C₈haloalkyl;
R² is aryl or aryl substituted by one to five R¹¹, or heteroaryl or heteroaryl substituted by one to five R¹¹;
R³ and R⁴ are each independently hydrogen, C₁-C₁₂alkyl or C₁-C₁₂alkyl substituted by one to five R⁸, C₃-C₈cycloalkyl or C₃-C₈cycloalkyl substituted by one to five R⁹, C₂-C₁₂alkenyl or C₂-C₁₂alkenyl substituted by one to five R⁸, C₂-C₁₂alkynyl or C₂-C₁₂alkynyl substituted by one to five R⁸, cyano, C₁-C₁₂alkoxycarbonyl or C₁-C₁₂alkoxycarbonyl substituted by one to five R⁸, C₁-C₁₂alkoxythiocarbonyl or C₁-C₁₂alkoxythiocarbonyl substituted by one to five R⁸, or R³ and R⁴ together with the carbon atom to which they are attached may form a 3 to 6-membered carbocyclic ring;
or when A¹ is C—R⁷, the R⁷ attached to A¹, R³ and fragment to which they are attached may together for a 5- to 7-membered carbocyclic ring optionally substituted by one to five R¹⁶;
R⁵ is hydrogen, NH₂, hydroxyl, C₁-C₁₂ alkoxy or C₁-C₁₂alkoxy substituted by one to five R⁸, C₁-C₁₂alkylcarbonylamino or C₁-C₁₂alkylcarbonylamino wherein the alkyl is substituted by one to five R⁸, C₁-C₁₂alkylamino or C₁-C₁₂alkylamino wherein the alkyl is substituted by one to five R⁸, C₁-C₁₂alkyl or C₁-C₁₂alkyl substituted by one to five R⁸, C₃-C₈cycloalkyl or C₃-C₈cycloalkyl substituted by one to five R⁹, cyano, C₂-C₁₂alkenyl or C₂-C₁₂alkenyl substituted by one to five R⁸, C₂-C₁₂alkynyl or C₂-C₁₂alkynyl substituted by one to five R⁸, C₁-C₁₂alkylcarbonyl or C₁-C₁₂alkylcarbonyl substituted by one to five R⁸, C₁-C₁₂alkoxycarbonyl or C₁-C₁₂alkoxycarbonyl substituted by one to five R⁸ or is selected from CH₂—R¹³, C(═O)R¹³ and C(═S)R¹³;
R⁶ is hydrogen, cyano, carbonyl, thiocarbonyl, C₁-C₁₂alkylcarbonyl or C₁-C₁₂ alkylcarbonyl substituted by one to five R⁸, C₁-C₁₂alkylthiocarbonyl or C₁-C₁₂alkylthiocarbonyl substituted by one to five R⁸, C₁-C₁₂alkylaminocarbonyl or C₁-C₁₂alkylaminocarbonyl wherein the alkyl is substituted by one to five R⁸, C₁-C₁₂alkylaminothiocarbonyl or C₁-C₁₂alkylaminothiocarbonyl wherein the alkyl is substituted by one to five R⁸, C₂-C₂₄ (total carbon number) dialkylaminocarbonyl or C₂-C₂₄ (total carbon number) dialkylaminocarbonyl wherein one or both alkyl is substituted by one to five R⁸, C₂-C₂₄ (total carbon number) dialkylaminothiocarbonyl or C₂-C₂₄ (total carbon number) dialkylaminothiocarbonyl wherein one or both alkyl is substituted by one to five R⁸, C₁-C₁₂alkoxyaminocarbonyl or C₁-C₁₂alkoxyaminocarbonyl wherein the alkoxy is substituted by one to five R⁸, C₁-C₁₂alkoxyaminothiocarbonyl or C₁-C₁₂alkoxyaminothiocarbonyl wherein the alkoxy is substituted by one to five R⁸, C₁-C₁₂alkoxycarbonyl or C₁-C₁₂alkoxycarbonyl substituted by one to five R⁸, C₁-C₁₂alkoxythiocarbonyl or C₁-C₁₂alkoxythiocarbonyl substituted by one to five R⁸, C₁-C₁₂thioalkoxycarbonyl or C₁-C₁₂thioalkoxycarbonyl substituted by one to five R⁸, C₁-C₁₂thioalkoxythiocarbonyl or C₁-C₁₂thioalkoxythiocarbonyl substituted by one to five R⁸, C₁-C₁₂alkylsulfonyl or C₁-C₁₂alkylsulfonyl substituted by one to five R⁸, C₃-C₁₂cycloalkylcarbonyl or C₃-C₁₂cycloalkylcarbonyl substituted by one to five R⁹, C₂-C₁₂alkenylcarbonyl or C₂-C₁₂alkenylcarbonyl substituted by one to five R⁸, C₂-C₁₂alkynylcarbonyl or C₂-C₁₂alkynylcarbonyl substituted by one to five R⁸, C₃-C₁₂cycloalkyl-C₁-C₁₂alkylcarbonyl or C₃-C₁₂cycloalkyl-C₁-C₁₂alkylcarbonyl substituted by one to five R⁹, C₁-C₁₂alkylsulfenyl-C₁-C₁₂alkylcarbonyl or C₁-C₁₂alkylsulfenyl-C₁-C₁₂alkylcarbonyl substituted by one to five R⁸, C₁-C₁₂alkylsulfinyl-C₁-C₁₂alkylcarbonyl or C₁-C₁₂alkylsulfinyl-C₁-C₁₂alkylcarbonyl substituted by one to five R⁸, C₁-C₁₂ alkylsulfonyl-C₁-C₁₂alkylcarbonyl or C₁-C₁₂alkylsulfonyl-C₁-C₁₂alkylcarbonyl substituted by one to five R⁸, C₁-C₁₂alkylcarbonyl-C₁-C₁₂alkylcarbonyl or C₁-C₁₂alkylcarbonyl-C₁-C₁₂alkylcarbonyl substituted by one to five R⁸, C₃-C₁₂cycloalkylaminocarbonyl or C₃-C₁₂cycloalkylaminocarbonyl wherein the cycloalkyl is substituted by one to five R⁹, C₂-C₁₂alkenylaminocarbonyl or C₂-C₁₂alkenylaminocarbonyl wherein the alkenyl is substituted by one to five R⁸, C₂-C₁₂alkynylaminocarbonyl or C₂-C₁₂alkynylaminocarbonyl wherein the alkynyl is substituted by one to five R⁸, or is selected from C(═O)R¹³ and C(═S)R¹³;
or R⁵ and R⁶ together with the nitrogen atom to which they are bound, form a 3- to 6-membered heterocyclic ring which may be substituted by one to five R¹⁴, or may be substituted with a keto, thioketo or nitroimino group;
each R⁷ is independently halogen, cyano, nitro, C₁-C₈alkyl, C₃-C₈cycloalkyl, C₁-C₈haloalkyl, C₂-C₈alkenyl, C₂-C₈haloalkenyl, C₂-C₈alkynyl, C₂-C₈haloalkynyl, C₁-C₈alkoxy, C₁-C₈haloalkoxy, C₁-C₈alkoxycarbonyl-, or two R⁷ on adjacent carbon atoms together form a —CH═CH—CH═CH— bridge or a —N═CH—CH═CH— bridge;
each R⁸ is independently halogen, cyano, nitro, hydroxy, NH₂, mercapto, C₁-C₈alkyl, C₁-C₈haloalkyl, C₁-C₈alkoxy, C₁-C₈haloalkoxy, C₁-C₈alkylthio, C₁-C₈haloalkylthio, C₁-C₈alkylsulfinyl, C₁-C₈haloalkylsulfinyl, C₁-C₈alkylsulfonyl, C₁-C₈haloalkylsulfonyl, C₁-C₈alkylamino, C₂-C₈dialkylamino, C₃-C₈cycloalkylamino, C₁-C₈alkylcarbonyl, C₁-C₈alkoxycarbonyl, C₁-C₈alkylaminocarbonyl, C₁-C₈dialkylaminocarbonyl, C₁-C₈haloalkylcarbonyl, C₁-C₈haloalkoxycarbonyl, C₁-C₈haloalkylaminocarbonyl, C₁-C₈halodialkylaminocarbonyl;
each R⁹ is independently halogen or C₁-C₈alkyl;
each R¹⁰ is independently halogen, cyano, nitro, C₁-C₈alkyl, C₁-C₈haloalkyl, C₂-C₈alkenyl, C₂-C₈haloalkenyl, C₂-C₈alkynyl, C₂-C₈haloalkynyl, hydroxy, C₁-C₈alkoxy, C₁-C₈haloalkoxy, mercapto, C₁-C₈alkylthio, C₁-C₈haloalkylthio, C₁-C₈alkylsulfinyl, C₁-C₈haloalkylsulfinyl, C₁-C₈alkylsulfonyl, C₁-C₈haloalkylsulfonyl, C₁-C₈alkylcarbonyl, C₁-C₈alkoxycarbonyl, aryl or aryl substituted by one to five R¹², or heterocyclyl or heterocyclyl substituted by one to five R¹²;
each R¹¹ is independently halogen, cyano, nitro, C₁-C₈alkyl, C₁-C₈haloalkyl, C₂-C₈alkenyl, C₂-C₈haloalkenyl, C₂-C₈alkynyl, C₂-C₈haloalkynyl, hydroxy, C₁-C₈alkoxy, C₁-C₈haloalkoxy, mercapto, C₁-C₈alkylthio, C₁-C₈haloalkylthio, C₁-C₈alkylsulfinyl, C₁-C₈haloalkylsulfinyl, C₁-C₈alkylsulfonyl, C₁-C₈haloalkylsulfonyl, C₁-C₈alkylcarbonyl, C₁-C₈alkoxycarbonyl, aryl or aryl substituted by one to five R¹², or heterocyclyl or heterocyclyl substituted by one to five R¹²;
each R¹² is independently halogen, cyano, nitro, C₁-C₄alkyl, C₁-C₄haloalkyl, C₁-C₄alkoxy-, or C₁-C₄haloalkoxy-;
R¹³ is aryl or aryl substituted by one to five R¹⁰, heterocyclyl or heterocyclyl substituted by one to five R¹⁰;
each R¹⁴ is independently halogen, cyano, nitro, C₁-C₈alkyl, C₁-C₈haloalkyl, C₁-C₈alkoxy, C₁-C₈haloalkoxy or C₁-C₈alkoxycarbonyl;
each R¹⁶ is independently hydrogen, halogen, cyano, nitro, C₁-C₈alkyl, C₁-C₈haloalkyl, C₂-C₈alkenyl, C₂-C₈haloalkenyl, C₂-C₈alkynyl, C₂-C₈haloalkynyl, hydroxy, C₁-C₈alkoxy, C₁-C₈haloalkoxy, mercapto, C₁-C₈alkylthio, C₁-C₈haloalkylthio, C₁-C₈alkylsulfinyl, C₁-C₈haloalkylsulfinyl, C₁-C₈alkylsulfonyl, C₁-C₈haloalkylsulfonyl, C₁-C₈alkylcarbonyl, C₁-C₈alkoxycarbonyl, aryl or aryl substituted by one to five R¹², or heterocyclyl or heterocyclyl substituted by one to five R¹²; or a salt or N-oxide thereof.

2. A compound according to claim 1, wherein R³ and R⁴ are each independently hydrogen, C₁-C₁₂alkyl or C₁-C₁₂alkyl substituted by one to five R⁸, C₃-C₈cycloalkyl or C₃-C₈cycloalkyl substituted by one to five R⁹, C₂-C₁₂alkenyl or C₂-C₁₂alkenyl substituted by one to five R⁸, C₂-C₁₂alkynyl or C₂-C₁₂alkynyl substituted by one to five R⁸, cyano, C₁-C₁₂alkoxycarbonyl or C₁-C₁₂alkoxycarbonyl substituted by one to five R⁸, C₁-C₁₂alkoxythiocarbonyl or C₁-C₁₂alkoxythiocarbonyl substituted by one to five R⁸, or R³ and R⁴ together with the carbon atom to which they are attached may form a 3 to 6-membered carbocyclic ring.

3. A compound according to claim 1, wherein when A¹ is C—R⁷, the R⁷ attached to A¹, R³ and fragment to which they are attached together form a 5- to 7-membered carbocyclic ring, optionally substituted by one to five R¹⁶.

4. A compound according to claim 1, wherein A¹ is C—R⁷, A² is C—H, C—R⁷ or nitrogen, A³ and A⁴ are independently C—H or nitrogen, wherein no more than two of A², A³ and A⁴ are nitrogen, and A³ and A⁴ are not both nitrogen, and wherein when A² is C—R⁷ then the R⁷ of A¹ and the R⁷ of A² together form a —CH═CH—CH═CH— bridge.

5. A compound according to claim 1, wherein R¹ is chlorodifluoromethyl, difluoromethyl or trifluoromethyl.

6. A compound according to claim 1, wherein R² is group P 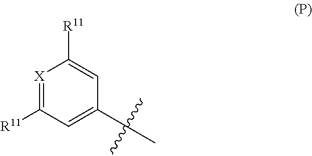
wherein X is N or C—R¹¹.

7. A compound according to claim 1, wherein R³ and R⁴ are each independently hydrogen, halogen, cyano, C₁-C₄alkyl, C₁-C₄haloalkyl or C₃-C₆ cycloalkyl or R³ and R⁴ together form a 3-6 membered carbocyclic ring.

8. A compound according to claim 1, wherein R⁵ is hydrogen, C₁-C₈alkyl, C₁-C₈haloalkyl, C₁-C₈alkoxy, C₁-C₈haloalkoxy, C₁-C₈alkylcarbonyl, C₁-C₈haloalkylcarbonyl, C₁-C₈alkoxycarbonyl, or C₁-C₈haloalkoxycarbonyl.

9. A compound according to claim 1, wherein R⁶ is hydrogen, cyano, carbonyl, thiocarbonyl, C₁-C₁₂alkylcarbonyl, C₁-C₁₂haloalkylcarbonyl, C₁-C₁₂alkylthiocarbonyl, C₁-C₁₂haloalkylthiocarbonyl, C₁-C₁₂alkylaminocarbonyl, C₁-C₁₂alkylaminothiocarbonyl, C₂-C₂₄ (total carbon number) dialkylaminocarbonyl, C₂-C₂₄ (total carbon number) dialkylaminothiocarbonyl, C₁-C₁₂alkoxyaminocarbonyl, C₁-C₁₂alkoxyaminothiocarbonyl, C₁-C₁₂alkoxycarbonyl, C₁-C₁₂haloalkoxycarbonyl, C₁-C₁₂alkoxythiocarbonyl, C₁-C₁₂haloalkoxythiocarbonyl, C₁-C₁₂thioalkoxycarbonyl, C₁-C₁₂thioalkoxythiocarbonyl, C₁-C₁₂alkoxy-C₁-C₄alkylcarbonyl, C₁-C₁₂haloalkoxy-C₁-C₄alkylcarbonyl, C₁-C₁₂alkylsulfonyl, C₁-C₁₂haloalkylsulfonyl, C₃-C₁₂cycloalkylcarbonyl, C₃-C₁₂halocycloalkylcarbonyl, C₂-C₁₂alkenylcarbonyl, C₂-C₁₂haloalkenylcarbonyl, C₂-C₁₂ alkynylcarbonyl, C₂-C₁₂haloalkynylcarbonyl, C₃-C₁₂cycloalkyl-C₁-C₁₂alkylcarbonyl, C₃-C₁₂halocycloalkyl-C₁-C₁₂alkylcarbonyl, C₂-C₁₂alkylsulfenyl-C₁-C₁₂alkylcarbonyl, C₁-C₁₂haloalkylsulfenyl-C₁-C₁₂alkylcarbonyl, C₁-C₁₂alkylsulfinyl-C₁-C₁₂alkylcarbonyl, C₁-C₁₂haloalkylsulfinyl-C₁-C₁₂alkylcarbonyl, C₁-C₁₂alkylsulfonyl-C₁-C₁₂alkylcarbonyl, C₁-C₁₂haloalkylsulfonyl-C₁-C₁₂alkylcarbonyl, C₁-C₁₂alkylcarbonyl-C₁-C₁₂alkylcarbonyl, C₁-C₁₂haloalkylcarbonyl-C₁-C₁₂alkylcarbonyl, C₃-C₁₂cycloalkylaminocarbonyl, C₂-C₁₂alkenylaminocarbonyl, C₂-C₁₂alkynylaminocarbonyl or C(═O)R¹³.

10. A compound according to claim 1, wherein R⁶ is C₁-C₈alkylcarbonyl, C₁-C₈haloalkylcarbonyl, C₃-C₈cycloalkylcarbonyl, C₃-C₈halocycloalkylcarbonyl, C₃-C₈cycloalkyl-CH₂-carbonyl, C₃-C₈halocycloalkyl-CH₂-carbonyl, C₁-C₈alkoxy-CH₂-carbonyl, C₁-C₈haloalkoxy-CH₂-carbonyl, C₁-C₈alkylsulfenyl-CH₂-carbonyl, C₁-C₈haloalkylsulfenyl-CH₂-carbonyl, C₁-C₈alkylsulfinyl-CH₂-carbonyl, C₁-C₈haloalkylsulfinyl-CH₂-carbonyl, C₁-C₈alkylsulfonyl-CH₂-carbonyl, or C₁-C₈haloalkylsulfonyl-CH₂-carbonyl, C₁-C₈alkylaminocarbonyl, C₃-C₈cycloalkylaminocarbonyl, or C(═O)R¹³ wherein R¹³ is phenyl or phenyl substituted by one to five R¹⁴, or pyridyl or pyridyl substituted by one to five R¹⁴, or tetrahydrofuranyl or tetrahydrofuranyl substituted by one to five R¹⁴.

11. A compound of formula Int-1 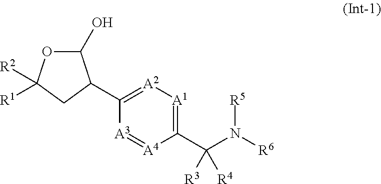
wherein A¹, A², A³, A⁴, R¹, R², R³, R⁴, R⁵ and R⁶ are as defined for compounds of formula I as defined in claim 1, or a salt of N-oxide thereof;
a compound of formula Int-2 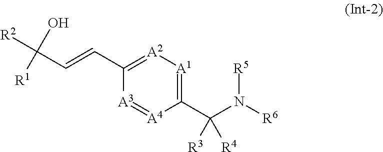
wherein A¹, A², A³, A⁴, R¹, R², R³, R⁴, R⁵ and R⁶ are as defined for compounds of formula I as defined in claim 1, or a salt of N-oxide thereof; or
a compound of formula Int-3 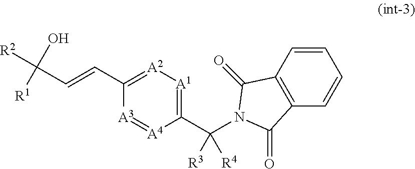
wherein A¹, A², A³, A⁴, R¹, R², R³ and R⁴ are as defined for compounds of formula I as defined in claim 1, or a salt of N-oxide thereof; or
a compound of formula Int-4 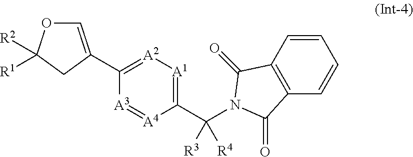
wherein A¹, A², A³, A⁴, R¹, R², R³ and R⁴ are as defined for compounds of formula I as defined in claim 1, or a salt of N-oxide thereof; or
a compound of formula Int-5 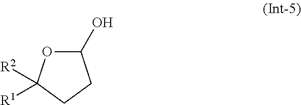
wherein R¹ and R² are as defined for compounds of formula I in claim 1; or
a compound of formula Int-6 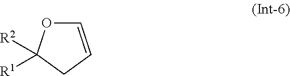
wherein R¹ and R² are as defined for compounds of formula I in claim 1; or
a compound of formula Int-7 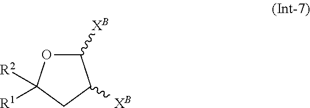
wherein R¹ and R² are as defined for compounds of formula I in claim 1 and each X^{B} independently represents Cl, Br, or I; or.
a compound of formula Int-8 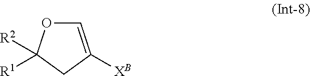
wherein R¹ and R² are as defined for compounds of formula I claim 1 and X^{B} represents Cl, Br or I; or
a compound of formula Int-9 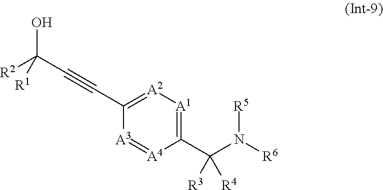
wherein A¹, A², A³, A⁴, R¹, R², R³, R⁴, R⁵ and R⁶ are as defined for compounds of formula I in claim 1, or a salt of N-oxide thereof; or
a compound of formula Int-10 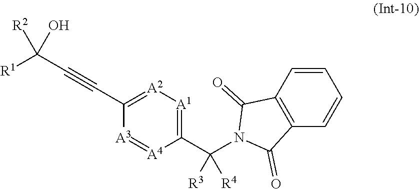
wherein A¹, A², A³, A⁴, R¹, R², R³ and R⁴ are as defined for compounds of formula I in claim 1, or a salt of N-oxide thereof; or
a compound of formula Int-11 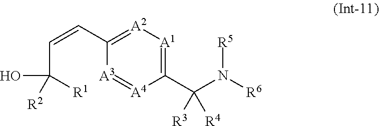
wherein A¹, A², A³, A⁴, R¹, R², R³, R⁴, R⁵ and R⁶ are as defined for compounds of formula I as defined in claim 1, or a salt of N-oxide thereof; or
a compound of formula Int-12 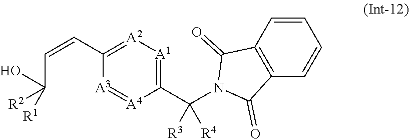
wherein A¹, A², A³, A⁴, R¹, R², R³ and R⁴ are as defined for compounds of formula I in claim 1, or a salt of N-oxide thereof; or
a compound of formula Int-13 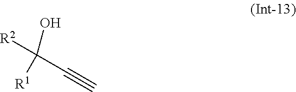
wherein R¹ and R² are as defined for compounds of formula I in claim 1; or
a compound of formula Int-14 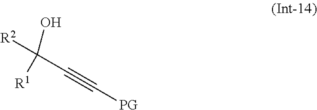
wherein R¹ and R² are as defined for compounds of formula I claim 1 and PG is an oragnosilicon, e.g. trialkylsilyl such as tri-C₁-C₄alkyl-silyl, e.g. trimethylsilyl; or
a compound of formula Int-15 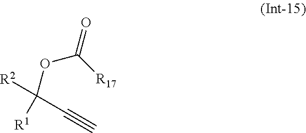
wherein R¹ and R² are as defined for compounds of formula I in claim 1 and R¹⁷ is C₁-C₁₂alkyl.

12. A compound of formula Int-2** 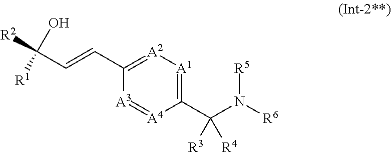
wherein A¹, A², A³, A⁴, R¹, R², R³, R⁴, R⁵ and R⁶ are as defined for compounds of formula I in claim 1, or a salt of N-oxide thereof; or
a compound of formula Int-3** 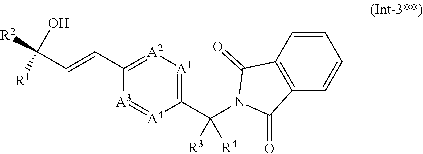
wherein A¹, A², A³, A⁴, R¹, R², R³ and R⁴ are as defined for compounds of formula I in claim 1, or a salt of N-oxide thereof; or
a compound of formula Int-9** 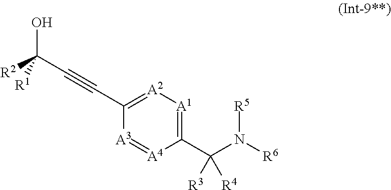
wherein A¹, A², A³, A⁴, R¹, R², R³, R⁴, R⁵ and R⁶ are as defined for compounds of formula I in claim 1, or a salt of N-oxide thereof; or
a compound of formula Int-10** 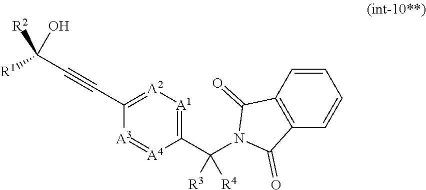
wherein A¹, A², A³, A⁴, R¹, R², R³ and R⁴ are as defined for compounds of formula I in claim 1, or a salt of N-oxide thereof; or
a compound of formula Int-11** 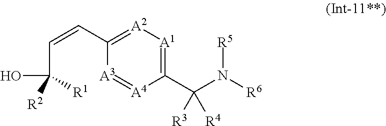
wherein A¹, A², A³, A⁴, R¹, R², R³, R⁴, R⁵ and R⁶ are as defined for compounds of formula I in claim 1, or a salt of N-oxide thereof; or
a compound of formula Int-12** 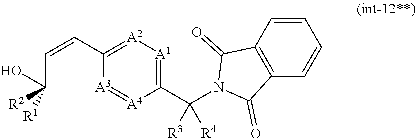
wherein A¹, A², A³, A⁴, R¹, R², R³ and R⁴ are as defined for compounds of formula I in claim 1, or a salt of N-oxide thereof; or
a compound of formula Int-13** 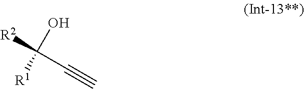
wherein R¹ and R² are as defined for compounds of formula I in claim 1; or
a compound of formula Int-14** 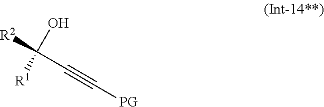
wherein R¹ and R² are as defined for compounds of formula I in claim 1 and PG is an oragnosilicon e.g. trialkylsilyl such as tri-C₁-C₄alkyl-silyl, e.g. trimethylsilyl; or
a compound of formula Int-15** 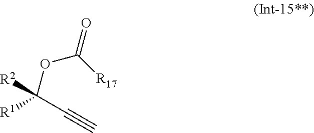
wherein R¹ and R² are as defined for compounds of formula I in claim 1 and R¹⁷ is C₁-C₁₂alkyl.

13. A mixture of compounds of formula Int-2* and Int-2**, wherein the molar amount of Int-2** in the mixture is more than 50% compared to the combined molar amount of Int-2* and Int-2** 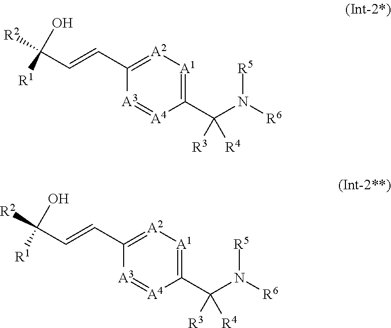
wherein A¹, A², A³, A⁴, R¹, R², R³, R⁴, R⁵ and R⁶ are as defined for compounds of formula I as defined in claim 1, or a salt of N-oxide thereof; or
a mixture of compounds of formula Int-3* and Int-3**, wherein the molar amount of Int-3** in the mixture is more than 50% compared to the combined molar amount of Int-3* and Int-3** 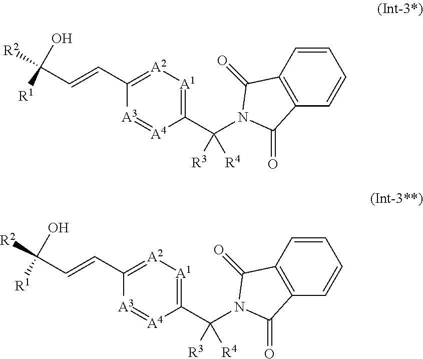
wherein A¹, A², A³, A⁴, R¹, R², R³ and R⁴ are as defined for compounds of formula I in claim 1, or a salt of N-oxide thereof; or
a mixture of compounds of formula Int-9* and Int-9**, wherein the molar amount of Int-9** in the mixture is more than 50% compared to the combined molar amount of Int-9* and Int-9** 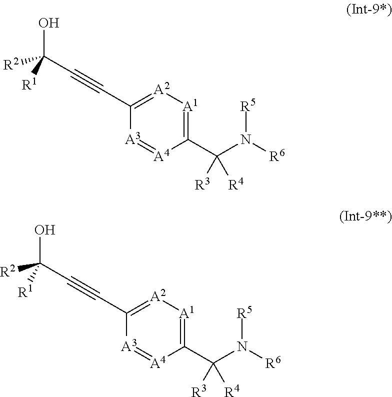
wherein A¹, A², A³, A⁴, R¹, R², R³, R⁴, R⁵ and R⁶ are as defined for compounds of formula I in claim 1, or a salt of N-oxide thereof; or
a mixture of compounds of formula Int-10* and Int-10**, wherein the molar amount of Int-10** in the mixture is more than 50% compared to the combined molar amount of Int-10* and Int-10** 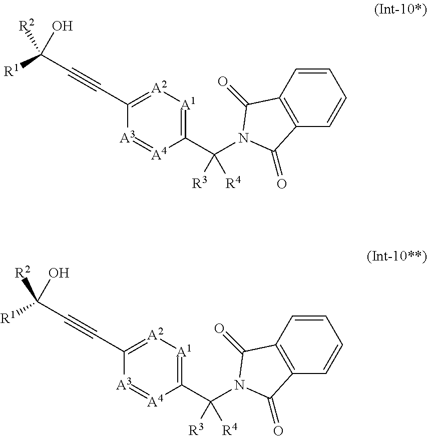
wherein A¹, A², A³, A⁴, R¹, R², R³ and R⁴ are as defined for compounds of formula I in claim 1, or a salt of N-oxide thereof; or
a compound of formula Int-11* and Int-11**, wherein the molar amount of Int-11** in the mixture is more than 50% compared to the combined molar amount of Int-11* and Int-11** 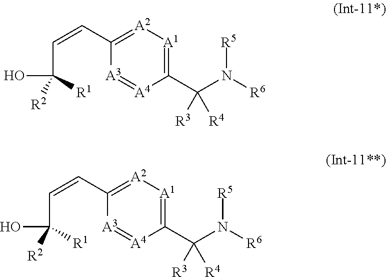
wherein A¹, A², A³, A⁴, R¹, R², R³, R⁴, R⁵ and R⁶ are as defined for compounds of formula I in claim 1, or a salt of N-oxide thereof; or
a mixture of compounds of formula Int-12* and Int-12**, wherein the molar amount of Int-12** in the mixture is more than 50% compared to the combined molar amount of Int-12* and Int-12** 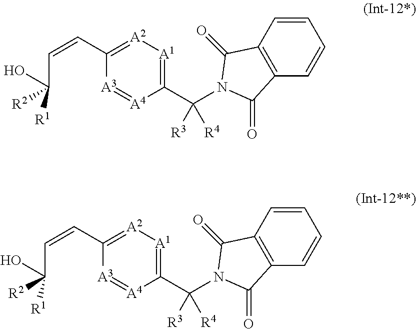
wherein A¹, A², A³, A⁴, R¹, R², R³ and R⁴ are as defined for compounds of formula I in claim 1, or a salt of N-oxide thereof; or
a mixture of compounds of formula Int-13* and Int-13**, wherein the molar amount of Int-13** in the mixture is more than 50% compared to the combined molar amount of Int-13* and Int-13** 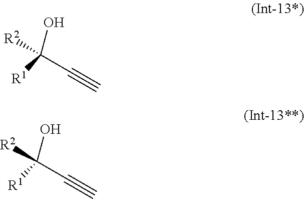
wherein R¹ and R² are as defined for compounds of formula I in claim 1; or
a mixture of compounds of formula Int-14* and Int-14**, wherein the molar amount of Int-14** in the mixture is more than 50% compared to the combined molar amount of Int-14* and Int-14** 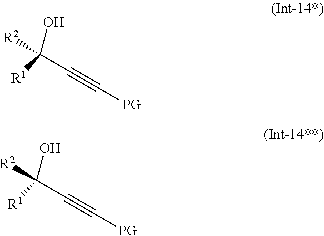
wherein R¹ and R² are as defined for compounds of formula I in claim 1 and PG is an oragnosilicon e.g. trialkylsilyl such as tri-C₁-C₄alkyl-silyl, e.g. trimethylsilyl; or
a mixture of compounds of formula Int-15* and Int-15**, wherein the molar amount of Int-15** in the mixture is more than 50% compared to the combined molar amount of Int-15* and Int-15** 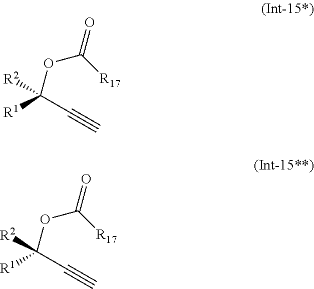
wherein R¹ and R² are as defined for compounds of formula I in claim 1 and R¹⁷ is C₁-C₁₂alkyl.

14. A method of controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I) as defined in claim 1.

15. An insecticidal, acaricidal, nematicidal or molluscicidal composition comprising an insecticidally, acaricidally, nematicidally or molluscicidally effective amount of a compound of formula (I) as defined in claim 1.

16. An insecticidal, acaricidal, nematicidal or molluscicidal composition according to claim 15 comprising at least one additional compound having biological activity.

17. A combination product comprising a pesticidally effective amount of a component A and a pesticidally effective amount of component B, wherein component A is a compound of formula (I) as defined in claim 1, and compound B is imidacloprid, enrofloxacin, praziquantel, pyrantel embonate, febantel, penethamate, moloxicam, cefalexin, kanamycin, pimobendan, clenbuterol, fipronil, ivermectin, omeprazole, tiamulin, benazepril, milbemycin, cyromazine, thiamethoxam, pyriprole, deltamethrin, cefquinome, florfenicol, buserelin, cefovecin, tulathromycin, ceftiour, selamectin, carprofen, metaflumizone, moxidectin, methoprene (including S-methoprene), clorsulon, pyrantel, amitraz, triclabendazole, avermectin, abamectin, emamectin, eprinomectin, doramectin, selamectin, nemadectin, albendazole, cambendazole, fenbendazole, flubendazole, mebendazole, oxfendazole, oxibendazole, parbendazole, tetramisole, levamisole, pyrantel pamoate, oxantel, morantel, triclabendazole, epsiprantel, fipronil, lufenuron, ecdysone or tebufenozide.

18. A process for preparing a compound of formula IA′ 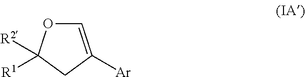
wherein
R¹ is C₁-C₈haloalkyl;
R^{2′} is optionally substituted aryl or optionally substituted heteroaryl;
Ar is optionally substituted aryl or optionally substituted heteroaryl;
comprising dehydrating a compound of formula II′ 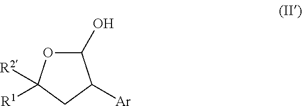
wherein R¹, R^{2′} and Ar are as defined for the compound of formula IA′;
with a suitable acidic catalyst or a suitable activation agent and a suitable base;
preferably the process comprises preparing the compound of formula II′ by reacting a compound of formula III′ 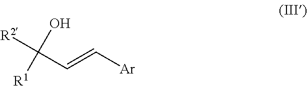
wherein R¹, R^{2′} and Ar are as defined for the compound of formula IA′;
with a source of CO and H₂ in the presence of a catalyst comprising a complex of a transition metal and a suitable ligand.

19. A process for the preparation of:
(1) a compound of formula II′ 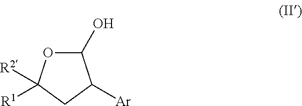
wherein R¹, R^{2′} and Ar are as defined for the compound of formula IA′;
comprising reacting a compound of formula III′ 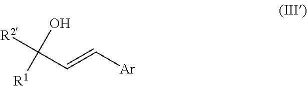
wherein R¹, R^{2′} and Ar are as defined for the compound of formula IA′ in claim 18; with a source of CO and H₂ in the presence of a catalyst comprising a complex of a transition metal and a suitable ligand; or
(2) a compound of formula X′ 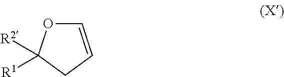
wherein
R¹ is C₁-C₈haloalkyl;
R^{2′} is optionally substituted aryl or optionally substituted heteroaryl;
comprising dehydrating a compound of formula IX′ 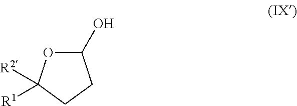
wherein R¹ and R^{2′} are as defined for the compound of formula X′;
with a suitable acidic catalyst or a suitable activation agent and a suitable base;
the process comprises preparing the compound of formula IX′ by reacting a compound of formula IX′ with a compound of formula VIII′ 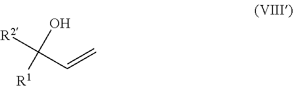
wherein R¹ and R^{2′} are as defined for the compound of formula X′;
with a source of H₂ and CO in the presence of a catalyst comprising a complex of a transition metal and a suitable ligand;
optionally the process includes the additional step of reacting the compound of formula X′ with chlorine, bromine or iodine to give a compound of formula XI′ 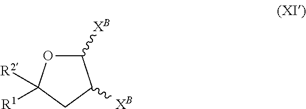
wherein R¹ and R^{2′} are as defined for the compound of formula X′ and X^{B} is Cl, Br or I;
optionally the process includes the additional step of eliminating HX^{B} from the compound of formula XI′, e.g. in the presence of a suitable base, to give a compound of compound of formula XII′ 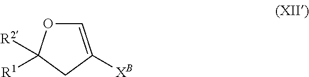
wherein R¹ and R^{2′} are as defined for the compound of formula X′ and X^{B} is Cl, Br or optionally the process includes the additional step of reacting a compound of formula XII′ with a compound of formula Ar-M, wherein Ar is optionally substituted aryl or optionally substituted heteroaryl and M is a derivative of B, Si, Sn, Mg, Zn, Mn, to give a compound of formula IA′ 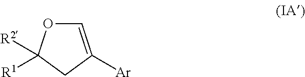
wherein R¹ and R^{2′} are as defined for the compound of formula X′ and Ar is optionally substituted aryl or optionally substituted heteroaryl; or
(3) a compound of formula IX′ 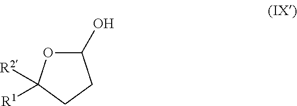
wherein
R¹ is C₁-C₈haloalkyl;
R^{2′} is optionally substituted aryl or optionally substituted heteroaryl;
comprising reacting a compound of formula VIII′ 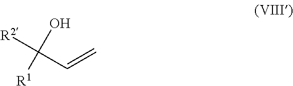
wherein R¹ and R^{2′} are as defined for the compound of formula IX′;
with a source of H₂ and CO in the presence of a catalyst comprising a complex of a transition metal and a suitable ligand.

20. (canceled)

21. (canceled)

22. A process according to claim 18, wherein R^{2′} is R² as defined in claim 1 and Ar stands for group A or group A1 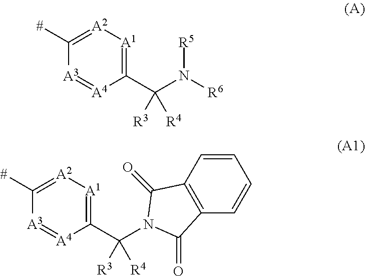
wherein A¹, A², A³, A⁴, R³, R⁴, R⁵ and R⁶ are as defined for compounds of formula I in claim 1.
